SEQUENCE LISTING (436 sequences)

SEQ ID NO: 1 - DNA, 87 bp, Homo sapien
gcgagaagatgacccagatcatgtttgagaccttcaacaccccagccatgtacgttgctatccaggctgtgctatccctgtacgcct

SEQ ID NO: 2 - DNA, 21 bp, Homo sapien
gcgagaagatgacccagatca

SEQ ID NO: 3 - DNA, 20 bp, Homo sapien
tgtgctatccctgtacgcct

SEQ ID NO: 4 - DNA, 25 bp, Homo sapien
accttcaacaccccagccatgtacg

SEQ ID NO: 5 - DNA, 81 bp, Homo sapien
agcgggcgcagtgattataggctttcgctctaagattaaaaatgccctagcccacttcttaccacaaggcacacctacacc

SEQ ID NO: 6 - DNA, 20 bp, Homo sapien
agcgggcgcagtgattatag

SEQ ID NO: 7 - DNA, 22 bp, Homo sapien
ggtgtaggtgtgccttgtggta

SEQ ID NO: 8 - DNA, 33 bp, Homo sapien
cgctctaagattaaaaatgccctagcccacttc

SEQ ID NO: 9 - DNA, 72 bp, Homo sapien
ctttgtcacagcccaagatagttaagtgggatcgagacatgtaagcagcatcatggaggtttgaagatgccg

SEQ ID NO: 10 - DNA, 23 bp, Homo sapien
ctttgtcacagcccaagatagtt

SEQ ID NO: 11 - DNA, 20 bp, Homo sapien
atggaggtttgaagatgccg

SEQ ID NO: 12 - DNA, 28 bp, Homo sapien
aagtgggatcgagacatgtaagcagcat

SEQ ID NO: 13 - DNA, 86 bp, Homo sapien
tgcgatctcagatcagtgtaccatttgcctcccgggctcaagcgattctcctgcctcagcctcccaagtagctgggattacaggca

SEQ ID NO: 14 - DNA, 23 bp, Homo sapien
tgcgatctcagatcagtgtacca

SEQ ID NO: 15 - DNA, 22 bp, Homo sapien
tgcctgtaatcccagctacttg

SEQ ID NO: 16 - DNA, 22 bp, Homo sapien
cccgggctcaagcgattctcct

SEQ ID NO: 17 - DNA, 81 bp, Homo sapien
gcagtttgcattgcagtcaacagtcgaagaaggtgtgggcagaagaaaaagctagtgatcaacagtggcaatggagctgtg

SEQ ID NO: 18 - DNA, 21 bp, Homo sapien
gcagtttgcattgcagtcaac

SEQ ID NO: 19 - DNA, 21 bp, Homo sapien
cacagctccattgccactgtt

SEQ ID NO: 20 - DNA, 32 bp, Homo sapien
agaaggtgtgggcagaagaaaaagctagtgat

SEQ ID NO: 21 - DNA, 79 bp, Homo sapien
tgagtgtcccccggtatcttccccgccctgccaatcccgatgaaattggaaattttattgatgaaaatctgaaagcggc

SEQ ID NO: 22 - DNA, 21 bp, Homo sapien
tgagtgtcccccggtatcttc

SEQ ID NO: 23 - DNA, 21 bp, Homo sapien
gccgctttcagattttcatca

SEQ ID NO: 24 - DNA, 28 bp, Homo sapien
tgccaatcccgatgaaattggaaatttt

SEQ ID NO: 25 - DNA, 91 bp, Homo sapien
ccaacacaccagagtctagcattaactatggagacaccccaaagtcttgtactaagtcttctaaaagctccactccagttccttcaaagca

SEQ ID NO: 26 - DNA, 24 bp, Homo sapien
ccaacacaccagagtctagcatta

SEQ ID NO: 27 - DNA, 21 bp, Homo sapien
cactccagttccttcaaagca

SEQ ID NO: 28 - DNA, 35 bp, Homo sapien
gacaccccaaagtcttgtactaagtcttctaaaag

SEQ ID NO: 29 - DNA, 77 bp, Homo sapien
tggttctggggaagcattcaaatggatgcccgagctggagaaactatgtatttatatgaaaaggcaaatactcctga

SEQ ID NO: 30 - DNA, 19 bp, Homo sapien
tggttctggggaagcattc

SEQ ID NO: 31 - DNA, 24 bp, Homo sapien
atatgaaaaggcaaatactcctga

SEQ ID NO: 32 - DNA, 24 bp, Homo sapien
aatggatgcccgagctggagaaac

SEQ ID NO: 33 - DNA, 74 bp, Homo sapien
cctcgtttgacatccactatggctcgggcagcctctccgggtacctgagccaggacactgtgtcggtgccctgc

SEQ ID NO: 34 - DNA, 22 bp, Homo sapien
cctcgtttgacatccactatgg

SEQ ID NO: 35 - DNA, 18 bp, Homo sapien
actgtgtcggtgccctgc

SEQ ID NO: 36 - DNA, 24 bp, Homo sapien
cagcctctccgggtacctgagcca

SEQ ID NO: 37 - DNA, 74 bp, Homo sapien
cagggtttcaggttccaatcagaactgttggcaaggtgacatttccatgcataaatgcgatccacagaaggtcc

SEQ ID NO: 38 - DNA, 21 bp, Homo sapien
cagggtttcaggttccaatca

SEQ ID NO: 39 - DNA, 21 bp, Homo sapien
ggaccttctgtggatcgcatt

SEQ ID NO: 40 - DNA, 28 bp, Homo sapien
ctgttggcaaggtgacatttccatgcat

SEQ ID NO: 41 - DNA, 80 bp, Homo sapien
aagcttgtgaggagacggactctatgttgcccaggctgttatggaactcctgagtcaagtgatcctcccaccttggcctc

SEQ ID NO: 42 - DNA, 23 bp, Homo sapien
aagcttgtgaggagacggactct

SEQ ID NO: 43 - DNA, 19 bp, Homo sapien
gaggccaaggtgggaggat

SEQ ID NO: 44 - DNA, 27 bp, Homo sapien
tgttgcccaggctgttatggaactcct

SEQ ID NO: 45 - DNA, 71 bp, Homo sapien
tatgccatgaccagctttcactacaccatcatggtgacagaggccatcgcatacctgagggcctgccttaa

SEQ ID NO: 46 - DNA, 22 bp, Homo sapien
tatgccatgaccagctttcact

SEQ ID NO: 47 - DNA, 20 bp, Homo sapien
ttaaggcaggccctcaggta

SEQ ID NO: 48 - DNA, 25 bp, Homo sapien
accatcatggtgacagaggccatcg

SEQ ID NO: 49 - DNA, 71 bp, Homo sapien
gaccggtacatcgccattgtacaggcgactaagtcattccggctccgatccagaacactaccgcgcagcaa

SEQ ID NO: 50 - DNA, 20 bp, Homo sapien
gaccggtacatcgccattgt

SEQ ID NO: 51 - DNA, 19 bp, Homo sapien
ttgctgcgcggtagtgttc

SEQ ID NO: 52 - DNA, 25 bp, Homo sapien
caggcgactaagtcattccggctcc

SEQ ID NO: 53 - DNA, 75 bp, Homo sapien
ccaggagccagggatgtggaggcgcagctgcggcggctgcaggaggagaggacgtgcaaggtgtgcctggaccgc

SEQ ID NO: 54 - DNA, 18 bp, Homo sapien
ccaggagccagggatgtg

SEQ ID NO: 55 - DNA, 17 bp, Homo sapien
gcggtccaggcacacct

SEQ ID NO: 56 - DNA, 21 bp, Homo sapien
tcctctcctcctgcagccgcc

SEQ ID NO: 57 - DNA, 62 bp, Homo sapien
ccgtgagcgaggctgtggtgagcagcgtcaacactgtggccaccaagaccgtggaggaggcg

SEQ ID NO: 58 - DNA, 17 bp, Homo sapien
ccgtgagcgaggctgtg

SEQ ID NO: 59 - DNA, 18 bp, Homo sapien
aagaccgtggaggaggcg

SEQ ID NO: 60 - DNA, 23 bp, Homo sapien
tgagcagcgtcaacactgtggcc

SEQ ID NO: 61 - DNA, 86 bp, Homo sapien
cagacatgcaccatatagaagagagtttccaagaaatcaaaagagccatccaagctaaggacaccttcccaaatgtcactatcctg

SEQ ID NO: 62 - DNA, 27 bp, Homo sapien
cagacatgcaccatatagaagagagtt

SEQ ID NO: 63 - DNA, 24 bp, Homo sapien
caggatagtgacatttgggaaggt

SEQ ID NO: 64 - DNA, 32 bp, Homo sapien
ccaagaaatcaaaagagccatccaagctaagg

SEQ ID NO: 65 - DNA, 85 bp, Homo sapien
tgggctgatggaagacaccacccagctggaaactgacaaactagctctcccgatgagccccagcccgcttagcccgagccccatc

SEQ ID NO: 66 - DNA, 19 bp, Homo sapien
tgggctgatggaagacacc

SEQ ID NO: 67 - DNA, 18 bp, Homo sapien
gatggggctcgggctaag

SEQ ID NO: 68 - DNA, 29 bp, Homo sapien
tggaaactgacaaactagctctcccgatg

SEQ ID NO: 69 - DNA, 71 bp, Homo sapien
ccacccactgctgcatcaaccctctcctgtatgcgtttcttgatgggacatttagcaaatacctctgccgc

SEQ ID NO: 70 - DNA, 18 bp, Homo sapien
ccacccactgctgcatca

SEQ ID NO: 71 - DNA, 21 bp, Homo sapien
gcggcagaggtatttgctaaa

SEQ ID NO: 72 - DNA, 28 bp, Homo sapien
ccctctcctgtatgcgtttcttgatggg

SEQ ID NO: 73 - DNA, 78 bp, Homo sapien
tggtttctggtggcctctatgaatcccatgtagggtgcagaccgtactccatccctccctgtgagcaccacgtcaacg

SEQ ID NO: 74 - DNA, 20 bp, Homo sapien
ggtttctggtggcctctatg

SEQ ID NO: 75 - DNA, 19 bp, Homo sapien
cgttgacgtggtgctcaca

SEQ ID NO: 76 - DNA, 28 bp, Homo sapien
atcccatgtagggtgcagaccgtactcc

SEQ ID NO: 77 - DNA, 76 bp, Homo sapien
gggcccttcattgtagatctgattaccttcacggaaggaagcggacgctcaccacgctatgccctctggttctgtg

SEQ ID NO: 78 - DNA, 22 bp, Homo sapien
gggcccttcattgtagatctga

SEQ ID NO: 79 - DNA, 21 bp, Homo sapien
cacagaaccagagggcatagc

SEQ ID NO: 80 - DNA, 23 bp, Homo sapien
accttcacggaaggaagcggacg

SEQ ID NO: 81 - DNA, 73 bp, Homo sapien
aacgctgtgcctggcagtatggctaaccgccattatggctaccatcccattgctagtgttttaccaagtggcc

SEQ ID NO: 82 - DNA, 20 bp, Homo sapien
aacgctgtgcctggcagtat

SEQ ID NO: 83 - DNA, 23 bp, Homo sapien
ggccacttggtaaaacactagca

SEQ ID NO: 84 - DNA, 27 bp, Homo sapien
ctaaccgccattatggctaccatccca

SEQ ID NO: 85 - DNA, 74 bp, Homo sapien
agctatgcaggtgacagagactcttgggatgacgcactgctgcatcaaccccatcatctatgcctttgtcgggg

SEQ ID NO: 86 - DNA, 25 bp, Homo sapien
agctatgcaggtgacagagactctt

SEQ ID NO: 87 - DNA, 20 bp, Homo sapien
ccccgacaaaggcatagatg

SEQ ID NO: 88 - DNA, 24 bp, Homo sapien
atgacgcactgctgcatcaacccc

SEQ ID NO: 89 - DNA, 74 bp, Homo sapien
gccaaaggcattggctacttcttcgttttgcagcccaaggttgtagatggtactccatgtagcccagattccac

SEQ ID NO: 90 - DNA, 21 bp, Homo sapien
gccaaaggcattggctacttc

SEQ ID NO: 91 - DNA, 21 bp, Homo sapien
gtggaatctgggctacatgga

SEQ ID NO: 92 - DNA, 28 bp, Homo sapien
cgttttgcagcccaaggttgtagatggt

SEQ ID NO: 93 - DNA, 71 bp, Homo sapien
gctcctggctgctttgatgtcagtgctgctactccacctctgcggcgaatcagaagcagcaagcaactttg

SEQ ID NO: 94 - DNA, 20 bp, Homo sapien
gctcctggctgctttgatgt

SEQ ID NO: 95 - DNA, 22 bp, Homo sapien
caaagttgcttgctgcttctga

SEQ ID NO: 96 - DNA, 22 bp, Homo sapien
tgctactccacctctgcggcga

SEQ ID NO: 97 - DNA, 77 bp, Homo sapien
gctgcctgcttttgacctttggctcatgggtacttgacgttttaaactcctaggcccaggatgaggaggagcttggc

SEQ ID NO: 98 - DNA, 20 bp, Homo sapien
gctgcctgcttttgaccttt

SEQ ID NO: 99 - DNA, 20 bp, Homo sapien
gccaagctcctcctcatcct

SEQ ID NO: 100 - DNA, 33 bp, Homo sapien
tcatgggtacttgacgttttaaactcctaggcc

SEQ ID NO: 101 - DNA, 73 bp, Homo sapien
aaacaccagttcctggatggattcccgccatccatgccccctctttaattagccggtcctctcaataatgtag

SEQ ID NO: 102 - DNA, 22 bp, Homo sapien
aaacaccagttcctggatggat

SEQ ID NO: 103 - DNA, 25 bp, Homo sapien
ctacattattgagaggaccggctaa

SEQ ID NO: 104 - DNA, 18 bp, Homo sapien
cccgccatccatgccccc

SEQ ID NO: 105 - DNA, 85 bp, Homo sapien
gctggtggtggctctccttgtcattttccaggtatgcctgtgtcaagatgaggtcacggacgattacatcggagacaacaccaca

SEQ ID NO: 106 - DNA, 19 bp, Homo sapien
gctggtggtggctctcctt

SEQ ID NO: 107 - DNA, 22 bp, Homo sapien
tgtggtgttgtctccgatgtaa

SEQ ID NO: 108 - DNA, 28 bp, Homo sapien
cgtgacctcatcttgacacaggcatacc

SEQ ID NO: 109 - DNA, 76 bp, Homo sapien
gctttcttccactgttgtctgaaccccatcctctatgctttccttggagccaaatttaaaacctctgcccagcacg

SEQ ID NO: 110 - DNA, 24 bp, Homo sapien
gctttcttccactgttgtctgaac

SEQ ID NO: 111 - DNA, 20 bp, Homo sapien
cgtgctgggcagaggtttta

SEQ ID NO: 112 - DNA, 27 bp, Homo sapien
ccatcctctatgctttccttggagcca

SEQ ID NO: 113 - DNA, 84 bp, Homo sapien
attcccgccaaggttgtccgcagctaccggaagcaggaaccaagcttaggctgctccatcccagctatcctgttcttgccccgc

SEQ ID NO: 114 - DNA, 18 bp, Homo sapien
attcccgccaaggttgtc

SEQ ID NO: 115 - DNA, 19 bp, Homo sapien
gcggggcaagaacaggata

SEQ ID NO: 116 - DNA, 24 bp, Homo sapien
cgcagctaccggaagcaggaacca

SEQ ID NO: 117 - DNA, 71 bp, Homo sapien
atgagcaaacgcatggacatcgccatccaagtcacagaaagcatcgcactctttcacagctgcctcaaccc

SEQ ID NO: 118 - DNA, 21 bp, Homo sapien
atgagcaaacgcatggacatc

SEQ ID NO: 119 - DNA, 20 bp, Homo sapien
gggttgaggcagctgtgaaa

SEQ ID NO: 120 - DNA, 26 bp, Homo sapien
ccatccaagtcacagaaagcatcgca

SEQ ID NO: 121 - DNA, 74 bp, Homo sapien
gcagccaagttaggagtgaagagatgcacggatcagatgtcccttcagaaacgaagcctcattgcggaagtcct

SEQ ID NO: 122 - DNA, 22 bp, Homo sapien
gcagccaagttaggagtgaaga

SEQ ID NO: 123 - DNA, 20 bp, Homo sapien
accaggacttccgcaatgag

SEQ ID NO: 124 - DNA, 25 bp, Homo sapien
cggatcagatgtcccttcagaaacg

SEQ ID NO: 125 - DNA, 90 bp, Homo sapien
ccctcctcctgcactgctatgcagattctggctgcaaactcctggaggacatggttgaaaagaccatcaattccgacatatctatacctg

SEQ ID NO: 126 - DNA, 18 bp, Homo sapien
ccctcctcctgcactgct

SEQ ID NO: 127 - DNA, 25 bp, Homo sapien
caggtatagatatgtcggaattgat

SEQ ID NO: 128 - DNA, 25 bp, Homo sapien
ttctggctgcaaactcctggaggac

SEQ ID NO: 129 - DNA, 77 bp, Homo sapien
cctcctcatcctctcccacatcaggcacatgagtaacaaaggcatggagcatctgtacagcatgaagtgcaagaacg

SEQ ID NO: 130 - DNA, 21 bp, Homo sapien
cctcctcatcctctcccacat

SEQ ID NO: 131 - DNA, 23 bp, Homo sapien
cgttcttgcacttcatgctgtac

SEQ ID NO: 132 - DNA, 28 bp, Homo sapien
aggcacatgagtaacaaaggcatggagc

SEQ ID NO: 133 - DNA, 77 bp, Homo sapien
ggacctgtctaagatacccctgatcaagaaggccactggtgtgcaggtgtgaggctcctacaggcccactctcttca

SEQ ID NO: 134 - DNA, 25 bp, Homo sapien
ggacctgtctaagatacccctgatc

SEQ ID NO: 135 - DNA, 22 bp, Homo sapien
tgaagagagtgggcctgtagga

SEQ ID NO: 136 - DNA, 24 bp, Homo sapien
aaggccactggtgtgcaggtgtga

SEQ ID NO: 137 - DNA, 77 bp, Homo sapien; identical to SEQ ID NO: 133

SEQ ID NO: 138 - DNA, 25 bp, Homo sapien
ggacctgtctaagatacccctgatc

SEQ ID NO: 139 - DNA, 22 bp, Homo sapien
tgaagagagtgggcctgtagga

SEQ ID NO: 140 - DNA, 24 bp, Homo sapien
aaggccactggtgtgcaggtgtga

SEQ ID NO: 141 - DNA, 86 bp, Homo sapien
agggctgcttttaactctggtaaagtggatattgttgccatcaatgaccccttcattgacctcaactacatggtttacatgttcca

SEQ ID NO: 142 - DNA, 24 bp, Homo sapien
agggctgcttttaactctggtaaa

SEQ ID NO: 143 - DNA, 26 bp, Homo sapien
tggaacatgtaaaccatgtagttgag

SEQ ID NO: 144 - DNA, 28 bp, Homo sapien
tggatattgttgccatcaatgacccctt

SEQ ID NO: 145 - DNA, 74 bp, Homo sapien
ggaattttgccgatttcatgactgaacagtcaccgacgagagtgctggggaataaaaaggggatcttcactcgg

SEQ ID NO: 146 - DNA, 21 bp, Homo sapien
ggaattttgccgatttcatga

SEQ ID NO: 147 - DNA, 21 bp, Homo sapien
ccgagtgaagatccccttttt

SEQ ID NO: 148 - DNA, 26 bp, Homo sapien
tgaacagtcaccgacgagagtgctgg

SEQ ID NO: 149 - DNA, 75 bp, Homo sapien
ttctgaagaccactccagcgatatgttcaactatgaagaatactgcaccgccaacgcagtcactgggccttgccg

SEQ ID NO: 150 - DNA, 23 bp, Homo sapien
ttctgaagaccactccagcgata

SEQ ID NO: 151 - DNA, 16 bp, Homo sapien
cggcaaggcccagtga

SEQ ID NO: 152 - DNA, 28 bp, Homo sapien
actatgaagaatactgcaccgccaacgc

SEQ ID NO: 153 - DNA, 71 bp, Homo sapien
cggtgtgagaagtgcagcaagccctgtgcccgagtgtgctatggtctgggcatggagcacttgcgagaggt

SEQ ID NO: 154 - DNA, 20 bp, Homo sapien
cggtgtgagaagtgcagcaa

SEQ ID NO: 155 - DNA, 20 bp, Homo sapien
acctctcgcaagtgctccat

SEQ ID NO: 156 - DNA, 23 bp, Homo sapien
ccctgtgcccgagtgtgctatgg

SEQ ID NO: 157 - DNA, 76 bp, Homo sapien
gctcagagaaagcgaaaaatggaacatgatggttcactttttcaagcagtaggaattggaacattattacagcagc

SEQ ID NO: 158 - DNA, 22 bp, Homo sapien
gctcagagaaagcgaaaaatgg

SEQ ID NO: 159 - DNA, 25 bp, Homo sapien
gctgctgtaataatgttccaattcc

SEQ ID NO: 160 - DNA, 25 bp, Homo sapien
acatgatggttcactttttcaagca

SEQ ID NO: 161 - DNA, 78 bp, Homo sapien
tcaagcagtaggaattggaacattattacagcagccagacgatcatgcagctactacatcactttcttggaaacgtgt

SEQ ID NO: 162 - DNA, 26 bp, Homo sapien
tcaagcagtaggaattggaacattat

SEQ ID NO: 163 - DNA, 26 bp, Homo sapien
acacgtttccaagaaagtgatgtagt

SEQ ID NO: 164 - DNA, 24 bp, Homo sapien
acagcagccagacgatcatgcagc

SEQ ID NO: 165 - DNA, 81 bp, Homo sapien
ccagctccctgcgaagagcagcccagctgcagagaaagttcccgcatctggagttcaggagtattcggggaaacctcaaca

SEQ ID NO: 166 - DNA, 18 bp, Homo sapien
ccagctccctgcgaagag

SEQ ID NO: 167 - DNA, 22 bp, Homo sapien
tgttgaggtttccccgaatact

SEQ ID NO: 168 - DNA, 26 bp, Homo sapien
tgcagagaaagttcccgcatctggag

SEQ ID NO: 169 - DNA, 64 bp, Homo sapien
ggcaatgcggctgcaacggcggaagaaaacagcccaaagatgagagtgattcgcgtgggtaccc

SEQ ID NO: 170 - DNA, 16 bp, Homo sapien
ggcaatgcggctgcaa

SEQ ID NO: 171 - DNA, 19 bp, Homo sapien
gtgattcgcgtgggtaccc

SEQ ID NO: 172 - DNA, 27 bp, Homo sapien
gcggaagaaaacagcccaaagatgaga

SEQ ID NO: 173 - DNA, 73 bp, Homo sapien
tttccttggtcaggcagtataatccaaagatggtcaaggtcgcaagcttgctggtgaaaaggaccccacgaag

SEQ ID NO: 174 - DNA, 23 bp, Homo sapien
tttccttggtcaggcagtataat

SEQ ID NO: 175 - DNA, 20 bp, Homo sapien
gtgaaaaggaccccacgaag

SEQ ID NO: 176 - DNA, 24 bp, Homo sapien
ccaaagatggtcaaggtcgcaagc

SEQ ID NO: 177 - DNA, 88 bp, Homo sapien
cggagtgtcaagaggtgtgcagaaaatccaaagaaggctgaggacaatgtgtgtgtcaagaaaataacaaccagaagtcatagggaca

SEQ ID NO: 178 - DNA, 20 bp, Homo sapien
cggagtgtcaagaggtgtgc

SEQ ID NO: 179 - DNA, 23 bp, Homo sapien
aacaaccagaagtcatagggaca

SEQ ID NO: 180 - DNA, 28 bp, Homo sapien
ccaaagaaggctgaggacaatgtgtgtg

SEQ ID NO: 181 - DNA, 90 bp, Homo sapien
tctgggttgctcttcatctattcccttggattcctttgttctgctattgctaaatgtattacatttttatatggtataggctgaacaaca

SEQ ID NO: 182 - DNA, 23 bp, Homo sapien
tctgggttgctcttcatctattc

SEQ ID NO: 183 - DNA, 26 bp, Homo sapien
tgttgttcagcctataccatataaaa

SEQ ID NO: 184 - DNA, 29 bp, Homo sapien
ccttggattcctttgttctgctattgcta

SEQ ID NO: 185 - DNA, 84 bp, Homo sapien
cctttccattgccatactgagaaactggcagcaggcagtgtgcctacaggtctacaaagaaacttcagatcatcttcttgaggg

SEQ ID NO: 186 - DNA, 23 bp, Homo sapien
cctttccattgccatactgagaa

SEQ ID NO: 187 - DNA, 25 bp, Homo sapien
ccctcaagaagatgatctgaagttt

SEQ ID NO: 188 - DNA, 28 bp, Homo sapien
cagcaggcagtgtgcctacaggtctaca

SEQ ID NO: 189 - DNA, 83 bp, Homo sapien
gagctgatacacccaacacttgagctcactgcaacagtaccagattttcaccgctatgcctcctttcactctgggagtcttcc

SEQ ID NO: 190 - DNA, 24 bp, Homo sapien
gagctgatacacccaacacttgag

SEQ ID NO: 191 - DNA, 23 bp, Homo sapien
ggaagactcccagagtgaaagga

SEQ ID NO: 192 - DNA, 30 bp, Homo sapien
tgcaacagtaccagattttcaccgctatgc

SEQ ID NO: 193 - DNA, 86 bp, Homo sapien
aaacctgccaaacccctacctaaggaaatggaggagtttgtacagagctctggagaaaatggtgttgtggtgttttctctggggtc

SEQ ID NO: 194 - DNA, 20 bp, Homo sapien
aaacctgccaaacccctacc

SEQ ID NO: 195 - DNA, 21 bp, Homo sapien
tgtggtgttttctctggggtc

SEQ ID NO: 196 - DNA, 31 bp, Homo sapien
taaggaaatggaggagtttgtacagagctct

SEQ ID NO: 197 - DNA, 88 bp, Homo sapien
ggttccaaatgtctgatatgaagaagtgggatcagttttacagtgaagttttaggaagacccactaccttatttgagacaatgggaaa

SEQ ID NO: 198 - DNA, 24 bp, Homo sapien
ggttccaaatgtctgatatgaaga

SEQ ID NO: 199 - DNA, 23 bp, Homo sapien
accttatttgagacaatgggaaa

SEQ ID NO: 200 - DNA, 32 bp, Homo sapien
tgggatcagttttacagtgaagttttaggaag

SEQ ID NO: 201 - DNA, 71 bp, Homo sapien
tgctccatctccacgaggttgtgaagagagaaaatgggccgcctgcactacagcatgagagccatcagtta

SEQ ID NO: 202 - DNA, 20 bp, Homo sapien
tgctccatctccacgaggtt

SEQ ID NO: 203 - DNA, 25 bp, Homo sapien
taactgatggctctcatgctgtagt

SEQ ID NO: 204 - DNA, 24 bp, Homo sapien
tgaagagagaaaatgggccgcctg

SEQ ID NO: 205 - DNA, 90 bp, Homo sapien
tcgtcttcctttatccaactcatttgttaactagttttcttctgtgagttcctttgcctataattgaagcagttctctgaaatcacccaa

SEQ ID NO: 206 - DNA, 23 bp, Homo sapien
tcgtcttcctttatccaactcat

SEQ ID NO: 207 - DNA, 22 bp, Homo sapien
gcagttctctgaaatcacccaa

SEQ ID NO: 208 - DNA, 34 bp, Homo sapien
ctagttttcttctgtgagttcctttgcctataat

SEQ ID NO: 209 - DNA, 80 bp, Homo sapien
gcctataattgaagcagttctctgaaatcacccaaactgattttatgaaagcccatgcttttggaaagatttgcacttcg

SEQ ID NO: 210 - DNA, 26 bp, Homo sapien
gcctataattgaagcagttctctgaa

SEQ ID NO: 211 - DNA, 22 bp, Homo sapien
cgaagtgcaaatctttccaaaa

SEQ ID NO: 212 - DNA, 30 bp, Homo sapien
tcacccaaactgattttatgaaagcccatg

SEQ ID NO: 213 - DNA, 82 bp, Homo sapien
ctgaagttagattctggacaaagtgtatgagaaagtttacggctataaaaggtttgctgagagtttttccttaaataacgca

SEQ ID NO: 214 - DNA, 25 bp, Homo sapien
ctgaagttagattctggacaaagtg

SEQ ID NO: 215 - DNA, 24 bp, Homo sapien
tgcgttatttaaggaaaaactctc

SEQ ID NO: 216 - DNA, 33 bp, Homo sapien
tatgagaaagtttacggctataaaaggtttgct

SEQ ID NO: 217 - DNA, 72 bp, Homo sapien
cctatcaatcagagatcggaccatggcaggcacactcagcaaagtcccccatgatccagaagacatgtgcga

SEQ ID NO: 218 - DNA, 24 bp, Homo sapien
cctatcaatcagagatcggaccat

SEQ ID NO: 219 - DNA, 21 bp, Homo sapien
tcgcacatgtcttctggatca

SEQ ID NO: 220 - DNA, 24 bp, Homo sapien
caggcacactcagcaaagtccccc

SEQ ID NO: 221 - DNA, 86 bp, Homo sapien
cagtggtgaggttgtaaatcaaatgagagaggaggaactgatccgggtagcaggaacacatttccaagtaaaatttgcaacagagc

SEQ ID NO: 222 - DNA, 25 bp, Homo sapien
cagtggtgaggttgtaaatcaaatg

SEQ ID NO: 223 - DNA, 24 bp, Homo sapien
gctctgttgcaaattttacttgga

SEQ ID NO: 224 - DNA, 27 bp, Homo sapien
agaggaggaactgatccgggtagcagg

SEQ ID NO: 225 - DNA, 86 bp, Homo sapien
cccagcaccgactactaccaagagctgcagagagacatttctgaaatggctgtctgtcagtgccgccgaaagaactacgggcagct

SEQ ID NO: 226 - DNA, 21 bp, Homo sapien
cccagcaccgactactaccaa

SEQ ID NO: 227 - DNA, 20 bp, Homo sapien
agctgcccgtagttctttcg

SEQ ID NO: 228 - DNA, 27 bp, Homo sapien
agagagacatttctgaaatggctgtct

SEQ ID NO: 229 - DNA, 85 bp, Homo sapien
tcagtatgaattctggaaatgcaaaggaaaagagtctcttcaactaagagtctttgctgggatggaagatttgggccgtgtggtg

SEQ ID NO: 230 - DNA, 26 bp, Homo sapien
tcagtatgaattctggaaatgcaaag

SEQ ID NO: 231 - DNA, 18 bp, Homo sapien
caccacacggcccaaatc

SEQ ID NO: 232 - DNA, 37 bp, Homo sapien
aaaagagtctcttcaactaagagtctttgctgggatg

SEQ ID NO: 233 - DNA, 79 bp, Homo sapien
cagaaattggcagagcagtttgtcctcctcaatctggtttatgaaacaactgacaaacacctttctcctgatggccagt

SEQ ID NO: 234 - DNA, 22 bp, Homo sapien
cagaaattggcagagcagtttg

SEQ ID NO: 235 - DNA, 21 bp, Homo sapien
actggccatcaggagaaaggt

SEQ ID NO: 236 - DNA, 34 bp, Homo sapien
cctcctcaatctggtttatgaaacaactgacaaa

SEQ ID NO: 237 - DNA, 84 bp, Homo sapien
gcaggcactcagctcttcatgaactacacagcgtactatcccagccccccaggttcgcctaatagtcttggctacttccctaca

SEQ ID NO: 238 - DNA, 21 bp, Homo sapien
gcaggcactcagctcttcatg

SEQ ID NO: 239 - DNA, 27 bp, Homo sapien
tgtagggaagtagccaagactattagg

SEQ ID NO: 240 - DNA, 25 bp, Homo sapien
cgtactatcccagccccccaggttc

SEQ ID NO: 241 - DNA, 79 bp, Homo sapien
gctcttggatggaccttgcactctagaagggacaatggacttctggctttggccactttacttcctgccagtatcgggg

SEQ ID NO: 242 - DNA, 20 bp, Homo sapien
gctcttggatggaccttgca

SEQ ID NO: 243 - DNA, 18 bp, Homo sapien
ccccgatactggcaggaa

SEQ ID NO: 244 - DNA, 26 bp, Homo sapien
agggacaatggacttctggctttggc

SEQ ID NO: 245 - DNA, 77 bp, Homo sapien
cctgaatgtccacagtgaatacgagccatcatgggaagagcagccaatgcctgagactccaaaatggtttcatctgc

SEQ ID NO: 246 - DNA, 23 bp, Homo sapien
cctgaatgtccacagtgaatacg

SEQ ID NO: 247 - DNA, 24 bp, Homo sapien
gcagatgaaaccattttggagtct

SEQ ID NO: 248 - DNA, 23 bp, Homo sapien
tcatgggaagagcagccaatgcc

SEQ ID NO: 249 - DNA, 86 bp, Homo sapien
gaatctgttcctagtggagaatacgagccatcatgggaagagcagccaatgcctgagactccaaaatggtttcatctgccctattt

SEQ ID NO: 250 - DNA, 26 bp, Homo sapien
gaatctgttcctagtggagaatacga

SEQ ID NO: 251 - DNA, 24 bp, Homo sapien
aaatagggcagatgaaaccatttt

SEQ ID NO: 252 - DNA, 22 bp, Homo sapien
catgggaagagcagccaatgcc

SEQ ID NO: 253 - DNA, 71 bp, Homo sapien
tgtatgccgctgttcttgctgtcgctcccgacacctccgtccgcttctggtcatgagaggagacagaggcc

SEQ ID NO: 254 - DNA, 20 bp, Homo sapien
tgtatgccgctgttcttgct

SEQ ID NO: 255 - DNA, 21 bp, Homo sapien
ggcctctgtctcctctcatga

SEQ ID NO: 256 - DNA, 21 bp, Homo sapien
tcgctcccgacacctccgtcc

SEQ ID NO: 257 - DNA, 84 bp, Homo sapien
acccgaacaatcctccctcgtcagaggttctccgcatccagaacattctatctgagaagcctacagtgactacagtttatgcca

SEQ ID NO: 258 - DNA, 19 bp, Homo sapien
acccgaacaatcctccctc

SEQ ID NO: 259 - DNA, 25 bp, Homo sapien
tggcataaactgtagtcactgtagg

SEQ ID NO: 260 - DNA, 27 bp, Homo sapien
cagaggttctccgcatccagaacattc

SEQ ID NO: 261 - DNA, 79 bp, Homo sapien
ccctttcacggcaagacgtttatcctgaaggtgagtgaggggagcgggtgtcctggagccccagacagcacaggaggct

SEQ ID NO: 262 - DNA, 18 bp, Homo sapien
ccctttcacggcaagacg

SEQ ID NO: 263 - DNA, 19 bp, Homo sapien
agcctcctgtgctgtctgg

SEQ ID NO: 264 - DNA, 26 bp, Homo sapien
tatcctgaaggtgagtgaggggagcg

SEQ ID NO: 265 - DNA, 81 bp, Homo sapien
caggaagagaactggaagtttgagaagaataatgaatatggggacaccgtgtacaccattgaagttccctttcacggcaag

SEQ ID NO: 266 - DNA, 23 bp, Homo sapien
caggaagagaactggaagtttga

SEQ ID NO: 267 - DNA, 20 bp, Homo sapien
cttgccgtgaaagggaactt

SEQ ID NO: 268 - DNA, 31 bp, Homo sapien
aagaataatgaatatggggacaccgtgtaca

SEQ ID NO: 269 - DNA, 84 bp, Homo sapien
gcctgaaacactacccaataattcctgctattcagctggcattgcaaaactggaagaaggagatgaactccaacttgcaatacc

SEQ ID NO: 270 - DNA, 25 bp, Homo sapien
gcctgaaacactacccaataattcc

SEQ ID NO: 271 - DNA, 25 bp, Homo sapien
ggtattgcaagttggagttcatctc

SEQ ID NO: 272 - DNA, 30 bp, Homo sapien
ctattcagctggcattgcaaaactggaaga

SEQ ID NO: 273 - DNA, 73 bp, Homo sapien
tccgccagaagaatgcagttctgaacaaactgaaaactgcaattggagcagtggagaaagacgtgggcctgtc

SEQ ID NO: 274 - DNA, 20 bp, Homo sapien
tccgccagaagaatgcagtt

SEQ ID NO: 275 - DNA, 20 bp, Homo sapien
gacaggcccacgtctttctc

SEQ ID NO: 276 - DNA, 30 bp, Homo sapien
tgaacaaactgaaaactgcaattggagcag

SEQ ID NO: 277 - DNA, 89 bp, Homo sapien
gggctgtttgctcttttgtggatattgtgcctgtcaggattcttgaagttttttttttatagtgagataatggagttggtcttagccgc

SEQ ID NO: 278 - DNA, 20 bp, Homo sapien
gggctgtttgctcttttgtg

SEQ ID NO: 279 - DNA, 19 bp, Homo sapien
gcggctaagaccaactcca

SEQ ID NO: 280 - DNA, 30 bp, Homo sapien
tattgtgcctgtcaggattcttgaagtttt

SEQ ID NO: 281 - DNA, 92 bp, Homo sapien
ccatagatgaattgaaggaatgttttcttaaccaaacggatgaaactctgagcaatgttgaggtgtttatgcaattaatatatgacagcagt

SEQ ID NO: 282 - DNA, 27 bp, Homo sapien
ccatagatgaattgaaggaatgttttc

SEQ ID NO: 283 - DNA, 30 bp, Homo sapien
actgctgtcatatattaattgcataaacac

SEQ ID NO: 284 - DNA, 30 bp, Homo sapien
ccaaacggatgaaactctgagcaatgttga

SEQ ID NO: 285 - DNA, 68 bp, Homo sapien
ctgccaggaccgcttctactggcgcgtgagttcccggagtgagttgaaccaggtggaccaagtgggct

SEQ ID NO: 286 - DNA, 20 bp, Homo sapien
ctgccaggaccgcttctact

SEQ ID NO: 287 - DNA, 19 bp, Homo sapien
agcccacttggtccacctg

SEQ ID NO: 288 - DNA, 25 bp, Homo sapien
gcgtgagttcccggagtgagttgaa

SEQ ID NO: 289 - DNA, 82 bp, Homo sapien
acgatcaacaacacatactcctgtatgattgaaaatgacattgccaaagcaacaggggatatcaaagtgacagaatcggaga

SEQ ID NO: 290 - DNA, 24 bp, Homo sapien
acgatcaacaacacatactcctgt

SEQ ID NO: 291 - DNA, 24 bp, Homo sapien
tctccgattctgtcactttgatat

SEQ ID NO: 292 - DNA, 26 bp, Homo sapien
atgattgaaaatgacattgccaaagc

SEQ ID NO: 293 - DNA, 91 bp, Homo sapien
gctccaatatgaacaagataaatctatcttcaaagacatattagaagttgggaaaataattcatgtgaactagagtcaactgtgtcagggc

SEQ ID NO: 294 - DNA, 27 bp, Homo sapien
gctccaatatgaacaagataaatctat

SEQ ID NO: 295 - DNA, 23 bp, Homo sapien
gccctgacacagttgactctagt

SEQ ID NO: 296 - DNA, 34 bp, Homo sapien
tcaaagacatattagaagttgggaaaataattca

SEQ ID NO: 297 - DNA, 73 bp, Homo sapien
tggctttggtatttcagaagtctctgtctggctttcagcaatgaagggtttggttgtagaagttccaaggctt

SEQ ID NO: 298 - DNA, 23 bp, Homo sapien
tggctttggtatttcagaagtct

SEQ ID NO: 299 - DNA, 22 bp, Homo sapien
aagccttggaacttctacaacc

SEQ ID NO: 300 - DNA, 25 bp, Homo sapien
tgtctggctttcagcaatgaagggt

SEQ ID NO: 301 - DNA, 79 bp, Homo sapien
ccggactctcatgaacttgcacaacaacgaggcaggccgaaagatcctggaggagaacatgaagctggaatgtaagtgc

SEQ ID NO: 302 - DNA, 21 bp, Homo sapien
ccggactctcatgaacttgca

SEQ ID NO: 303 - DNA, 24 bp, Homo sapien
gcacttacattccagcttcatgtt

SEQ ID NO: 304 - DNA, 24 bp, Homo sapien
aggcaggccgaaagatcctggagg

SEQ ID NO: 305 - DNA, 75 bp, Homo sapien
tctgggctgctggtctcttctccttggcctgagctccctttctctgccggcagccatctcagccctgcagttgtc

SEQ ID NO: 306 - DNA, 20 bp, Homo sapien
tctgggctgctggtctcttc

SEQ ID NO: 307 - DNA, 21 bp, Homo sapien
gacaactgcagggctgagatg

SEQ ID NO: 308 - DNA, 23 bp, Homo sapien
ctgagctccctttctctgccggc

SEQ ID NO: 309 - DNA, 86 bp, Homo sapien
cggcccaccttcctgaagatcaagaagccactgtcgtaccgcaagcccatggacacggacctggtgtacatcgagaagtcgcccaa

SEQ ID NO: 310 - DNA, 17 bp, Homo sapien
cggcccaccttcctgaa

SEQ ID NO: 311 - DNA, 21 bp, Homo sapien
ttgggcgacttctcgatgtac

SEQ ID NO: 312 - DNA, 24 bp, Homo sapien
atcaagaagccactgtcgtaccgc

SEQ ID NO: 313 - DNA, 72 bp, Homo sapien
aagcggcccaccttcctgaagatcaagaagccactgtcgtaccgcaagcccatggacacggacctggtgtac

SEQ ID NO: 314 - DNA, 17 bp, Homo sapien
aagcggcccaccttcct

SEQ ID NO: 315 - DNA, 21 bp, Homo sapien
gtacaccaggtccgtgtccat

SEQ ID NO: 316 - DNA, 28 bp, Homo sapien
aagatcaagaagccactgtcgtaccgca

SEQ ID NO: 317 - DNA, 75 bp, Homo sapien; identical to SEQ ID NO: 305

SEQ ID NO: 318 - DNA, 20 bp, Homo sapien
tctgggctgctggtctcttc

SEQ ID NO: 319 - DNA, 21 bp, Homo sapien
gacaactgcagggctgagatg

SEQ ID NO: 320 - DNA, 23 bp, Homo sapien
ctgagctccctttctctgccggc

SEQ ID NO: 321 - DNA, 86 bp, Homo sapien
tgccaggcactgttcatctcagtttttctgtccctttgctcccggcaagctttctgctgaaagttcatatctggagcctgatgtct

SEQ ID NO: 322 - DNA, 20 bp, Homo sapien
tgccaggcactgttcatctc

SEQ ID NO: 323 - DNA, 25 bp, Homo sapien
agttcatatctggagcctgatgtct

SEQ ID NO: 324 - DNA, 20 bp, Homo sapien
ctttgctcccggcaagcttt

SEQ ID NO: 325 - DNA, 81 bp, Homo sapien
ccagctctgctgaggagtacgtgggcctgtcccagcaagggctctggcagctgacagggctttgtctgggacagctgcaaa

SEQ ID NO: 326 - DNA, 22 bp, Homo sapien
ccagctctgctgaggagtacgt

SEQ ID NO: 327 - DNA, 20 bp, Homo sapien
tttgcagctgtcccagacaa

SEQ ID NO: 328 - DNA, 23 bp, Homo sapien
cagcaagggctctggcagctgac

SEQ ID NO: 329 - DNA, 77 bp, Homo sapien
cagcctgaaaaagggcaagaaatgcagcaagaccaagaaatcccccgaaccagtcaggtttacttacgctggatgtt

SEQ ID NO: 330 - DNA, 20 bp, Homo sapien
cagcctgaaaaagggcaaga

SEQ ID NO: 331 - DNA, 26 bp, Homo sapien
aacatccagcgtaagtaaacctgact

SEQ ID NO: 332 - DNA, 25 bp, Homo sapien
cagcaagaccaagaaatcccccgaa

SEQ ID NO: 333 - DNA, 74 bp, Homo sapien
gagctgtggcttaccagatgtggcaaaatctggcaaaggcagagaccaaacttcatacctcaaactctgggggg

SEQ ID NO: 334 - DNA, 22 bp, Homo sapien
gagctgtggcttaccagatgtg

SEQ ID NO: 335 - DNA, 21 bp, Homo sapien
ccccccagagtttgaggtatg

SEQ ID NO: 336 - DNA, 28 bp, Homo sapien
caaaatctggcaaaggcagagaccaaac

SEQ ID NO: 337 - DNA, 71 bp, Homo sapien
gatccgcactcattccactgatgccagctgcccctgaaggatgccagtactgtggtgtgtgagtctcagca

SEQ ID NO: 338 - DNA, 21 bp, Homo sapien
gatccgcactcattccactga

SEQ ID NO: 339 - DNA, 24 bp, Homo sapien
tgctgagactcacacaccacagta

SEQ ID NO: 340 - DNA, 22 bp, Homo sapien
ccagctgcccctgaaggatgcc

SEQ ID NO: 341 - DNA, 71 bp, Homo sapien
agccacacaaaccgcctagagtgtgccaaacttgtgtgggaggatacccttgacaaatactgtgaccggga

SEQ ID NO: 342 - DNA, 20 bp, Homo sapien
agccacacaaaccgcctaga

SEQ ID NO: 343 - DNA, 22 bp, Homo sapien
tcccggtcacagtatttgtcaa

SEQ ID NO: 344 - DNA, 27 bp, Homo sapien
tgtgccaaacttgtgtgggaggatacc

SEQ ID NO: 345 - DNA, 90 bp, Homo sapien
tgaggtcagagaaaataagacaggtcgaatgagtgatttgagtgtaattggtcatccaatagattcagaatctaaagaagatgaaccttg

SEQ ID NO: 346 - DNA, 24 bp, Homo sapien
tgaggtcagagaaaataagacagg

SEQ ID NO: 347 - DNA, 26 bp, Homo sapien
caaggttcatcttctttagattctga

SEQ ID NO: 348 - DNA, 31 bp, Homo sapien
cgaatgagtgatttgagtgtaattggtcatc

SEQ ID NO: 349 - DNA, 76 bp, Homo sapien
aacaaccccatcagcaacgtcagttcttgccccatccctgatgggcccttctatgcagatcctaactatgcttctg

SEQ ID NO: 350 - DNA, 20 bp, Homo sapien
aacaaccccatcagcaacgt

SEQ ID NO: 351 - DNA, 27 bp, Homo sapien
cagaagcatagttaggatctgcataga

SEQ ID NO: 352 - DNA, 24 bp, Homo sapien
agttcttgccccatccctgatggg

SEQ ID NO: 353 - DNA, 88 bp, Homo sapien
ggcgtagacggtgcctgcaggccctgcagcgacgctgagctgctcctggccgcatgcaccagcgacttcgtaattcacgggatcatcc

SEQ ID NO: 354 - DNA, 19 bp, Homo sapien
ggcgtagacggtgcctgca

SEQ ID NO: 355 - DNA, 20 bp, Homo sapien
cgtaattcacgggatcatcc

SEQ ID NO: 356 - DNA, 22 bp, Homo sapien
cagcgacgctgagctgctcctg

SEQ ID NO: 357 - DNA, 71 bp, Homo sapien
cgcctgcaagtgtgtttcctgagtacaggtgtcgccgagggcgtgcacatctgctgtgtagctctctggga

SEQ ID NO: 358 - DNA, 20 bp, Homo sapien
cgcctgcaagtgtgtttcct

SEQ ID NO: 359 - DNA, 23 bp, Homo sapien
tcccagagagctacacagcagat

SEQ ID NO: 360 - DNA, 22 bp, Homo sapien
agtacaggtgtcgccgagggcg

SEQ ID NO: 361 - DNA, 80 bp, Homo sapien
ctgtgtatgtgtgattgaggtgttggggatgtcctttgcactgaccctgaactgccagattgacaaaaccagccagacca

SEQ ID NO: 362 - DNA, 25 bp, Homo sapien
ctgtgtatgtgtgattgaggtgttg

SEQ ID NO: 363 - DNA, 20 bp, Homo sapien
tggtctggctggttttgtca

SEQ ID NO: 364 - DNA, 27 bp, Homo sapien
atgtcctttgcactgaccctgaactgc

SEQ ID NO: 365 - DNA, 86 bp, Homo sapien
tcgagtattttggtatcagggagtctctgtctcccataagccccatgaacttaaaagtttgatgtggtttttgttttgtggtaaga

SEQ ID NO: 366 - DNA, 26 bp, Homo sapien
tcgagtattttggtatcagggagtct

SEQ ID NO: 367 - DNA, 26 bp, Homo sapien
tcttaccacaaaacaaaaaccacatc

SEQ ID NO: 368 - DNA, 27 bp, Homo sapien
ctcccataagccccatgaacttaaaag

SEQ ID NO: 369 - DNA, 75 bp, Homo sapien
aactgctgaagtatgtcctgtttttcttcaacttgctcttttggatctgtggctgctgcattttgggctttggga

SEQ ID NO: 370 - DNA, 26 bp, Homo sapien
aactgctgaagtatgtcctgtttttc

SEQ ID NO: 371 - DNA, 18 bp, Homo sapien
tcccaaagcccaaaatgc

SEQ ID NO: 372 - DNA, 28 bp, Homo sapien
caacttgctcttttggatctgtggctgc

SEQ ID NO: 373 - DNA, 66 bp, Homo sapien
cactccgaagactccagattccgagagttgcggaaacgctacgaggacctgctaaccaggctgcgg

SEQ ID NO: 374 - DNA, 22 bp, Homo sapien
cactccgaagactccagattcc

SEQ ID NO: 375 - DNA, 17 bp, Homo sapien
ccgcagcctggttagca

SEQ ID NO: 376 - DNA, 24 bp, Homo sapien
agagttgcggaaacgctacgagga

SEQ ID NO: 377 - DNA, 86 bp, Homo sapien
tacacaccaccttcgctgaaagctgtgcagcgcattgctgagtctcacctgcagtctatcagcaatttgaatgagaaccaggcctc

SEQ ID NO: 378 - DNA, 22 bp, Homo sapien
tacacaccaccttcgctgaaag

SEQ ID NO: 379 - DNA, 21 bp, Homo sapien
atttgaatgagaaccaggcct

SEQ ID NO: 380 - DNA, 24 bp, Homo sapien
tgtgcagcgcattgctgagtctca

SEQ ID NO: 381 - DNA, 76 bp, Homo sapien
gtgggagctgtaaggtcttctttaagagggcaatggaagggcagcacaactacttatgtgctggaagaaatgactg

SEQ ID NO: 382 - DNA, 25 bp, Homo sapien
gtgggagctgtaaggtcttctttaa

SEQ ID NO: 383 - DNA, 27 bp, Homo sapien
cagtcatttcttccagcacataagtag

SEQ ID NO: 384 - DNA, 22 bp, Homo sapien
agggcaatggaagggcagcaca

SEQ ID NO: 385 - DNA, 71 bp, Homo sapien
accttctcctcccccaacttcgccaccatcccgcaggacacggtgaccgagataacgtcctcctctcccag

SEQ ID NO: 386 - DNA, 21 bp, Homo sapien
accttctcctcccccaacttc

SEQ ID NO: 387 - DNA, 23 bp, Homo sapien
ctgggagaggaggacgttatctc

SEQ ID NO: 388 - DNA, 22 bp, Homo sapien
accatcccgcaggacacggtga

SEQ ID NO: 389 - DNA, 67 bp, Homo sapien
ctcccgccccagtcctgcccagcagggacaatgagattgtagacagcgcctcagttccagaaacgcc

SEQ ID NO: 390 - DNA, 16 bp, Homo sapien
ctcccgccccagtcct

SEQ ID NO: 391 - DNA, 19 bp, Homo sapien
ggcgtttctggaactgagg

SEQ ID NO: 392 - DNA, 27 bp, Homo sapien
cgctgtctacaatctcattgtccctgc

SEQ ID NO: 393 - DNA, 76 bp, Homo sapien
tggaggagaagaggaaagagaaagccaagatccactaccggaagaagaaacagctcatgaggctacggaaacaggc

SEQ ID NO: 394 - DNA, 23 bp, Homo sapien
tggaggagaagaggaaagagaaa

SEQ ID NO: 395 - DNA, 20 bp, Homo sapien
atgaggctacggaaacaggc

SEQ ID NO: 396 - DNA, 28 bp, Homo sapien
ccaagatccactaccggaagaagaaaca

SEQ ID NO: 397 - DNA, 74 bp, Homo sapien
gcagcatctacaaccctgaagtgcttgatatcacagaggaaactctgcattctcgcttcctggagggtgtccgc

SEQ ID NO: 398 - DNA, 22 bp, Homo sapien
gcagcatctacaaccctgaagt

SEQ ID NO: 399 - DNA, 17 bp, Homo sapien
tcctggagggtgtccgc

SEQ ID NO: 400 - DNA, 31 bp, Homo sapien
ttgatatcacagaggaaactctgcattctcg

SEQ ID NO: 401 - DNA, 84 bp, Homo sapien
aggcgcggaaggagtcacggggcgcgcatgccagggaagactacaaggtgcggattgatgagtacgattactccaagcccatcc

SEQ ID NO: 402 - DNA, 17 bp, Homo sapien
aggcgcggaaggagtca

SEQ ID NO: 403 - DNA, 20 bp, Homo sapien
ggatgggcttggagtaatcg

SEQ ID NO: 404 - DNA, 29 bp, Homo sapien
agggaagactacaaggtgcggattgatga

SEQ ID NO: 405 - DNA, 80 bp, Homo sapien
tgggaacaagagggcatctgctaaagtttcagattccatttctgctcagtatccagtagtggatcatgaatttgatgcag

SEQ ID NO: 406 - DNA, 20 bp, Homo sapien
tgggaacaagagggcatctg

SEQ ID NO: 407 - DNA, 22 bp, Homo sapien
catgaatttgatgcagtggtgg

SEQ ID NO: 408 - DNA, 32 bp, Homo sapien
agattccatttctgctcagtatccagtagtgg

SEQ ID NO: 409 - DNA, 75 bp, Homo sapien; identical to SEQ ID NO: 149

SEQ ID NO: 410 - DNA, 23 bp, Homo sapien
ttctgaagaccactccagcgata

SEQ ID NO: 411 - DNA, 16 bp, Homo sapien
cggcaaggcccagtga

SEQ ID NO: 412 - DNA, 28 bp, Homo sapien
actatgaagaatactgcaccgccaacgc

SEQ ID NO: 413 - DNA, 86 bp, Homo sapien
gctgtgatgtgaagtttcctataaggttagaaggccttgtgctcacccaccaacaatttagtagttatgagccagagttatttcct

SEQ ID NO: 414 - DNA, 26 bp, Homo sapien
gctgtgatgtgaagtttcctataagg

SEQ ID NO: 415 - DNA, 31 bp, Homo sapien
aggaaataactctggctcataactactaaat

SEQ ID NO: 416 - DNA, 24 bp, Homo sapien
aaggccttgtgctcacccaccaac

SEQ ID NO: 417 - DNA, 67 bp, Homo sapien
cagagcgactactccagctatgcccggacctccatcagagccagtctcaccttcaaccgcggcttca

SEQ ID NO: 418 - DNA, 23 bp, Homo sapien
cagagcgactactccagctatgc

SEQ ID NO: 419 - DNA, 17 bp, Homo sapien
tgaagccgcggttgaag

SEQ ID NO: 420 - DNA, 25 bp, Homo sapien
cggacctccatcagagccagtctca

SEQ ID NO: 421 - DNA, 83 bp, Homo sapien
gctgctttccctttccttgcatattctggaatcccagcagtttctttctgtttttgcgaggacacagattatccttatttggg

SEQ ID NO: 422 - DNA, 19 bp, Homo sapien
gctgctttccctttccttg

SEQ ID NO: 423 - DNA, 24 bp, Homo sapien
ggacacagattatccttatttggg

SEQ ID NO: 424 - DNA, 27 bp, Homo sapien
tcccagcagtttctttctgtttttgcg

SEQ ID NO: 425 - DNA, 73 bp, Homo sapien
tcgcagccgggatttgggtcgcggttcttgtttgtggatcgctgtgatcgtcacttgacaatgcagatcttcg

SEQ ID NO: 426 - DNA, 16 bp, Homo sapien
tcgcagccgggatttg

SEQ ID NO: 427 - DNA, 23 bp, Homo sapien
cgaagatctgcattgtcaagtga

SEQ ID NO: 428 - DNA, 24 bp, Homo sapien
tcgcggttcttgtttgtggatcgc

SEQ ID NO: 429 - DNA, 64 bp, Homo sapien
tacctccaccatgccaagtggtcccaggctgcacccatggcagaaggaggagggcagaatcatc

SEQ ID NO: 430 - DNA, 20 bp, Homo sapien
tacctccaccatgccaagtg

SEQ ID NO: 431 - DNA, 21 bp, Homo sapien
gatgattctgccctcctcctt

SEQ ID NO: 432 - DNA, 20 bp, Homo sapien
tcccaggctgcacccatggc

SEQ ID NO: 433 - DNA, 78 bp, Homo sapien
gcttctaggagataaaaagaacatccagtcatggataaaaatgagctggttcagaaggccaaactggccgagcaggct

SEQ ID NO: 434 - DNA, 27 bp, Homo sapien
gcttctaggagataaaaagaacatcca

SEQ ID NO: 435 - DNA, 17 bp, Homo sapien
agcctgctcggccagtt

SEQ ID NO: 436 - DNA, 29 bp, Homo sapien
tggataaaaatgagctggttcagaaggcc

(19) United States
(12) Patent Application Publication
(10) Pub. No.: US 2009/0118175 A1
(43) Pub. Date: May 7, 2009

(54) Compositions and Methods for Detection, Prognosis and Treatment of Breast Cancer

(76) Inventor: Roberto A. Macina, San Jose, CA (US)

Correspondence Address: LICATA & TYRRELL P.C., 66 E. MAIN STREET, MARLTON, NJ, 08053, US

(21) Appl. No.: 11/913,603

(22) PCT Filed: May 8, 2006
(86) PCT No.: PCT/US06/17653

§ 371 Date: Jul. 29, 2008

Related U.S. Application Data
(60) Provisional application No. 60/681,536, filed on May 6, 2005.
(60) Provisional application No. 60/696,164, filed on Jun. 29, 2005.
(60) Provisional application No. 60/749,287, filed on Dec. 9, 2005.

(51) Int. Cl. A61K 38/17; C12Q 1/68; C07H 21/04; C12N 15/12; A61P 35/00; A61K 31/7105; C07H 21/02; A61K 31/711

(52) U.S. Cl. 514/12; 435/6; 536/23.1; 514/44

(57) ABSTRACT

The present invention relates to methods of detection, prognosis and treatment of breast cancer using a plurality genes or gene products present in normal and neoplastic cells, tissues and bodily fluids. Gene products relate to compositions comprising the nucleic acids, polypeptides, antibodies, post translational modifications (PTMs), variants, derivatives, agonists and antagonists of the invention and methods for the use of these compositions. Additional uses include identifying, monitoring, staging, imaging and treating cancer and non-cancerous disease states in breast as well as determining the effectiveness of therapies alone or in combination for an individual. Therapies include gene therapy, therapeutic molecules including but not limited to antibodies, small molecules and antisense molecules.

This patent application claims the benefit of priority from U.S. Provisional Application Ser. No. 60/749,287, filed Dec. 9, 2005, U.S. Provisional Application Ser. No. 60/696,164, filed Jun. 29, 2005, and U.S. Provisional Application Ser. No. 60/681,536, filed May 6, 2005, teachings of each of which are herein incorporated by reference in their entirety.

### FIELD OF THE INVENTION

The present invention relates to methods of detection, prognosis and treatment of breast cancer using a plurality genes or gene products present in normal and neoplastic cells, tissues and bodily fluids. Gene products relate to compositions comprising the nucleic acids, polypeptides, antibodies, post translational modifications (PTMs), variants, derivatives, agonists and antagonists of the invention and methods for the use of these compositions. Additional uses include identifying, monitoring, staging, imaging and treating cancer and non-cancerous disease states in breast as well as determining the effectiveness of therapies alone or in combination for an individual. Therapies include gene therapy, therapeutic molecules including but not limited to antibodies, small molecules and antisense molecules.

### BACKGROUND OF THE INVENTION

### Breast Cancer

Breast cancer, also referred to as mammary cancer, is the second most common cancer among women, accounting for a third of the cancers diagnosed in the United States. One in nine women will develop breast cancer in her lifetime and about 192,000 new cases of breast cancer are diagnosed annually with about 42,000 deaths. Bevers, Primary Prevention of Breast Cancer, in Breast Cancer, 20-54 (Kelly K Hunt et al., ed., 2001); Kochanek et al., 49 Nat'l. Vital Statistics Reports 1, 14 (2001). Breast cancer is rare in women younger than 20 and uncommon in women under 30. The incidence of breast cancer rises with age and becomes significant by age 50. White non-Hispanic women have the highest incidence rate for breast cancer and Korean women have the lowest. Increased prevalence of the genetic mutations BRCA1 and BRCA2 that promote breast and other cancers are found in Ashkenazi Jews. African American women have the highest mortality rate for breast cancer among these same groups (31 per 100,000), while Chinese women have the lowest at 11 per 100,000. Although men can get breast cancer, this is rare. In the United States it is estimated there will be 214,640 new cases of breast cancer and 41,430 deaths due to breast cancer in 2006. (American Cancer Society Website: cancer with the extension .org of the world wide web). With the exception of those cases with associated genetic factors, precise causes of breast cancer are not known.

In the treatment of breast cancer, there is considerable emphasis on detection and risk assessment because early and accurate staging of breast cancer has a significant impact on survival. For example, breast cancer detected at an early stage (stage T0, discussed below) has a five-year survival rate of 92%. Conversely, if the cancer is not detected until a late stage (i.e., stage T4 (IV)), the five-year survival rate is reduced to 13%. AJCC Cancer Staging Handbook pp. 164-65 (Irvin D. Fleming et al. eds., 5^{th} ed. 1998).

Current methods for predicting or detecting breast cancer risk are not optimal. One method for predicting the relative risk of breast cancer is by examining a patient's risk factors and pursuing aggressive diagnostic and treatment regiments for high risk patients. A patient's risk of breast cancer has been positively associated with increasing age, nulliparity, family history of breast cancer, personal history of breast cancer, early menarche, late menopause, late age of first full term pregnancy, prior proliferative breast disease, irradiation of the breast at an early age and a personal history of malignancy. Lifestyle factors such as fat consumption, alcohol consumption, education, and socioeconomic status have also been associated with an increased incidence of breast cancer although a direct cause and effect relationship has not been established. While these risk factors are statistically significant, their weak association with breast cancer limits their usefulness. Most women who develop breast cancer have none of the risk factors listed above, other than the risk that comes with growing older. NIH Publication No. 00-1556 (2000).

Current screening methods for detecting cancer, such as breast self exam, ultrasound, and mammography have drawbacks that reduce their effectiveness or prevent their widespread adoption. Breast self exams, while useful, are unreliable for the detection of breast cancer in the initial stages where the tumor is small and difficult to detect by palpation. Ultrasound measurements require skilled operators at an increased expense. Mammography, while sensitive, is subject to over diagnosis in the detection of lesions that have questionable malignant potential. There is also the fear of the radiation used in mammography because prior chest radiation is a factor associated with an increased incidence of breast cancer.

At this time, current methods of breast cancer prevention are highly problematic. Specifically, these methods of breast cancer prevention involve either prophylactic mastectomy (mastectomy performed before cancer diagnosis) or chemoprevention (chemotherapy before cancer diagnosis), drastic measures that limit their adoption even among women with increased risk of breast cancer. Bevers, supra.

A number of markers have been associated with breast cancer. Examples of these markers include carcinoembryonic antigen (CEA) (Mughal et al., JAMA 249:1881 (1983)), MUC-1 (Frische and Liu, J. Clin. Ligand 22:320 (2000)), HER-2/neu (Haris et al., Proc. Am. Soc. Clin. Oncology 15:A96 (1996)), uPA, PAI-1, LPA, LPC, RAK and BRCA (Esteva and Fritsche, Serum and Tissue Markers for Breast Cancer, in Breast Cancer, 286-308 (2001)). These markers have problems with limited sensitivity, low correlation, and false negatives which limit their use for initial diagnosis. For example, while the BRCA1 gene mutation is useful as an indicator of an increased risk for breast cancer, it has limited use in cancer diagnosis because only 6.2% of breast cancers are BRCA1 positive. Malone et al., JAMA 279:922 (1998). See also, Mewman et al., JAMA 279:915 (1998) (correlation of only 3.3%). Another breast cancer marker is mammaglobin which has been reported in serum. Fanger et al., Tumor Biol. 23: 212-221 (2002).

There are four primary classifications of breast cancer varying by the site of origin and the extent of disease development.
I. Ductal carcinoma in situ (DCIS): Malignant transformation of ductal epithelial cells that remain in their normal position. By definition, DCIS is a localized disease, incapable of metastasis.
II. Invasive ductal carcinoma (IDC): Malignancy of the ductal epithelial cells breaking through the basal membrane and into the supporting tissue of the breast. IDC may eventually spread elsewhere in the body.
III. Lobular carcinoma in situ (LCIS): Malignancy arising in a single lobule of the breast that fails to extend through the lobule wall, it generally remains localized.
IV. Infiltrating lobular carcinoma (ILC): Malignancy arising in a single lobule of the breast and invading directly through the lobule wall into adjacent tissues.
By virtue of its invasion beyond the lobule wall, ILC may penetrate lymphatics and blood vessels and spread to distant sites.

For purposes of determining prognosis and treatment, these four breast cancer types have been staged according to the size of the primary tumor (T), the involvement of lymph nodes (N), and the presence of metastasis (M). Although DCIS by definition represents localized stage I disease, the other forms of breast cancer may range from stage II to stage IV. There are additional prognostic factors that further serve to guide surgical and medical intervention. The most common ones are total number of lymph nodes involved, ER (estrogen receptor) status, PR (progesterone receptor) status, Her2/neu receptor status and histologic grades.

Recently, researchers have reported a number of additional molecular markers and their association with breast cancer. Esteva et al., Seminars in Radiation Oncology 12(4): 319-322, (2002) reported molecular factors for breast cancer metastasis and survival. Specifically, they describe established markers, ER, PR, Ki-67, HER-2, and investigational markers, pS2, PCNA, Mitosin, EGFR, IGF, P53, BCL-2, Cyclins, uPA, PAI, VEGF, PD-ECGF, FGF. Haffy, Seminars in Radiation Oncology 12(4): 329-340 (2002), reviewed molecular factors relevant for breast cancer management including ER, PR, HER-2/neu, P53, Ki-67, thymidine kinase (TK), IGF, IGFR, cFMS proto-oncogene, cyclin D, VEGF, and germline mutations of BRCA1, BRCA2, p53, PTEN, and ATM. Chang et al., PNAS 102: 3738-3743 (2005), reported that patients with tumors expressing a wound response gene signature have a worse prognosis for survival and increased likelihood of metastasis. Other workers have reported chemokine receptors are implicated in metastasis, e.g., Wang et al., Cancer Research 64:1861-1866 (2004), CCR6 and CCR7, for carcinoma of the head and neck., Muller et al., Nature 410:50-56 (2001) with CXCR4 and CCR7 for breast cancer.

Other researchers have reported the use of gene expression as predictor of breast cancer outcomes. For example, Van de Vijver et al, N. Engl. J. Med. 347(25): 1999-2009 (2002) disclose the use of complementary DNA (cDNA) microarray to establish signatures associated with either a good or a poor prognosis. Van't Veer et al., Nature 415:530-536 (2002) describe a 70 gene profile associated with metastasis in young patients with lymph-node negative breast cancer. See also, US Patent Appn. 2003/0224374 (Dai et al.), the contents of which are hereby incorporated by reference. Specifically, Dai et al. describe in Table 2 (genes that can be used as surrogates for ER status), Table 4 (genes that distinguish BRCA-1 tumors from non-germline tumors), Table 6 (preferred prognosis markers).

Zehentner et al., Clin. Chem. 48(8): 1225-1231 (2002) report a multigene PCR panel to detect breast cancer cells in lymph nodes. Specifically, they describe a panel of the following genes: mammaglobin (U33147.1), B305D, B726P (AL357148.22 GI:30348856) which appears to be an isoform of NY-BR-1 (NM_{—}052997.1 GI:16506284), GABA(A) receptor pi subunit (U95367.1 GI:2197000).

Traditionally, breast cancers are diagnosed based on pathologic staging recognizing that different treatments are more effective for different stages of cancer. Stage TX indicates that primary tumor cannot be assessed (i.e., tumor was removed or breast tissue was removed). Stage T0 is characterized by abnormalities such as hyperplasia but with no evidence of primary tumor. Stage Tis is characterized by carcinoma in situ, intraductal carcinoma, lobular carcinoma in situ, or Paget's disease of the nipple with no tumor. Stage T1 (I) is characterized as having a tumor of 2 cm or less in the greatest dimension. Within stage T1, Tmic indicates microinvasion of 0.1 cm or less, T1a indicates a tumor of between 0.1 to 0.5 cm, T1b indicates a tumor of between 0.5 to 1 cm, and T1c indicates tumors of between 1 cm to 2 cm. Stage T2 (II) is characterized by tumors from 2 cm to 5 cm in the greatest dimension. Tumors greater than 5 cm in size are classified as stage T3 (III). Stage T4 (IV) indicates a tumor of any size with extension to the chest wall or skin. Within stage T4, T4a indicates extension of the tumor to the chess wall, T4b indicates edema or ulceration of the skin of the breast or satellite skin nodules confined to the same breast, T4c indicates a combination of T4a and T4b, and T4d indicates inflammatory carcinoma. AJCC Cancer Staging Handbook pp. 159-70 (Irvin D. Fleming et al. eds., 5^{th} ed. 1998). In addition to standard staging, as described above, breast tumors may be classified according to their estrogen receptor and progesterone receptor protein status. Fisher et al., Breast Cancer Research and Treatment 7:147 (1986). Additional pathological status, such as HER2/neu status may also be useful. Thor et al., J. Nat'l Cancer Inst. 90:1346 (1998); Paik et al., J. Nat'l. Cancer Inst. 90:1361 (1998); Hutchins et al, Proc. Am. Soc. Clin. Oncology 17:A2 (1998); and Simpson et al., J. Clin. Oncology 18:2059 (2000).

In addition to the staging of the primary tumor, breast cancer metastases to regional lymph nodes may be staged. Stage NX indicates that the lymph nodes cannot be assessed (e.g., previously removed). Stage N0 indicates no regional lymph node metastasis. Stage N1 indicates metastasis to movable ipsilateral axillary lymph nodes. Stage N2 indicates metastasis to ipsilateral axillary lymph nodes fixed to one another or to other structures. Stage N3 indicates metastasis to ipsilateral internal mammary lymph nodes. Id.

Stage determination has potential prognostic value and provides criteria for designing optimal therapy. Simpson et al, J. Clin. Oncology 18:2059 (2000). Generally, pathological staging of breast cancer is preferable to clinical staging because the former gives a more accurate prognosis. However, quality clinical staging would be advantageous if it were as accurate as pathological staging because it does not depend on invasive procedures to obtain tissue for pathological evaluation. Staging of breast cancer would be greatly improved by use of specific molecular markers in cells, tissues, or bodily fluids which could differentiate between different stages of invasion. Progress in this field will allow for more rapid and reliable methods for treating breast cancer patients.

Treatment of breast cancer is generally decided after an accurate staging of the primary tumor. Primary treatment options include breast conserving therapy (lumpectomy, breast irradiation, and surgical staging of the axilla), and modified radical mastectomy. Additional treatments include chemotherapy, regional irradiation, and, in extreme cases, terminating estrogen production by ovarian ablation.

Until recently, the customary treatment for all breast cancer was mastectomy. Fonseca et al., Annals of Internal Medicine 127:1013 (1997). However, recent data indicate that less radical procedures may be equally effective, in terms of survival, for early stage breast cancer. Fisher et al., J. of Clinical Oncology 16:441 (1998). Fisher et al. have reported the use of tamoxifen and chemotherapy for patients with node negative, estrogen receptor positive breast cancer. Fisher et al., N. Engl. J. Med. 320:479-484 (1989); Fisher et al., Lancet 364:858-868 (2004); Fisher et al., J. Natl. Cancer Inst. 89: 1673-1682 (1997). The treatment options for a patient with early stage breast cancer (i.e., stage Tis) may be breast-sparing surgery followed by localized radiation therapy at the breast. Alternatively, mastectomy optionally coupled with radiation or breast reconstruction may be employed. These treatment methods are equally effective in the early stages of breast cancer.

Patients with Stage I and Stage II breast cancer require surgery with chemotherapy and/or hormonal therapy. Surgery is of limited use in Stage III and Stage IV patients. Thus, these patients are better candidates for chemotherapy and radiation therapy with surgery limited to biopsy to permit initial staging or subsequent restaging because cancer is rarely curative at this stage of the disease. AJCC Cancer Staging Handbook 84, 164-65 (Irvin D. Fleming et al. eds., 5^{th} ed. 1998).

In an effort to provide more treatment options to patients, efforts are underway to define an earlier stage of breast cancer with low recurrence which could be treated with lumpectomy without postoperative radiation treatment. While a number of attempts have been made to classify early stage breast cancer, no consensus recommendation on postoperative radiation treatment has been obtained from these studies. Page et al., Cancer 75:1219 (1995); Fisher et al., Cancer 75:1223 (1995); Silverstein et al., Cancer 77:2267 (1996). Recently, Paik et al., N. Engl. J. Med. 351(27):2817-2826 (2004) reported that a 21 gene panel could be used to predict breast cancer recurrence in tamoxifen treated, node negative women. Specifically, Paik et al. describe the use of proliferation genes (Ki67, STK15, Survivin, CCNB1 (cyclin B1), MYBL2), invasion genes (MMP11 (stromolysin 3), CTSL2 (cathepsin L2)), estrogen receptor genes (ER, PGR, BCL2, SCUBE2), HER2, GRB7, GSTM1, CD68 and BAG1 in an algorithm for recurrence. They used the following genes as reference genes: ACTB (b-actin), GAPDH, RPLPO, GUS and TFRC. See also, Cronin et al., Amer. J. Path. 164(1):35-42 (2004) and WO 2004/065583 (Genomic Health) the contents of which are hereby incorporated by reference, particularly Table 1 (for invasive carcinoma), Table 2 (for ER (+) outcomes), Table 3 (for ER (−) outcomes), Table 4 (multivariate analysis), Tables 5A and 5B (for PCR amplicons), Tables 6A-6F (for PCR primers and probes).

### Angiogenesis in Cancer

Growth and metastasis of solid tumors are also dependent on angiogenesis. Folkman, J., Cancer Research, 46: 467-473 (1986); Folkman, J., Journal of the National Cancer Institute, 82: 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone. Weidner, N., et al., The New England Journal of Medicine, 324(1): 1-8 (1991).

Angiogenesis, defined as the growth or sprouting of new blood vessels from existing vessels, is a complex process that primarily occurs during embryonic development. The process is distinct from vasculogenesis, in that the new endothelial cells lining the vessel arise from proliferation of existing cells, rather than differentiating from stem cells. The process is invasive and dependent upon proteolysis of the extracellular matrix (ECM), migration of new endothelial cells, and synthesis of new matrix components. Angiogenesis occurs during embryogenic development of the circulatory system; however, in adult humans, angiogenesis only occurs as a response to a pathological condition (except during the reproductive cycle in women).

Under normal physiological conditions in adults, angiogenesis takes place only in very restricted situations such as hair growth and wounding healing. Auerbach, W. and Auerbach, R., Pharmacol Ther. 63(3):265-311 (1994); Ribatti et al., Haematologica 76(4):3 11-20 (1991); Risau, Nature 386(6626):67 1-4 (1997). Angiogenesis progresses by a stimulus which results in the formation of a migrating column of endothelial cells. Proteolytic activity is focused at the advancing tip of this “vascular sprout”, which breaks down the ECM sufficiently to permit the column of cells to infiltrate and migrate. Behind the advancing front, the endothelial cells differentiate and begin to adhere to each other, thus forming a new basement membrane. The cells then cease proliferation and finally define a lumen for the new arteriole or capillary.

Unregulated angiogenesis has gradually been recognized to be responsible for a wide range of disorders, including, but not limited to, cancer, cardiovascular disease, rheumatoid arthritis, psoriasis and diabetic retinopathy. Folkman, Nat. Med. 1(1):27-31 (1995); Isner, Circulation 99(13): 1653-5 (1999); Koch, Arthritis Rheum. 41(6):951-62 (1998); Walsh, Rheumatology (Oxford) 38(2):103-12 (1999); Ware and Simons, Nat. Med. 3(2): 158-64 (1997).

Of particular interest is the observation that angiogenesis is required by solid tumors for their growth and metastases. Folkman, 1986 supra; Folkman, J. Natl. Cancer Inst., 82(1) 4-6 (1990); Folkman, Semin. Cancer Biol. 3(2):65-71 (1992); Zetter, Annu. Rev. Med. 49:407-24 (1998). A tumor usually begins as a single aberrant cell which can proliferate only to a size of a few cubic millimeters due to the distance from available capillary beds, and it can stay ‘dormant’ without further growth and dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. The precise mechanisms that control the angiogenic switch is not well understood, but it is believed that neovascularization of tumor mass results from the net balance of a multitude of angiogenesis stimulators and inhibitors. Folkman, 1995, supra.

A potent angiogenesis inhibitor is endostatin identified by O'Reilly and Folkman. O'Reilly et al., Cell 88(2):277-85 (1997); O'Reilly et al., Cell 79(2):3 15-28 (1994). Its discovery was based on the phenomenon that certain primary tumors can inhibit the growth of distant metastases. O'Reilly and Folkman hypothesized that a primary tumor initiates angiogenesis by generating angiogenic stimulators in excess of inhibitors. However, angiogenic inhibitors, by virtue of their longer half life in the circulation, reach the site of a secondary tumor in excess of the stimulators. The net result is the growth of primary tumor and inhibition of secondary tumor. Endostatin is one of a growing list of such angiogenesis inhibitors produced by primary tumors. It is a proteolytic fragment of a larger protein: endostatin is a 20 kDa fragment of collagen XVIII (amino acid H1132-K1315 in murine collagen XVIII). Endostatin has been shown to specifically inhibit endothelial cell proliferation in vitro and block angiogenesis in vivo. More importantly, administration of endostatin to tumor-bearing mice leads to significant tumor regression, and no toxicity or drug resistance has been observed even after multiple treatment cycles. Boehm et al., Nature 390(6658):404-407 (1997). The fact that endostatin targets genetically stable endothelial cells and inhibits a variety of solid tumors makes it a very attractive candidate for anticancer therapy. Fidler and Ellis, Cell 79(2):185-8 (1994); Gastl et al., Oncology 54(3):177-84 (1997); Hinsbergh et al., Ann. Oncol. 10 Suppl. 4:60-3 (1999). In addition, angiogenesis inhibitors have been shown to be more effective when combined with radiation and chemotherapeutic agents. Klement, J. Clin. Invest., 105(8) R15-24 (2000). Browder, Cancer Res. 6-(7) 1878-86 (2000); Arap et al., Science 279(5349):377-80 (1998); Mauceri et al., Nature 394(6690):287-91 (1998).

### SUMMARY OF THE INVENTION

In one aspect, the invention concerns a method for determining the prognosis for an individual having breast cancer where the expression level of a plurality of gene products in Table 2a is determined, and where the differential expression of a plurality of gene products relative to a control is indicative of the individual's prognosis.

In a particular embodiment, the expression level of a plurality of gene products of the genes in Table 2b is also determined, and the differential expression of a plurality of gene products relative to a control is indicative of the individual's prognosis.

In another particular embodiment, the plurality of gene products comprises at least two, or at least four, or at least six, or at least eight gene products.

In another embodiment, the plurality of gene products are selected from the group comprising RAD54L, CYR61, ECT2, CCR8, BXMAS2-10, ESR1, CXCR6, B7-H4, TERT, CDH1 and CTSD. In a further embodiment, the gene products are selected from the group comprising RAD54L, CCR8, BXMAS2-10, CXCR6, CYR61, CDH1 and B7-H4. In another embodiment, the over-expression of CYR61, ECT2, CCR8, ESR1, B7-H4, TERT, and CDH1 gene products are indicative of a poor prognosis. In a further specific embodiment, the over-expression of RAD54L, CCR8, BXMAS2-10, CXCR6, CYR61, CDH1 and B7-H4 gene products are indicative of a poor prognosis. In another specific embodiment, the under-expression of ER and CTSD gene products are indicative of a poor prognosis.

In another embodiment, the over-expression of some gene products from Table 2a or the under-expression of some gene products from Table 2a are indicative of a good prognosis. In a different embodiment, the over-expression of some gene products from Table 2a or the under-expression of some gene products from Table 2a are indicative of a poor prognosis.

In a particular embodiment, the gene product is RNA. In a further embodiment, the gene product expression level is determined by quantitative PCR.

In another particular embodiment, the gene product is a polypeptide. In a further embodiment, the gene product expression level is determined by an assay comprising one or more antibodies.

In another particular embodiment, the sample of gene products is selected from the group consisting of tissues, cells and bodily fluids. In a further embodiment, the sample of gene products is selected where the tissues or cells are from a fixed, waxed, embedded specimen from said individual.

In another aspect, the invention provides a method for improving the prognosis for an individual comprising modulating levels of a plurality of gene products of Table 2a.

In a particular embodiment, the plurality of gene products comprises at least two, or at least four, or at least six, or at least eight gene products.

In another embodiment, modulating levels of gene products comprises increasing levels of gene products whose over-expression is associated with a good prognosis. In a further embodiment, the method includes increasing levels of gene products whose over-expression is associated with a good prognosis where the gene products are selected from the group comprising the gene products of Table 2a.

In another embodiment, modulating levels of gene products comprises decreasing levels of gene products whose under-expression is associated with a good prognosis. In a further embodiment, the method includes decreasing levels of gene products whose under-expression is associated with a good prognosis where the gene products are selected from the group comprising the gene products of Table 2a.

In another embodiment, modulating levels of gene products comprises decreasing levels of gene products whose over-expression is associated with a poor prognosis. In another embodiment, modulating levels of gene products comprises increasing levels of gene products whose under-expression is associated with a poor prognosis.

In another embodiment, the individual is administered an appropriate agonist or antagonist for a gene product of Table 2a which will improve the prognosis of the individual.

The invention further concerns an isolated nucleic acid molecule comprising (a) a nucleic acid molecule consisting essentially of a nucleic acid sequence that encodes an amino acid sequence of the gene products in Table 7; (b) a nucleic acid molecule that selectively hybridizes to the nucleic acid molecule of (a); or (c) a nucleic acid molecule having at least 95% sequence identity to the nucleic acid molecule of (a).

In a particular embodiment, the nucleic acid molecule is cDNA, genomic DNA, RNA, a mammalian nucleic acid molecule, or a human nucleic acid molecule.

The invention further concerns a set of three isolated nucleic acid molecules wherein: (a) each nucleic acid molecule consists essentially of a nucleic acid sequence encoding a portion of gene product described in Table 2a or Table 2b and (i) the first nucleic acid molecule is a forward primer 15 to 30 base pairs in length; (ii) the second nucleic acid molecule is reverse primer 15 to 30 base pairs in length; (iii) the third nucleic acid molecule is a probe 15-30 base pairs in length; such that the forward primer and reverse primer produce an amplicon detectable by the probe wherein the amplicon bridges two exons and is 60 to 100 base pairs in length; (b) a nucleic acid molecule that selectively hybridizes to one of the three nucleic acid molecules of (a); or (c) a nucleic acid molecule having at least 95% sequence identity to one of the three nucleic acid molecules of (a).

In another aspect, the invention concerns a method for determining the presence of a gene product of Table 2a in a sample, comprising the steps of: (a) contacting the sample with the nucleic acid molecule of Table 7 under conditions in which the nucleic acid molecule will selectively hybridize to a gene product of Table 2a; and (b) detecting hybridization of the nucleic acid molecule to a gene product of Table 2a in the sample, wherein the detection of the hybridization indicates the presence of a gene product of Table 2a in the sample.

In another aspect, the invention concerns a method for determining the presence of cancer specific protein in a sample, comprising the steps of: (a) contacting the sample with a suitable reagent under conditions in which the reagent will selectively interact with the cancer specific protein comprising an amino acid sequence with at least 95% sequence identity to the polypeptide encoded by a gene product in Table 2a; and (b) detecting the interaction of the reagent with a cancer specific protein in the sample, wherein the detection of the binding indicates the presence of a cancer specific protein in the sample.

Another aspect of the invention concerns a method for diagnosing or monitoring the presence and metastases of breast cancer in an individual, comprising the steps of: (a) determining an amount of (i) a nucleic acid molecule consisting essentially of a nucleic acid sequence that encodes an amino acid sequence of a gene product in Table 2a; (ii) a nucleic acid molecule consisting essentially of a nucleic acid sequence of a gene product in Table 2a; (iii) a nucleic acid molecule consisting essentially of a nucleic acid sequence of Table 7; (iv) a nucleic acid molecule that selectively hybridizes to the nucleic acid molecule of (i), (ii) or (iii); (v) a nucleic acid molecule having at least 95% sequence identity to the nucleic acid molecule of (i), (ii) or (iii); (vi) a polypeptide comprising an amino acid sequence with at least 95% sequence identity to the polypeptide encoded by a gene product in Table 2; or (vii) a polypeptide comprising an amino acid sequence encoded by a nucleic acid molecule having at least 95% sequence identity to a nucleic acid molecule comprising a nucleic acid sequence of a gene product of Table 2a; and (b) comparing the amount of the determined nucleic acid molecule or the polypeptide in the sample of the individual to the amount of the cancer specific marker in a normal control; wherein a difference in the amount of the nucleic acid molecule or the polypeptide in the sample compared to the amount of the nucleic acid molecule or the polypeptide in the normal control is associated with the presence of breast cancer.

In another aspect, the invention concerns a kit for detecting a risk of cancer or presence of cancer in a individual, where it is a kit comprising a means for determining the presence of: (a) a nucleic acid molecule consisting essentially of a nucleic acid sequence that encodes an amino acid sequence of the polypeptide encoded by a gene product in Table 2a; (b) a nucleic acid molecule consisting essentially of a nucleic acid sequence of a gene product in Table 2a; (c) a nucleic acid molecule consisting essentially of a nucleic acid sequence of Table 7; (d) a nucleic acid molecule that selectively hybridizes to the nucleic acid molecule of (a), (b) or (c); or (e) a nucleic acid molecule having at least 95% sequence identity to the nuclei acid molecule of (a), (b) or (c); or (f) a polypeptide comprising an amino acid sequence with at least 95% sequence identity to the polypeptide encoded by a gene product in Table 2a; or (g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a.

In another aspect, the invention concerns a method of treating an individual with breast cancer, comprising the step of administering a composition consisting of: (a) a nucleic acid molecule consisting essentially of a nucleic acid sequence that encodes an amino acid sequence of the polypeptide encoded by a gene product in Table 2a; (b) a nucleic acid molecule consisting essentially of a nucleic acid sequence of a gene product in Table 2a; (c) a nucleic acid molecule consisting essentially of a nucleic acid sequence of Table 7; (d) a nucleic acid molecule that selectively hybridizes to the nucleic acid molecule of (a), (b) or (c); or (e) a nucleic acid molecule having at least 95% sequence identity to the nucleic acid molecule of (a), (b) or (c); or (f) a polypeptide comprising an amino acid sequence with at least 95% sequence identity to the polypeptide encoded by a gene product in Table 2a; or (g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a; (h) an appropriate agonist or antagonist for a gene product of Table 2a; to an individual in need thereof, wherein said administration induces an immune response against the breast cancer cell expressing the nucleic acid molecule or polypeptide.

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2000); Ausubel et al., Short Protocols in Molecular Biology: A Compendium of Methods from Current Protocols in Molecular Biology-4^{th} Ed., Wiley & Sons (1999); Harlow and Lane, Antibodies: A Laboratory Manual, Cold Spring Harbor Laboratory Press (1990); and Harlow and Lane, Using Antibodies: A Laboratory Manual, Cold Spring Harbor Laboratory Press (1999).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery and/or treatment of patients.

The following terms, unless otherwise indicated, shall be understood to have the following meanings:

A “nucleic acid molecule” of this invention refers to a polymeric form of nucleotides and includes both sense and antisense strands of RNA (e.g. mRNA, siRNA), cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” The term “nucleic acid molecule” usually refers to a molecule of at least 10 bases in length, unless otherwise specified. The term includes single and double stranded forms of DNA. In addition, a polynucleotide may include either or both naturally occurring and modified nucleotides linked together by naturally occurring and/or non-naturally occurring nucleotide linkages.

Nucleotides are represented by single letter symbols in nucleic acid molecule sequences. The following table lists symbols identifying nucleotides or groups of nucleotides which may occupy the symbol position on a nucleic acid molecule. See Nomenclature Committee of the International Union of Biochemistry (NC-IUB), Nomenclature for incompletely specified bases in nucleic acid sequences, Recommendations 1984., Eur J. Biochem. 150(1):1-5 (1985).

| | | | Complementary |
|---|---|---|---|
| Symbol | Meaning | Group/Origin of Designation | Symbol |
| a | a | Adenine | t/u |
| g | g | Guanine | c |
| c | c | Cytosine | g |
| t | t | Thymine | a |
| u | u | Uracil | a |
| r | g or a | puRine | y |
| y | t/u or c | pYrimidine | r |
| m | a or c | aMino | k |
| k | g or t/u | Keto | m |
| s | g or c | Strong interactions 3H-bonds | w |
| w | a or t/u | Weak interactions 2H-bonds | s |
| b | g or c or t/u | not a | v |
| d | a or g or t/u | not c | h |
| h | a or c or t/u | not g | d |
| v | a or g or c | not t, not u | b |
| n | a or g or c | aNy | n |
| | or t/u, | | |
| | unknown, or | | |
| | other | | |

The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) The term “nucleic acid molecule” also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

A “gene” is defined as a nucleic acid molecule that comprises a nucleic acid sequence that encodes an RNA molecule and the expression control sequences that surround the nucleic acid sequence that encodes the RNA molecule. The encoded RNA molecule may a functional RNA (e.g. tRNA, rRNA), regulatory RNA (e.g. siRNA, miRNA, tncRNA, smRNA, snRNA) or messenger RNA (mRNA). Messenger RNA molecules may be transcribed into polypeptides which are also considered as being encoded for by the gene. For instance, a gene may comprise a promoter, one or more enhancers, a nucleic acid sequence that encodes an RNA molecule, downstream regulatory sequences and, possibly, other nucleic acid sequences involved in regulation of the expression of an RNA. As is well known in the art, eukaryotic genes usually contain both exons and introns. The term “exon” refers to a nucleic acid sequence found in genomic DNA that is bioinformatically predicted and/or experimentally confirmed to contribute contiguous sequence to a mature RNA transcript. The term “intron” refers to a nucleic acid sequence found in genomic DNA that is predicted and/or confirmed to not contribute to a mature RNA transcript, but rather to be “spliced out” during processing of the transcript.

A “gene product” is defined as a molecule expressed or encoded directly or indirectly by a gene. For example, gene products include pre-mRNA, mature mRNA, tRNA, rRNA, snRNA, ulRNA, siRNA, miRNA, tncRNA, smRNA, pre-polypeptides, pro-polypeptides, mature polypeptides, post translationally modified polypeptides, processed polypeptides, functionally active polypeptides, functionally inactive polypeptides, and complexed polypeptides. A single gene product may have several molecular functions and different gene products may share a single or similar molecular function.

The term “level(s) of gene product” is defined as a quantifiable measurement of the gene product. The measurement may be an assay to determine the amount or mass of the product in a sample, the amount of chemically or enzymatically active product in a sample, or the amount of biologically functional product in a sample. Examples of these assays include determining relative and total RNA expression, gene copies, pre-mRNA and mature mRNA levels, knockdown levels, regulatory or surrogate marker levels, ISH, FISH, immunoassays, IHC, proteomic assays and other assays described below.

A nucleic acid molecule or polypeptide is “derived” from a particular species if the nucleic acid molecule or polypeptide has been isolated from the particular species, or if the nucleic acid molecule or polypeptide is homologous to a nucleic acid molecule or polypeptide isolated from a particular species.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, or genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, (4) does not occur in nature as part of a larger sequence or (5) includes nucleotides or internucleoside bonds that are not found in nature. The term “isolated” or “substantially pure” also can be used in reference to recombinant or cloned DNA isolates, chemically synthesized polynucleotide analogs, or polynucleotide analogs that are biologically synthesized by heterologous systems. The term “isolated nucleic acid molecule” includes nucleic acid molecules that are integrated into a host cell chromosome at a heterologous site, recombinant fusions of a native fragment to a heterologous sequence, recombinant vectors present as episomes or as integrated into a host cell chromosome.

A “part” of a nucleic acid molecule refers to a nucleic acid molecule that comprises a partial contiguous sequence of at least 10 bases of the reference nucleic acid molecule and can range in length from at least 10 bases up to the full length reference nucleic acid sequence minus one nucleotide base. Thus, for example, when the full length reference nucleic acid molecule contains 1000 nucleotide bases, the part may contain from at least 10 up to 999 nucleotide bases of that reference nucleic acid molecule. Preferably, a part comprises at least 15-20, 20-30, 30-40, 40-50, 50-60, 60-70, 70-80 or 90-100 bases of a reference nucleic acid molecule. In theory, a nucleic acid sequence of 17 nucleotides is of sufficient length to occur at random less frequently than once in the three gigabase human genome, and thus to provide a nucleic acid probe that can uniquely identify the reference sequence in a nucleic acid mixture of genomic complexity. A preferred part is thus one which comprises at least 17 nucleotides and provides a nucleic acid probe specific for a reference nucleic acid molecule of the present invention. A further preferred part is one which comprises at least 17 nucleotides and spans an exon-exon junction of a mature RNA molecule. Another preferred part is one comprising a nucleic acid sequence, the expression of which is indicative of breast cancer. Another preferred part is one that comprises a nucleic acid sequence that can encode at least 6 contiguous amino acid sequences (fragments of at least 18 nucleotides) because they are useful in directing the expression or synthesis of peptides that are useful in mapping the epitopes of the polypeptide encoded by the reference nucleic acid. See, e.g., Geysen et al., Proc. Natl. Acad. Sci. USA 81:3998-4002 (1984); and U.S. Pat. Nos. 4,708,871 and 5,595,915, the disclosures of which are incorporated herein by reference in their entireties. Preferably the 6 contiguous amino acids comprise a contiguous region of amino acids identical to a portion of a CaSP of the present invention. A part may also comprise at least 25, 30, 35 or 40 nucleotides of a reference nucleic acid molecule, or at least 50, 60, 70, 80, 90, 100, 150, 200, 250, 300, 350, 400 or 500 nucleotides of a reference nucleic acid molecule. A part of a nucleic acid molecule may comprise no other nucleic acid sequences. Alternatively, a part of a nucleic acid may comprise other nucleic acid sequences from other nucleic acid molecules.

The term “oligonucleotide” refers to a nucleic acid molecule generally comprising a length of 200 bases or fewer. A nucleoside, as known by those skilled in the art, is a base-sugar combination. The base portion of a nucleoside is typically a heterocyclic base, the two most common classes of which are purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In some embodiments, the respective ends of this linear polymeric structure can be further joined to form a circular structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage. The term “oligonucleotide” often refers to single-stranded deoxyribonucleotides, but it can refer as well to single- or double-stranded ribonucleotides, RNA:DNA hybrids and double-stranded DNAs, among others.

Preferably, oligonucleotides are 10 to 60 bases in length and most preferably 12, 13, 14, 15, 16, 17, 18, 19 or 20 bases in length. Other preferred oligonucleotides are 25, 30, 35, 40, 45, 50, 55 or 60 bases in length. Oligonucleotides may be single-stranded, e.g. for use as probes or primers, or may be double-stranded, e.g. for use in the construction of a mutant gene. Oligonucleotides of the invention can be either sense or antisense oligonucleotides. An oligonucleotide can be derivatized or modified as discussed herein for nucleic acid molecules.

Thus, in the context of the present invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for a reference nucleic acid molecule and increased stability in the presence of nucleases.

Oligonucleotides, such as single-stranded DNA probe oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well known, this reaction can be prevented selectively, where desired, by removing the 5′ phosphates of the other polynucleotide(s) prior to ligation.

Oligonucleotides of the present invention may further include ribozymes, external guide sequence (EGS), oligozymes, and other short catalytic RNAs or catalytic oligonucleotides which hybridize to the reference nucleic acid molecules.

The term “naturally occurring nucleotide” referred to herein includes naturally occurring deoxyribonucleotides and ribonucleotides. The term “modified nucleotides” referred to herein includes nucleotides with modified or substituted sugar groups and the like. The term “nucleotide linkages” referred to herein includes nucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081-9093 (1986); Stein et al., Nucl. Acids Res. 16:3209-3221 (1988); Zon et al., Anti-Cancer Drug Design 6:539-568 (1991); Zon et al., in Eckstein (ed.) Oligonucleotides and Analogues: A Practical Approach, pp. 87-108, Oxford University Press (1991); Uhlmann and Peyman, Chemical Reviews 90:543 (1990), and U.S. Pat. No. 5,151,510, the disclosure of which is hereby incorporated by reference in its entirety.

Unless specified otherwise, the left hand end of a polynucleotide sequence in sense orientation is the 5′ end and the right hand end of the sequence is the 3′ end. In addition, the left hand direction of a polynucleotide sequence in sense orientation is referred to as the 5′ direction, while the right hand direction of the polynucleotide sequence is referred to as the 3′ direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

The term “allelic variant” refers to one of two or more alternative naturally occurring forms of a gene, wherein each gene possesses a unique nucleotide sequence. In a preferred embodiment, different alleles of a given gene have similar or identical biological properties.

The term “percent sequence identity” in the context of nucleic acid sequences refers to the residues in two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998)). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1.

A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, double stranded RNA (dsRNA) inhibition (RNAi), combination of triplex and antisense, hybridization probes and PCR primers.

In the molecular biology art, researchers use the terms “percent sequence identity”, “percent sequence similarity” and “percent sequence homology” interchangeably. In this application, these terms shall have the same meaning with respect to nucleic acid sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, more preferably at least about 95-99%, and most preferably at least about 99.5-99.9% of the nucleotide bases, as measured by any well known algorithm of sequence identity, such as FASTA, BLAST or Gap, as discussed above.

Alternatively, substantial similarity exists between a first and second nucleic acid sequence when the first nucleic acid sequence or fragment thereof hybridizes to an antisense strand of the second nucleic acid, under selective hybridization conditions. Typically, selective hybridization will occur between the first nucleic acid sequence and an antisense strand of the second nucleic acid sequence when there is at least about 55% sequence identity between the first and second nucleic acid sequences—preferably at least about 65%, more preferably at least about 75%, more preferably at least about 90%, even more preferably at least about 95%, further preferably at least about 98%, and most preferably at least about 99%, 99.5%, 99.8% or 99.9%—over a stretch of at least about 14 nucleotides, more preferably at least 17 nucleotides, even more preferably at least 20, 25, 30, 35, 40, 50, 60, 70, 80, 90 or 100 nucleotides, and most preferably at least 200, 300, 400, 500 or 1000 or greater nucleotides.

Alternatively, substantial similarity exists between a first and second nucleic acid sequence when the second nucleic acid sequence or fragment thereof hybridizes to an antisense strand of the first nucleic acid. Preferably, there is at least about 70% sequence identity between the first and second nucleic acid sequences—more preferably at least about 80%, more preferably at least about 90%, even more preferably at least about 95%, further preferably at least about 98%, and most preferably at least about 99%, 99.5%, 99.8% or 99.9% sequence identity—over the entire length of the second nucleic acid.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook (1989), supra, p. 9.51.

The Tₘ for a particular DNA-DNA hybrid can be estimated by the formula:

Tₘ=81.5° C.+16.6(log₁₀[Na⁺])+0.41 (fraction G+C)−0.63 (% formamide)−(600/1) where 1 is the length of the hybrid in base pairs.

The Tₘ for a particular RNA-RNA hybrid can be estimated by the formula:

Tₘ=79.8° C.+18.5(log₁₀[Na⁺])+0.58 (fraction G+C)+11.8 (fraction G+C)₂−0.35(% formamide)—(820/1).

The Tₘ for a particular RNA-DNA hybrid can be estimated by the formula:

Tₘ=79.8° C.+18.5(log₁₀[Na⁺])+0.58 (fraction G+C)+11.8 (fraction G+C)²−0.50(% formamide)−(820/1).

In general, the Tₘ decreases by 1-1.5° C. for each 1% of mismatch between two nucleic acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tₘ of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

An example of stringent hybridization conditions for hybridization of complementary nucleic acid sequences having more than 100 complementary residues on a filter in a Southern or Northern blot or for screening a library is 50% formamide/6×SSC at 42° C. for at least ten hours and preferably overnight (approximately 16 hours). Another example of stringent hybridization conditions is 6×SSC at 68° C. without formamide for at least ten hours and preferably overnight. An example of moderate stringency hybridization conditions is 6×SSC at 55° C. without formamide for at least ten hours and preferably overnight. An example of low stringency hybridization conditions for hybridization of complementary nucleic acid sequences having more than 100 complementary residues on a filter in a Southern or northern blot or for screening a library is 6×SSC at 42° C. for at least ten hours. Hybridization conditions to identify nucleic acid sequences that are similar but not identical can be identified by experimentally changing the hybridization temperature from 68° C. to 42° C. while keeping the salt concentration constant (6×SSC), or keeping the hybridization temperature and salt concentration constant (e.g. 42° C. and 6×SSC) and varying the formamide concentration from 50% to 0%. Hybridization buffers may also include blocking agents to lower background. These agents are well known in the art. See Sambrook et al. (1989), supra, pages 8.46 and 9.46-9.58. See also Ausubel (1992), supra, Ausubel (1999), supra, and Sambrook (2001), supra.

Wash conditions can also be altered to change stringency conditions. An example of stringent wash conditions is a 0.2×SSC wash at 65° C. for 15 minutes (see Sambrook (1989), supra, for SSC buffer). Often the high stringency wash is preceded by a low stringency wash to remove excess probe. An exemplary medium stringency wash for duplex DNA of more than 100 base pairs is 1×SSC at 45° C. for 15 minutes. An exemplary low stringency wash for such a duplex is 4×SSC at 40° C. for 15 minutes. In general, signal-to-noise ratio of 2× or higher than that observed for an unrelated probe in the particular hybridization assay indicates detection of a specific hybridization.

As defined herein, nucleic acids that do not hybridize to each other under stringent conditions are still substantially similar to one another if they encode polypeptides that are substantially identical to each other. This occurs, for example, when a nucleic acid is created synthetically or recombinantly using a high codon degeneracy as permitted by the redundancy of the genetic code.

Hybridization conditions for nucleic acid molecules that are shorter than 100 nucleotides in length (e.g., for oligonucleotide probes) may be calculated by the formula:

Tₘ=81.5° C.+16.6(log₁₀[Na⁺])+0.41(fraction G+C)−(600/N), wherein N is change length and the [Na⁺] is 1 M or less. See Sambrook (1989), supra, p. 11.46. For hybridization of probes shorter than 100 nucleotides, hybridization is usually performed under stringent conditions (5-10° C. below the Tₘ) using high concentrations (0.1-1.0 pmol/ml) of probe. Id. at p. 11.45. Determination of hybridization using mismatched probes, pools of degenerate probes or “guessmers,” as well as hybridization solutions and methods for empirically determining hybridization conditions are well known in the art. See, e.g., Ausubel (1999), supra; Sambrook (1989), supra, pp. 11.45-11.57.

The term “digestion” or “digestion of DNA” refers to catalytic cleavage of the DNA with a restriction enzyme that acts only at certain sequences in the DNA. The various restriction enzymes referred to herein are commercially available and their reaction conditions, cofactors and other requirements for use are known and routine to the skilled artisan. For analytical purposes, typically, 1 μg of plasmid or DNA fragment is digested with about 2 units of enzyme in about 20 μl of reaction buffer. For the purpose of isolating DNA fragments for plasmid construction, typically 5 to 50 μg of DNA are digested with 20 to 250 units of enzyme in proportionately larger volumes. Appropriate buffers and substrate amounts for particular restriction enzymes are described in standard laboratory manuals, such as those referenced below, and are specified by commercial suppliers. Incubation times of about 1 hour at 37° C. are ordinarily used, but conditions may vary in accordance with standard procedures, the supplier's instructions and the particulars of the reaction. After digestion, reactions may be analyzed, and fragments may be purified by electrophoresis through an agarose or polyacrylamide gel, using well known methods that are routine for those skilled in the art.

The term “ligation” refers to the process of forming phosphodiester bonds between two or more polynucleotides, which most often are double-stranded DNAs. Techniques for ligation are well known to the art and protocols for ligation are described in standard laboratory manuals and references, such as, e.g., Sambrook (1989), supra.

Genome-derived “single exon probes,” are probes that comprise at least part of an exon (“reference exon”) and can hybridize detectably under high stringency conditions to transcript-derived nucleic acids that include the reference exon but do not hybridize detectably under high stringency conditions to nucleic acids that lack the reference exon. Single exon probes typically further comprise, contiguous to a first end of the exon portion, a first intronic and/or intergenic sequence that is identically contiguous to the exon in the genome, and may contain a second intronic and/or intergenic sequence that is identically contiguous to the exon in the genome. The minimum length of genome-derived single exon probes is defined by the requirement that the exonic portion be of sufficient length to hybridize under high stringency conditions to transcript-derived nucleic acids, as discussed above. The maximum length of genome-derived single exon probes is defined by the requirement that the probes contain portions of no more than one exon. The single exon probes may contain priming sequences not found in contiguity with the rest of the probe sequence in the genome, which priming sequences are useful for PCR and other amplification-based technologies. In another aspect, the invention is directed to single exon probes based on the CaSNAs disclosed herein.

In one embodiment, the term “microarray” refers to a “nucleic acid microarray” having a substrate-bound plurality of nucleic acids, hybridization to each of the plurality of bound nucleic acids being separately detectable. The substrate can be solid or porous, planar or non-planar, unitary or distributed. Nucleic acid microarrays include all the devices so called in Schena (ed.), DNA Microarrays: A Practical Approach (Practical Approach Series), Oxford University Press (1999); Nature Genet. 21(1)(suppl.):1-60 (1999); Schena (ed.), Microarray Biochip: Tools and Technology, Eaton Publishing Company/BioTechniques Books Division (2000). Additionally, these nucleic acid microarrays include substrate-bound plurality of nucleic acids in which the plurality of nucleic acids are disposed on a plurality of beads, rather than on a unitary planar substrate, as is described, inter alia, in Brenner et al., Proc. Natl. Acad. Sci. USA 97(4):1665-1670 (2000). Examples of nucleic acid microarrays may be found in U.S. Pat. Nos. 6,391,623, 6,383,754, 6,383,749, 6,380,377, 6,379,897, 6,376,191, 6,372,431, 6,351,712 6,344,316, 6,316,193, 6,312,906, 6,309,828, 6,309,824, 6,306,643, 6,300,063, 6,287,850, 6,284,497, 6,284,465, 6,280,954, 6,262,216, 6,251,601, 6,245,518, 6,263,287, 6,251,601, 6,238,866, 6,228,575, 6,214,587, 6,203,989, 6,171,797, 6,103,474, 6,083,726, 6,054,274, 6,040,138, 6,083,726, 6,004,755, 6,001,309, 5,958,342, 5,952,180, 5,936,731, 5,843,655, 5,814,454, 5,837,196, 5,436,327, 5,412,087, 5,405,783, the disclosures of which are incorporated herein by reference in their entireties.

In an alternative embodiment, a “microarray” may also refer to a “peptide microarray” or “protein microarray” having a substrate-bound collection of plurality of polypeptides, the binding to each of the plurality of bound polypeptides being separately detectable. Alternatively, the peptide microarray may have a plurality of binders, including but not limited to monoclonal antibodies, polyclonal antibodies, phage display binders, yeast 2 hybrid binders, aptamers, which can specifically detect the binding of the polypeptides of this invention. The array may be based on autoantibody detection to the polypeptides of this invention, see Robinson et al., Nature Medicine 8(3):295-301 (2002). Examples of peptide arrays may be found in WO 02/31463, WO 02/25288, WO 01/94946, WO 01/88162, WO 01/68671, WO 01/57259, WO 00/61806, WO 00/54046, WO 00/47774, WO 99/40434, WO 99/39210, WO 97/42507 and U.S. Pat. Nos. 6,268,210, 5,766,960, 5,143,854, the disclosures of which are incorporated herein by reference in their entireties.

In addition, determination of the levels of the CaSNA or CaSP may be made in a multiplex manner using techniques described in WO 02/29109, WO 02/24959, WO 01/83502, WO01/73113, WO 01/59432, WO 01/57269, WO 99/67641, the disclosures of which are incorporated herein by reference in their entireties.

The term “mutant”, “mutated”, or “mutation” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. In a preferred embodiment of the present invention, the nucleic acid sequence is the wild type nucleic acid sequence encoding a CaSP or is a CaSNA. The nucleic acid sequence may be mutated by any method known in the art including those mutagenesis techniques described infra.

The term “error-prone PCR” refers to a process for performing PCR under conditions where the copying fidelity of the DNA polymerase is low, such that a high rate of point mutations is obtained along the entire length of the PCR product. See, e.g., Leung et al., Technique 1: 11-15 (1989) and Caldwell et al., PCR Methods Applic. 2: 28-33 (1992).

The term “oligonucleotide-directed mutagenesis” refers to a process which enables the generation of site-specific mutations in any cloned DNA segment of interest. See, e.g., Reidhaar-Olson et al., Science 241: 53-57 (1988).

The term “assembly PCR” refers to a process which involves the assembly of a PCR product from a mixture of small DNA fragments. A large number of different PCR reactions occur in parallel in the same vial, with the products of one reaction priming the products of another reaction.

The term “sexual PCR mutagenesis” or “DNA shuffling” refers to a method of error-prone PCR coupled with forced homologous recombination between DNA molecules of different but highly related DNA sequence in vitro, caused by random fragmentation of the DNA molecule based on sequence similarity, followed by fixation of the crossover by primer extension in an error-prone PCR reaction. See, e.g., Stemmer, Proc. Natl. Acad. Sci. U.S.A. 91: 10747-10751 (1994). DNA shuffling can be carried out between several related genes (“Family shuffling”).

The term “in vivo mutagenesis” refers to a process of generating random mutations in any cloned DNA of interest which involves the propagation of the DNA in a strain of bacteria such as E. coli that carries mutations in one or more of the DNA repair pathways. These “mutator” strains have a higher random mutation rate than that of a wild-type parent. Propagating the DNA in a mutator strain will eventually generate random mutations within the DNA.

The term “cassette mutagenesis” refers to any process for replacing a small region of a double-stranded DNA molecule with a synthetic oligonucleotide “cassette” that differs from the native sequence. The oligonucleotide often contains completely and/or partially randomized native sequence.

The term “recursive ensemble mutagenesis” refers to an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. See, e.g., Arkin et al., Proc. Natl. Acad. Sci. U.S.A. 89: 7811-7815 (1992).

The term “exponential ensemble mutagenesis” refers to a process for generating combinatorial libraries with a high percentage of unique and functional mutants, wherein small groups of residues are randomized in parallel to identify, at each altered position, amino acids which lead to functional proteins. See, e.g., Delegrave et al., Biotechnology Research 11: 1548-1552 (1993); Arnold, Current Opinion in Biotechnology 4: 450-455 (1993).

“Operatively linked” expression control sequences refers to a linkage in which the expression control sequence is either contiguous with the gene of interest to control the gene of interest, or acts in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome. Viral vectors that infect bacterial cells are referred to as bacteriophages. Certain vectors are capable of autonomous replication in a host cell into which they are introduced (e.g., bacterial vectors having a bacterial origin of replication). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors that serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the phrase “open reading frame” and the equivalent acronym “ORF” refers to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence is meant to be inclusive of all nucleic acid sequences that can be directly translated, using the standard genetic code, to provide an amino acid sequence identical to that translated from the reference nucleic acid sequence.

The term “polypeptide” encompasses both naturally occurring and non-naturally occurring proteins and polypeptides, as well as polypeptide fragments and polypeptide mutants, derivatives and analogs thereof. A polypeptide may be monomeric or polymeric. Further, a polypeptide may comprise a number of different modules within a single polypeptide each of which has one or more distinct activities. A preferred polypeptide in accordance with the invention comprises a CaSP encoded by a nucleic acid molecule of the instant invention, or a fragment, mutant, analog, isoform, allelic variant and derivative thereof.

The term “isolated protein” or “isolated polypeptide” is a protein or polypeptide that by virtue of its origin or source of derivation (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art.

A protein or polypeptide is “substantially pure,” “substantially homogeneous” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be determined by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “fragment” when used herein with respect to polypeptides of the present invention refers to a polypeptide that has an amino-terminal and/or carboxy-terminal deletion compared to a full-length CaSP. In a preferred embodiment, the fragment is a contiguous sequence in which the amino acid sequence of the fragment is identical to the corresponding positions in the naturally occurring polypeptide. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “derivative” when used herein with respect to polypeptides of the present invention refers to a polypeptide which is substantially similar in primary structural sequence to a CaSP but which include, e.g., in vivo or in vitro chemical and biochemical modifications that are not found in the CaSP. Such modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Other modifications include, e.g., labeling with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, ¹⁴C and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See Ausubel (1992), supra; Ausubel (1999), supra.

The term “fusion protein” refers to polypeptides of the present invention coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusion proteins can be produced recombinantly by constructing a nucleic acid sequence that encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “analog” refers to both polypeptide analogs and non-peptide analogs. The term “polypeptide analog” as used herein refers to a polypeptide that is comprised of a segment of at least 25 amino acids that has substantial identity to a portion of an amino acid sequence but which contains non-natural amino acids or non-natural inter-residue bonds. In a preferred embodiment, the analog has the same or similar biological activity as the native polypeptide. Typically, polypeptide analogs comprise a conservative amino acid substitution (or insertion or deletion) with respect to the naturally occurring sequence. Analogs typically are at least 20 amino acids long, preferably at least 50 amino acids long or longer, and can often be as long as a full-length naturally occurring polypeptide.

The term “non-peptide analog” refers to a compound with properties that are analogous to those of a reference polypeptide. A non-peptide compound may also be termed a “peptide mimetic” or a “peptidomimetic.” Such compounds are often developed with the aid of computerized molecular modeling. Peptide mimetics that are structurally similar to useful peptides may be used to produce an equivalent effect. Generally, peptidomimetics are structurally similar to a paradigm polypeptide (i.e., a polypeptide that has a desired biochemical property or pharmacological activity), but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo et al., Ann. Rev. Biochem. 61:387-418 (1992)). For example, one may add internal cysteine residues capable of forming intramolecular disulfide bridges which cyclize the peptide.

The term “mutant” or “mutein” when referring to a polypeptide of the present invention relates to an amino acid sequence containing substitutions, insertions or deletions of one or more amino acids compared to the amino acid sequence of a CaSP. A mutein may have one or more amino acid point substitutions, in which a single amino acid at a position has been changed to another amino acid, one or more insertions and/or deletions, in which one or more amino acids are inserted or deleted, respectively, in the sequence of the naturally occurring protein, and/or truncations of the amino acid sequence at either or both the amino or carboxy termini. Further, a mutein may have the same or different biological activity as the naturally occurring protein. For instance, a mutein may have an increased or decreased biological activity. A mutein has at least 50% sequence similarity to the wild type protein, preferred is 60% sequence similarity, more preferred is 70% sequence similarity. Even more preferred are muteins having 80%, 85% or 90% sequence similarity to a CaSP. In an even more preferred embodiment, a mutein exhibits 95% sequence identity, even more preferably 97%, even more preferably 98% and even more preferably 99%. Sequence similarity may be measured by any common sequence analysis algorithm, such as GAP or BESTFIT or other variation Smith-Waterman alignment. See, T. F. Smith and M. S. Waterman, J. Mol. Biol. 147:195-197 (1981) and W. R. Pearson, Genomics 11:635-650 (1991).

Preferred amino acid substitutions are those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs. For example, single or multiple amino acid substitutions (preferably conservative amino acid substitutions) may be made in the naturally occurring sequence (preferably in the portion of the polypeptide outside the domain(s) forming intermolecular contacts. In a preferred embodiment, the amino acid substitutions are moderately conservative substitutions or conservative substitutions. In a more preferred embodiment, the amino acid substitutions are conservative substitutions. A conservative amino acid substitution should not substantially change the structural characteristics of the parent sequence (e.g., a replacement amino acid should not tend to disrupt a helix that occurs in the parent sequence, or disrupt other types of secondary structure that characterizes the parent sequence). Examples of art-recognized polypeptide secondary and tertiary structures are described in Creighton (ed.), Proteins, Structures and Molecular Principles, W. H. Freeman and Company (1984); Branden et al. (ed.), Introduction to Protein Structure, Garland Publishing (1991); Thornton et al., Nature 354:105-106 (1991).

As used herein, the twenty conventional amino acids and their abbreviations follow conventional usage. See Golub et al. (eds.), Immunology—A Synthesis 2^{nd} Ed., Sinauer Associates (1991). Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the right hand direction is the carboxy-terminal direction, in accordance with standard usage and convention.

By “homology” or “homologous” when referring to a polypeptide of the present invention, it is meant polypeptides from different organisms with a similar sequence to the encoded amino acid sequence of a CaSP and a similar biological activity or function. Although two polypeptides are said to be “homologous,” this does not imply that there is necessarily an evolutionary relationship between the polypeptides. Instead, the term “homologous” is defined to mean that the two polypeptides have similar amino acid sequences and similar biological activities or functions. In a preferred embodiment, a homologous polypeptide is one that exhibits 50% sequence similarity to CaSP, preferred is 60% sequence similarity, more preferred is 70% sequence similarity. Even more preferred are homologous polypeptides that exhibit 80%, 85% or 90% sequence similarity to a CaSP. In a yet more preferred embodiment, a homologous polypeptide exhibits 95%, 97%, 98% or 99% sequence similarity.

When “sequence similarity” is used in reference to polypeptides, it is recognized that residue positions that are not identical often differ by conservative amino acid substitutions. In a preferred embodiment, a polypeptide that has “sequence similarity” comprises conservative or moderately conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson, Methods Mol. Biol. 24: 307-31 (1994).

For instance, the following six groups each contain amino acids that are conservative substitutions for one another:

1) Serine (S), Threonine (T);

2) Aspartic Acid (D), Glutamic Acid (E);

3) Asparagine (N), Glutamine (Q);

4) Arginine (R), Lysine (K);

5) Isoleucine (1), Leucine (L), Methionine (M), Alanine (A), Valine (V), and

6) Phenylalanine (F), Tyrosine (Y), Tryptophan (W).

Alternatively, a conservative replacement is any change having a positive value in the PAM250 log-likelihood matrix disclosed in Gonnet et al., Science 256: 1443-45 (1992). A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Other programs include FASTA, discussed supra.

A preferred algorithm when comparing a sequence of the invention to a database containing a large number of sequences from different organisms is the computer program BLAST, especially blastp or tblastn. See, e.g., Altschul et al., J. Mol. Biol. 215: 403-410 (1990); Altschul et al., Nucleic Acids Res. 25:3389-402 (1997). Preferred parameters for blastp are:

| | | |
|---|---|---|
| Expectation value: | 10 | (default) |
| Filter: | seg | (default) |
| Cost to open a gap: | 11 | (default) |
| Cost to extend a gap: | 1 | (default) |
| Max. alignments: | 100 | (default) |
| Word size: | 11 | (default) |
| No. of descriptions: | 100 | (default) |

| | |
|---|---|
| Penalty Matrix: | BLOSUM62 |

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

Algorithms other than blastp for database searching using amino acid sequences are known in the art. For instance, polypeptide sequences can be compared using FASTA, a program in GCG Version 6.1. FASTA (e.g., FASTA2 and FASTA3) provides alignments and percent sequence identity of the regions of the best overlap between the query and search sequences (Pearson (1990), supra; Pearson (2000), supra). For example, percent sequence identity between amino acid sequences can be determined using FASTA with its default or recommended parameters (a word size of 2 and the PAM250 scoring matrix), as provided in GCG Version 6.1.

An “antibody” refers to an intact immunoglobulin, or to an antigen-binding portion thereof that competes with the intact antibody for specific binding to a molecular species, e.g., a polypeptide of the instant invention. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. A Fab fragment is a monovalent fragment consisting of the VL, VH, CL and CH1 domains; a F(ab′)₂ fragment is a bivalent fragment comprising two Fab fragments linked by a disulfide bridge at the hinge region; a Fd fragment consists of the VH and CH1 domains; a Fv fragment consists of the VL and VH domains of a single arm of an antibody; and a dAb fragment consists of a VH domain. See, e.g., Ward et al., Nature 341: 544-546 (1989).

By “bind specifically” and “specific binding” as used herein it is meant the ability of the antibody to bind to a first molecular species in preference to binding to other molecular species with which the antibody and first molecular species are admixed. An antibody is said specifically to “recognize” a first molecular species when it can bind specifically to that first molecular species.

A single-chain antibody (scFv) is an antibody in which VL and VH regions are paired to form a monovalent molecule via a synthetic linker that enables them to be made as a single protein chain. See, e.g., Bird et al., Science 242: 423-426 (1988); Huston et al., Proc. Natl. Acad. Sci. USA 85: 5879-5883 (1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. See e.g., Holliger et al., Proc. Natl. Acad. Sci. USA 90: 6444-6448 (1993); Poljak et al., Structure 2: 1121-1123 (1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. It is known that purified proteins, including purified antibodies, may be stabilized with non-naturally-associated components. The non-naturally-associated component may be a protein, such as albumin (e.g., BSA) or a chemical such as polyethylene glycol (PEG).

A “neutralizing antibody” or “an inhibitory antibody” is an antibody that inhibits the activity of a polypeptide or blocks the binding of a polypeptide to a ligand that normally binds to it. An “activating antibody” is an antibody that increases the activity of a polypeptide.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is less than 1 μM, preferably less than 100 nM and most preferably less than 10 nM.

The terms “patient” and “individual” includes human and veterinary subjects.

Throughout this specification and claims, the word “comprise,” or variations such as “comprises” or “comprising,” will be understood to imply the inclusion of a stated integer or group of integers but not the exclusion of any other integer or group of integers.

The term “cancer specific” refers to a nucleic acid molecule or polypeptide that is differentially expressed predominantly in the breast cancer as compared to other tissues in the body. In a preferred embodiment, a “cancer specific” nucleic acid molecule or polypeptide is detected at a level that is 1.5-fold higher than any other tissue in the body. In a more preferred embodiment, the “cancer specific” nucleic acid molecule or polypeptide is detected at a level that is 1.8-fold higher than any other tissue in the body, more preferably 2-fold higher, still more preferably at least 2.5-fold, 3-fold, 4-fold, 5-fold, 10-fold, 15-fold, 20-fold, 25-fold, 50-fold or 100-fold higher than any other tissue in the body. In another preferred embodiment, a “cancer specific” nucleic acid molecule or polypeptide is detected at a level that is 1.5-fold lower than any other tissue in the body. In a more preferred embodiment, the “cancer specific” nucleic acid molecule or polypeptide is detected at a level that is 1.8-fold lower than any other tissue in the body, more preferably 2-fold lower, still more preferably at least 2.5-fold, 3-fold, 4-fold, 5-fold, 10-fold, 15-fold, 20-fold, 25-fold, 50-fold or 100-fold lower than any other tissue in the body. Nucleic acid molecule levels may be measured by nucleic acid hybridization, such as Northern blot hybridization, microarray analysis or quantitative PCR. Polypeptide levels may be measured by any method known to accurately quantitate protein levels, such as Western blot analysis, ELISA, IHC, protein chip array, mass-spec and flow cytometry.

The term “prognosis” defines a forecast as to the probable outcome of a disease, the prospect as to recovery from a disease, or the potential recurrence of a disease as indicated by the nature and symptoms of the case. In general, prognosis is defined as “good” when there is a probable favorable outcome of a disease, recovery from a disease or low potential for disease recurrence. A “poor” prognosis is generally defined as a non-favorable outcome of a disease, non-recovery from a disease, or greater potential for disease recurrence. Prognosis may be determined using clinical factors, pathological evaluation, genotypic or phenotypic molecular profiling.

### Nucleic Acid Molecules, Regulatory Sequences, Vectors, Host Cells and Recombinant Methods of Making Polypeptides

Nucleic Acid Molecules

One aspect of the invention provides isolated nucleic acid molecules that are specific to cancer or to cancer cells or tissue or that are derived from such nucleic acid molecules. These isolated cancer specific nucleic acids (CaSNAs) may comprise cDNA genomic DNA, RNA, or a combination thereof, a fragment of one of these nucleic acids, or may be a non-naturally occurring nucleic acid molecule. A CaSNA may be derived from an animal. In a preferred embodiment, the CaSNA is derived from a human or other mammal. In a more preferred embodiment, the CaSNA is derived from a human or other primate. In an even more preferred embodiment, the CaSNA is derived from a human.

In a preferred embodiment, the nucleic acid molecule encodes a polypeptide that is specific to cancer, a cancer-specific polypeptide (CaSP). In a more preferred embodiment, the nucleic acid molecule encodes a polypeptide that comprises an amino acid sequence of the gene products of Table 2a or Table 2b. In another highly preferred embodiment, the nucleic acid molecule comprises a nucleic acid sequence of the gene products of Table 2a, Table 2b or Table 7. Nucleotide sequences of the instantly-described nucleic acid molecules were determined by assembling several DNA molecules from either public or proprietary databases. Some of the underlying DNA sequences are the result, directly or indirectly, of at least one enzymatic polymerization reaction (e.g., reverse transcription and/or polymerase chain reaction) using an automated sequencer (such as the MegaBACE™ 1000, Amersham Biosciences, Sunnyvale, Calif., USA).

Nucleic acid molecules of the present invention may also comprise sequences that selectively hybridizes to a nucleic acid molecule encoding a CaSNA or a complement or antisense thereof. The hybridizing nucleic acid molecule may or may not encode a polypeptide or may or may not encode a CaSP. However, in a preferred embodiment, the hybridizing nucleic acid molecule encodes a CaSP. In a more preferred embodiment, the invention provides a nucleic acid molecule that selectively hybridizes to a nucleic acid molecule or the antisense sequence of a nucleic acid molecule that encodes a polypeptide comprising an amino acid sequence of the gene products of Table 2a or Table 2b. In an even more preferred embodiment, the invention provides a nucleic acid molecule that selectively hybridizes to a nucleic acid molecule comprising the nucleic acid sequence of the gene products of Table 2a, Table 2b or Table 7 or the antisense sequence thereof. Preferably, the nucleic acid molecule selectively hybridizes to a nucleic acid molecule or the antisense sequence of a nucleic acid molecule encoding a CaSP under low stringency conditions. More preferably, the nucleic acid molecule selectively hybridizes to a nucleic acid molecule or the antisense sequence of a nucleic acid molecule encoding a CaSP under moderate stringency conditions. Most preferably, the nucleic acid molecule selectively hybridizes to a nucleic acid molecule or the antisense sequence of a nucleic acid molecule encoding a CaSP under high stringency conditions. In a preferred embodiment, the nucleic acid molecule hybridizes under low, moderate or high stringency conditions to a nucleic acid molecule or the antisense sequence of a nucleic acid molecule encoding a polypeptide comprising an amino acid sequence of the gene products of Table 2a or Table 2b. In a more preferred embodiment, the nucleic acid molecule hybridizes under low, moderate or high stringency conditions to a nucleic acid molecule or the antisense sequence of a nucleic acid molecule comprising a nucleic acid sequence selected from the gene products of Table 2a, Table 2b or Table 7.

Nucleic acid molecules of the present invention may also comprise nucleic acid sequences that exhibit substantial sequence similarity to a nucleic acid encoding a CaSP or a complement of the encoding nucleic acid molecule. In this embodiment, it is preferred that the nucleic acid molecule exhibit substantial sequence similarity to a nucleic acid molecule encoding human CaSP. More preferred is a nucleic acid molecule exhibiting substantial sequence similarity to a nucleic acid molecule encoding a polypeptide having an amino acid sequence of the gene products of Table 2a or Table 2b. By substantial sequence similarity it is meant a nucleic acid molecule having at least 60% sequence identity with a nucleic acid molecule encoding a CaSP, such as a polypeptide having an amino acid sequence of the gene products of Table 2a or Table 2b, more preferably at least 70%, even more preferably at least 80% and even more preferably at least 85%. In a more preferred embodiment, the similar nucleic acid molecule is one that has at least 90% sequence identity with a nucleic acid molecule encoding a CaSP, more preferably at least 95%, more preferably at least 97%, even more preferably at least 98%, and still more preferably at least 99%. Most preferred in this embodiment is a nucleic acid molecule that has at least 99.5%, 99.6%, 99.7%, 99.8% or 99.9% sequence identity with a nucleic acid molecule encoding a CaSP.

The nucleic acid molecules of the present invention are also inclusive of those exhibiting substantial sequence similarity to a CaSNA or its complement. In this embodiment, it is preferred that the nucleic acid molecule exhibit substantial sequence similarity to a nucleic acid molecule having a nucleic acid sequence of the gene products of Table 2a, Table 2b or Table 7. By substantial sequence similarity it is meant a nucleic acid molecule that has at least 60% sequence identity with a CaSNA, such as one having a nucleic acid sequence of the gene products of Table 2a, Table 2b or Table 7, more preferably at least 70%, even more preferably at least 80% and even more preferably at least 85%. More preferred is a nucleic acid molecule that has at least 90% sequence identity with a CaSNA, more preferably at least 95%, more preferably at least 97%, even more preferably at least 98%, and still more preferably at least 99%. Most preferred is a nucleic acid molecule that has at least 99.5%, 99.6%, 99.7%, 99.8% or 99.9% sequence identity with a CaSNA.

Nucleic acid molecules that exhibit substantial sequence similarity are inclusive of sequences that exhibit sequence identity over their entire length to a CaSNA or to a nucleic acid molecule encoding a CaSP, as well as sequences that are similar over only a part of its length. In this case, the part is at least 50 nucleotides of the CaSNA or the nucleic acid molecule encoding a CaSP, preferably at least 100 nucleotides, more preferably at least 150 or 200 nucleotides, even more preferably at least 250 or 300 nucleotides, still more preferably at least 400 or 500 nucleotides.

The substantially similar nucleic acid molecule may be a naturally occurring one that is derived from another species, especially one derived from another primate, wherein the similar nucleic acid molecule encodes an amino acid sequence that exhibits significant sequence identity to that of the gene products of Table 2a or Table 2b or demonstrates significant sequence identity to the nucleotide sequence of the gene products of Table 2a, Table 2b or Table 7. The similar nucleic acid molecule may also be a naturally occurring nucleic acid molecule from a human, when the CaSNA is a member of a gene family. The similar nucleic acid molecule may also be a naturally occurring nucleic acid molecule derived from a non-primate, mammalian species, including without limitation, domesticated species, e.g., dog, cat, mouse, rat, rabbit, hamster, cow, horse and pig; and wild animals, e.g., monkey, fox, lions, tigers, bears, giraffes, zebras, etc. The substantially similar nucleic acid molecule may also be a naturally occurring nucleic acid molecule derived from a non-mammalian species, such as birds or reptiles. The naturally occurring substantially similar nucleic acid molecule may be isolated directly from humans or other species. In another embodiment, the substantially similar nucleic acid molecule may be one that is experimentally produced by random mutation of a nucleic acid molecule. In another embodiment, the substantially similar nucleic acid molecule may be one that is experimentally produced by directed mutation of a CaSNA. In a preferred embodiment, the substantially similar nucleic acid molecule is an CaSNA.

The nucleic acid molecules of the present invention are also inclusive of allelic variants of a CaSNA or a nucleic acid encoding a CaSP. For example, single nucleotide polymorphisms (SNPs) occur frequently in eukaryotic genomes and the sequence determined from one individual of a species may differ from other allelic forms present within the population. More than 1.4 million SNPs have already identified in the human genome, International Human Genome Sequencing Consortium, Nature 409: 860-921 (2001). Variants with small deletions and insertions of more than a single nucleotide are also found in the general population, and often do not alter the function of the protein. In addition, amino acid substitutions occur frequently among natural allelic variants, and often do not substantially change protein function.

In a preferred embodiment, the allelic variant is a variant of a gene, wherein the gene is transcribed into an RNA molecule. In a more preferred embodiment, the RNA molecule is an mRNA that encodes a CaSP. In a more preferred embodiment, the gene is transcribed into an mRNA that encodes a CaSP comprising an amino acid sequence of the gene products of Table 2a or Table 2b. In another preferred embodiment, the allelic variant is a variant of a gene, wherein the gene is transcribed into an mRNA that is a CaSNA. In a more preferred embodiment, the gene is transcribed into an mRNA that comprises the nucleic acid sequence of the gene products of Table 2a, Table 2b or Table 7. Also preferred is that the allelic variant is a naturally occurring allelic variant in the species of interest, particularly human.

Nucleic acid molecules of the present invention are also inclusive of nucleic acid sequences comprising a part of a nucleic acid sequence of the instant invention. The part may or may not encode a polypeptide, and may or may not encode a polypeptide that is a CaSP. In a preferred embodiment, the part encodes a CaSP. In one embodiment, the nucleic acid molecule comprises a part of a CaSNA. In another embodiment, the nucleic acid molecule comprises a part of a nucleic acid molecule that hybridizes or exhibits substantial sequence similarity to a CaSNA. In another embodiment, the nucleic acid molecule comprises a part of a nucleic acid molecule that is an allelic variant of a CaSNA. In another embodiment, the nucleic acid molecule comprises a part of a nucleic acid molecule that bridges an exon-exon junction of a CaSNA. In yet another embodiment, the nucleic acid molecule comprises a part of a nucleic acid molecule that encodes a CaSP. A part comprises at least 10 nucleotides, more preferably at least 15, 17, 18, 20, 25, 30, 35, 40, 50, 60, 70, 80, 90, 100, 150, 200, 250, 300, 350, 400 or 500 nucleotides. The maximum size of a nucleic acid part is one nucleotide shorter than the sequence of the nucleic acid molecule encoding the full-length protein.

Nucleic acid molecules of the present invention are also inclusive of nucleic acid sequences that encode fusion proteins, homologous proteins, polypeptide fragments, muteins and polypeptide analogs, as described infra.

Nucleic acid molecules of the present invention are also inclusive of nucleic acid sequences containing modifications of the native nucleic acid molecule. Examples of such modifications include, but are not limited to, normative internucleoside bonds, post-synthetic modifications or altered nucleotide analogues. One having ordinary skill in the art would recognize that the type of modification that may be made will depend upon the intended use of the nucleic acid molecule. For instance, when the nucleic acid molecule is used as a hybridization probe, the range of such modifications will be limited to those that permit sequence-discriminating base pairing of the resulting nucleic acid. When used to direct expression of RNA or protein in vitro or in vivo, the range of such modifications will be limited to those that permit the nucleic acid to function properly as a polymerization substrate. When the isolated nucleic acid is used as a therapeutic agent, the modifications will be limited to those that do not confer toxicity upon the isolated nucleic acid.

Accordingly, in one embodiment, a nucleic acid molecule may include nucleotide analogues that incorporate labels that are directly detectable, such as radiolabels or fluorophores, or nucleotide analogues that incorporate labels that can be visualized in a subsequent reaction, such as biotin or various haptens. The labeled nucleic acid molecules are particularly useful as hybridization probes.

Common radiolabeled analogues include those labeled with ³³P, ³²P, and ³⁵S, such as α-³²P-dATP, α-³²P-dCTP, α-³²P-dGTP, α-³²P-dTTP, α-³²P-3′dATP, α-³²P-ATP, α-³²P-CTP, α-³²P-GTP, α-³²P-UTP, α-³⁵S-dATP, γ-³⁵S-GTP, γ-³³P-dATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Biosciences, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor®1546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA). One may also custom synthesize nucleotides having other fluorophores. See Henegariu et al, Nature Biotechnol. 18: 345-348 (2000).

Haptens that are commonly conjugated to nucleotides for subsequent labeling include biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

Nucleic acid molecules of the present invention can be labeled by incorporation of labeled nucleotide analogues into the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach. Analogues can also be incorporated during automated solid phase chemical synthesis. Labels can also be incorporated after nucleic acid synthesis, with the 5′ phosphate and 3′ hydroxyl providing convenient sites for post-synthetic covalent attachment of detectable labels.

Other post-synthetic approaches also permit internal labeling of nucleic acids. For example, fluorophores can be attached using a cisplatin reagent that reacts with the N7 of guanine residues (and, to a lesser extent, adenine bases) in DNA, RNA, and Peptide Nucleic Acids (PNA) to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer 25: 301-305 (1999); Jelsma et al., J. NIH Res. 5: 82 (1994); Van Belkum et al, BioTechniques 16: 148-153 (1994). Alternatively, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

One or more independent or interacting labels can be incorporated into the nucleic acid molecules of the present invention. For example, both a fluorophore and a moiety that in proximity thereto acts to quench fluorescence can be included to report specific hybridization through release of fluorescence quenching or to report exonucleotidic excision. See, e.g., Tyagi et al, Nature Biotechnol. 14: 303-308 (1996); Tyagi et al., Nature Biotechnol. 16: 49-53 (1998); Sokol et al., Proc. Natl. Acad. Sci. USA 95: 11538-11543 (1998); Kostrikis et al., Science 279: 1228-1229 (1998); Marras et al., Genet. Anal. 14: 151-156 (1999); Holland et al., Proc. Natl. Acad. Sci. USA 88: 7276-7280 (1991); Heid et al., Genome Res. 6(10): 986-94 (1996); Kuimelis et al., Nucleic Acids Symp. Ser. (37): 255-6 (1997); and U.S. Pat. Nos. 5,846,726, 5,925,517, 5,925,517, 5,723,591 and 5,538,848, the disclosures of which are incorporated herein by reference in their entireties.

Nucleic acid molecules of the present invention may also be modified by altering one or more native phosphodiester internucleoside bonds to more nuclease-resistant, internucleoside bonds. See Hartmann et al. (eds.), Manual of Antisense Methodology: Perspectives in Antisense Science, Kluwer Law International (1999); Stein et al. (eds.), Applied Antisense Oligonucleotide Technology, Wiley-Liss (1998); Chadwick et al (eds.), Oligonucleotides as Therapeutic Agents—Symposium No. 209, John Wiley & Son Ltd (1997). Such altered internucleoside bonds are often desired for techniques or for targeted gene correction, Gamper et al., Nucl. Acids Res. 28(21): 4332-4339 (2000). For double stranded RNA inhibition which may utilize either natural ds RNA or ds RNA modified in its, sugar, phosphate or base, see Hannon, Nature 418(11): 244-251 (2002); Fire et al. in WO 99/32619; Tuschl et al. in US2002/0086356; Kruetzer et al. in WO 00/44895, the disclosures of which are incorporated herein by reference in their entirety. For circular antisense, see Kool in U.S. Pat. No. 5,426,180, the disclosure of which is incorporated herein by reference in its entirety.

Modified oligonucleotide backbones include, without limitation, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties. In a preferred embodiment, the modified internucleoside linkages may be used for antisense techniques.

Other modified oligonucleotide backbones do not include a phosphorus atom, but have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437 and 5,677,439; the disclosures of which are incorporated herein by reference in their entireties.

In other preferred nucleic acid molecules, both the sugar and the internucleoside linkage are replaced with novel groups, such as peptide nucleic acids (PNA). In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages. PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference in its entirety. Automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.). PNA molecules are advantageous for a number of reasons. First, because the PNA backbone is uncharged, PNA/DNA and PNA/RNA duplexes have a higher thermal stability than is found in DNA/DNA and DNA/RNA duplexes. The Tm of a PNA/DNA or PNA/RNA duplex is generally 1° C. higher per base pair than the Tm of the corresponding DNA/DNA or DNA/RNA duplex (in 100 mM NaCl). Second, PNA molecules can also form stable PNA/DNA complexes at low ionic strength, under conditions in which DNA/DNA duplex formation does not occur. Third, PNA also demonstrates greater specificity in binding to complementary DNA because a PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater. Fourth, PNA oligomers are resistant to degradation by enzymes, and the lifetime of these compounds is extended both in vivo and in vitro because nucleases and proteases do not recognize the PNA polyamide backbone with nucleobase sidechains. See, e.g., Ray et al., FASEB J. 14(9): 1041-60 (2000); Nielsen et al, Pharmacol Toxicol. 86(1): 3-7 (2000); Larsen et al., Biochim Biophys Acta. 1489(1): 159-66 (1999); Nielsen, Curr. Opin. Struct. Biol. 9(3): 353-7 (1999), and Nielsen, Curr. Opin. Biotechnol. 10(1): 71-5 (1999).

Nucleic acid molecules may be modified compared to their native structure throughout the length of the nucleic acid molecule or can be localized to discrete portions thereof. As an example of the latter, chimeric nucleic acids can be synthesized that have discrete DNA and RNA domains and that can be used for targeted gene repair and modified PCR reactions, as further described in, Misra et al., Biochem. 37: 1917-1925 (1998); and Finn et al., Nucl. Acids Res. 24: 3357-3363 (1996), and U.S. Pat. Nos. 5,760,012 and 5,731,181, the disclosures of which are incorporated herein by reference in their entireties.

Unless otherwise specified, nucleic acid molecules of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Banér et al, Curr. Opin. Biotechnol. 12: 11-15 (2001); Escude et al., Proc. Natl. Acad. Sci. USA 14: 96(19):10603-7 (1999); and Nilsson et al., Science 265(5181): 2085-8 (1994). Triplex and quadruplex conformations, and their utilities, are reviewed in Praseuth et al., Biochim. Biophys. Acta. 1489(1): 181-206 (1999); Fox, Curr. Med. Chem. 7(1): 17-37 (2000); Kochetkova et al, Methods Mol. Biol. 130: 189-201 (2000); Chan et al., J. Mol. Med. 75(4): 267-82 (1997); Rowley et al., Mol Med 5(10): 693-700 (1999); Kool, Annu Rev Biophys Biomol Struct. 25: 1-28 (1996).

SNP Polymoiphisms

Commonly, sequence differences between individuals involve differences in single nucleotide positions. SNPs may account for 90% of human DNA polymorphism. Collins et al., 8 Genome Res. 1229-31 (1998). SNPs include single base pair positions in genomic DNA at which different sequence alternatives (alleles) exist in a population. In addition, the least frequent allele generally must occur at a frequency of 1% or greater. DNA sequence variants with a reasonably high population frequency are observed approximately every 1,000 nucleotide across the genome, with estimates as high as 1 SNP per 350 base pairs. Wang et al., 280 Science 1077-82 (1998); Harding et al, 60 μm. J. Human Genet. 772-89 (1997); Taillon-Miller et al., Genome Res. 8:748-54 (1998); Cargill et al., Nat. Genet. 22:231-38 (1999); and Semple et al., Bioinform. Disc. Note 16:735-38 (2000). The frequency of SNPs varies with the type and location of the change. In base substitutions, two-thirds of the substitutions involve the C-T and G-A type. This variation in frequency can be related to 5-methylcytosine deamination reactions that occur frequently, particularly at CpG dinucleotides. Regarding location, SNPs occur at a much higher frequency in non-coding regions than in coding regions. Information on over one million variable sequences is already publicly available via the Internet and more such markers are available from commercial providers of genetic information. Kwok and Gu, Med. Today 5:538-53 (1999).

Several definitions of SNPs exist. See, e.g., Brooks, 235 Gene 177-86 (1999). As used herein, the term “single nucleotide polymorphism” or “SNP” includes all single base variants, thus including nucleotide insertions and deletions in addition to single nucleotide substitutions. There are two types of nucleotide substitutions. A transition is the replacement of one purine by another purine or one pyrimidine by another pyrimidine. A transversion is the replacement of a purine for a pyrimidine, or vice versa.

Numerous methods exist for detecting SNPs within a nucleotide sequence. A review of many of these methods can be found in Landegren et al., 8 Genome Res. 769-76 (1998). For example, a SNP in a genomic sample can be detected by preparing a Reduced Complexity Genome (RCG) from the genomic sample, then analyzing the RCG for the presence or absence of a SNP. See, e.g., WO 00/18960. Multiple SNPs in a population of target polynucleotides in parallel can be detected using, for example, the methods of WO 00/50869. Other SNP detection methods include the methods of U.S. Pat. Nos. 6,297,018 and 6,322,980. Furthermore, SNPs can be detected by restriction fragment length polymorphism (RFLP) analysis. See, e.g., U.S. Pat. Nos. 5,324,631; 5,645,995. RFLP analysis of SNPs, however, is limited to cases where the SNP either creates or destroys a restriction enzyme cleavage site. SNPs can also be detected by direct sequencing of the nucleotide sequence of interest. In addition, numerous assays based on hybridization have also been developed to detect SNPs and mismatch distinction by polymerases and ligases. Several web sites provide information about SNPs including Ensembl (ensembl with the extension org of the world wide web), Sanger Institute (sanger with the extension .ac.uk/genetics/exon/of the world wide web), National Center for Biotechnology Information (NCBI) (ncbi with the extension .nlm.nih.gov/SNP/of the world wide web), The SNP Consortium Ltd. (snp with the extension .cshl.org/of the world wide web). The chromosomal locations for the compositions disclosed herein are provided below. In addition, one of ordinary skill in the art could perform a search against the genome or any of the databases cited above using BLAST to find the chromosomal location or locations of SNPs. Another preferred method to find the genomic coordinates and associated SNPs would be to use the BLAT tool (genome.ucsc.edu, Kent et al. 2001, The Human Genome Browser at UCSC, Genome Research 996-1006 or Kent 2002 BLAT, The BLAST-Like Alignment Tool Genome Research, 1-9). All web sites above were accessed Dec. 3, 2003.

RNA Interference

RNA interference refers to the process of sequence-specific transcriptional or post transcriptional gene silencing in animals mediated by various RNAi species including short interfering RNAs (siRNA), microRNAs (miRNA), tiny non-coding RNAs (tncRNA) and small modulatory RNA (smRNA). Fire et al., Nature 391:806 (1998) and Novina et al., Nature 430:161-164(2004). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla. Fire et al., Trends Genet. 15: 358 (1999). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA). Berstein et al., Nature 409: 363 (2001). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control. Hutvagner et al., Science 293: 834 (2001). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex. Elbashir et al., Genes Dev. 15: 188 (2001); Novina, 2004 supra.

Short interfering RNA mediated RNAi has been studied in a variety of systems. Fire et al., Nature, 391: 806 (1998), were the first to observe RNAi in C. Elegans. Wianny and Goetz, Nature Cell Biol. 2: 70 (1999), describe RNAi mediated by dsRNA in mouse embryos. Hammond et al., Nature 404: 293 (2000), describe RNAi in Drosophila cells transfected with dsRNA. Elbashir et al., Nature 411: 494 (2001), describe RNAi induced by introduction of duplexes of synthetic 21-nucleotide RNAs in cultured mammalian cells including human embryonic kidney and HeLa cells. Recent work in Drosophila embryonic lysates (Elbashir et al., EMBO J. 20: 6877 (2001) has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end. Elbashir et al., EMBO J. 20:6877 (2001). Other studies have indicated that a 5′-phosphate on the target-complementary strand of a siRNA duplex is required for siRNA activity and that ATP is utilized to maintain the 5′-phosphate moiety on the siRNA. Nykanen et al, Cell 107: 309 (2001).

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity. Elbashir et al., EMBO J. 20: 6877 (2001). In addition, Elbashir et al., supra, also report that substitution of siRNA with 2′-O-methyl nucleotides completely abolishes RNAi activity. Li et al., WO 00/44914, and Beach et al., WO 01/68836 both suggest that siRNA “may include modifications to either the phosphate-sugar back bone or the nucleoside to include at least one of a nitrogen or sulfur heteroatom”, however neither application teaches to what extent these modifications are tolerated in siRNA molecules nor provide any examples of such modified siRNA. Kreutzer and Limmer, Canadian Patent Application No. 2,359,180, also describe certain chemical modifications for use in dsRNA constructs in order to counteract activation of double stranded-RNA-dependent protein kinase PKR, specifically 2′-amino or 2′-O-methyl nucleotides, and nucleotides containing a 2′-O or 4′-C methylene bridge. However, Kreutzer and Limmer similarly fail to show to what extent these modifications are tolerated in siRNA molecules nor do they provide any examples of such modified siRNA.

Parrish et al., Molecular Cell 6:1977-1087 (2000), tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that “RNAs with two [phosphorothioate] modified bases also had substantial decreases in effectiveness as RNAi triggers; [phosphorothioate] modification of more than two residues greatly destabilized the RNAs in vitro and we were not able to assay interference activities.” Id. at 1081. The authors also tested certain modifications at the 2′-position of the nucleotide sugar in the long siRNA transcripts and observed that substituting deoxynucleotides for ribonucleotides “produced a substantial decrease in interference activity”, especially in the case of Uridine to Thymidine and/or Cytidine to deoxy-Cytidine substitutions. Id. In addition, the authors tested certain base modifications, including substituting 4-thiouracil, 5-bromouracil, 5-iodouracil, 3-(aminoallyl)uracil for uracil, and inosine for guanosine in sense and antisense strands of the siRNA, and found that whereas 4-thiouracil and 5-bromouracil were all well tolerated, inosine “produced a substantial decrease in interference activity” when incorporated in either strand. Incorporation of 5-iodouracil and 3-(aminoallyl)uracil in the antisense strand resulted in substantial decrease in RNAi activity as well.

Beach et al., WO 01/68836, describes specific methods for attenuating gene expression using endogenously derived dsRNA. Tuschl et al., WO 01/75164, describes a Drosophila in vitro RNAi system and the use of specific siRNA molecules for certain functional genomic and certain therapeutic applications; although Tuschl, Chem. Biochem. 2:239-245 (2001), doubts that RNAi can be used to cure genetic diseases or viral infection due “to the danger of activating interferon response”. Li et al., WO 00/44914, describes the use of specific dsRNAs for use in attenuating the expression of certain target genes. Zernicka-Goetz et al., WO 01/36646, describes certain methods for inhibiting the expression of particular genes in mammalian cells using certain dsRNA molecules. Fire et al., WO 99/32619, U.S. Pat. No. 6,506,559, the contents of which are hereby incorporated by reference, describes particular methods for introducing certain dsRNA molecules into cells for use in inhibiting gene expression. Plaetinck et al., WO 00/01846, describes certain methods for identifying specific genes responsible for conferring a particular phenotype in a cell using specific dsRNA molecules. Mello et al., WO 01/29058, describes the identification of specific genes involved in dsRNA mediated RNAi. Deschamps Depaillette et al., International PCT Publication No. WO 99/07409, describes specific compositions consisting of particular dsRNA molecules combined with certain anti-viral agents. Driscoll et al., International PCT Publication No. WO 01/49844, describes specific DNA constructs for use in facilitating gene silencing in targeted organisms. Parrish et al., Molecular Cell 6: 1977-1087 (2000), describes specific chemically modified siRNA constructs targeting the unc-22 gene of C. elegans. Tuschl et al., International PCT Publication No. WO 02/44321, describe certain synthetic siRNA constructs.

Methods for Using Nucleic Acid Molecules as Probes and Primers

The isolated nucleic acid molecules of the present invention can be used as hybridization probes to detect, characterize, and quantify hybridizing nucleic acids in, and isolate hybridizing nucleic acids from, both genomic and transcript-derived nucleic acid samples. When free in solution, such probes are typically, but not invariably, detectably labeled; bound to a substrate, as in a microarray, such probes are typically, but not invariably unlabeled.

In one embodiment, the isolated nucleic acid molecules of the present invention can be used as probes to detect and characterize gross alterations in the gene of a CaSNA, such as deletions, insertions, translocations, and duplications of the CaSNA genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999). The isolated nucleic acid molecules of the present invention can be used as probes to assess smaller genomic alterations using, e.g., Southern blot detection of restriction fragment length polymorphisms. The isolated nucleic acid molecules of the present invention can be used as probes to isolate genomic clones that include a nucleic acid molecule of the present invention, which thereafter can be restriction mapped and sequenced to identify deletions, insertions, translocations, and substitutions (single nucleotide polymorphisms, SNPs) at the sequence level. Alternatively, detection techniques such as molecular beacons may be used, see Kostrikis et al., Science 279:1228-1229 (1998).

The isolated nucleic acid molecules of the present invention can be also be used as probes to detect, characterize, and quantify CaSNA in, and isolate CaSNA from, transcript-derived nucleic acid samples. In one embodiment, the isolated nucleic acid molecules of the present invention can be used as hybridization probes to detect, characterize by length, and quantify mRNA by Northern blot of total or poly-A⁺-selected RNA samples. In another embodiment, the isolated nucleic acid molecules of the present invention can be used as hybridization probes to detect, characterize by location, and quantify mRNA by in situ hybridization to tissue sections. See, e.g., Schwarchzacher et al, In Situ Hybridization, Springer-Verlag New York (2000). In another preferred embodiment, the isolated nucleic acid molecules of the present invention can be used as hybridization probes to measure the representation of clones in a cDNA library or to isolate hybridizing nucleic acid molecules acids from cDNA libraries, permitting sequence level characterization of mRNAs that hybridize to CaSNAs, including, without limitations, identification of deletions, insertions, substitutions, truncations, alternatively spliced forms and single nucleotide polymorphisms. In yet another preferred embodiment, the nucleic acid molecules of the instant invention may be used in microarrays.

All of the aforementioned probe techniques are well within the skill in the art, and are described at greater length in standard texts such as Sambrook (2001), supra; Ausubel (1999), supra; and Walker et al (eds.), The Nucleic Acids Protocols Handbook, Humana Press (2000).

In another embodiment, a nucleic acid molecule of the invention may be used as a probe or primer to identify and/or amplify a second nucleic acid molecule that selectively hybridizes to the nucleic acid molecule of the invention. In this embodiment, it is preferred that the probe or primer be derived from a nucleic acid molecule encoding a CaSP. More preferably, the probe or primer is derived from a nucleic acid molecule encoding a polypeptide having an amino acid sequence of the gene products of Table 2a or Table 2b. Also preferred are probes or primers derived from a CaSNA. More preferred are probes or primers derived from a nucleic acid molecule having a nucleotide sequence of the gene products of Table 2a, Table 2b or Table 7.

In general, a probe or primer is at least 10 nucleotides in length, more preferably at least 12, more preferably at least 14 and even more preferably at least 16 or 17 nucleotides in length. In an even more preferred embodiment, the probe or primer is at least 18 nucleotides in length, even more preferably at least 20 nucleotides and even more preferably at least 22 nucleotides in length. Primers and probes may also be longer in length. For instance, a probe or primer may be 25 nucleotides in length, or may be 30, 40 or 50 nucleotides in length. Methods of performing nucleic acid hybridization using oligonucleotide probes are well known in the art. See, e.g., Sambrook et al., 1989, supra, Chapter 11 and pp. 11.31-11.32 and 11.40-11.44, which describes radiolabeling of short probes, and pp. 11.45-11.53, which describe hybridization conditions for oligonucleotide probes, including specific conditions for probe hybridization (pp. 11.50-11.51).

Methods of performing primer-directed amplification are also well known in the art. Methods for performing the polymerase chain reaction (PCR) are compiled, inter alia, in McPherson, PCR Basics: From Background to Bench, Springer Verlag (2000); Innis et al. (eds.), PCR Applications: Protocols for Functional Genomics, Academic Press (1999); Gelfand et al. (eds.), PCR Strategies, Academic Press (1998); Newton et al., PCR, Springer-Verlag New York (1997); Burke (ed.), PCR: Essential Techniques, John Wiley & Son Ltd (1996); White (ed.), PCR Cloning Protocols: From Molecular Cloning to Genetic Engineering, Vol. 67, Humana Press (1996); and McPherson et al. (eds.), PCR 2: A Practical Approach, Oxford University Press, Inc. (1995). Methods for performing RT-PCR are collected, e.g., in Siebert et al. (eds.), Gene Cloning and Analysis by RT-PCR, Eaton Publishing Company/Bio Techniques Books Division, 1998; and Siebert (ed.), PCR Technique:RT-PCR, Eaton Publishing Company/BioTechniques Books (1995).

PCR and hybridization methods may be used to identify and/or isolate nucleic acid molecules of the present invention including allelic variants, homologous nucleic acid molecules and fragments. PCR and hybridization methods may also be used to identify, amplify and/or isolate nucleic acid molecules of the present invention that encode homologous proteins, analogs, fusion protein or muteins of the invention. Nucleic acid primers as described herein can be used to prime amplification of nucleic acid molecules of the invention, using transcript-derived or genomic DNA as template.

These nucleic acid primers can also be used, for example, to prime single base extension (SBE) for SNP detection (See, e.g., U.S. Pat. No. 6,004,744, the disclosure of which is incorporated herein by reference in its entirety).

Isothermal amplification approaches, such as rolling circle amplification, are also now well-described. See, e.g., Schweitzer et al, Curr. Opin. Biotechnol. 12(1): 21-7 (2001); international patent publications WO 97/19193 and WO 00/15779, and U.S. Pat. Nos. 5,854,033 and 5,714,320, the disclosures of which are incorporated herein by reference in their entireties. Rolling circle amplification can be combined with other techniques to facilitate SNP detection. See, e.g., Lizardi et al, Nature Genet. 19(3): 225-32 (1998).

Nucleic acid molecules of the present invention may be bound to a substrate either covalently or noncovalently. The substrate can be porous or solid, planar or non-planar, unitary or distributed. The bound nucleic acid molecules may be used as hybridization probes, and may be labeled or unlabeled. In a preferred embodiment, the bound nucleic acid molecules are unlabeled.

In one embodiment, the nucleic acid molecule of the present invention is bound to a porous substrate, e.g., a membrane, typically comprising nitrocellulose, nylon, or positively charged derivatized nylon. The nucleic acid molecule of the present invention can be used to detect a hybridizing nucleic acid molecule that is present within a labeled nucleic acid sample, e.g., a sample of transcript-derived nucleic acids. In another embodiment, the nucleic acid molecule is bound to a solid substrate, including, without limitation, glass, amorphous silicon, crystalline silicon or plastics. Examples of plastics include, without limitation, polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof. The solid substrate may be any shape, including rectangular, disk-like and spherical. In a preferred embodiment, the solid substrate is a microscope slide or slide-shaped substrate.

The nucleic acid molecule of the present invention can be attached covalently to a surface of the support substrate or applied to a derivatized surface in a chaotropic agent that facilitates denaturation and adherence by presumed noncovalent interactions, or some combination thereof. The nucleic acid molecule of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that comprise one or more of the nucleic acid molecules of the present invention.

In yet another embodiment, the invention is directed to single exon probes based on the CaSNAs disclosed herein.

Expression Vectors, Host Cells and Recombinant Methods of Producing Polypeptides

Another aspect of the present invention provides vectors that comprise one or more of the isolated nucleic acid molecules of the present invention, and host cells in which such vectors have been introduced.

The vectors can be used, inter alia, for propagating the nucleic acid molecules of the present invention in host cells (cloning vectors), for shuttling the nucleic acid molecules of the present invention between host cells derived from disparate organisms (shuttle vectors), for inserting the nucleic acid molecules of the present invention into host cell chromosomes (insertion vectors), for expressing sense or antisense RNA transcripts of the nucleic acid molecules of the present invention in vitro or within a host cell, and for expressing polypeptides encoded by the nucleic acid molecules of the present invention, alone or as fusion proteins with heterologous polypeptides (expression vectors). Vectors are by now well known in the art, and are described, inter alia, in Jones et al. (eds.), Vectors: Cloning Applications: Essential Techniques (Essential Techniques Series), John Wiley & Son Ltd. (1998); Jones et al. (eds.), Vectors: Expression Systems: Essential Techniques (Essential Techniques Series), John Wiley & Son Ltd. (1998); Gacesa et al, Vectors: Essential Data, John Wiley & Sons Ltd. (1995); Cid-Arregui (eds.), Viral Vectors: Basic Science and Gene Therapy, Eaton Publishing Co. (2000); Sambrook (2001), supra; Ausubel (1999), supra. Furthermore, a variety of vectors are available commercially. Use of existing vectors and modifications thereof are well within the skill in the art. Thus, only basic features need be described here.

Nucleic acid sequences may be expressed by operatively linking them to an expression control sequence in an appropriate expression vector and employing that expression vector to transform an appropriate unicellular host. Expression control sequences are sequences that control the transcription, post-transcriptional events and translation of nucleic acid sequences. Such operative linking of a nucleic sequence of this invention to an expression control sequence, of course, includes, if not already part of the nucleic acid sequence, the provision of a translation initiation codon, ATG or GTG, in the correct reading frame upstream of the nucleic acid sequence.

A wide variety of host/expression vector combinations may be employed in expressing the nucleic acid sequences of this invention. Useful expression vectors, for example, may consist of segments of chromosomal, non-chromosomal and synthetic nucleic acid sequences.

In one embodiment, prokaryotic cells may be used with an appropriate vector. Prokaryotic host cells are often used for cloning and expression. In a preferred embodiment, prokaryotic host cells include E. coli, Pseudomonas, Bacillus and Streptomyces. In a preferred embodiment, bacterial host cells are used to express the nucleic acid molecules of the instant invention. Useful expression vectors for bacterial hosts include bacterial plasmids, such as those from E. coli, Bacillus or Streptomyces, including pBluescript, pGEX-2T, pUC vectors, col E1, pCR1, pBR322, pMB9 and their derivatives, wider host range plasmids, such as RP4, phage DNAs, e.g., the numerous derivatives of phage lambda, e.g., NM989, λGT10 and λGT11, and other phages, e.g., M13 and filamentous single stranded phage DNA. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin and zeocin; auxotrophic markers can also be used.

In other embodiments, eukaryotic host cells, such as yeast, insect, mammalian or plant cells, may be used. Yeast cells, typically S. cerevisiae, are useful for eukaryotic genetic studies, due to the ease of targeting genetic changes by homologous recombination and the ability to easily complement genetic defects using recombinantly expressed proteins. Yeast cells are useful for identifying interacting protein components, e.g. through use of a two-hybrid system. In a preferred embodiment, yeast cells are useful for protein expression. Vectors of the present invention for use in yeast will typically, but not invariably, contain an origin of replication suitable for use in yeast and a selectable marker that is functional in yeast. Yeast vectors include Yeast Integrating plasmids (e.g., YIp5) and Yeast Replicating plasmids (the YRp and YEp series plasmids), Yeast Centromere plasmids (the YCp series plasmids), Yeast Artificial Chromosomes (YACs) which are based on yeast linear plasmids, denoted YLp, pGPD-2, 2μ plasmids and derivatives thereof, and improved shuttle vectors such as those described in Gietz et al., Gene, 74: 527-34 (1988) (YIplac, YEplac and YCplac). Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trpl-D1 and lys2-201.

Insect cells may be chosen for high efficiency protein expression. Where the host cells are from Spodoptera frugiperda, e.g., Sf9 and Sf21 cell lines, and expresSF™ cells (Protein Sciences Corp., Meriden, Conn., USA), the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following co-transfection with AcMNPV DNA, a homologous recombination event occurs between these sequences resulting in a recombinant virus carrying the gene of interest and the polyhedrin or p10 promoter. Selection can be based upon visual screening for lacZ fusion activity.

The host cells may also be mammalian cells, which are particularly useful for expression of proteins intended as pharmaceutical agents, and for screening of potential agonists and antagonists of a protein or a physiological pathway. Mammalian vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy. Selectable markers for use in mammalian cells include, include but are not limited to, resistance to neomycin (G418), blasticidin, hygromycin and zeocin, and selection based upon the purine salvage pathway using HAT medium. Expression in mammalian cells can be achieved using a variety of plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses). Useful vectors for insect cells include baculoviral vectors and pVL 941.

Plant cells can also be used for expression, with the vector replicon typically derived from a plant virus (e.g., cauliflower mosaic virus, CaMV; tobacco mosaic virus, TMV) and selectable markers chosen for suitability in plants.

It is known that codon usage of different host cells may be different. For example, a plant cell and a human cell may exhibit a difference in codon preference for encoding a particular amino acid. As a result, human mRNA may not be efficiently translated in a plant, bacteria or insect host cell. Therefore, another embodiment of this invention is directed to codon optimization. The codons of the nucleic acid molecules of the invention may be modified to resemble, as much as possible, genes naturally contained within the host cell without altering the amino acid sequence encoded by the nucleic acid molecule.

Any of a wide variety of expression control sequences may be used in these vectors to express the nucleic acid molecules of this invention. Such useful expression control sequences include the expression control sequences associated with structural genes of the foregoing expression vectors. Expression control sequences that control transcription include, e.g., promoters, enhancers and transcription termination sites. Expression control sequences in eukaryotic cells that control post-transcriptional events include splice donor and acceptor sites and sequences that modify the half-life of the transcribed RNA, e.g., sequences that direct poly(A) addition or binding sites for RNA-binding proteins. Expression control sequences that control translation include ribosome binding sites, sequences which direct targeted expression of the polypeptide to or within particular cellular compartments, and sequences in the 5′ and 3′ untranslated regions that modify the rate or efficiency of translation.

Examples of useful expression control sequences for a prokaryote, e.g., E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), the TAC or TRC system, the major operator and promoter regions of phage lambda, the control regions of fd coat protein, and the araBAD operon. Prokaryotic expression vectors may further include transcription terminators, such as the aspA terminator, and elements that facilitate translation, such as a consensus ribosome binding site and translation termination codon, Schomer et al., Proc. Natl. Acad. Sci. USA 83: 8506-8510 (1986).

Expression control sequences for yeast cells, typically S. cerevisiae, will include a yeast promoter, such as the CYC1 promoter, the GAL1 promoter, the GAL10 promoter, ADH1 promoter, the promoters of the yeast α-mating system, or the GPD promoter, and will typically have elements that facilitate transcription termination, such as the transcription termination signals from the CYC1 or ADH1 gene.

Expression vectors useful for expressing proteins in mammalian cells will include a promoter active in mammalian cells. These promoters include, but are not limited to, those derived from mammalian viruses, such as the enhancer-promoter sequences from the immediate early gene of the human cytomegalovirus (CMV), the enhancer-promoter sequences from the Rous sarcoma virus long terminal repeat (RSV LTR), the enhancer-promoter from SV40 and the early and late promoters of adenovirus. Other expression control sequences include the promoter for 3-phosphoglycerate kinase or other glycolytic enzymes, the promoters of acid phosphatase. Other expression control sequences include those from the gene comprising the CaSNA of interest. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Preferred nucleic acid vectors also include a selectable or amplifiable marker gene and means for amplifying the copy number of the gene of interest. Such marker genes are well known in the art. Nucleic acid vectors may also comprise stabilizing sequences (e.g., ori- or ARS-like sequences and telomere-like sequences), or may alternatively be designed to favor directed or non-directed integration into the host cell genome. In a preferred embodiment, nucleic acid sequences of this invention are inserted in frame into an expression vector that allows a high level expression of an RNA which encodes a protein comprising the encoded nucleic acid sequence of interest. Nucleic acid cloning and sequencing methods are well known to those of skill in the art and are described in an assortment of laboratory manuals, including Sambrook (1989), supra, Sambrook (2000), supra; and Ausubel (1992), supra, Ausubel (1999), supra. Product information from manufacturers of biological, chemical and immunological reagents also provide useful information.

Expression vectors may be either constitutive or inducible. Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PLtetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline. Vectors may also be inducible because they contain hormone response elements, such as the glucocorticoid response element (GRE) and the estrogen response element (ERE), which can confer hormone inducibility where vectors are used for expression in cells having the respective hormone receptors. To reduce background levels of expression, elements responsive to ecdysone, an insect hormone, can be used instead, with coexpression of the ecdysone receptor.

In one embodiment of the invention, expression vectors can be designed to fuse the expressed polypeptide to small protein tags that facilitate purification and/or visualization. Such tags include a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). The fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the polypeptides of the present invention can be expressed as a fusion to glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione. Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG® antibody (Stratagene, La Jolla, Calif., USA), and the HA epitope, detectable by anti-HA antibody.

For secretion of expressed polypeptides, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides that are larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Vectors for phage display fuse the encoded polypeptide to, e.g., the gene III protein (pIII) or gene VIII protein (PIII) for display on the surface of filamentous phage, such as M13. See Barbas et al., Phage Display: A Laboratory Manual, Cold Spring Harbor Laboratory Press (2001); Kay et al. (eds.), Phage Display of Peptides and Proteins: A Laboratory Manual, Academic Press, Inc., (1996); Abelson et al (eds.), Combinatorial Chemistry (Methods in Enzymology, Vol. 267) Academic Press (1996). Vectors for yeast display, e.g. the pYD1 yeast display vector (Invitrogen, Carlsbad, Calif., USA), use the α-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea Victoria (“GFP”) and its variants. The GFP-like chromophore can be selected from GFP-like chromophores found in naturally occurring proteins, such as A. Victoria GFP (GenBank accession number AAA27721), Renilla renifommis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. See Li et al., J. Biol. Chem. 272: 28545-28549 (1997). Alternatively, the GFP-like chromophore can be selected from GFP-like chromophores modified from those found in nature. The methods for engineering such modified GFP-like chromophores and testing them for fluorescence activity, both alone and as part of protein fusions, are well known in the art. See Heim et al., Curr. Biol. 6: 178-182 (1996) and Palm et al., Methods Enzymol. 302: 378-394 (1999). A variety of such modified chromophores are now commercially available and can readily be used in the fusion proteins of the present invention. These include EGFP (“enhanced GFP”), EBFP (“enhanced blue fluorescent protein”), BFP2, EYFP (“enhanced yellow fluorescent protein”), ECFP (“enhanced cyan fluorescent protein”) or Citrine. EGFP (see, e.g, Cormack et al., Gene 173: 33-38 (1996); U.S. Pat. Nos. 6,090,919 and 5,804,387, the disclosures of which are incorporated herein by reference in their entireties) is found on a variety of vectors, both plasmid and viral, which are available commercially (Clontech Labs, Palo Alto, Calif., USA); EBFP is optimized for expression in mammalian cells whereas BFP2, which retains the original jellyfish codons, can be expressed in bacteria (see, e.g., Heim et al., Curr. Biol. 6: 178-182 (1996) and Cormack et al., Gene 173: 33-38 (1996)). Vectors containing these blue-shifted variants are available from Clontech Labs (Palo Alto, Calif., USA). Vectors containing EYFP, ECFP (see, e.g., Heim et al., Curr. Biol. 6: 178-182 (1996); Miyawaki et al., Nature 388: 882-887 (1997)) and Citrine (see, e.g., Heikal et al., Proc. Natl. Acad. Sci. USA 97: 11996-12001 (2000)) are also available from Clontech Labs. The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein (Methods in Enzymology, Vol. 302), Academic Press, Inc. (1999); Yang, et al., J Biol Chem, 273: 8212-6 (1998); Bevis et al., Nature Biotechnology, 20:83-7 (2002). The GFP-like chromophore of each of these GFP variants can usefully be included in the fusion proteins of the present invention.

Fusions to the IgG Fc region increase serum half-life of protein pharmaceutical products through interaction with the FcRn receptor (also denominated the FcRp receptor and the Brambell receptor, FcRb), further described in International Patent Application nos. WO 97/43316, WO 97/34631, WO 96/32478, WO 96/18412, the disclosures of which are incorporated herein by reference in their entireties.

For long-term, high-yield recombinant production of the polypeptides of the present invention, stable expression is preferred. Stable expression is readily achieved by integration into the host cell genome of vectors having selectable markers, followed by selection of these integrants. Vectors such as pUB6/V5-His A, B, and C (Invitrogen, Carlsbad, Calif., USA) are designed for high-level stable expression of heterologous proteins in a wide range of mammalian tissue types and cell lines. pUB6/V5-His uses the promoter/enhancer sequence from the human ubiquitin C gene to drive expression of recombinant proteins: expression levels in 293, CHO, and NIH3T3 cells are comparable to levels from the CMV and human EF-1a promoters. The bsd gene permits rapid selection of stably transfected mammalian cells with the potent antibiotic blasticidin.

Replication incompetent retroviral vectors, typically derived from Moloney murine leukemia virus, also are useful for creating stable transfectants having integrated provirus. The highly efficient transduction machinery of retroviruses, coupled with the availability of a variety of packaging cell lines such as RetroPack™ PT 67, EcoPack2™-293, AmphoPack-293, and GP2-293 cell lines (all available from Clontech Laboratories, Palo Alto, Calif., USA) allow a wide host range to be infected with high efficiency; varying the multiplicity of infection readily adjusts the copy number of the integrated provirus.

Of course, not all vectors and expression control sequences will function equally well to express the nucleic acid molecules of this invention. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must be replicated in it. The vector's copy number, the ability to control that copy number, the ability to control integration, if any, and the expression of any other proteins encoded by the vector, such as antibiotic or other selection markers, should also be considered. The present invention further includes host cells comprising the vectors of the present invention, either present episomally within the cell or integrated, in whole or in part, into the host cell chromosome. Among other considerations, some of which are described above, a host cell strain may be chosen for its ability to process the expressed polypeptide in the desired fashion. Such post-translational modifications of the polypeptide include, but are not limited to, acetylation, carboxylation, glycosylation, phosphorylation, lipidation, and acylation, and it is an aspect of the present invention to provide CaSPs with such post-translational modifications.

In selecting an expression control sequence, a variety of factors should also be considered. These include, for example, the relative strength of the sequence, its controllability, and its compatibility with the nucleic acid molecules of this invention, particularly with regard to potential secondary structures. Unicellular hosts should be selected by consideration of their compatibility with the chosen vector, the toxicity of the product coded for by the nucleic acid sequences of this invention, their secretion characteristics, their ability to fold the polypeptide correctly, their fermentation or culture requirements, and the ease of purification from them of the products coded for by the nucleic acid molecules of this invention.

The recombinant nucleic acid molecules and more particularly, the expression vectors of this invention may be used to express the polypeptides of this invention as recombinant polypeptides in a heterologous host cell. The polypeptides of this invention may be full-length or less than full-length polypeptide fragments recombinantly expressed from the nucleic acid molecules according to this invention. Such polypeptides include analogs, derivatives and muteins that may or may not have biological activity.

Vectors of the present invention will also often include elements that permit in vitro transcription of RNA from the inserted heterologous nucleic acid. Such vectors typically include a phage promoter, such as that from T7, T3, or SP6, Ranking the nucleic acid insert. Often two different such promoters flank the inserted nucleic acid, permitting separate in vitro production of both sense and antisense strands.

Transformation and other methods of introducing nucleic acids into a host cell (e.g., conjugation, protoplast transformation or fusion, transfection, electroporation, liposome delivery, membrane fusion techniques, high velocity DNA-coated pellets, viral infection and protoplast fusion) can be accomplished by a variety of methods which are well known in the art (See, for instance, Ausubel, supra, and Sambrook et al., supra). Bacterial, yeast, plant or mammalian cells are transformed or transfected with an expression vector, such as a plasmid, a cosmid, or the like, wherein the expression vector comprises the nucleic acid of interest. Alternatively, the cells may be infected by a viral expression vector comprising the nucleic acid of interest. Depending upon the host cell, vector, and method of transformation used, transient or stable expression of the polypeptide will be constitutive or inducible. One having ordinary skill in the art will be able to decide whether to express a polypeptide transiently or stably, and whether to express the protein constitutively or inducibly.

A wide variety of unicellular host cells are useful in expressing the DNA sequences of this invention. These hosts may include well known eukaryotic and prokaryotic hosts, such as strains of, fungi, yeast, insect cells such as Spodoptera frugiperda (SF9), animal cells such as CHO, as well as plant cells in tissue culture. Representative examples of appropriate host cells include, but are not limited to, bacterial cells, such as E. coli, Caulobacter crescentus, Streptoinyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF™ cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include BHK cells, BSC 1 cells, BSC 40 cells, BMT 10 cells, VERO cells, COS1 cells, COS7 cells, Chinese hamster ovary (CHO) cells, 3T3 cells, NIH 3T3 cells, 293 cells, HEPG2 cells, HeLa cells, L cells, MDCK cells, HEK293 cells, WI38 cells, murine ES cell lines (e.g., from strains 129/SV, C57/BL6, DBA-1, 129/SVJ), K562 cells, Jurkat cells, and BW5147 cells. Other mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA). Cells or cell lines derived from breast, intestine, colon, lung, ovarian or prostate tissue are particularly preferred because they may provide a more native post-translational processing. Particularly preferred are human breast cells.

Particular details of the transfection, expression and purification of recombinant proteins are well documented and are understood by those of skill in the art. Further details on the various technical aspects of each of the steps used in recombinant production of foreign genes in bacterial cell expression systems can be found in a number of texts and laboratory manuals in the art. See, e.g., Ausubel (1992), supra, Ausubel (1999), supra, Sambrook (1989), supra, and Sambrook (2001), supra.

Methods for introducing the vectors and nucleic acid molecules of the present invention into the host cells are well known in the art; the choice of technique will depend primarily upon the specific vector to be introduced and the host cell chosen.

Nucleic acid molecules and vectors may be introduced into prokaryotes, such as E. coli, in a number of ways. For instance, phage lambda vectors will typically be packaged using a packaging extract (e.g., Gigapack® packaging extract, Stratagene, La Jolla, Calif., USA), and the packaged virus used to infect E. coli.

Plasmid vectors will typically be introduced into chemically competent or electrocompetent bacterial cells. E. coli cells can be rendered chemically competent by treatment, e.g., with CaCl₂, or a solution of Mg²⁺, Mn²⁺, Ca²⁺, Rb⁺ or K⁺, dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian Coli® XL10-Gold® Ultracompetent Cells (Stratagene, La Jolla, Calif., USA); DH5α competent cells (Clontech Laboratories, Palo Alto, Calif., USA); and TOP10 Chemically Competent E. coli Kit (Invitrogen, Carlsbad, Calif., USA)). Bacterial cells can be rendered electrocompetent to take up exogenous DNA by electroporation by various pre-pulse treatments; vectors are introduced by electroporation followed by subsequent outgrowth in selected media. An extensive series of protocols is provided by BioRad (Richmond, Calif., USA).

Vectors can be introduced into yeast cells by spheroplasting, treatment with lithium salts, electroporation, or protoplast fusion. Spheroplasts are prepared by the action of hydrolytic enzymes such as a snail-gut extract, usually denoted Glusulase or Zymolyase, or an enzyme from Arthrobacter luteus to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol.

For lithium-mediated transformation, yeast cells are treated with lithium acetate to permeabilize the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6): 339-46 (1989).

For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194: 182-187 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means. For chemical transfection, DNA can be coprecipitated with CaPO₄ or introduced using liposomal and nonliposomal lipid-based agents. Commercial kits are available for CaPO₄ transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Protocols for electroporating mammalian cells can be found in, for example, Norton et al. (eds.), Gene Transfer Methods: Introducing DNA into Living Cells and Organisms, BioTechniques Books, Eaton Publishing Co. (2000). Other transfection techniques include transfection by particle bombardment and microinjection. See, e.g., Cheng et al., Proc. Natl. Acad. Sci. USA 90(10): 4455-9 (1993); Yang et al., Proc. Natl. Acad. Sci. USA 87(24): 9568-72 (1990).

Production of the recombinantly produced proteins of the present invention can optionally be followed by purification.

Purification of recombinantly expressed proteins is now well within the skill in the art and thus need not be detailed here. See, e.g., Thorner et al. (eds.), Applications of Chimeric Genes and Hybrid Proteins, Part A: Gene Expression and Protein Purification (Methods in Enzymology, Vol. 326), Academic Press (2000); Harbin (ed.), Cloning, Gene Expression and Protein Purification: Experimental Procedures and Process Rationale, Oxford Univ. Press (2001); Marshak et al., Strategies for Protein Purification and Characterization: A Laboratory Course Manual, Cold Spring Harbor Laboratory Press (1996); and Roe (ed.), Protein Purification Applications, Oxford University Press (2001).

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Polypeptides including Fragments Muteins, Homologous Proteins Allelic Variants, Analogs and Derivatives

Another aspect of the invention relates to polypeptides encoded by the nucleic acid molecules described herein. In a preferred embodiment, the polypeptide is a cancer specific polypeptide (CaSP). In an even more preferred embodiment, the polypeptide comprises an amino acid sequence of the gene products of Table 2a or Table 2b or is derived from a polypeptide having the amino acid sequence of the gene products of Table 2a or Table 2b. A polypeptide as defined herein may be produced recombinantly, as discussed supra, may be isolated from a cell that naturally expresses the protein, or may be chemically synthesized following the teachings of the specification and using methods well known to those having ordinary skill in the art.

Polypeptides of the present invention may also comprise a part or fragment of a CaSP. In a preferred embodiment, the fragment is derived from a polypeptide having an amino acid sequence selected from the group consisting of the gene products of Table 2a or Table 2b. Polypeptides of the present invention comprising a part or fragment of an entire CaSP may or may not be CaSPs. For example, a full-length polypeptide may be cancer-specific, while a fragment thereof may be found in normal breast, intestine, colon, lung, ovarian or prostate tissues as well as in breast, intestine, colon, lung, ovarian or prostate cancer. A polypeptide that is not a CaSP, whether it is a fragment, analog, mutein, homologous protein or derivative, is nevertheless useful, especially for immunizing animals to prepare anti-CaSP antibodies. In a preferred embodiment, the part or fragment is a CaSP. Methods of determining whether a polypeptide of the present invention is a CaSP are described infra.

Polypeptides of the present invention comprising fragments of at least 6 contiguous amino acids are also useful in mapping B cell and T cell epitopes of the reference protein. See, e.g., Geysen et al., Proc. Natl. Acad. Sci. USA 81: 3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of a polypeptide of the present invention have utility in such a study.

Polypeptides of the present invention comprising fragments of at least 8 contiguous amino acids, often at least 15 contiguous amino acids, are useful as immunogens for raising antibodies that recognize polypeptides of the present invention. See, e.g., Lerner, Nature 299: 592-596 (1982); Shinnick et al., Annu. Rev. Microbiol. 37: 425-46 (1983); Sutcliffe et al., Science 219: 660-6 (1983). As further described in the above-cited references, virtually all 8-mers, conjugated to a carrier, such as a protein, prove immunogenic and are capable of eliciting antibodies for the conjugated peptide; accordingly, all fragments of at least 8 amino acids of the polypeptides of the present invention have utility as immunogens.

Polypeptides comprising fragments of at least 8, 9, 10 or 12 contiguous amino acids are also useful as competitive inhibitors of binding of the entire polypeptide, or a portion thereof, to antibodies (as in epitope mapping), and to natural binding partners, such as subunits in a multimeric complex or to receptors or ligands of the subject protein; this competitive inhibition permits identification and separation of molecules that bind specifically to the polypeptide of interest. See U.S. Pat. Nos. 5,539,084 and 5,783,674, incorporated herein by reference in their entireties.

The polypeptide of the present invention thus preferably is at least 6 amino acids in length, typically at least 8, 9, 10 or 12 amino acids in length, and often at least 15 amino acids in length. Often, the polypeptide of the present invention is at least 20 amino acids in length, even 25 amino acids, 30 amino acids, 35 amino acids, or 50 amino acids or more in length. Of course, larger polypeptides having at least 75 amino acids, 100 amino acids, or even 150 amino acids are also useful, and at times preferred.

One having ordinary skill in the art can produce fragments by truncating the nucleic acid molecule, e.g., a CaSNA, encoding the polypeptide and then expressing it recombinantly. Alternatively, one can produce a fragment by chemically synthesizing a portion of the full-length polypeptide. One may also produce a fragment by enzymatically cleaving either a recombinant polypeptide or an isolated naturally occurring polypeptide. Methods of producing polypeptide fragments are well known in the art. See, e.g., Sambrook (1989), supra; Sambrook (2001), supra; Ausubel (1992), supra; and Ausubel (1999), supra. In one embodiment, a polypeptide comprising only a fragment, preferably a fragment of a CaSP, may be produced by chemical or enzymatic cleavage of a CaSP polypeptide. In a preferred embodiment, a polypeptide fragment is produced by expressing a nucleic acid molecule of the present invention encoding a fragment, preferably of a CaSP, in a host cell.

Polypeptides of the present invention are also inclusive of mutants, fusion proteins, homologous proteins and allelic variants.

A mutant protein, or mutein, may have the same or different properties compared to a naturally occurring polypeptide and comprises at least one amino acid insertion, duplication, deletion, rearrangement or substitution compared to the amino acid sequence of a native polypeptide. Small deletions and insertions can often be found that do not alter the function of a protein. Muteins may or may not be cancer-specific. Preferably, the mutein is cancer-specific. More preferably the mutein is specific for breast cancer. Even more preferably the mutein is a polypeptide that comprises at least one amino acid insertion, duplication, deletion, rearrangement or substitution compared to the amino acid sequence of the gene products of Table 2a or Table 2b. Accordingly, in a preferred embodiment, the mutein is one that exhibits at least 50% sequence identity, more preferably at least 60% sequence identity, even more preferably at least 70%, yet more preferably at least 80% sequence identity to a CaSP comprising an amino acid sequence of the gene products of Table 2a or Table 2b. In a yet more preferred embodiment, the mutein exhibits at least 85%, more preferably 90%, even more preferably 95% or 96%, and yet more preferably at least 97%, 98%, 99% or 99.5% sequence identity to a CaSP comprising an amino acid sequence of the gene products of Table 2a or Table 2b.

A mutein may be produced by isolation from a naturally occurring mutant cell, tissue or organism. A mutein may be produced by isolation from a cell, tissue or organism that has been experimentally mutagenized. Alternatively, a mutein may be produced by chemical manipulation of a polypeptide, such as by altering the amino acid residue to another amino acid residue using synthetic or semi-synthetic chemical techniques. In a preferred embodiment, a mutein is produced from a host cell comprising a mutated nucleic acid molecule compared to the naturally occurring nucleic acid molecule. For instance, one may produce a mutein of a polypeptide by introducing one or more mutations into a nucleic acid molecule of the invention and then expressing it recombinantly. These mutations may be targeted, in which particular encoded amino acids are altered, or may be untargeted, in which random encoded amino acids within the polypeptide are altered. Muteins with random amino acid alterations can be screened for a particular biological activity or property, particularly whether the polypeptide is cancer-specific, as described below. Multiple random mutations can be introduced into the gene by methods well known to the art, e.g., by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis and site-specific mutagenesis. Methods of producing muteins with targeted or random amino acid alterations are well known in the art. See, e.g., Sambrook (1989), supra; Sambrook (2001), supra; Ausubel (1992), supra; and Ausubel (1999), as well as U.S. Pat. No. 5,223,408, which is herein incorporated by reference in its entirety.

The invention also contemplates polypeptides that are homologous to a polypeptide of the invention. In a preferred embodiment, the polypeptide is homologous to a CaSP. In an even more preferred embodiment, the polypeptide is homologous to a CaSP selected from the group having an amino acid sequence of the gene products of Table 2a or Table 2b. By homologous polypeptide it is means one that exhibits significant sequence identity to a CaSP, preferably a CaSP having an amino acid sequence of the gene products of Table 2a or Table 2b. By significant sequence identity it is meant that the homologous polypeptide exhibits at least 50% sequence identity, more preferably at least 60% sequence identity, even more preferably at least 70%, yet more preferably at least 80% sequence identity to a CaSP comprising an amino acid sequence of the gene products of Table 2a or Table 2b. More preferred are homologous polypeptides exhibiting at least 85%, more preferably 90%, even more preferably 95% or 96%, and yet more preferably at least 97% or 98% sequence identity to a CaSP comprising an amino acid sequence of the gene products of Table 2a or Table 2b. Most preferably, the homologous polypeptide exhibits at least 99%, more preferably 99.5%, even more preferably 99.6%, 99.7%, 99.8% or 99.9% sequence identity to a CaSP comprising an amino acid sequence of the gene products of Table 2a or Table 2b. In a preferred embodiment, the amino acid substitutions of the homologous polypeptide are conservative amino acid substitutions as discussed above.

Homologous polypeptides of the present invention also comprise polypeptide encoded by a nucleic acid molecule that selectively hybridizes to a CaSNA or an antisense sequence thereof. In this embodiment, it is preferred that the homologous polypeptide be encoded by a nucleic acid molecule that hybridizes to a CaSNA under low stringency, moderate stringency or high stringency conditions, as defined herein. More preferred is a homologous polypeptide encoded by a nucleic acid sequence which hybridizes to a CaSNA selected from the group consisting of the gene products of Table 2a, Table 2b or Table 7, or a homologous polypeptide encoded by a nucleic acid molecule that hybridizes to a nucleic acid molecule that encodes a CaSP, preferably an CaSP of the gene products of Table 2a or Table 2b under low stringency, moderate stringency or high stringency conditions, as defined herein.

Homologous polypeptides of the present invention may be naturally occurring and derived from another species, especially one derived from another primate, such as chimpanzee, gorilla, rhesus macaque, or baboon, wherein the homologous polypeptide comprises an amino acid sequence that exhibits significant sequence identity to that of the gene products of Table 2a or Table 2b. The homologous polypeptide may also be a naturally occurring polypeptide from a human, when the CaSP is a member of a family of polypeptides. The homologous polypeptide may also be a naturally occurring polypeptide derived from a non-primate, mammalian species, including without limitation, domesticated species, e.g., dog, cat, mouse, rat, rabbit, guinea pig, hamster, cow, horse, goat or pig. The homologous polypeptide may also be a naturally occurring polypeptide derived from a non-mammalian species, such as birds or reptiles. The naturally occurring homologous protein may be isolated directly from humans or other species. Alternatively, the nucleic acid molecule encoding the naturally occurring homologous polypeptide may be isolated and used to express the homologous polypeptide recombinantly. The homologous polypeptide may also be one that is experimentally produced by random mutation of a nucleic acid molecule and subsequent expression of the nucleic acid molecule. Alternatively, the homologous polypeptide may be one that is experimentally produced by directed mutation of one or more codons to alter the encoded amino acid of a CaSP. In a preferred embodiment, the homologous polypeptide encodes a polypeptide that is a CaSP.

Relatedness of proteins can also be characterized using a second functional test, the ability of a first protein competitively to inhibit the binding of a second protein to an antibody. It is, therefore, another aspect of the present invention to provide isolated polypeptide not only identical in sequence to those described with particularity herein, but also to provide isolated second polypeptide (“cross-reactive protein”) that competitively inhibits the binding of antibodies to all or to a portion of various of the isolated polypeptides of the present invention. Such competitive inhibition can readily be determined using immunoassays well known in the art.

As discussed above, single nucleotide polymorphisms (SNPs) occur frequently in eukaryotic genomes, and the sequence determined from one individual of a species may differ from other allelic forms present within the population. Thus, polypeptides of the present invention are also inclusive of those encoded by an allelic variant of a nucleic acid molecule encoding a CaSP. In this embodiment, it is preferred that the polypeptide be encoded by an allelic variant of a gene that encodes a polypeptide having the amino acid sequence selected from the group consisting of the gene products of Table 2a or Table 2b. More preferred is that the polypeptide be encoded by an allelic variant of a gene that has the nucleic acid sequence selected from the group consisting of the gene products of Table 2a, Table 2b or Table 7.

Polypeptides of the present invention are also inclusive of derivative polypeptides encoded by a nucleic acid molecule according to the instant invention. In this embodiment, it is preferred that the polypeptide be a CaSP. Also preferred are derivative polypeptides having an amino acid sequence selected from the group consisting of the gene products of Table 2a and Table 2b and which has been acetylated, carboxylated, phosphorylated, glycosylated, ubiquitinated or other PTMs. In another preferred embodiment, the derivative has been labeled with, e.g., radioactive isotopes such as ¹²⁵I, ³²P, ³⁵S, and ³H. In another preferred embodiment, the derivative has been labeled with fluorophores, chemiluminescent agents, enzymes, and antiligands that can serve as specific binding pair members for a labeled ligand.

Polypeptide modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance Creighton, Protein Structure and Molecular Properties, 2nd ed., W. H. Freeman and Company (1993). Many detailed reviews are available on this subject, such as, for example, those provided by Wold, in Johnson (ed.), Posttranslational Covalent Modification of Proteins, pgs. 1-12, Academic Press (1983); Seifter et al., Meth. Enzymol. 182: 626-646 (1990) and Rattan et al., Ann. N.Y. Acad. Sci. 663: 48-62 (1992).

One may determine whether a polypeptide of the invention is likely to be post-translationally modified by analyzing the sequence of the polypeptide to determine if there are peptide motifs indicative of sites for post-translational modification. There are a number of computer programs that permit prediction of post-translational modifications. See, e.g., expasy with the extension .org of the world wide web (accessed Nov. 11, 2002), which includes PSORT, for prediction of protein sorting signals and localization sites, SignalP, for prediction of signal peptide cleavage sites, MITOPROT and Predotar, for prediction of mitochondrial targeting sequences, NetOGlyc, for prediction of type O-glycosylation sites in mammalian proteins, big-PI Predictor and DGPI, for prediction of prenylation-anchor and cleavage sites, and NetPhos, for prediction of Ser, Thr and Tyr phosphorylation sites in eukaryotic proteins. Other computer programs, such as those included in GCG, also may be used to determine post-translational modification peptide motifs.

General examples of types of post-translational modifications include, but are not limited to: (Z)-dehydrobutyrine; 1-chondroitin sulfate-L-aspartic acid ester; 1′-glycosyl-L-tryptophan; 1′-phospho-L-histidine; 1-thioglycine; 2′-(S-L-cysteinyl)-L-histidine; 2′-[3-carboxamido (trimethylammonio)propyl]-L-histidine; 2′-alpha-mannosyl-L-tryptophan; 2-methyl-L-glutamine; 2-oxobutanoic acid; 2-pyrrolidone carboxylic acid; 3′-(1′-L-histidyl)-L-tyrosine; 3′-(8alpha-FAD)-L-histidine; 3′-(S-L-cysteinyl)-L-tyrosine; 3′, 3″,5′-triiodo-L-thyronine; 3′-4′-phospho-L-tyrosine; 3-hydroxy-L-proline; 3′-methyl-L-histidine; 3-methyl-L-lanthionine; 3′-phospho-L-histidine; 4′-(L-tryptophan)-L-tryptophyl quinone; 42 N-cysteinyl-glycosylphosphatidylinositolethanolamine; 43-(T-L-histidyl)-L-tyrosine; 4-hydroxy-L-arginine; 4-hydroxy-L-lysine; 4-hydroxy-L-proline; 5′-(N-6-L-lysine)-L-topaquinone; 5-hydroxy-L-lysine; 5-methyl-L-arginine; alpha-1-microglobulin-Ig alpha complex chromophore; bis-L-cysteinyl bis-L-histidino diiron disulfide; bis-L-cysteinyl-L-N3′-histidino-L-serinyl tetrairon′ tetrasulfide; chondroitin sulfate D-glucuronyl-D-galactosyl-D-galactosyl-D-xylosyl-L-serine; D-alanine; D-allo-isoleucine; D-asparagine; dehydroalanine; dehydrotyrosine; dermatan 4-sulfate D-glucuronyl-D-galactosyl-D-galactosyl-D-xylosyl-L-serine; D-glucuronyl-N-glycine; dipyrrolylmethanemethyl-L-cysteine; D-leucine; D-methionine; D-phenylalanine; D-serine; D-tryptophan; glycine amide; glycine oxazolecarboxylic acid; glycine thiazolecarboxylic acid; heme P450-bis-L-cysteine-L-tyrosine; heme-bis-L-cysteine; hemediol-L-aspartyl ester-L-glutamyl ester; hemediol-L-aspartyl ester-L-glutamyl ester-L-methionine sulfonium; heme-L-cysteine; heme-L-histidine; heparan sulfate D-glucuronyl-D-galactosyl-D-galactosyl-D-xylosyl-L-serine; heme P450-bis-L-cysteine-L-lysine; hexakis-L-cysteinyl hexairon hexasulfide; keratan sulfate D-glucuronyl-D-galactosyl-D-galactosyl-D-xylosyl-L-threonine; L oxoalanine-lactic acid; L phenyllacetic acid; 1′-(8alpha-FAD)-L-histidine; L-2′.4′,5′-topaquinone; L-3′,4′-dihydroxyphenylalanine; L-3′.4′.5′-trihydroxyphenylalanine; L-4′-bromophenylalanine; L-6′-bromotryptophan; L-alanine amide; L-alanyl imidazolinone glycine; L-allysine; L-arginine amide; L-asparagine amide; L-aspartic 4-phosphoric anhydride; L-aspartic acid 1-amide; L-beta-methylthioaspartic acid; L-bromohistidine; L-citrulline; L-cysteine amide; L-cysteine glutathione disulfide; L-cysteine methyl disulfide; L-cysteine methyl ester; L-cysteine oxazolecarboxylic acid; L-cysteine oxazolinecarboxylic acid; L-cysteine persulfide; L-cysteine sulfenic acid; L-cysteine sulfinic acid; L-cysteine thiazolecarboxylic acid; L-cysteinyl homocitryl molybdenum-heptairon-nonasulfide; L-cysteinyl imidazolinone glycine; L-cysteinyl molybdopterin; L-cysteinyl molybdopterin guanine dinucleotide; L-cystine; L-erythro-beta-hydroxyasparagine; L-erythro-beta-hydroxyaspartic acid; L-gamma-carboxyglutamic acid; L-glutamic acid 1-amide; L-glutamic acid 5-methyl ester; L-glutamine amide; L-glutamyl 5-glycerylphosphorylethanolamine; L-histidine amide; L-isoglutamyl-polyglutamic acid; L-isoglutamyl-polyglycine; L-isoleucine amide; L-lanthionine; L-leucine amide; L-lysine amide; L-lysine thiazolecarboxylic acid; L-lysinoalanine; L-methionine amide; L-methionine sulfone; L-phenyalanine thiazolecarboxylic acid; L-phenylalanine amide; L-proline amide; L-selenocysteine; L-selenocysteinyl molybdopterin guanine dinucleotide; L-serine amide; L-serine thiazolecarboxylic acid; L-seryl imidazolinone glycine; L-T-bromophenylalanine; L-T-bromophenylalanine; L-threonine amide; L-thyroxine; L-tryptophan amide; L-tryptophyl quinone; L-tyrosine amide; L-valine amide; meso-lanthionine; N-(L-glutamyl)-L-tyrosine; N-(L-isoaspartyl)-glycine; N-(L-isoaspartyl)-L-cysteine; N,N,N-trimethyl-L-alanine; N,N-dimethyl-L-proline; N2-acetyl-L-lysine; N2-succinyl-L-tryptophan; N4-(ADP-ribosyl)-L-asparagine; N4-glycosyl-L-asparagine; N4-hydroxymethyl-L-asparagine; N4-methyl-L-asparagine; N5-methyl-L-glutamine; N6-1-carboxyethyl-L-lysine; N6-(4-amino hydroxybutyl)-L-lysine; N6-(L-isoglutamyl)-L-lysine; N6-(phospho-5′-adenosine)-L-lysine; N6-(phospho-5′-guanosine)-L-lysine; N6,N6,N6-trimethyl-L-lysine; N6,N6-dimethyl-L-lysine; N6-acetyl-L-lysine; N6-biotinyl-L-lysine; N6-carboxy-L-lysine; N6-formyl-L-lysine; N6-glycyl-L-lysine; N6-lipoyl-L-lysine; N6-methyl-L-lysine; N6-methyl-N6-poly(N-methyl-propylamine)-L-lysine; N6-mureinyl-L-lysine; N6-myristoyl-L-lysine; N6-palmitoyl-L-lysine; N6-pyridoxal phosphate-L-lysine; N6-pyruvic acid 2-iminyl-L-lysine; N6-retinal-L-lysine; N-acetylglycine; N-acetyl-L-glutamine; N-acetyl-L-alanine; N-acetyl-L-aspartic acid; N-acetyl-L-cysteine; N-acetyl-L-glutamic acid; N-acetyl-L-isoleucine; N-acetyl-L-methionine; N-acetyl-L-proline; N-acetyl-L-serine; N-acetyl-L-threonine; N-acetyl-L-tyrosine; N-acetyl-L-valine; N-alanyl-glycosylphosphatidylinositolethanolamine; N-asparaginyl-glycosylphosphatidylinositolethanolamine; N-aspartyl-glycosylphosphatidylinositolethanolamine; N-formylglycine; N-formyl-L-methionine; N-glycyl-glycosylphosphatidylinositolethanolamine; N-L-glutamyl-poly-L-glutamic acid; N-methylglycine; N-methyl-L-alanine; N-methyl-L-methionine; N-methyl-L-phenylalanine; N-myristoyl-glycine; N-palmitoyl-L-cysteine; N-pyruvic acid 2-iminyl-L-cysteine; N-pyruvic acid 2-iminyl-L-valine; N-seryl-glycosylphosphatidylinositolethanolamine; N-seryl-glycosyCaSPhingolipidinositolethanolamine; O-(ADP-ribosyl)-L-serine; O-(phospho-5′-adenosine)-L-threonine; 0-(phospho-5′-DNA)-L-serine; 0-(phospho-5′-DNA)-L-threonine; 0-(phospho-5′rRNA)-L-serine; 0-(phosphoribosyl depliospho-coenzyme A)-L-serine; 0-(sn-1-glycerophosphoryl)-L-serine; O4′-(8alpha-FAD)-L-tyrosine; O4′-(phospho-5′-adenosine)-L-tyrosine; O4′-(phospho-5′-DNA)-L-tyrosine; O4′-(phospho-5′-RNA)-L-tyrosine; O4′-(phospho-5′-uridine)-L-tyrosine; O4-glycosyl-L-hydroxyproline; O4′-glycosyl-L-tyrosine; O4′-sulfo-L-tyrosine; O5-glycosyl-L-hydroxylysine; O-glycosyl-L-serine; O-glycosyl-L-threonine; omega-N-(ADP-ribosyl)-L-arginine; omega-N-omega-N′-dimethyl-L-arginine; omega-N-methyl-L-arginine; omega-N-omega-N-dimethyl-L-arginine; omega-N-phospho-L-arginine; O'octanoyl-L-serine; O-palmitoyl-L-serine; O-palmitoyl-L-threonine; O-phospho-L-serine; O-phospho-L-threonine; O-phosphopantetheine-L-serine; phycoerythrobilin-bis-L-cysteine; phycourobilin-bis-L-cysteine; pyrroloquinoline quinone; pyruvic acid; S hydroxycinnamyl-L-cysteine; S-(2-aminovinyl) methyl-D-cysteine; S-(2-aminovinyl)-D-cysteine; S-(6-FW-L-cysteine; S-(8alpha-FAD)-L-cysteine; S-(ADP-ribosyl)-L-cysteine; S-(L-isoglutamyl)-L-cysteine; S-12-hydroxyfarnesyl-L-cysteine; S-acetyl-L-cysteine; S-diacylglycerol-L-cysteine; S-diphytanylglycerot diether-L-cysteine; S-farnesyl-L-cysteine; S-geranylgeranyl-L-cysteine; S-glycosyl-L-cysteine; S-glycyl-L-cysteine; S-methyl-L-cysteine; S-nitrosyl-L-cysteine; S-palmitoyl-L-cysteine; S-phospho-L-cysteine; S-phycobiliviolin-L-cysteine; S-phycocyanobilin-L-cysteine; S-phycoerythrobilin-L-cysteine; S-phytochromobilin-L-cysteine; S-selenyl-L-cysteine; S-sulfo-L-cysteine; tetrakis-L-cysteinyl diiron disulfide; tetrakis-L-cysteinyl iron; tetrakis-L-cysteinyl tetrairon tetrasulfide; trans-2,3-cis 4-dihydroxy-L-proline; tris-L-cysteinyl triiron tetrasulfide; tris-L-cysteinyl triiron trisulfide; tris-L-cysteinyl-L-aspartato tetrairon tetrasulfide; tris-L-cysteinyl-L-cysteine persulfido-bis-L-glutamato-L-histidino tetrairon disulfide trioxide; tris-L-cysteinyl-L-N3′-histidino tetrairon tetrasulfide; tris-L-cysteinyl-L-N1′-histidino tetrairon tetrasulfide; and tris-L-cysteinyl-L-serinyl tetrairon tetrasulfide.

Additional examples of PTMs may be found in web sites such as the Delta Mass database based on Krishna, R. G. and F. Wold (1998). Posttranslational Modifications. Proteins—Analysis and Design. R. H. Angeletti. San Diego, Academic Press. 1: 121-206; Methods in Enzymology, 193, J. A. McClosky (ed) (1990), pages 647-660; Methods in Protein Sequence Analysis edited by Kazutomo Imahori and Fumio Sakiyama, Plenum Press, (1993) “Post-translational modifications of proteins” R. G. Krishna and F. Wold pages 167-172; “GlycoSuiteDB: a new curated relational database of glycoprotein glycan structures and their biological sources” Cooper et al. Nucleic Acids Res. 29; 332-335 (2001) “O-GLYCBASE version 4.0: a revised database of O-glycosylated proteins” Gupta et al. Nucleic Acids Research, 27: 370-372 (1999); and “PhosphoBase, a database of phosphorylation sites: release 2.0.”, Kreegipuu et al. Nucleic Acids Res 27(1):237-239 (1999) see also, WO 02/21139A2, the disclosure of which is incorporated herein by reference in its entirety.

Tumorigenesis is often accompanied by alterations in the post-translational modifications of proteins. Thus, in another embodiment, the invention provides polypeptides from cancerous cells or tissues that have altered post-translational modifications compared to the post-translational modifications of polypeptides from normal cells or tissues. A number of altered post-translational modifications are known. One common alteration is a change in phosphorylation state, wherein the polypeptide from the cancerous cell or tissue is hyperphosphorylated or hypophosphorylated compared to the polypeptide from a normal tissue, or wherein the polypeptide is phosphorylated on different residues than the polypeptide from a normal cell. Another common alteration is a change in glycosylation state, wherein the polypeptide from the cancerous cell or tissue has more or less glycosylation than the polypeptide from a normal tissue, and/or wherein the polypeptide from the cancerous cell or tissue has a different type of glycosylation than the polypeptide from a noncancerous cell or tissue. Changes in glycosylation may be critical because carbohydrate-protein and carbohydrate-carbohydrate interactions are important in cancer cell progression, dissemination and invasion. See, e.g., Barchi, Curr. Pharm. Des. 6: 485-501 (2000), Verma, Cancer Biochem. Biophys. 14: 151-162 (1994) and Dennis et al., Bioessays 5: 412-421 (1999).

Another post-translational modification that may be altered in cancer cells is prenylation. Prenylation is the covalent attachment of a hydrophobic prenyl group (either farnesyl or geranylgeranyl) to a polypeptide. Prenylation is required for localizing a protein to a cell membrane and is often required for polypeptide function. For instance, the Ras superfamily of GTPase signaling proteins must be prenylated for function in a cell. See, e.g., Prendergast et al., Semin. Cancer Biol. 10: 443-452 (2000) and Khwaja et al., Lancet 355: 741-744 (2000).

Other post-translation modifications that may be altered in cancer cells include, without limitation, polypeptide methylation, acetylation, arginylation or racemization of amino acid residues. In these cases, the polypeptide from the cancerous cell may exhibit either increased or decreased amounts of the post-translational modification compared to the corresponding polypeptides from noncancerous cells.

Other polypeptide alterations in cancer cells include abnormal polypeptide cleavage of proteins and aberrant protein-protein interactions. Abnormal polypeptide cleavage may be cleavage of a polypeptide in a cancerous cell that does not usually occur in a normal cell, or a lack of cleavage in a cancerous cell, wherein the polypeptide is cleaved in a normal cell. Aberrant protein-protein interactions may be either covalent cross-linking or non-covalent binding between proteins that do not normally bind to each other. Alternatively, in a cancerous cell, a protein may fail to bind to another protein to which it is bound in a noncancerous cell. Alterations in cleavage or in protein-protein interactions may be due to over- or underproduction of a polypeptide in a cancerous cell compared to that in a normal cell, or may be due to alterations in post-translational modifications (see above) of one or more proteins in the cancerous cell. See, e.g., Henschen-Edman, Ann. N.Y. Acad. Sci. 936: 580-593 (2001).

Alterations in polypeptide post-translational modifications, as well as changes in polypeptide cleavage and protein-protein interactions, may be determined by any method known in the art. For instance, alterations in phosphorylation may be determined by using anti-phosphoserine, anti-phosphothreonine or anti-phosphotyrosine antibodies or by amino acid analysis. Glycosylation alterations may be determined using antibodies specific for different sugar residues, by carbohydrate sequencing, or by alterations in the size of the glycoprotein, which can be determined by, e.g., SDS polyacrylamide gel electrophoresis (PAGE). Other alterations of post-translational modifications, such as prenylation, racemization, methylation, acetylation and arginylation, may be determined by chemical analysis, protein sequencing, amino acid analysis, or by using antibodies specific for the particular post-translational modifications. Changes in protein-protein interactions and in polypeptide cleavage may be analyzed by any method known in the art including, without limitation, non-denaturing PAGE (for non-covalent protein-protein interactions), SDS PAGE (for covalent protein-protein interactions and protein cleavage), chemical cleavage, protein sequencing or immunoassays.

In another embodiment, the invention provides polypeptides that have been post-translationally modified. In one embodiment, polypeptides may be modified enzymatically or chemically, by addition or removal of a post-translational modification. For example, a polypeptide may be glycosylated or deglycosylated enzymatically. Similarly, polypeptides may be phosphorylated using a purified kinase, such as a MAP kinase (e.g, p38, ERK, or JNK) or a tyrosine kinase (e.g., Src or erbB2). A polypeptide may also be modified through synthetic chemistry. Alternatively, one may isolate the polypeptide of interest from a cell or tissue that expresses the polypeptide with the desired post-translational modification. In another embodiment, a nucleic acid molecule encoding the polypeptide of interest is introduced into a host cell that is capable of post-translationally modifying the encoded polypeptide in the desired fashion. If the polypeptide does not contain a motif for a desired post-translational modification, one may alter the post-translational modification by mutating the nucleic acid sequence of a nucleic acid molecule encoding the polypeptide so that it contains a site for the desired post-translational modification. Amino acid sequences that may be post-translationally modified are known in the art. See, e.g., the programs described above on the website expasy with the extension .org of the world wide web. The nucleic acid molecule may also be introduced into a host cell that is capable of post-translationally modifying the encoded polypeptide. Similarly, one may delete sites that are post-translationally modified by either mutating the nucleic acid sequence so that the encoded polypeptide does not contain the post-translational modification motif, or by introducing the native nucleic acid molecule into a host cell that is not capable of post-translationally modifying the encoded polypeptide.

It will be appreciated, as is well known and as noted above, that polypeptides are not always entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available from Pierce, Rockford, Ill., USA).

Polypeptides of the present invention, including full length polypeptides, fragments and fusion proteins, can be conjugated, using such cross-linking reagents, to fluorophores that are not amine- or thiol-reactive. Other labels that usefully can be conjugated to polypeptides of the present invention include radioactive labels, echosonographic contrast reagents, and MRI contrast agents.

Polypeptides of the present invention, including full length polypeptide, fragments and fusion proteins, can also usefully be conjugated using cross-linking agents to carrier proteins, such as KLH, bovine thyroglobulin, and even bovine serum albumin (BSA), to increase immunogenicity for raising anti-CaSP antibodies.

Polypeptides of the present invention, including full length polypeptide, fragments and fusion proteins, can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4): 249-304 (1992); Scott et al., Curr. Pharm. Des. 4(6): 423-38 (1998); DeSantis et al., Curr. Opin. Biotechnol. 10(4): 324-30 (1999). PEG monomers can be attached to the protein directly or through a linker, with PEGylation using PEG monomers activated with tresyl chloride (2,2,2-trifluoroethanesulphonyl chloride) permitting direct attachment under mild conditions.

Polypeptides of the present invention are also inclusive of analogs of a polypeptide encoded by a nucleic acid molecule according to the instant invention. In a preferred embodiment, this polypeptide is a CaSP. In a more preferred embodiment, this polypeptide is derived from a polypeptide having part or all of the amino acid sequence of the gene products of Table 2a or Table 2b. Also preferred is an analog polypeptide comprising one or more substitutions of non-natural amino acids or non-native inter-residue bonds compared to the naturally occurring polypeptide. In one embodiment, the analog is structurally similar to a CaSP, but one or more peptide linkages is replaced by a linkage selected from the group consisting of —CH₂NH—, —CH₂S—, —CH₂—CH₂—, —CH═CH—(cis and trans), —COCH₂—, —CH(OH)CH₂— and —CH₂SO—. In another embodiment, the analog comprises substitution of one or more amino acids of a CaSP with a D-amino acid of the same type or other non-natural amino acid in order to generate more stable peptides. D-amino acids can readily be incorporated during chemical peptide synthesis: peptides assembled from D-amino acids are more resistant to proteolytic attack; incorporation of D-amino acids can also be used to confer specific three-dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (see, e.g., Kole et al., Biochem. Biophys. Res. Com. 209: 817-821 (1995)), and various halogenated phenylalanine derivatives.

Non-natural amino acids can be incorporated during solid phase chemical synthesis or by recombinant techniques, although the former is typically more common. Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000); Jones, Amino Acid and Peptide Synthesis (Oxford Chemistry Primers, No 7), Oxford Univ. Press (1992); and Bodanszky, Principles of Peptide Synthesis (Springer Laboratory), Springer Verlag (1993).

Amino acid analogues having detectable labels are also usefully incorporated during synthesis to provide derivatives and analogs. Biotin, for example can be added using biotinoyl—(9-fluorenylmethoxycarbonyl)-L-lysine (FMOC biocytin) (Molecular Probes, Eugene, Oreg., USA). Biotin can also be added enzymatically by incorporation into a fusion protein of a E. coli BirA substrate peptide. The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)—TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-5-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-β-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, all available from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl Acad. Sci. USA 96(9): 4780-5 (1999); Wang et al., Science 292(5516): 498-500 (2001).

Fusion Proteins

Another aspect of the present invention relates to the fusion of a polypeptide of the present invention to heterologous polypeptides. In a preferred embodiment, the polypeptide of the present invention is a CaSP. In a more preferred embodiment, the polypeptide of the present invention that is fused to a heterologous polypeptide comprises part or all of the amino acid sequence of the gene products of Table 2a or Table 2b, or is a mutein, homologous polypeptide, analog or derivative thereof. In an even more preferred embodiment, the fusion protein is encoded by a nucleic acid molecule comprising all or part of the nucleic acid sequence of the gene products of Table 2a, Table 2b or Table 7, or comprises all or part of a nucleic acid sequence that selectively hybridizes or is homologous to a nucleic acid molecule comprising a nucleic acid sequence of the gene products of Table 2a, Table 2b or Table 7.

The fusion proteins of the present invention will include at least one fragment of a polypeptide of the present invention, which fragment is at least 6, typically at least 8, often at least 15, and usefully at least 16, 17, 18, 19, or 20 amino acids long. The fragment of the polypeptide of the present to be included in the fusion can usefully be at least 25 amino acids long, at least 50 amino acids long, and can be at least 75, 100, or even 150 amino acids long. Fusions that include the entirety of a polypeptide of the present invention have particular utility.

The heterologous polypeptide included within the fusion protein of the present invention is at least 6 amino acids in length, often at least 8 amino acids in length, and preferably at least 15, 20, or 25 amino acids in length. Fusions that include larger polypeptides, such as the IgG Fc region, and even entire proteins (such as GFP chromophore-containing proteins) are particularly useful.

As described above in the description of vectors and expression vectors of the present invention, which discussion is incorporated here by reference in its entirety, heterologous polypeptides to be included in the fusion proteins of the present invention can usefully include those designed to facilitate purification and/or visualization of recombinantly-expressed proteins. See, e.g., Ausubel, Chapter 16, (1992), supra. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of the presence of a polypeptide of the invention.

As also discussed above, heterologous polypeptides to be included in the fusion proteins of the present invention can usefully include those that facilitate secretion of recombinantly expressed proteins into the periplasmic space or extracellular milieu for prokaryotic hosts or into the culture medium for eukaryotic cells through incorporation of secretion signals and/or leader sequences. For example, a His⁶ tagged protein can be purified on a Ni affinity column and a GST fusion protein can be purified on a glutathione affinity column. Similarly, a fusion protein comprising the Fc domain of IgG can be purified on a Protein A or Protein G column and a fusion protein comprising an epitope tag such as myc can be purified using an immunoaffinity column containing an anti-c-myc antibody. It is preferable that the epitope tag be separated from the protein encoded by the essential gene by an enzymatic cleavage site that can be cleaved after purification. See also the discussion of nucleic acid molecules encoding fusion proteins that may be expressed on the surface of a cell.

Other useful fusion proteins of the present invention include those that permit use of the polypeptide of the present invention as bait in a yeast two-hybrid system. See Bartel et al. (eds.), The Yeast Two-Hybrid System, Oxford University Press (1997); Zhu et al., Yeast Hybrid Technologies, Eaton Publishing (2000); Fields et al., Trends Genet. 10(8): 286-92 (1994); Mendelsohn et al., Curr. Opin. Biotechnol. 5(5): 482-6 (1994); Luban et al., Curr. Opin. Biotechnol. 6(1): 59-64 (1995); Allen et al., Trends Biochem. Sci. 20(12): 511-6 (1995); Drees, Curr. Opin. Chem. Biol. 3(1): 64-70 (1999); Topcu et al., Pharm. Res. 17(9): 1049-55 (2000); Fashena et al., Gene 250(1-2): 1-14 (2000); Colas et al., Nature 380, 548-550 (1996); Norman, T. et al., Science 285, 591-595 (1999); Fabbrizio et al., Oncogene 18, 4357-4363 (1999); Xu et al., Proc Natl Acad Sci USA. 94, 12473-12478 (1997); Yang, et al., Nuc. Acids Res. 23, 1152-1156 (1995); Kolonin et al., Proc Natl Acad Sci USA 95, 14266-14271 (1998); Cohen et al., Proc Natl Acad Sci U S A 95, 14272-14277 (1998); Uetz, et al. Nature 403, 623-627 (2000); Ito, et al., Proc Natl Acad Sci USA 98, 4569-4574 (2001). Typically, such fusion is to either E. coli LexA or yeast GAL4 DNA binding domains. Related bait plasmids are available that express the bait fused to a nuclear localization signal.

Other useful fusion proteins include those that permit display of the encoded polypeptide on the surface of a phage or cell, fusions to intrinsically fluorescent proteins, such as green fluorescent protein (GFP), and fusions to the IgG Fc region, as described above.

The polypeptides of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

Fusion partners include, inter alia, myc, hemagglutinin (HA), GST, immunoglobulins, β-galactosidase, biotin trpE, protein A, β-lactamase, α-amylase, maltose binding protein, alcohol dehydrogenase, polyhistidine (for example, six histidine at the amino and/or carboxyl terminus of the polypeptide), lacZ, green fluorescent protein (GFP), yeast α mating factor, GAL4 transcription activation or DNA binding domain, luciferase, and serum proteins such as ovalbumin, albumin and the constant domain of IgG. See, e.g., Ausubel (1992), supra and Ausubel (1999), supra. Fusion proteins may also contain sites for specific enzymatic cleavage, such as a site that is recognized by enzymes such as Factor XIII, trypsin, pepsin, or any other enzyme known in the art. Fusion proteins will typically be made by either recombinant nucleic acid methods, as described above, chemically synthesized using techniques well known in the art (e.g., a Merrifield synthesis), or produced by chemical cross-linking.

Another advantage of fusion proteins is that the epitope tag can be used to bind the fusion protein to a plate or column through an affinity linkage for screening binding proteins or other molecules that bind to the CaSP.

As further described below, the polypeptides of the present invention can readily be used as specific immunogens to raise antibodies that specifically recognize polypeptides of the present invention including CaSPs and their allelic variants and homologues. The antibodies, in turn, can be used, inter alia, specifically to assay for the polypeptides of the present invention, particularly CaSPs, e.g. by ELISA for detection of protein fluid samples, such as serum, by immunohistochemistry or laser scanning cytometry, for detection of protein in tissue samples, or by flow cytometry, for detection of intracellular protein in cell suspensions, for specific antibody-mediated isolation and/or purification of CaSPs, as for example by immunoprecipitation, and for use as specific agonists or antagonists of CaSPs.

One may determine whether polypeptides of the present invention including CaSPs, muteins, homologous proteins or allelic variants or fusion proteins of the present invention are functional by methods known in the art. For instance, residues that are tolerant of change while retaining function can be identified by altering the polypeptide at known residues using methods known in the art, such as alanine scanning mutagenesis, Cunningham et al., Science 244(4908): 1081-5 (1989); transposon linker scanning mutagenesis, Chen et al., Gene 263(1-2): 39-48 (2001); combinations of homolog- and alanine-scanning mutagenesis, Jin et al., J. Mol. Biol. 226(3): 851-65 (1992); combinatorial alanine scanning, Weiss et al., Proc. Natl. Acad. Sci. USA 97(16): 8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, (Epicentre Technologies Corporation, Madison, Wis., USA)).

Purification of the polypeptides or fusion proteins of the present invention is well known and within the skill of one having ordinary skill in the art. See, e.g., Scopes, Protein Purification, 2d ed. (1987). Purification of recombinantly expressed polypeptides is described above. Purification of chemically-synthesized peptides can readily be effected, e.g., by HPLC.

Accordingly, it is an aspect of the present invention to provide the isolated polypeptides or fusion proteins of the present invention in pure or substantially pure form in the presence or absence of a stabilizing agent. Stabilizing agents include both proteinaceous and non-proteinaceous material and are well known in the art. Stabilizing agents, such as albumin and polyethylene glycol (PEG) are known and are commercially available.

Although high levels of purity are preferred when the isolated polypeptide or fusion protein of the present invention are used as therapeutic agents, such as in vaccines and replacement therapy, the isolated polypeptides of the present invention are also useful at lower purity. For example, partially purified polypeptides of the present invention can be used as immunogens to raise antibodies in laboratory animals.

In a preferred embodiment, the purified and substantially purified polypeptides of the present invention are in compositions that lack detectable ampholytes, acrylamide monomers, bis-acrylamide monomers, and polyacrylamide.

The polypeptides or fusion proteins of the present invention can usefully be attached to a substrate. The substrate can be porous or solid, planar or non-planar; the bond can be covalent or noncovalent. For example, the peptides of the invention may be stabilized by covalent linkage to albumin. See, U.S. Pat. No. 5,876,969, the contents of which are hereby incorporated in its entirety.

For example, the polypeptides or fusion proteins of the present invention can usefully be bound to a porous substrate, commonly a membrane, typically comprising nitrocellulose, polyvinylidene fluoride (PVDF), or cationically derivatized, hydrophilic PVDF; so bound, the polypeptides or fusion proteins of the present invention can be used to detect and quantify antibodies, e.g. in serum, that bind specifically to the immobilized polypeptide or fusion protein of the present invention.

As another example, the polypeptides or fusion proteins of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in a standard microtiter dish, the plastic is typically polystyrene.

The polypeptides and fusion proteins of the present invention can also be attached to a substrate suitable for use as a surface enhanced laser desorption ionization source; so attached, the polypeptide or fusion protein of the present invention is useful for binding and then detecting secondary proteins that bind with sufficient affinity or avidity to the surface-bound polypeptide or fusion protein to indicate biologic interaction there between. The polypeptides or fusion proteins of the present invention can also be attached to a substrate suitable for use in surface plasmon resonance detection; so attached, the polypeptide or fusion protein of the present invention is useful for binding and then detecting secondary proteins that bind with sufficient affinity or avidity to the surface-bound polypeptide or fusion protein to indicate biological interaction there between.

### Alternative Transcripts

In another aspect, the present invention provides splice variants of genes and proteins encoded thereby. The identification of a novel splice variant which encodes an amino acid sequence with a novel region can be targeted for the generation of reagents for use in detection and/or treatment of cancer. The novel amino acid sequence may lead to a unique protein structure, protein subcellular localization, biochemical processing or function of the splice variant. This information can be used to directly or indirectly facilitate the generation of additional or novel therapeutics or diagnostics. The nucleotide sequence in this novel splice variant can be used as a nucleic acid probe for the diagnosis and/or treatment of cancer.

Specifically, the newly identified sequences may enable the production of new antibodies or compounds directed against the novel region for use as a therapeutic or diagnostic. Alternatively, the newly identified sequences may alter the biochemical or biological properties of the encoded protein in such a way as to enable the generation of improved or different therapeutics targeting this protein.

### Antibodies

In another aspect, the invention provides antibodies, including fragments and derivatives thereof, which bind specifically to polypeptides encoded by the nucleic acid molecules of the invention. In a preferred embodiment, the antibodies are specific for a polypeptide that is a CaSP, or a fragment, mutein, derivative, analog, isoform, allelic variant or fusion protein thereof. In a more preferred embodiment, the antibodies are specific for a polypeptide that comprises the gene products of Table 2a or Table 2b, or a fragment, mutein, derivative, analog, isoform, allelic variant or fusion protein thereof.

The antibodies of the present invention can be specific for linear epitopes, discontinuous epitopes, or conformational epitopes of such proteins or protein fragments, either as present on the protein in its native conformation or, in some cases, as present on the proteins as denatured, as, e.g., by solubilization in SDS. New epitopes may be also due to a difference in post translational modifications (PTMs) in disease versus normal tissue. For example, a particular site on a CaSP may be glycosylated in cancerous cells, but not glycosylated in normal cells or vice versa. In addition, alternative splice forms of a CaSP may be indicative of cancer. Differential degradation of the C or N-terminus of a CaSP may also be a marker or target for anticancer therapy. For example, an CaSP may be N-terminal degraded in cancer cells exposing new epitopes to which antibodies may selectively bind for diagnostic or therapeutic uses.

As is well known in the art, the degree to which an antibody can discriminate as among molecular species in a mixture will depend, in part, upon the conformational relatedness of the species in the mixture; typically, the antibodies of the present invention will discriminate over adventitious binding to non-CaSP polypeptides by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the polypeptide of the present invention in samples derived from bodily fluids and normal or cancerous human breast, lymph, intestine, colon, lung, ovarian or prostate tissue.

Typically, the affinity or avidity of an antibody (or antibody multimer, as in the case of an IgM pentanier) of the present invention for a protein or protein fragment of the present invention will be at least about 1×10⁻⁶ molar (M), typically at least about 5×10⁻⁷ M, 1×10⁻⁷ M, with affinities and avidities of at least 1×10⁻⁸ M, 5×10⁻⁹ M, 1×10⁻¹⁰ M and up to 1×10⁻¹³ M proving especially useful.

The antibodies of the present invention can be naturally occurring forms, such as IgG, IgM, IgD, IgE, IgY, and IgA, from any avian, reptilian, or mammalian species.

Human antibodies can, but will infrequently, be drawn directly from human donors or human cells. In such case, antibodies to the polypeptides of the present invention will typically have resulted from fortuitous immunization, such as autoimmune immunization, with the polypeptide of the present invention. Such antibodies will typically, but will not invariably, be polyclonal. In addition, individual polyclonal antibodies may be isolated and cloned to generate monoclonals.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE, IgY, and IgA antibodies of the present invention are also usefully obtained from other species, including mammals such as rodents (typically mouse, but also rat, guinea pig, and hamster), lagomorphs (typically rabbits), and also larger mammals, such as sheep, goats, cows, and horses; or egg laying birds or reptiles such as chickens or alligators. In such cases, as with the transgenic human-antibody-producing non-human mammals, fortuitous immunization is not required, and the non-human mammal is typically affirmatively immunized, according to standard immunization protocols, with the polypeptide of the present invention. One form of avian antibodies may be generated using techniques described in WO 00/29444, published 25 May 2000.

As discussed above, virtually all fragments of 8 or more contiguous amino acids of a polypeptide of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the polypeptide of the present invention to other moieties. For example, polypeptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85: 5409-5413 (1988); Posnett et al., J. Biol. Chem. 263: 1719-1725 (1988).

Protocols for immunizing non-human mammals or avian species are well-established in the art. See Harlow et al. (eds.), Using Antibodies: A Laboratory Manual, Cold Spring Harbor Laboratory (1998); Coligan et al. (eds.), Current Protocols in hmunology, John Wiley & Sons, Inc. (2001); Zola, Monoclonal Antibodies: Preparation and Use of Monoclonal Antibodies and Engineered Antibody Derivatives (Basics: From Background to Bench), Springer Verlag (2000); Gross M, Speck J. Dtsch. Tierarztl. Wochenschr. 103: 417-422 (1996). Immunization protocols often include multiple immunizations, either with or without adjuvants such as Freund's complete adjuvant and Freund's incomplete adjuvant, and may include naked DNA immunization (Moss, Semin. Immunol. 2: 317-327 (1990).

Antibodies from non-human mammals and avian species can be polyclonal or monoclonal, with polyclonal antibodies having certain advantages in immunohistochemical detection of the polypeptides of the present invention and monoclonal antibodies having advantages in identifying and distinguishing particular epitopes of the polypeptides of the present invention. Antibodies from avian species may have particular advantage in detection of the polypeptides of the present invention, in human serum or tissues (Vikinge et al., Biosens. Bioelectron. 13: 1257-1262 (1998). Following immunization, the antibodies of the present invention can be obtained using any art-accepted technique. Such techniques are well known in the art and are described in detail in references such as Coligan, supra; Zola, supra; Howard et al. (eds.), Basic Methods in Antibody Production and Characterization, CRC Press (2000); Harlow, supra; Davis (ed.), Monoclonal Antibody Protocols, Vol. 45, Humana Press (1995); Delves (ed.), Antibody Production Essential Techniques, John Wiley & Son Ltd (1997); and Kenney, Antibody Solution An Antibody Methods Manual, Chapman & Hall (1997).

Briefly, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the polypeptides of the present invention can be cloned from hybridomas and thereafter expressed in other host cells. Nor need the two necessarily be performed together: e.g., genes encoding antibodies specific for the polypeptides of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production of whole antibodies, antibody fragments, or antibody derivatives can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established. See, e.g., Sidhu, Curr. Opin. Biotechnol. 11(6): 610-6 (2000); Griffiths et al., Curr. Opin. Biotechnol. 9(1): 102-8 (1998); Hoogenboom et al., Immunotechnology, 4(1): 1-20 (1998); Rader et al., Current Opinion in Biotechnology 8: 503-508 (1997); Aujame et al., Human Antibodies 8: 155-168 (1997); Hoogenboom, Trends in Biotechnol. 15: 62-70 (1997); de Kruif et al., 17: 453-455 (1996); Barbas et al., Trends in Biotechnol. 14: 230-234 (1996); Winter et al., Ann. Rev. Immunol. 433-455 (1994). Techniques and protocols required to generate, propagate, screen (pan), and use the antibody fragments from such libraries have recently been compiled. See, e.g., Barbas (2001), supra; Kay, supra; and Abelson, supra.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell. Eukaryotic cells are also useful for expression of the antibodies, antibody fragments, and antibody derivatives of the present invention. For example, antibody fragments of the present invention can be produced in Pichia pastoris and in Saccharomyces cerevisiae. See, e.g., Takahashi et al., Biosci. Biotechnol. Biochem. 64(10): 2138-44 (2000); Freyre et al., J. Biotechnol. 76(2-3):157-63 (2000); Fischer et al., Biotechnol. Appl. Biochem. 30 (Pt 2): 117-20 (1999); Pennell et al., Res. Immunol. 149(6): 599-603 (1998); Eldin et al., J. Immunol. Methods. 201(1): 67-75 (1997); Frenken et al., Res. Immunol. 149(6): 589-99 (1998); and Shusta et al., Nature Biotechnol. 16(8): 773-7 (1998).

Antibodies, including antibody fragments and derivatives, of the present invention can also be produced in insect cells. See, e.g., Li et al., Protein Expr. Purif. 21(1): 121-8 (2001); Ailor et al., Biotechnol. Bioeng. 58(2-3): 196-203 (1998); Hsu et al., Biotechnol. Prog. 13(1): 96-104 (1997); Edelman et al., Immunology 91(1): 13-9 (1997); and Nesbit et al., J. Immunol. Methods 151(1-2): 201-8 (1992).

Antibodies and fragments and derivatives thereof of the present invention can also be produced in plant cells, particularly maize or tobacco, Giddings et al., Nature Biotechnol. 18(11): 1151-5 (2000); Gavilondo et al., Biotechniques 29(1): 128-38 (2000); Fischer et al., J. Biol. Regul. Homeost. Agents 14(2): 83-92 (2000); Fischer et al., Biotechnol. Appl. Biochem. 30 (Pt 2): 113-6 (1999); Fischer et al., Biol. Chem. 380(7-8): 825-39 (1999); Russell, Curr. Top. Microbiol. Immunol. 240: 119-38 (1999); and Ma et al., Plant Physiol. 109(2): 341-6 (1995).

Antibodies, including antibody fragments and derivatives, of the present invention can also be produced in transgenic, non-human, mammalian milk. See, e.g. Pollock et al., Immunol Methods. 231: 147-57 (1999); Young et al., Res. Immunol. 149: 609-10 (1998); and Limonta et al., Immunotechnology 1: 107-13 (1995).

Mammalian cells useful for recombinant expression of antibodies, antibody fragments, and antibody derivatives of the present invention include CHO cells, COS cells, 293 cells, and myeloma cells. Verma et al., J. Immunol. Methods 216(1-2):165-81 (1998) review and compare bacterial, yeast, insect and mammalian expression systems for expression of antibodies. Antibodies of the present invention can also be prepared by cell free translation, as further described in Merk et al., J. Biochem. (Tokyo) 125(2): 328-33 (1999) and Ryabova et al., Nature Biotechnol. 15(1): 79-84 (1997), and in the milk of transgenic animals, as further described in Pollock et al., J. Immunol. Methods 231(1-2): 147-57 (1999).

The invention further provides antibody fragments that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention. Among such useful fragments are Fab, Fab′, Fv, F(ab)′₂, and single chain Fv (scFv) fragments. Other useful fragments are described in Hudson, Curr. Opin. Biotechnol. 9(4): 395-402 (1998).

The present invention also relates to antibody derivatives that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention.

Among such useful derivatives are chimeric, primatized, and humanized antibodies; such derivatives are less immunogenic in human beings, and thus are more suitable for in vivo administration, than are unmodified antibodies from non-human mammalian species. Another useful method is PEGylation to increase the serum half life of the antibodies.

Chimeric antibodies typically include heavy and/or light chain variable regions (including both CDR and framework residues) of immunoglobulins of one species, typically mouse, fused to constant regions of another species, typically human. See, e.g., Morrison et al., Proc. Natl. Acad. Sci. USA. 81(21): 6851-5 (1984); Sharon et al., Nature 309(5966): 364-7 (1984); Takeda et al., Nature 314(6010): 452-4 (1985); and U.S. Pat. No. 5,807,715 the disclosure of which is incorporated herein by reference in its entirety. Primatized and humanized antibodies typically include heavy and/or light chain CDRs from a murine antibody grafted into a non-human primate or human antibody V region framework, usually further comprising a human constant region, Riechmann et al., Nature 332(6162): 323-7 (1988); Co et al., Nature 351(6326): 501-2 (1991); and U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties. Other useful antibody derivatives of the invention include heteromeric antibody complexes and antibody fusions, such as diabodies (bispecific antibodies), single-chain diabodies, and intrabodies.

It is contemplated that the nucleic acids encoding the antibodies of the present invention can be operably joined to other nucleic acids forming a recombinant vector for cloning or for expression of the antibodies of the invention. Accordingly, the present invention includes any recombinant vector containing the coding sequences, or part thereof, whether for eukaryotic transduction, transfection or gene therapy. Such vectors may be prepared using conventional molecular biology techniques, known to those with skill in the art, and would comprise DNA encoding sequences for the immunoglobulin V-regions including framework and CDRs or parts thereof, and a suitable promoter either with or without a signal sequence for intracellular transport. Such vectors may be transduced or transfected into eukaryotic cells or used for gene therapy (Marasco et al., Proc. Natl. Acad. Sci. (USA) 90: 7889-7893 (1993); Duan et al., Proc. Natl. Acad. Sci. (USA) 91: 5075-5079 (1994), by conventional techniques, known to those with skill in the art.

The antibodies of the present invention, including fragments and derivatives thereof, can usefully be labeled. It is, therefore, another aspect of the present invention to provide labeled antibodies that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention. The choice of label depends, in part, upon the desired use.

For example, when the antibodies of the present invention are used for immunohistochemical staining of tissue samples, the label can usefully be an enzyme that catalyzes production and local deposition of a detectable product. Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopyranoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133: 331-53 (1986); Kricka et al., J. Immunoassay 17(1): 67-83 (1996); and Lundqvist et al., J. Biolumin. Chemilumin. 10(6): 353-9 (1995). Kits for such enhanced chemiluminescent detection (ECL) are available commercially. The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores. There are a wide variety of fluorophore labels that can usefully be attached to the antibodies of the present invention. For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention. For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as ³³P, ³²P, ³⁵S, ³H, and ¹²⁵I. As another example, when the antibodies of the present invention are used for radioimmunotherapy, the label can usefully be ²²⁸Th, ²²⁷Ac, ²²⁵Ac, Ra, ²¹³Bi, ²¹²Pb, ²¹²Bi, ²¹¹At, ²⁰³Pb ¹⁹⁴Os, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y, ⁹⁰Sr, 88Y, ⁷²Se, ⁶⁷Cu, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2): 529-38 (1998), or by radioisotopic labeling.

As would be understood, use of the labels described above is not restricted to the application as for which they were mentioned.

The antibodies of the present invention, including fragments and derivatives thereof, can also be conjugated to toxins, in order to target the toxin's ablative action to cells that display and/or express the polypeptides of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, vol. 166), Humana Press (2000); and Frankel et al. (eds.), Clinical Applications of Immunotoxins, Springer-Verlag (1998).

The antibodies of the present invention can usefully be attached to a substrate, and it is, therefore, another aspect of the invention to provide antibodies that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention, attached to a substrate. Substrates can be porous or nonporous, planar or nonplanar. For example, the antibodies of the present invention can usefully be conjugated to filtration media, such as NHS-activated Sepharose or CNBr-activated Sepharose for purposes of immunoaffinity chromatography. For example, the antibodies of the present invention can usefully be attached to paramagnetic microspheres, typically by biotin-streptavidin interaction, which microsphere can then be used for isolation of cells that express or display the polypeptides of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the CaSPs of the present invention or to polypeptides encoded by the CaSNAs of the invention.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

### Transgenic Animals and Cells

In another aspect, the invention provides transgenic cells and non-human organisms comprising nucleic acid molecules of the invention. In a preferred embodiment, the transgenic cells and non-human organisms comprise a nucleic acid molecule encoding a CaSP. In a preferred embodiment, the CaSP comprises an amino acid sequence selected from the gene products of Table 2a or Table 2b, or a fragment, mutein, homologous protein or allelic variant thereof. In another preferred embodiment, the transgenic cells and non-human organism comprise a CaSNA of the invention, preferably a CaSNA comprising a nucleotide sequence selected from the group consisting of the gene products of Table 2a, Table 2b or Table 7, or a part, substantially similar nucleic acid molecule, allelic variant or hybridizing nucleic acid molecule thereof.

In another embodiment, the transgenic cells and non-human organisms have a targeted disruption or replacement of the endogenous orthologue of the human CaSG. The transgenic cells can be embryonic stem cells or somatic cells. The transgenic non-human organisms can be chimeric, nonchimeric heterozygotes, and nonchimeric homozygotes. Methods of producing transgenic animals are well known in the art. See, e.g., Hogan et al., Manipulating the Mouse Embryo: A Laboratory Manual, 2d ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999).

Any technique known in the art may be used to introduce a nucleic acid molecule of the invention into an animal to produce the founder lines of transgenic animals. Such techniques include, but are not limited to, pronuclear microinjection. (see, e.g., Paterson et al., Appl. Microbiol. Biotechnol. 40: 691-698 (1994); Carver et al., Biotechnology 11: 1263-1270 (1993); Wright et al., Biotechnology 9: 830-834 (1991); and U.S. Pat. No. 4,873,191, herein incorporated by reference in its entirety); retrovirus-mediated gene transfer into germ lines, blastocysts or embryos (see, e.g., Van der Putten et al., Proc. Natl. Acad. Sci., USA 82: 6148-6152 (1985)); gene targeting in embryonic stem cells (see, e.g., Thompson et al., Cell 56: 313-321 (1989)); electroporation of cells or embryos (see, e.g., Lo, 1983, Mol. Cell. Biol. 3: 1803-1814 (1983)); introduction using a gene gun (see, e.g., Ulmer et al., Science 259: 1745-49 (1993); introducing nucleic acid constructs into embryonic pleuripotent stem cells and transferring the stem cells back into the blastocyst; and sperm-mediated gene transfer (see, e.g., Lavitrano et al., Cell 57: 717-723 (1989)).

Other techniques include, for example, nuclear transfer into enucleated oocytes of nuclei from cultured embryonic, fetal, or adult cells induced to quiescence (see, e.g., Campell et al., Nature 380: 64-66 (1996); Wilmut et al., Nature 385: 810-813 (1997)). The present invention provides for transgenic animals that carry the transgene (i.e., a nucleic acid molecule of the invention) in all their cells, as well as animals which carry the transgene in some, but not all their cells, i.e., mosaic animals or chimeric animals.

The transgene may be integrated as a single transgene or as multiple copies, such as in concatamers, e.g., head-to-head tandems or head-to-tail tandems. The transgene may also be selectively introduced into and activated in a particular cell type by following, e.g., the teaching of Lasko et al. et al., Proc. Natl. Acad. Sci. USA 89: 6232-6236 (1992). The regulatory sequences required for such a cell-type specific activation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

Methods for creating a transgenic animal with a disruption of a targeted gene are also well known in the art. In general, a vector is designed to comprise some nucleotide sequences homologous to the endogenous targeted gene. The vector is introduced into a cell so that it may integrate, via homologous recombination with chromosomal sequences, into the endogenous gene, thereby disrupting the function of the endogenous gene. The transgene may also be selectively introduced into a particular cell type, thus inactivating the endogenous gene in only that cell type. See, e.g., Gu et al., Science 265: 103-106 (1994). The regulatory sequences required for such a cell-type specific inactivation will depend upon the particular cell type of interest, and will be apparent to those of skill in the art. See, e.g., Smithies et al., Nature 317: 230-234 (1985); Thomas et al., Cell 51: 503-512 (1987); Thompson et al., Cell 5: 313-321 (1989).

In one embodiment, a mutant, non-functional nucleic acid molecule of the invention (or a completely unrelated DNA sequence) flanked by DNA homologous to the endogenous nucleic acid sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene. See, e.g., Thomas, supra and Thompson, supra. However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from an animal or patient or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc.

The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. See, e.g., U.S. Pat. Nos. 5,399,349 and 5,460,959, each of which is incorporated by reference herein in its entirety.

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

### Computer Readable Means

A further aspect of the invention is a computer readable means for storing the nucleic acid and amino acid sequences of the instant invention. In a preferred embodiment, the invention provides a computer readable means for storing the gene products of Table 2a and Table 2b and the gene products of Table 2a, Table 2b or Table 7 as described herein, as the complete set of sequences or in any combination. The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

The nucleic acid and amino acid sequences of the invention are particularly useful as components in databases useful for search analyses as well as in sequence analysis algorithms. As used herein, the terms “nucleic acid sequences of the invention” and “amino acid sequences of the invention” mean any detectable chemical or physical characteristic of a polynucleotide or polypeptide of the invention that is or may be reduced to or stored in a computer readable form. These include, without limitation, chromatographic scan data or peak data, photographic data or scan data therefrom, and mass spectrographic data.

This invention provides computer readable media having stored thereon sequences of the invention. A computer readable medium may comprise one or more of the following: a nucleic acid sequence comprising a sequence of a nucleic acid sequence of the invention; an amino acid sequence comprising an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of one or more nucleic acid sequences of the invention; a data set representing a nucleic acid sequence encoding an amino acid sequence comprising the sequence of an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of a nucleic acid sequence of the invention; a data set representing a nucleic acid sequence encoding an amino acid sequence comprising the sequence of an amino acid sequence of the invention. The computer readable medium can be any composition of matter used to store information or data, including, for example, commercially available floppy disks, tapes, hard drives, compact disks, and video disks.

Also provided by the invention are methods for the analysis of character sequences, particularly genetic sequences. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, and sequencing chromatogram peak analysis.

A computer-based method is provided for performing nucleic acid sequence identity or similarity identification. This method comprises the steps of providing a nucleic acid sequence comprising the sequence of a nucleic acid of the invention in a computer readable medium; and comparing said nucleic acid sequence to at least one nucleic acid or amino acid sequence to identify sequence identity or similarity.

A computer-based method is also provided for performing amino acid homology identification, said method comprising the steps of: providing an amino acid sequence comprising the sequence of an amino acid of the invention in a computer readable medium; and comparing said amino acid sequence to at least one nucleic acid or an amino acid sequence to identify homology.

A computer-based method is still further provided for assembly of overlapping nucleic acid sequences into a single nucleic acid sequence, said method comprising the steps of: providing a first nucleic acid sequence comprising the sequence of a nucleic acid of the invention in a computer readable medium; and screening for at least one overlapping region between said first nucleic acid sequence and a second nucleic acid sequence. In addition, the invention includes a method of using patterns of expression associated with either the nucleic acids or proteins in a computer-based method to diagnose disease.

### Diagnostic Methods for Breast Cancer

The present invention also relates to quantitative and qualitative diagnostic assays and methods for detecting, diagnosing, monitoring, staging and predicting cancers by comparing the expression of a CaSNA or a CaSP in a human patient that has or may have breast cancer, or who is at risk of developing breast cancer, with the expression of a CaSNA or a CaSP in a normal human control. For purposes of the present invention, “expression of a CaSNA” or “CaSNA expression” means the quantity of CaSNA mRNA that can be measured by any method known in the art or the level of transcription that can be measured by any method known in the art in a cell, tissue, bodily fluid, organ or whole patient. Similarly, the term “expression of a CaSP” or “CaSP expression” means the amount of CaSP that can be measured by any method known in the art or the level of translation of a CaSNA that can be measured by any method known in the art.

The present invention provides methods for diagnosing breast cancer in a patient, by analyzing for changes in levels of CaSNA or CaSP in cells, tissues, organs or bodily fluids compared with levels of CaSNA or CaSP in cells, tissues, organs or bodily fluids of preferably the same type from a control, wherein an increase, or decrease in certain cases, in levels of a CaSNA or CaSP in the patient versus the control is associated with the presence of breast cancer or with a predilection to the disease. In a preferred embodiment the control is a normal human control or recombinant standard control. In another preferred embodiment, the present invention provides methods for diagnosing breast cancer in a patient by analyzing changes in the structure of the mRNA of a CaSG compared to the mRNA from a normal control. These changes include, without limitation, aberrant splicing, alterations in polyadenylation and/or alterations in 5′ nucleotide capping. In yet another preferred embodiment, the present invention provides methods for diagnosing breast cancer in a patient by analyzing changes in a CaSP compared to a CaSP from a normal patient. These changes include, e.g., alterations, including post translational modifications such as glycosylation and/or phosphorylation of the CaSP or changes in the subcellular CaSP localization.

For purposes of the present invention, diagnosing means that CaSNA or CaSP levels are used to determine the presence, absence, recurrence, metastases or prognosis of disease in a patient. As will be understood by those of skill in the art, measurement of other diagnostic parameters may be required for definitive diagnosis, prognosis or determination of the appropriate treatment for the disease. The determination may be made by a clinician, a doctor, a testing laboratory, or a patient using an over the counter test. The patient may have symptoms of disease or may be asymptomatic. In addition, the CaSNA or CaSP levels of the present invention may be used as screening marker to determine whether further tests or biopsies are warranted. In addition, the CaSNA or CaSP levels may be used to determine the vulnerability or susceptibility to disease.

In a preferred embodiment, the expression of a CaSNA is measured by determining the amount of a mRNA that encodes an amino acid sequence selected from the gene products of Table 2a and Table 2b, a homolog, an allelic variant, or a fragment thereof. In a more preferred embodiment, the CaSNA expression that is measured is the level of expression of a CaSNA mRNA selected from the gene products of Table 2a, Table 2b or Table 7, or a hybridizing nucleic acid, homologous nucleic acid or allelic variant thereof, or a part of any of these nucleic acid molecules. CaSNA expression may be measured by any method known in the art, such as those described supra, including measuring mRNA expression by Northern blot, quantitative or qualitative reverse transcriptase PCR (RT-PCR), microarray, dot or slot blots or in situ hybridization. See, e.g., Ausubel (1992), supra; Ausubel (1999), supra; Sambrook (1989), supra; and Sambrook (2001), supra. CaSNA transcription may be measured by any method known in the art including using a reporter gene hooked up to the promoter of a CaSG of interest or doing nuclear run-off assays. Alterations in mRNA structure, e.g., aberrant splicing variants, may be determined by any method known in the art, including, RT-PCR followed by sequencing or restriction analysis. As necessary, CaSNA expression may be compared to a known control, such as a normal breast nucleic acid, to detect a change in expression.

In another preferred embodiment, the expression of a CaSP is measured by determining the level of a CaSP having an amino acid sequence selected from the group consisting of the gene products of Table 2a and Table 2b, a homolog, an allelic variant, or a fragment thereof. Such levels are preferably determined in at least one of cells, tissues, organs and/or bodily fluids, including determination of normal and abnormal levels. Thus, for instance, a diagnostic assay in accordance with the invention for diagnosing over- or underexpression of a CaSNA or CaSP compared to normal control bodily fluids, cells, tissue samples or recombinant standards may be used to diagnose the presence of breast cancer. The expression level of a CaSP may be determined by any method known in the art, such as those described supra. In a preferred embodiment, the CaSP expression level may be determined by radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. See, e.g, Harlow (1999), supra; Ausubel (1992), supra; and Ausubel (1999), supra. Alterations in the CaSP structure may be determined by any method known in the art, including, e.g., using antibodies that specifically recognize phosphoserine, phosphothreonine or phosphotyrosine residues, two-dimensional polyacrylamide gel electrophoresis (2D PAGE) and/or chemical analysis of amino acid residues of the protein. Id.

In a preferred embodiment, a radioimmunoassay (RIA) or an ELISA is used. An antibody specific to a CaSP is prepared if one is not already available. In a preferred embodiment, the antibody is a monoclonal antibody. The anti-CaSP antibody is bound to a solid support and any free protein binding sites on the solid support are blocked with a protein such as bovine serum albumin. A sample of interest is incubated with the antibody on the solid support under conditions in which the CaSP will bind to the anti-CaSP antibody. The sample is removed, the solid support is washed to remove unbound material, and an anti-CaSP antibody that is linked to a detectable reagent (a radioactive substance for RIA and an enzyme for ELISA) is added to the solid support and incubated under conditions in which binding of the CaSP to the labeled antibody will occur. After binding, the unbound labeled antibody is removed by washing. For an ELISA, one or more substrates are added to produce a colored reaction product that is based upon the amount of an CaSP in the sample. For an RIA, the solid support is counted for radioactive decay signals by any method known in the art. Quantitative results for both RIA and ELISA typically are obtained by reference to a standard curve.

Other methods to measure CaSP levels are known in the art. For instance, a competition assay may be employed wherein an anti-CaSP antibody is attached to a solid support and an allocated amount of a labeled CaSP and a sample of interest are incubated with the solid support. The amount of labeled CaSP attached to the solid support can be correlated to the quantity of a CaSP in the sample.

Of the proteomic approaches, 2D PAGE is a well known technique. Isolation of individual proteins from a sample such as serum is accomplished using sequential separation of proteins by isoelectric point and molecular weight. Typically, polypeptides are first separated by isoelectric point (the first dimension) and then separated by size using an electric current (the second dimension). In general, the second dimension is perpendicular to the first dimension. Because no two proteins with different sequences are identical on the basis of both size and charge, the result of 2D PAGE is a roughly square gel in which each protein occupies a unique spot. Analysis of the spots with chemical or antibody probes, or subsequent protein microsequencing can reveal the relative abundance of a given protein and the identity of the proteins in the sample.

Expression levels of a CaSNA can be determined by any method known in the art, including PCR and other nucleic acid methods, such as ligase chain reaction (LCR) and nucleic acid sequence based amplification (NASBA), can be used to detect malignant cells for diagnosis and monitoring of various malignancies. For example, reverse-transcriptase PCR(RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction.

Hybridization to specific DNA molecules (e.g., oligonucleotides) arrayed on a solid support can be used to both detect the expression of and quantitate the level of expression of one or more CaSNAs of interest. In this approach, all or a portion of one or more CaSNAs is fixed to a substrate. A sample of interest, which may comprise RNA, e.g., total RNA or polyA-selected mRNA, or a complementary DNA (cDNA) copy of the RNA is incubated with the solid support under conditions in which hybridization will occur between the DNA on the solid support and the nucleic acid molecules in the sample of interest. Hybridization between the substrate-bound DNA and the nucleic acid molecules in the sample can be detected and quantitated by several means, including, without limitation, radioactive labeling or fluorescent labeling of the nucleic acid molecule or a secondary molecule designed to detect the hybrid.

The above tests can be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva, peritoneal wash, lymphatic fluid, nipple aspirate, breast milk, mammary gland secretions or any other bodily secretion or derivative thereof. As used herein “blood” includes whole blood, plasma, serum, circulating epithelial cells, constituents, or any derivative of blood.

In addition to detection in bodily fluids, the proteins and nucleic acids of the invention are suitable to detection by cell capture technology. Whole cells may be captured by a variety methods for example magnetic separation, U.S. Pat. Nos. 5,200,084; 5,186,827; 5,108,933; 4,925,788, the disclosures of which are incorporated herein by reference in their entireties. Epithelial cells may be captured using such products as Dynabeads® or CELLection™ (Dynal Biotech, Oslo, Norway). Alternatively, fractions of blood may be captured, e.g., the buffy coat fraction (50 mm cells isolated from 5 ml of blood) containing epithelial cells. In addition, cancer cells may be captured using the techniques described in WO 00/47998, the disclosure of which is incorporated herein by reference in its entirety. Once the cells are captured or concentrated, the proteins or nucleic acids are detected by the means described in the subject application. Alternatively, nucleic acids may be captured directly from blood samples, see U.S. Pat. Nos. 6,156,504, 5,501,963; or WO 01/42504, the disclosures of which are incorporated herein by reference in their entireties.

In a preferred embodiment, the specimen tested for expression of CaSNA or CaSP includes without limitation normal or cancerous breast, intestine, colon, lung, ovarian, prostate, lymph or bone marrow tissue; normal or cancerous breast, intestine, colon, lung, ovarian, prostate, lymph or bone marrow cells grown in cell culture; blood, serum, lymph node tissue, fecal samples, colonocytes, BAL, sputum and lymphatic fluid. In another preferred embodiment, especially when metastasis of a primary breast cancer is known or suspected, specimens include, without limitation, tissues from brain, bone, bone marrow, liver, lungs, lymphatic system, colon, and adrenal glands. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration.

All the methods of the present invention may optionally include determining the expression levels of one or more other cancer markers in addition to determining the expression level of a CaSNA or CaSP. In many cases, the use of another cancer marker will decrease the likelihood of false positives or false negatives. In one embodiment, the one or more other cancer markers include other CaSNA or CaSPs as disclosed herein. Other cancer markers useful in the present invention will depend on the cancer being tested and are known to those of skill in the art. In a preferred embodiment, at least one other cancer marker in addition to a particular CaSNA or CaSP is measured. In a more preferred embodiment, at least two other additional cancer markers are used. In an even more preferred embodiment, at least three, more preferably at least five, even more preferably at least ten additional cancer markers are used.

Colonocytes represent an important source of the CaSP or CaSNAs because they provide a picture of the immediate past metabolic history of the GI tract of a subject. In addition, such cells are representative of the cell population from a statistically large sampling frame reflecting the state of the colonic mucosa along the entire length of the colon in a non-invasive manner, in contrast to a limited sampling by colonic biopsy using an invasive procedure involving endoscopy. Specific examples of patents describing the isolation of colonocytes include U.S. Pat. Nos. 6,335,193; 6,020,137 5,741,650; 6,258,541; US 2001 0026925 A1WO 00/63358 A1, the disclosures of which are incorporated herein by reference in their entireties.

For metastases of breast cancer in the prostate, the progress of therapy can be assessed by routine methods, usually by measuring serum PSA (prostate specific antigen) levels; the higher the level of PSA in the blood, the more extensive the cancer.

Commercial assays for detecting PSA are available, e.g, Hybitech Tandem-E and Tandem-R PSA assay kits, the Yang ProsCheck polyclonal assay (Yang Labs, Bellevue, Wash.), Abbott Imx (Abbott Labs, Abbott Park, Ill.), etc. Metastasis can be determined by staging tests and by bone scan and tests for calcium levels and other enzymes to determine spread to the bone, CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

For bladder cancer, which is a more localized cancer, methods to determine progress of disease include urinary cytologic evaluation by cystoscopy, monitoring for presence of blood in the urine, visualization of the urothelial tract by sonography or an intravenous pyelogram, computed tomography (CT) and magnetic resonance imaging (MRI). The presence of distant metastases can be assessed by CT of the abdomen, chest x-rays, or radionuclide imaging of the skeleton.

Diagnosing

In one aspect, the invention provides a method for determining the expression levels and/or structural alterations of one or more CaSNA and/or CaSP in a sample from a patient suspected of having breast cancer. In general, the method comprises the steps of obtaining the sample from the patient, determining the expression level or structural alterations of a CaSNA and/or CaSP and then ascertaining whether the patient has breast cancer from the expression level of the CaSNA or CaSP. In general, if high expression relative to a control of a CaSNA or CaSP is indicative of breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is indicative of breast cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

In another aspect, the present invention provides a method of determining the expression levels and/or structural alteration of a plurality of CaSNAs and/or CaSPs in a sample from a patient suspected of having breast cancer. In general, the method comprises the steps of obtaining the sample from the patient, determining the expression level or structural alterations of the CaSNAs and/or CaSPs and then ascertaining whether the patient has breast cancer from the expression level of the CaSNAs or CaSPs. In general, if high expression relative to a control of a CaSNA or CaSP is indicative of breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is indicative of breast cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

The present invention also provides a method of determining whether breast cancer has metastasized in a patient. One may identify whether the breast cancer has metastasized by measuring the expression levels and/or structural alterations of one or more CaSNAs and/or CaSPs in a variety of tissues. The presence of a CaSNA or CaSP in a certain tissue at levels higher than that of corresponding noncancerous tissue (e.g., the same tissue from another individual) is indicative of metastasis if high level expression of a CaSNA or CaSP is associated with breast cancer. Similarly, the presence of a CaSNA or CaSP in a tissue at levels lower than that of corresponding noncancerous tissue is indicative of metastasis if low level expression of a CaSNA or CaSP is associated with breast cancer. Further, the presence of a structurally altered CaSNA or CaSP that is associated with breast cancer is also indicative of metastasis.

In general, if high expression relative to a control of a CaSNA or CaSP is indicative of metastasis, an assay for metastasis is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human control. In contrast, if low expression relative to a control of a CaSNA or CaSP is indicative of metastasis, an assay for metastasis is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human control.

In another aspect, the present invention provides a method of determining whether breast cancer has metastasized in a patient based on the expression levels and/or structural alteration of a plurality of CaSNAs and/or CaSPs in a sample from the patient. In general, the method comprises the steps of obtaining the sample from the patient, determining the expression level or structural alterations of a CaSNAs and/or CaSPs and then ascertaining whether the patient has metastatic breast cancer from the expression level of the CaSNAs or CaSPs. In general, if high expression relative to a control of a CaSNA or CaSP is indicative of metastatic breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is indicative of metastatic breast cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

Staging

The invention also provides a method of staging breast cancer in a human patient. The method comprises identifying a human patient having breast cancer and analyzing cells, tissues or bodily fluids from such human patient for expression levels and/or structural alterations of one or more CaSNAs or CaSPs. First, one or more tumors from a variety of patients are staged according to procedures well known in the art, and the expression levels of one or more CaSNAs or CaSPs is determined for each stage to obtain a standard expression level for each CaSNA and CaSP. Then, the CaSNA or CaSP expression levels of the CaSNA or CaSP are determined in a biological sample from a patient whose stage of cancer is not known. The CaSNA or CaSP expression levels from the patient are then compared to the standard expression level. By comparing the expression level of the CaSNAs and CaSPs from the patient to the standard expression levels, one may determine the stage of the tumor. The same procedure may be followed using structural alterations of a CaSNA or CaSP to determine the stage of a breast cancer.

In another aspect, the present invention provides a method of staging breast cancer in a patient based on the expression levels and/or structural alteration of a plurality of CaSNAs and/or CaSPs in a sample from the patient. In general, the method comprises the steps of obtaining the sample from the patient, determining the expression level or structural alterations of a CaSNAs and/or CaSPs and then ascertaining the stage of the breast cancer from the expression level of the CaSNA or CaSP. In general, if high expression relative to a control of a CaSNA or CaSP is useful for staging breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is useful for staging breast cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

Monitoring

Further provided is a method of monitoring breast cancer in a human patient. One may monitor a human patient to determine whether there has been metastasis and, if there has been, when metastasis began to occur. One may also monitor a human patient to determine whether a preneoplastic lesion has become cancerous. One may also monitor a human patient to determine whether a therapy, e.g., chemotherapy, radiotherapy or surgery, has decreased or eliminated the breast cancer. The monitoring may determine if there has been a reoccurrence and, if so, determine its nature. The method comprises identifying a human patient that one wants to monitor for breast cancer, periodically analyzing cells, tissues or bodily fluids from such human patient for expression levels of one or more CaSNAs or CaSPs, and comparing the CaSNA or CaSP levels over time to those CaSNA or CaSP expression levels obtained previously. Patients may also be monitored by measuring one or more structural alterations in a CaSNA or CaSP that are associated with breast cancer.

If increased expression of a CaSNA or CaSP is associated with metastasis, treatment failure, or conversion of a preneoplastic lesion to a cancerous lesion, then detecting an increase in the expression level of a CaSNA or CaSP indicates that the tumor is metastasizing, that treatment has failed or that the lesion is cancerous, respectively. One having ordinary skill in the art would recognize that if this were the case, then a decreased expression level would be indicative of no metastasis, effective therapy or failure to progress to a neoplastic lesion. If decreased expression of a CaSNA or CaSP is associated with metastasis, treatment failure, or conversion of a preneoplastic lesion to a cancerous lesion, then detecting a decrease in the expression level of a CaSNA or CaSP indicates that the tumor is metastasizing, that treatment has failed or that the lesion is cancerous, respectively. In a preferred embodiment, the levels of CaSNAs or CaSPs are determined from the same cell type, tissue or bodily fluid as prior patient samples. Monitoring a patient for onset of breast cancer metastasis is periodic and preferably is done on a quarterly basis, but may be done more or less frequently.

In another aspect, the present invention provides a method of monitoring breast cancer in a patient based on the expression levels and/or structural alteration of a plurality of CaSNAs and/or CaSPs in a sample from the patient. In general, the method comprises the steps of obtaining the sample from the patient, determining the expression level or structural alterations of a CaSNAs and/or CaSPs and then monitoring the breast cancer from the expression level of the CaSNA or CaSP. In general, if high expression relative to a control of a CaSNA or CaSP is useful for monitoring breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is useful for monitoring breast cancer, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

The methods described herein can further be utilized as prognostic assays to identify subjects having or at risk of developing a disease or disorder associated with increased or decreased expression levels of a CaSNA and/or CaSP. The present invention provides a method in which a test sample is obtained from a human patient and one or more CaSNAs and/or CaSPs are detected. The presence of higher (or lower) CaSNA or CaSP levels as compared to normal human controls is diagnostic for the human patient being at risk for developing cancer, particularly breast cancer. The effectiveness of therapeutic agents to decrease (or increase) expression or activity of one or more CaSNAs and/or CaSPs of the invention can also be monitored by analyzing levels of expression of the CaSNAs and/or CaSPs in a human patient in clinical trials or in in vitro screening assays such as in human cells. In this way, the gene product expression pattern can serve as a marker, indicative of the physiological response of the human patient or cells, as the case may be, to the agent being tested.

Detection of Genetic Lesions or Mutations

The methods of the present invention can also be used to detect genetic lesions or mutations in a CaSG, thereby determining if a human with the genetic lesion is susceptible to developing breast cancer or to determine what genetic lesions are responsible, or are partly responsible, for a person's existing breast cancer. Genetic lesions can be detected, for example, by ascertaining the existence of a deletion, insertion and/or substitution of one or more nucleotides from the CaSGs of this invention, a chromosomal rearrangement of a CaSG, an aberrant modification of a CaSG (such as of the methylation pattern of the genomic DNA), or allelic loss of a CaSG. Methods to detect such lesions in the CaSG of this invention are known to those having ordinary skill in the art following the teachings of the specification.

### Methods of Detecting Noncancerous Breast Diseases

The present invention also provides methods for determining the expression levels and/or structural alterations of one or more CaSNAs and/or CaSPs in a sample from a patient suspected of having or known to have a noncancerous breast disease. In general, the method comprises the steps of obtaining a sample from the patient, determining the expression level or structural alterations of a CaSNA and/or CaSP, comparing the expression level or structural alteration of the CaSNA or CaSP to a normal breast control, and then ascertaining whether the patient has a noncancerous breast disease. In general, if high expression relative to a control of a CaSNA or CaSP is indicative of a particular noncancerous breast disease, a diagnostic assay is considered positive if the level of expression of the CaSNA or CaSP is at least two times higher, and more preferably are at least five times higher, even more preferably at least ten times higher, than in preferably the same cells, tissues or bodily fluid of a normal human control. In contrast, if low expression relative to a control of a CaSNA or CaSP is indicative of a noncancerous breast disease, a diagnostic assay is considered positive if the level of expression of the CaSNA or CaSP is at least two times lower, more preferably are at least five times lower, even more preferably at least ten times lower than in preferably the same cells, tissues or bodily fluid of a normal human control. The normal human control may be from a different patient or from uninvolved tissue of the same patient.

In another aspect, the present invention provides a method of detecting non-cancerous breast diseases in a patient based on the expression levels and/or structural alteration of a plurality of CaSNAs and/or CaSPs in a sample from the patient. In general, the method comprises the steps of obtaining the sample from the patient, determining the expression level or structural alterations of a CaSNAs and/or CaSPs and then ascertaining whether the patient has a non-cancerous breast disease from the expression level of the CaSNA or CaSP. In general, if high expression relative to a control of a CaSNA or CaSP is useful for staging breast cancer, a diagnostic assay is considered positive if the level of expression of the CaSNA or CaSP is at least one and a half times higher, and more preferably are at least two times higher, still more preferably five times higher, even more preferably at least ten times higher, than in preferably the same cells, tissues or bodily fluid of a normal human or standard control. In contrast, if low expression relative to a control of a CaSNA or CaSP is useful for ascertaining whether the patient has a non-cancerous breast disease, a diagnostic assay is considered positive if the level of expression of the CaSNA or CaSP is at least one and a half times lower, and more preferably are at least two times lower, still more preferably five times lower, even more preferably at least ten times lower than in preferably the same cells, tissues or bodily fluid of a normal human or standard control. The normal human control may be from a different patient or from uninvolved tissue of the same patient.

One having ordinary skill in the art may determine whether a CaSNA and/or CaSP is associated with a particular noncancerous breast disease by obtaining breast tissue from a patient having a noncancerous breast disease of interest and determining which CaSNAs and/or CaSPs are expressed in the tissue at either a higher or a lower level than in normal breast tissue. In another embodiment, one may determine whether a CaSNA or CaSP exhibits structural alterations in a particular noncancerous breast disease state by obtaining breast tissue from a patient having a noncancerous breast disease of interest and determining the structural alterations in one or more CaSNAs and/or CaSPs relative to normal breast tissue.

### Methods for Identifying Breast Tissue

In another aspect, the invention provides methods for identifying breast tissue. These methods are particularly useful in, e.g., forensic science, breast cell differentiation and development, and in tissue engineering.

In one embodiment, the invention provides a method for determining whether a sample is breast tissue or has breast tissue-like characteristics. The method comprises the steps of providing a sample suspected of comprising breast tissue or having breast tissue-like characteristics, determining whether the sample expresses one or more CaSNAs and/or CaSPs, and, if the sample expresses one or more CaSNAs and/or CaSPs, concluding that the sample comprises breast tissue. In a preferred embodiment, the CaSNA encodes a polypeptide having an amino acid sequence selected from the gene products of Table 2a and Table 2b, or a homolog, allelic variant or fragment thereof. In a more preferred embodiment, the CaSNA has a nucleotide sequence selected from the gene products of Table 2a, Table 2b or Table 7, or a hybridizing nucleic acid, an allelic variant or a part thereof. Determining whether a sample expresses a CaSNA can be accomplished by any method known in the art. Preferred methods include hybridization to microarrays, Northern blot hybridization, and quantitative or qualitative RT-PCR. In another preferred embodiment, the method can be practiced by determining whether a CaSP is expressed. Determining whether a sample expresses a CaSP can be accomplished by any method known in the art. Preferred methods include Western blot, ELISA, RIA and 2D PAGE. In one embodiment, the CaSP has an amino acid sequence selected from the gene products of Table 2a and Table 2b, or a homolog, allelic variant or fragment thereof. In another preferred embodiment, the expression of at least two CaSNAs and/or CaSPs is determined. In a more preferred embodiment, the expression of at least three, more preferably four and even more preferably five CaSNAs and/or CaSPs are determined.

In one embodiment, the method can be used to determine whether an unknown tissue is breast tissue. This is particularly useful in forensic science, in which small, damaged pieces of tissues that are not identifiable by microscopic or other means are recovered from a crime or accident scene. In another embodiment, the method can be used to determine whether a tissue is differentiating or developing into breast tissue. This is important in monitoring the effects of the addition of various agents to cell or tissue culture, e.g., in producing new breast tissue by tissue engineering. These agents include, e.g., growth and differentiation factors, extracellular matrix proteins and culture medium. Other factors that may be measured for effects on tissue development and differentiation include gene transfer into the cells or tissues, alterations in pH, aqueous: air interface and various other culture conditions.

### Methods for Producing and Modifying Breast Tissue

In another aspect, the invention provides methods for producing engineered breast tissue or cells. In one embodiment, the method comprises the steps of providing cells, introducing a CaSNA or a CaSG into the cells, and growing the cells under conditions in which they exhibit one or more properties of breast tissue cells. In a preferred embodiment, the cells are pleuripotent. As is well known in the art, normal breast tissue comprises a large number of different cell types. Thus, in one embodiment, the engineered breast tissue or cells comprises one of these cell types. In another embodiment, the engineered breast tissue or cells comprises more than one breast cell type. Further, the culture conditions of the cells or tissue may require manipulation in order to achieve full differentiation and development of the breast cell tissue. Methods for manipulating culture conditions are well known in the art.

Nucleic acid molecules encoding one or more CaSPs are introduced into cells, preferably pleuripotent cells. In a preferred embodiment, the nucleic acid molecules encode CaSPs having amino acid sequences selected from the gene products of Table 2a and Table 2b, or homologous proteins, analogs, allelic variants or fragments thereof. In a more preferred embodiment, the nucleic acid molecules have a nucleotide sequence selected from the gene products of Table 2a, Table 2b or Table 7, or hybridizing nucleic acids, allelic variants or parts thereof. In another highly preferred embodiment, a CaSG is introduced into the cells. Expression vectors and methods of introducing nucleic acid molecules into cells are well known in the art and are described in detail, supra.

Artificial breast tissue may be used to treat patients who have lost some or all of their breast function.

### Pharmaceutical Compositions

In another aspect, the invention provides pharmaceutical compositions comprising the nucleic acid molecules, polypeptides, fusion proteins, antibodies, antibody derivatives, antibody fragments, agonists, antagonists, or inhibitors of the present invention. In a preferred embodiment, the pharmaceutical composition comprises a CaSNA or part thereof. In a preferred embodiment, the pharmaceutical composition comprises a plurality of CaSNAs or parts thereof. In a more preferred embodiment, the CaSNA has a nucleotide sequence selected from the group consisting of the gene products of Table 2a, Table 2b or Table 7, a nucleic acid that hybridizes thereto, an allelic variant thereof, or a nucleic acid that has substantial sequence identity thereto. In another preferred embodiment, the pharmaceutical composition comprises a CaSP or fragment thereof. In a preferred embodiment, the pharmaceutical composition comprises a plurality of CaSPs or fragments thereof. In a more preferred embodiment, the pharmaceutical composition comprises a CaSP having an amino acid sequence that is selected from the group consisting of the gene products of Table 2a and Table 2b, a polypeptide that is homologous thereto, a fusion protein comprising all or a portion of the polypeptide, or an analog or derivative thereof. In another preferred embodiment, the pharmaceutical composition comprises an anti-CaSP antibody, preferably an antibody that specifically binds to a CaSP having an amino acid sequence that is selected from the group consisting of the gene products of Table 2a and Table 2b, or an antibody that binds to a polypeptide that is homologous thereto, a fusion protein comprising all or a portion of the polypeptide, or an analog, isoform, allelic variant or derivative thereof. In another preferred embodiment, the pharmaceutical composition comprises a plurality of anti-CaSP antibodies, preferably antibodies that specifically bind to a CaSPs having an amino acid sequences that are selected from the group consisting of the gene products of Table 2a and Table 2b, or antibodies that bind to polypeptides that are homologous thereto, fusion proteins comprising all or a portion of the polypeptides, or analogs, isoforms, allelic variants or derivatives thereof. In another preferred embodiment, the pharmaceutical composition comprises a plurality of CaSP agonist molecules, preferably agonist molecules that are agonistic to CaSPs having an amino acid sequences that are selected from the group consisting of the gene products of Table 2a and Table 2b, or agonists that are agonistic to polypeptides that are homologous thereto, fusion proteins comprising all or a portion of the polypeptides, or analogs, isoforms, allelic variants or derivatives thereof. In another preferred embodiment, the pharmaceutical composition comprises a plurality of CaSP antagonist molecules, preferably antagonist molecules that are antagonistic to CaSPs having an amino acid sequences that are selected from the group consisting of the gene products of Table 2a and Table 2b, or antagonists that are antagonistic to polypeptides that are homologous thereto, fusion proteins comprising all or a portion of the polypeptides, or analogs, isoforms, allelic variants or derivatives thereof. In another preferred embodiment, the pharmaceutical composition comprises a plurality of CaSP antagonist and agonist molecules, preferably antagonist and agonist molecules that are antagonistic and agonistic to CaSPs having an amino acid sequences that are selected from the group consisting of the gene products of Table 2a and Table 2b, or antagonists and agonists that are antagonistic or agonistic to polypeptides that are homologous thereto, fusion proteins comprising all or a portion of the polypeptides, or analogs, isoforms, allelic variants or derivatives thereof.

Due to the association of angiogenesis with cancer vascularization there is great need of new markers and methods for diagnosing angiogenesis activity to identify developing tumors and angiogenesis related diseases. Furthermore, great need is also present for new molecular targets useful in the treatment of angiogenesis and angiogenesis related diseases such as cancer. In addition known modulators of angiogenesis such as endostatin or vascular endothelial growth factor (VEGF). Use of the methods and compositions disclosed herein in combination with anti-angiogenesis drugs, drugs that block the matrix breakdown (such as BMS-275291, Dalteparin (Fragmin®), Suramin), drugs that inhibit endothelial cells (2-methoxyestradiol (2-ME), CC-5013 (Thalidomide Analog), Combretastatin A4 Phosphate, LY317615 (Protein Kinase C Beta Inhibitor), Soy Isoflavone (Genistein; Soy Protein Isolate), Thalidomide), drugs that block activators of angiogenesis (AE-941 (Neovastat™; GW786034), Anti-VEGF Antibody (Bevacizumab; Avastin™), Interferon-alpha, PTK787/ZK 222584, VEGF-Trap, ZD6474), drugs that inhibit endothelial-specific integrin/survival signaling (EMD 121974, Anti-Anb3 Integrin Antibody (Medi-522; Vitaxin™)).

Such a composition typically contains from about 0.1 to 90% by weight of a therapeutic agent of the invention formulated in and/or with a pharmaceutically acceptable carrier or excipient.

Pharmaceutical formulation is a well-established art that is further described in Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins (1999); and Kibbe (ed.), Handbook of Pharmaceutical Excipients American Pharmaceutical Association, 3^{rd} ed. (2000) and thus need not be described in detail herein.

Briefly, formulation of the pharmaceutical compositions of the present invention will depend upon the route chosen for administration. The pharmaceutical compositions utilized in this invention can be administered by various routes including both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

Oral dosage forms can be formulated as tablets, pills, dragees, capsules, liquids, gels, syrups, slurries, suspensions, and the like, for ingestion by the patient.

Solid formulations of the compositions for oral administration can contain suitable carriers or excipients, such as carbohydrate or protein fillers; sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid.

Agents that facilitate disintegration and/or solubilization can be added, such as the cross-linked polyvinyl pyrrolidone, agar, alginic acid, or a salt thereof, such as sodium alginate, microcrystalline cellulose, cornstarch, sodium starch glycolate, and alginic acid.

Tablet binders that can be used include acacia, methylcellulose, sodium carboxymethylcellulose, polyvinylpyrrolidone (Povidone™), hydroxypropyl methylcellulose, sucrose, starch and ethylcellulose.

Lubricants that can be used include magnesium stearates, stearic acid, silicone fluid, talc, waxes, oils, and colloidal silica.

Fillers, agents that facilitate disintegration and/or solubilization, tablet binders and lubricants, including the aforementioned, can be used singly or in combination.

Solid oral dosage forms need not be uniform throughout. For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Oral dosage forms of the present invention include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Additionally, dyestuffs or pigments can be added to the tablets or dragee coatings for product identification or to characterize the quantity of active compound, i.e., dosage.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents.

The pharmaceutical compositions of the present invention can also be formulated for parenteral administration. Formulations for parenteral administration can be in the form of aqueous or non-aqueous isotonic sterile injection solutions or suspensions.

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution. Intravenous formulations may include carriers, excipients or stabilizers including, without limitation, calcium, human serum albumin, citrate, acetate, calcium chloride, carbonate, and other salts.

Intramuscular preparations, e.g. a sterile formulation of a suitable soluble salt form of the compounds of the present invention, can be dissolved and administered in a pharmaceutical excipient such as Water-for-Injection, 0.9% saline, or 5% glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Parenteral formulations of the compositions can contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like).

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical compositions of the present invention can also be formulated to permit injectable, long-term, deposition. Injectable depot forms may be made by forming microencapsulated matrices of the compound in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in microemulsions that are compatible with body tissues.

The pharmaceutical compositions of the present invention can be administered topically. For topical use the compounds of the present invention can also be prepared in suitable forms to be applied to the skin, or mucus membranes of the nose and throat, and can take the form of lotions, creams, ointments, liquid sprays or inhalants, drops, tinctures, lozenges, or throat paints. Such topical formulations further can include chemical compounds such as dimethylsulfoxide (DMSO) to facilitate surface penetration of the active ingredient. In other transdermal formulations, typically in patch-delivered formulations, the pharmaceutically active compound is formulated with one or more skin penetrants, such as 2-N-methyl-pyrrolidone (NMP) or Azone. A topical semi-solid ointment formulation typically contains a concentration of the active ingredient from about 1 to 20%, e.g., 5 to 10%, in a carrier such as a pharmaceutical cream base.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

Inhalation formulations can also readily be formulated. For inhalation, various powder and liquid formulations can be prepared. For aerosol preparations, a sterile formulation of the compound or salt form of the compound may be used in inhalers, such as metered dose inhalers, and nebulizers. Aerosolized forms may be especially useful for treating respiratory disorders.

Alternatively, the compounds of the present invention can be in powder form for reconstitution in the appropriate pharmaceutically acceptable carrier at the time of delivery.

The pharmaceutically active compound in the pharmaceutical compositions of the present invention can be provided as the salt of a variety of acids, including but not limited to hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art.

A “therapeutically effective dose” refers to that amount of active ingredient, for example CaSP polypeptide, fusion protein, or fragments thereof, antibodies specific for CaSP, agonists, antagonists or inhibitors of CaSP, which ameliorates the signs or symptoms of the disease or prevent progression thereof; as would be understood in the medical arts, cure, although desired, is not required.

The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial preferred concentration range and route of administration.

For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are preferred.

The data obtained from cell culture assays and animal studies are used in formulating an initial dosage range for human use, and preferably provide a range of circulating concentrations that includes the ED50 with little or no toxicity. After administration, or between successive administrations, the circulating concentration of active agent varies within this range depending upon pharmacokinetic factors well known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions.

### Therapeutic Methods

The present invention further provides methods of treating subjects having defects in a gene of the invention, e.g., in expression, activity, distribution, localization, and/or solubility, which can manifest as a disorder of breast function. As used herein, “treating” includes all medically-acceptable types of therapeutic intervention, including palliation and prophylaxis (prevention) of disease. The term “treating” encompasses any improvement of a disease, including minor improvements. These methods are discussed below.

Gene Therapy and Vaccines

The isolated nucleic acids of the present invention can also be used to drive in vivo expression of the polypeptides of the present invention. In vivo expression can be driven from a vector, typically a viral vector, often a vector based upon a replication incompetent retrovirus, an adenovirus, or an adeno-associated virus (AAV), for the purpose of gene therapy. In vivo expression can also be driven from signals endogenous to the nucleic acid or from a vector, often a plasmid vector, such as pVAX1 (Invitrogen, Carlsbad, Calif., USA), for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; 6,204,250, the disclosures of which are incorporated herein by reference in their entireties. For cancer therapy, it is preferred that the vector also be tumor-selective. See, e.g., Doronin et al., J. Virol. 75: 3314-24 (2001).

In another embodiment of the therapeutic methods of the present invention, a therapeutically effective amount of a pharmaceutical composition comprising a nucleic acid molecule of the present invention is administered. The nucleic acid molecule can be delivered in a vector that drives expression of a CaSP, fusion protein, or fragment thereof, or without such vector. Nucleic acid compositions that can drive expression of a CaSP are administered, for example, to complement a deficiency in the native CaSP, or as DNA vaccines. Expression vectors derived from virus, replication deficient retroviruses, adenovirus, adeno-associated (AAV) virus, herpes virus, or vaccinia virus can be used as plasmids. See, e.g., Cid-Arregui, supra. In a preferred embodiment, the nucleic acid molecule encodes a CaSP having the amino acid sequence of the gene products of Table 2a and Table 2b, or a fragment, fusion protein, allelic variant or homolog thereof.

In still other therapeutic methods of the present invention, pharmaceutical compositions comprising host cells that express a CaSP, fusions, or fragments thereof can be administered. In such cases, the cells are typically autologous, so as to circumvent xenogeneic or allotypic rejection, and are administered to complement defects in CaSP production or activity. In a preferred embodiment, the nucleic acid molecules in the cells encode a CaSP having the amino acid sequence of the gene products of Table 2a and Table 2b, or a fragment, fusion protein, allelic variant or homolog thereof.

Antisense Administration

Antisense nucleic acid compositions, or vectors that drive expression of a CaSG antisense nucleic acid, are administered to downregulate transcription and/or translation of a CaSG in circumstances in which excessive production, or production of aberrant protein, is the pathophysiologic basis of disease.

Antisense compositions useful in therapy can have a sequence that is complementary to coding or to noncoding regions of a CaSG. For example, oligonucleotides derived from the transcription initiation site, e.g., between positions −10 and +10 from the start site, are preferred.

Catalytic antisense compositions, such as ribozymes, that are capable of sequence-specific hybridization to CaSG transcripts, are also useful in therapy. See, e.g., Phylactou, Adv. Drug Deliv. Rev. 44(2-3): 97-108 (2000); Phylactou et al., Hum. Mol. Genet. 7(10): 1649-53 (1998); Rossi, Ciba Found. Symp. 209: 195-204 (1997); and Sigurdsson et al., Trends Biotechnol. 13(8): 286-9 (1995).

Other nucleic acids useful in the therapeutic methods of the present invention are those that are capable of triplex helix formation in or near the CaSG genomic locus. Such triplexing oligonucleotides are able to inhibit transcription. See, e.g., Intody et al., Nucleic Acids Res. 28(21): 4283-90 (2000); and McGuffie et al., Cancer Res. 60(14): 3790-9 (2000). Pharmaceutical compositions comprising such triplex forming oligos (TFOs) are administered in circumstances in which excessive production, or production of aberrant protein, is a pathophysiologic basis of disease.

In a preferred embodiment, the antisense molecule is derived from a nucleic acid molecule encoding a CaSP, preferably a CaSP comprising an amino acid sequence of the gene products of Table 2a and Table 2b, or a fragment, allelic variant or homolog thereof. In a more preferred embodiment, the antisense molecule is derived from a nucleic acid molecule having a nucleotide sequence of the gene products of Table 2a, Table 2b or Table 7, or a part, allelic variant, substantially similar or hybridizing nucleic acid thereof.

Polypeptide Administration

In one embodiment of the therapeutic methods of the present invention, a therapeutically effective amount of a pharmaceutical composition comprising a CaSP, a fusion protein, fragment, analog or derivative thereof is administered to a subject with a clinically-significant CaSP defect.

Protein compositions are administered, for example, to complement a deficiency in native CaSP. In other embodiments, protein compositions are administered as a vaccine to elicit a humoral and/or cellular immune response to CaSP. The immune response can be used to modulate activity of CaSP or, depending on the immunogen, to immunize against aberrant or aberrantly expressed forms, such as mutant or inappropriately expressed isoforms. In yet other embodiments, protein fusions having a toxic moiety are administered to ablate cells that aberrantly accumulate CaSP.

In a preferred embodiment, the polypeptide administered is a CaSP comprising an amino acid sequence of the gene products of Table 2a and Table 2b, or a fusion protein, allelic variant, homolog, analog or derivative thereof. In a more preferred embodiment, the polypeptide is encoded by a nucleic acid molecule having a nucleotide sequence of the gene products of Table 2a, Table 2b or Table 7, or a part, allelic variant, substantially similar or hybridizing nucleic acid thereof.

Antibody, Agonist and Antagonist Administration

In another embodiment of the therapeutic methods of the present invention, a therapeutically effective amount of a pharmaceutical composition comprising an antibody (including a fragment or derivative thereof) of the present invention is administered. As is well known, antibody compositions are administered, for example, to antagonize activity of CaSP, or to target therapeutic agents to sites of CaSP presence and/or accumulation. In a preferred embodiment, the antibody specifically binds to a CaSP comprising an amino acid sequence of the gene products of Table 2a and Table 2b, or a fusion protein, allelic variant, homolog, analog or derivative thereof. In a more preferred embodiment, the antibody specifically binds to a CaSP encoded by a nucleic acid molecule having a nucleotide sequence of the gene products of Table 2a, Table 2b or Table 7, or a part, allelic variant, substantially similar or hybridizing nucleic acid thereof.

The present invention also provides methods for identifying modulators which bind to a CaSP or have a modulatory effect on the expression or activity of a CaSP. Modulators which decrease the expression or activity of CaSP (antagonists) are believed to be useful in treating breast cancer. Such screening assays are known to those of skill in the art and include, without limitation, cell-based assays and cell-free assays. Small molecules predicted via computer imaging to specifically bind to regions of a CaSP can also be designed, synthesized and tested for use in the imaging and treatment of breast cancer. Further, libraries of molecules can be screened for potential anticancer agents by assessing the ability of the molecule to bind to the CaSPs identified herein. Molecules identified in the library as being capable of binding to a CaSP are key candidates for further evaluation for use in the treatment of breast cancer. In a preferred embodiment, these molecules will downregulate expression and/or activity of a CaSP in cells.

In another embodiment of the therapeutic methods of the present invention, a pharmaceutical composition comprising a non-antibody antagonist of CaSP is administered. Antagonists of CaSP can be produced using methods generally known in the art. In particular, purified CaSP can be used to screen libraries of pharmaceutical agents, often combinatorial libraries of small molecules, to identify those that specifically bind and antagonize at least one activity of a CaSP.

In other embodiments a pharmaceutical composition comprising an agonist of a CaSP is administered. Agonists can be identified using methods analogous to those used to identify antagonists.

In a preferred embodiment, the antagonist or agonist specifically binds to and antagonizes or agonizes, respectively, a CaSP comprising an amino acid sequence of the gene products of Table 2a and Table 2b, or a fusion protein, allelic variant, homolog, analog or derivative thereof. In a more preferred embodiment, the antagonist or agonist specifically binds to and antagonizes or agonizes, respectively, a CaSP encoded by a nucleic acid molecule having a nucleotide sequence of the gene products of Table 2a, Table 2b or Table 7, or a part, allelic variant, substantially similar or hybridizing nucleic acid thereof.

Targeting Breast Tissue

The invention also provides a method in which a polypeptide of the invention, or an antibody thereto, is linked to a therapeutic agent such that it can be delivered to the breast, intestine, colon, lung, ovarian or prostate; or to specific cells in the breast, colon, lung, ovarian or prostate; or metastatic breast cancer cells in lymphatic tissues, bone and bone marrow. In a preferred embodiment, an anti-CaSP antibody is linked to a therapeutic agent and is administered to a patient in need of such therapeutic agent. The therapeutic agent may be a toxin, if breast tissue needs to be selectively destroyed. This is useful for targeting and killing local or metastatic breast cancer cells. In another embodiment, the therapeutic agent may be a growth or differentiation factor, which is useful for promoting breast cell function.

In another embodiment, an anti-CaSP antibody may be linked to an imaging agent that can be detected using, e.g., magnetic resonance imaging, CT or PET. This would be useful for determining and monitoring breast function, identifying local and metastasized breast cancer tumors, and identifying non-cancerous breast diseases.

### EXAMPLES

### Example 1a

### Differentially Expressed Gene Products in Breast Cancer

For the detection of cancer or stratification of individuals into groups predicted to have different disease outcomes, the expression levels of gene products were determined. Genes were selected based on individual expression profiles and functional relevance of the encoded protein as described by gene ontology and the literature. Genes within the functionally relevant groups below are likely to be useful for (1) detection of cancer, (2) stratification of individuals into groups predicted to have different disease outcomes; (3) selection of individuals for a particular therapeutic intervention; or (4) identification of individuals responding to a therapeutic regimen.

TABLE 1

Extracellular matrix
Cell adhesion
Estrogen receptor signaling pathway
Regulation of transcription
Ubiquitination
Lipid metabolism
Signal transduction
DNA repair
Immune response
Transport
Estrogen metabolism
Chemotaxis
G-protein couple receptor
Apoptosis
Cell recognition
Anti-apoptosis

A gene product associated with one or more of the functional categories above will be particularly useful if it has one or more of the following properties: structural and/or physical, chemical or enzymatic, regulatory, signal transduction, or ligand, receptor or substrate binding. In addition, genes or gene products directly involved in the sequential and organ specific development of cancer are of interest.

Based on the criteria above, we identified a set of genes and associated gene products. Table 2a and Table 2b below provide a summary of these genes including: the GenBank Accessions (ncbi with the extension .nlm.nih.gov of the world wide web), the abbreviated common name for the genes, internal identifiers, functional association(s) for the gene product and annotation of the gene from public databases (e.g. GenBank).

In addition, Table 3 below contains the GenBank Accession, the chromosomal location of the gene (with amplification or loss of homology annotation), Gene Ontology (GO) ID/classifications including: Cellular Component Ontology, Molecular Function Ontology and Biological Process Ontology. Also included is a description of gene product function derived from the literature. References supporting GO and functional annotations of the GenBank Accession in Table 3 are available in public databases such as GenBank and Swissprot.

TABLE 2a

| GenBank | | DDXS | |
|---|---|---|---|
| Accession | Abbreviated Name | Amplicon | Annotation |
| NM_018098.4 | ECT2 | Cln176a, | Homo sapiens epithelial cell transforming sequence 2 oncogene (ECT2), |
| | | Cln176b | mRNA. |
| NM_006564.1 | CXCR6 | DSH105 | Homo sapiens chemokine (C-X-C motif) receptor 6 (CXCR6), mRNA. |
| NM_004367.3 | CCR6 | DSH106 | Homo sapiens chemokine (C-C motif) receptor 6 (CCR6), transcript |
| | | | variant 1, mRNA. |
| NM_022161.2 | BIRC7 | DSH127 | Homo sapiens baculoviral IAP repeat-containing 7 (livin) (BIRC7), |
| | | | transcript variant 2, mRNA. |
| BC014098.2 | SNCG | DSH140 | Homo sapiens synuclein, gamma (breast cancer-specific protein 1), |
| | | | mRNA (cDNA clone MGC:20132 IMAGE:4546444), complete cds |
| NM_153758.1 | IL19 | DSH141 | Homo sapiens interleukin 19 (IL19), transcript variant 1, mRNA. |
| NM_014452.3 | TNFRSF21 | DSH176 | Homo sapiens tumor necrosis factor receptor superfamily, member 21 |
| | | | (TNFRSF21), mRNA. |
| NM_003965.3 | CCRL2 | DSH209 | Homo sapiens chemokine (C-C motif) receptor-like 2 (CCRL2), mRNA. |
| NM_001908.3 | CTSB | DSH223 | Homo sapiens cathepsin B (CTSB), transcript variant 1, mRNA |
| NM_001571.2 | IRF3 | DSH371 | Homo sapiens interferon regulatory factor 3 (IRF3), mRNA. |
| NM_005201.2 | CCR8 | DSH375 | Homo sapiens chemokine (C-C motif) receptor 8 (CCR8), mRNA. |
| NM_000579.1 | CCR5 | DSH51 | Homo sapiens chemokine (C-C motif) receptor 5 (CCR5), mRNA. |
| NM_006988.3 | ADAMTS1 | DSH607 | Homo sapiens a disintegrin-like and metalloprotease (reprolysin |
| | | | type) with thrombospondin type 1 motif, 1 (ADAMTS1), mRNA. |
| NM_004591.1 | CCL20 | DSH73 | Homo sapiens chemokine (C-C motif) ligand 20 (CCL20), mRNA. |
| NM_003579.2 | RAD54L | DSH811 | Homo sapiens RAD54-like (S. cerevisiae) (RAD54L), mRNA. |
| AY623117.1 | RAD54-like | DSH811a | Homo sapiens RAD54-like (S. cerevisiae) (RAD54L) gene, complete cds. |
| NM_001838.2 | CCR7 | DSH859 | Homo sapiens chemokine (C-C motif) receptor 7 (CCR7), mRNA. |
| NM_001008540.1 | CXCR4 | DSH862 | Homo sapiens chemokine (C-X-C motif) receptor 4 (CXCR4), |
| | | | transcript variant 1, mRNA. |
| NM_002989.2 | CCL21 | DSH89 | Homo sapiens chemokine (C-C motif) ligand 21 (CCL21), mRNA. |
| NM_178445.1 | CCRL1 | DSH97 | Homo sapiens chemokine (C-C motif) receptor-like 1 (CCRL1), |
| | | | transcript variant 1, mRNA. |
| NM_006551.2 | SCGB1D2 | ECM2 | Homo sapiens secretoglobin, family 1D, member 2 (SCGB1D2), mRNA |
| NM_002407.1 | SCGB2A1 | ECM3 | Homo sapiens secretoglobin, family 2A, member 1 (SCGB2A1), mRNA. |
| AL136163.15 | | Lng186 | Human DNA sequence from clone RP1-185N19 on chromosome |
| | | | 6q22.1-22.33. Contains part of the gene for endoplasmic |
| | | | reticulum associated protein 140 kDa (ERAP140) (similar to |
| | | | nucleolar protein C7B), complete sequence. |
| NM_145016.2 | BXMAS2-10 | Mam004 | Homo sapiens BXMAS2-10 (BXMAS2-10), mRNA. |
| AL157387.9 | | Mam005 | Human DNA sequence from clone RP11-20F24 on chromosome |
| | | | 10p11.21-12.1 Contains the 3′ end of the NY-BR-1 gene for |
| | | | breast cancer antigen NY-BR-1, two novel genes, a pseudogene |
| | | | similar to part of ATP8A2 (ATPase, aminophospholipid |
| | | | transporter-like, Class I, type 8A, member 2), a novel pseudogene |
| | | | and a vomeronasal receptor pseudogene, complete sequence |
| NM_001075.2, | UGT2B10, UGT2B11 | Mam013 | Homo sapiens UDP glycosyltransferase 2 family, polypeptide B10, |
| NM_001073.1 | | | Homo sapiens UDP glycosyltransferase 2 family, polypeptide B11 |
| NM_001073.1 | UGT2B11 | Mam013b | Homo sapiens UDP glycosyltransferase 2 family, polypeptide B11 |
| AC018686.7 | | Mam018 | Homo sapiens BAC clone RP11-458M16 from 2, complete sequence. |
| AC079414.6 | | Mam021, | Homo sapiens chromosome 16 clone RP11-358L22, complete sequence |
| | | Mam021a | |
| AL356432.17 | | Mam028 | Human DNA sequence from clone RP11-325O24 on chromosome 6 |
| | | | Contains a novel pseudogene, a pseudogene similar to part of |
| | | | transducin (beta)-like 1X-linked TBL1X and the 3′ end of |
| | | | a novel gene, complete sequence. |
| AL391686.10 | | Mam029 | Human DNA sequence from clone CTB-BR164I22 on chromosome 10, |
| | | | complete sequence. |
| AC093297.3 | | Mam053 | Homo sapiens chromosome 5 clone RP11-53O19, complete sequence |
| AY327584.1 | MUC1/S2 | Mam096 | Homo sapiens mucin short variant S2 (MUC1) mRNA, complete cds. |
| XM_378356.1 | LOC283400 | Mam109 | PREDICTED: Homo sapiens hypothetical protein LOC283400 |
| | | | (LOC283400), mRNA |
| NM_006408.2 | AGR2 | Mam111 | Homo sapiens anterior gradient 2 homolog (Xenopus laevis) |
| | | | (AGR2), mRNA |
| NM_017697.2 | FLJ20171 | Mam113 | Homo sapiens hypothetical protein FLJ20171 (FLJ20171), mRNA. |
| NM_005449.3 | TOSO | Mam121 | Homo sapiens regulator of Fas-induced apoptosis (TOSO), mRNA. |
| NM_005449.3 | TOSO | Mam121, | Homo sapiens regulator of Fas-induced apoptosis (TOSO), mRNA. |
| | | Mam121a | |
| NM_005449.3 | TOSO | Mam121V1 | Homo sapiens regulator of Fas-induced apoptosis (TOSO), mRNA. |
| NM_016125.2 | LOC51136. PTD016 | Mam122b | Homo sapiens PTD016 protein (LOC51136), mRNA. |
| | protein | | |
| NM_000305.1 | PON2 | Mam123 | Homo sapiens paraoxonase 2 (PON2), mRNA. |
| AL831952.1 | RZPD:DKFZp761B0319 | Mam125 | Homo sapiens mRNA; cDNA DKFZp761B0319 (from clone DKFZp761B0319). |
| NM_006804.2 | STARD3 | Mam125a | Homo sapiens START domain containing 3 (STARD3), mRNA. |
| NM_006573.3 | TNFSF13B | Mam127 | Homo sapiens tumor necrosis factor (ligand) superfamily, |
| | | | member 13b (TNFSF13B), mRNA. |
| AY358768.1 | C13orf11 | Mam128V2 | Homo sapiens clone DNA100902 C13orf11 (UNQ2419) mRNA, complete cds. |
| NM_016056.1 | CGI-119 | Mam131 | Homo sapiens CGI-119 protein (CGI-119), mRNA. |
| NM_024626.1 | B7-H4 | Ovr110a, | Homo sapiens immune costimulatory protein B7-H4 (B7-H4), mRNA |
| | | Ovr110b | |
| BC065717.1 | B7-H4 | Ovr110V1 | Homo sapiens immune costimulatory protein B7-H4, mRNA (cDNA |
| | | | clone MGC:71941 IMAGE:4295775), complete cds. |
| NM_004625.3 | WNT7A | Ovr212 | Homo sapiens wingless-type MMTV integration site family, |
| | | | member 7A (WNT7A), mRNA |
| NM_004625.3 | WNT7A | Ovr212c | Homo sapiens wingless-type MMTV integration site family, |
| | | | member 7A (WNT7A), mRNA. |
| AY216715.1 | RASSF4 | Ovr216 | Homo sapiens putative tumor suppressor RASSF4 isoform C |
| | | | (RASSF4) mRNA, complete cds; alternatively spliced. |
| L08044.1 | | Ovr226 | Human intestinal trefoil factor mRNA, complete cds |
| NM_003226.2 | TFF3 | Ovr226v1 | Homo sapiens trefoil factor 3 (intestinal) (TFF3), mRNA |
| NM_001554.3 | CYR61 | Ovr235c | Homo sapiens cysteine-rich, angiogenic inducer, 61 (CYR61), mRNA |
| BC021275.2 | PACAP | Pcan039b | Homo sapiens proapoptotic caspase adaptor protein, mRNA |
| | | | (cDNA clone MGC:29506 IMAGE:4853250), complete cds. |
| NM_206925.1 | CA12 | Pcan040 | Homo sapiens carbonic anhydrase XII (CA12), transcript variant 2, mRNA |
| NM_022664.1, | ECM1 | Pcan043 | Homo sapiens extracellular matrix protein 1 (ECM1), transcript |
| U68187.1 | | | variant 2, mRNA, Human extracellular matrix protein 1 mRNA, |
| | | | alternative splice variant, complete cds |
| NM_004385.2 | CSPG2 | Pcan045 | Homo sapiens chondroitin sulfate proteoglycan 2 (versican) |
| | | | (CSPG2), mRNA. |
| NM_033438.1 | SLAMF9 | Pcan047V1 | Homo sapiens SLAM family member 9 (SLAMF9), mRNA |
| NM_024042.2 | METRN | Pcan052 | Homo sapiens meteorin, glial cell differentiation regulator |
| | | | (METRN), mRNA. |
| AE006464.1 | | Pcan052V1 | Homo sapiens 16p13.3 sequence section 3 of 8. |
| NM_000560.2 | CD53 | Pcan056 | Homo sapiens CD53 antigen (CD53), mRNA. |
| AL391064.23 | | Pcan056a | Human DNA sequence from clone RP11-392B1 on chromosome 1 |
| | | | Contains the 3′ end of the CD53 for CD53 antigen, complete |
| | | | sequence. |
| NM_000560.2 | CD53 | Pcan056b | Homo sapiens CD53 antigen (CD53), mRNA. |
| NM_004864.1 | GDF15 | Pcan065 | Homo sapiens growth differentiation factor 15 (GDF15), mRNA. |
| NM_032192.2 | DARPP32 | Pcan070 | Homo sapiens protein phosphatase 1, regulatory (inhibitor) |
| | | | subunit 1B (dopamine and cAMP regulated phosphoprotein, |
| | | | DARPP-32) (PPP1R1B), mRNA. |
| NM_012445.1 | SPON2 | Pro108 | Homo sapiens spondin 2, extracellular matrix protein (SPON2), mRNA |
| NM_012445.1 | SPON2 | Pro108a | Homo sapiens spondin 2, extracellular matrix protein (SPON2), mRNA. |

TABLE 2b

| GenBank | | DDXS | |
|---|---|---|---|
| Accession | Abbreviated Name | Amplicon | Annotation |
| NM_013230.1 | CD24 | CD24 | Homo sapiens CD24 antigen (small cell lung carcinoma cluster 4 antigen) (CD24), mRNA. |
| NM_000610.3 | CD44 | CD44 | Homo sapiens CD44 antigen (homing function and Indian blood group system) (CD44), |
| | | | transcript variant 1 , mRNA. |
| NM_004360.2 | CDH1 | CDH1 | Homo sapiens cadherin 1, type 1, E-cadherin (epithelial) (CDH1), mRNA |
| NM_001909.3 | CTSD | CTSD1 | Homo sapiens cathepsin D (lysosomal aspartyl protease) (CTSD), mRNA. |
| NM_199168.1 | CXCL12 | CXCL12 | Homo sapiens chemokine (C-X-C motif) ligand 12 (stromal cell-derived factor 1) |
| | | | (CXCL12), mRNA. |
| NM_022059.1 | CXCL16 | CXCL16 | Homo sapiens chemokine (C-X-C motif) ligand 16 (CXCL16), mRNA. |
| NM_000125.1 | ESR1 | ER | Homo sapiens estrogen receptor 1 (ESR1), mRNA. |
| NM_004475.1 | FLOT2 | ESA | Homo sapiens flotillin 2 (FLOT2), mRNA. |
| NM_021102.2 | SPINT2 | HAI-2 | Homo sapiens serine protease inhibitor, Kunitz type, 2 (SPINT2), mRNA. |
| NM_004448.2 | ERBB2 | Her2 | Homo sapiens v-erb-b2 erythroblastic leukemia viral oncogene homolog 2, |
| | | | neuro/glioblastoma derived oncogene homolog (avian) (ERBB2), transcript variant 1, mRNA. |
| NM_001530.2 | HIF1A | HIF1 alpha | Homo sapiens hypoxia-inducible factor 1, alpha subunit (basic helix-loop-helix |
| | | | transcription factor) (HIF1A), transcript variant 1, mRNA. |
| NM_001530.2 | HIF1A | HIF1 alpha 1 | Homo sapiens hypoxia-inducible factor 1, alpha subunit (basic helix-loop-helix |
| | | | transcription factor) (HIF1A), transcript variant 1, mRNA. |
| NM_002417.2 | MKI67 | Ki67 | Homo sapiens antigen identified by monoclonal antibody Ki-67 (MKI67), mRNA. |
| NM_002411.1 | Mmgb | Mammaglobin | Homo sapiens secretoglobin, family 2A, member 2 (SCGB2A2), mRNA. |
| NM_004994.1 | MMP9 | MMP9 | Homo sapiens matrix metalloproteinase 9 (gelatinase B, 92 kDa gelatinase, 92 kDa |
| | | | type IV collagenase) (MMP9), mRNA. |
| NM_000926.2 | PGR | PR | Homo sapiens progesterone receptor (PGR), mRNA. |
| NM_003219.1 | TERT | TERT | Homo sapiens telomerase reverse transcriptase (TERT), transcript variant 1, mRNA. |
| NM_003376.3 | VEGF | VEGF | Homo sapiens vascular endothelial growth factor (VEGF), mRNA. |

TABLE 3

| GenBank | | | | | |
|---|---|---|---|---|---|
| Accession | Chr Loc | Cellular Component Ontology | Molecular Function Ontology | Biological Process Ontology | Literature Function |
| NM_145016.2 | 11q12.1 | NA | NA | NA | NA |
| NM_033438.1 | 1q23.2 | GO: 0016021: integral to | NA | NA | function as coreceptors for |
| | | membrane; GO: 0005615: | | | lymphocyte activation and/or |
| | | extracellular space | | | adhesion via their extracellular |
| | | | | | and cytoplasmic domains |
| NM_006551.2 | 11q13 (Amp) | GO: 0005615: extracellular | NA | NA | Serum antibodies to lipophilin B |
| | | space | | | detected in late stage breast |
| | | | | | cancer patients. |
| NM_004385.2 | 5q14.3 | GO: 0005578: extracellular matrix | GO: 0005529: sugar binding; | GO: 0008037: cell recognition; | involved in the progression of |
| | | | GO: 0005540: hyaluronic acid binding; | GO: 0007275: development | melanomas and may be a reliable |
| | | | GO: 0005509: calcium ion binding | | marker for clinical diagnosis |
| NM_024626.1 | 1p13.1 | NA | NA | NA | costimulatory proteins. These |
| | | | | | proteins are expressed on the |
| | | | | | surface of antigen-presenting |
| | | | | | cells and interact with ligands |
| | | | | | (e.g., CD28; MIM 186760) on T |
| | | | | | lymphocytes. May participate in |
| | | | | | negative regulation of cell- |
| | | | | | mediated immunity in peripheral |
| | | | | | tissues |
| BC065717.1 | 1p13.1 | NA | NA | NA | costimulatory proteins. These |
| | | | | | proteins are expressed on the |
| | | | | | surface of antigen-presenting |
| | | | | | cells and interact with ligands |
| | | | | | (e.g., CD28; MIM 186760) on T |
| | | | | | lymphocytes. May participate in |
| | | | | | negative regulation of cell- |
| | | | | | mediated immunity in peripheral |
| | | | | | tissues |
| AC093297.3 | 5p12 (Amp) | NA | NA | NA | NA |
| NM_005449.3 | 1q32.1 | NA | NA | GO: 0006968: cellular defense | Toso, a cell surface, specific |
| | | | | response; GO: 0006916: anti- | regulator of Fas-induced |
| | | | | apoptosis | apoptosis in T cells. |
| NM_018098.4 | 3q26.1-q26.2 | GO: 0005622: intracellular activity; | GO: 0005085: guanyl-nucleotide exchange factor | GO: 0007242: intracellular | XRCC1, CLB6, and BRCT |
| | | | GO: 0004871: signal transducer activity | signaling cascade; GO: 0043123: | domains of ECT2 play a critical |
| | | | | positive regulation of I-kappaB | role in regulating cytokinesis |
| | | | | kinase/NF-kappaB cascade | |
| AL157387.9 | 10p11.21 | NA | NA | NA | NA |
| NM_016125.2 | 17q23.2 (Amp) | GO: 0000151: ubiquitin ligase complex | GO: 0004842: ubiquitin-protein | GO: 0016567: protein | NA |
| | | | ligase activity; GO: 0008270: zinc | ubiquitination | |
| | | | ion binding | | |
| NM_001075.2, | 4q13.2 | GO: 0005792: microsome; | GO: 0015020: | GO: 0006629: lipid metabolism | NA |
| NM_001073.1 | | GO: 0016021: integral to membrane | glucuronosyltransferase activity | | |
| NM_001073.1 | 4q13.2 | GO: 0005792: microsome; | GO: 0015020: | GO: 0008210: estrogen | NA |
| | | GO: 0016021: integral to | glucuronosyltransferase activity | metabolism; GO: 0006805: | |
| | | membrane | | xenobiotic metabolism | |
| AC079414.6 | 16q23.1 | NA | NA | NA | NA |
| NM_206925.1 | 15q22 | GO: 0016021: integral to | GO: 0016829: lyase activity; | GO: 0006730: one-carbon | CA XII showed no or weak |
| | | membrane | GO: 0008270: zinc ion binding; | compound metabolism | immunoreaction in the normal |
| | | | GO: 0004089: carbonate | | gastric mucosa and was slightly |
| | | | dehydratase activity | | increased in gastric tumors. |
| NM_022664.1, | 1q21 (Amp) | GO: 0005615: extracellular space; GO: | GO: 0005386: carrier activity; | GO: 0006810: transport; | These results indicate that ECM1 |
| U68187.1 | | 0005578: | GO: 0004871: signal transducer | GO: 0043123: positive regulation | tends to be preferentially |
| | | extracellular matrix; | activity; GO: 0005198: structural | of I-kappaB kinase/NF-kappaB | expressed by metastatic |
| | | | molecule activity | cascade; | epithelial tumors. |
| NM_024042.2 | 16p13.3 | NA | NA | GO: 0007399: neurogenesis | Promotes cell differentiation in |
| | | | | | neurogenesis |
| AE006464.1 | 16p13.3 | NA | NA | NA | NA |
| NM_000560.2 | 1p13 (Amp) | GO: 0005887: integral to plasma membrane | GO: 0007165: signal | Ligation of CD53 triggers a | NA |
| | | | transduction; GO: 0019735: | survival response and reduces | |
| | | | antimicrobial humoral response | the number of cells that enter | |
| | | | | apoptosis. The CD53 antigen | |
| | | | | interactions might contribute to | |
| | | | | cell survival in poorly vascular- | |
| | | | | ized regions of the tumor mass. | |
| XM_378356.1 | 12q13.13 | NA | NA | NA | NA |
| AL391064.23 | 1p13 (Amp) | NA | NA | NA | NA |
| NM_004864.1 | 19p13.1-13.2 | GO: 0005576: extracellular region | GO: 0005125: cytokine activity; | GO: 0007267: cell-cell signaling; | Microarray analysis identifies |
| | | | GO: 0008083: growth factor | GO: 0007165: signal | MIC-1 as being upregulated in |
| | | | activity | transduction; GO: 0007179: | cancer of breast, prostate, and |
| | | | | transforming growth factor beta | colon. Tissues from these |
| | | | | receptor signaling pathway | patients show increased MIC-1 |
| | | | | | by IHC and their serum shows |
| | | | | | elevated levels. |
| NM_032192.2 | 17q12 (Amp) | GO: 0005737: cytoplasm | GO: 0004860: protein kinase | GO: 0007165: signal transduction | DARPP 32 frequently are |
| | | | inhibitor activity; GO: 0004864: | | overexpressed in common |
| | | | protein phosphatase inhibitor | | subtypes of human |
| | | | activity | | adenocarcinomas suggest that |
| | | | | | these proteins may be important |
| | | | | | in tumorigenesis. |
| NM_000305.1 | 7q21.3 | GO: 0016020: membrane; | GO: 0016787: hydrolase activity; | NA | NA |
| | | GO: 0005576: extracellular region | GO: 0004064: arylesterase | | |
| | | | activity; GO: 0004063: | | |
| | | | aryldialkylphosphatase activity | | |
| NM_006573.3 | 13q32-34 | GO: 0005625: soluble fraction; | GO: 0005164: tumor necrosis | GO: 0006955: immune response; | serum levels of BAFF and APRIL |
| | | GO: 0005887: integral to plasma | factor receptor binding; | GO: 0008283: cell proliferation; | were increased about 5-fold in |
| | | membrane | | GO: 0007165: signal | patients with multiple myeloma |
| | | | | transduction; GO: 0008284: | as compared with healthy donors |
| | | | | positive regulation of cell | |
| | | | | proliferation | |
| AY358768.1 | 13q34 | NA | NA | NA | NA |
| NM_016056.1 | 12q14.1-q15 | NA | NA | NA | NA |
| AL136163.15 | 6q22.1-22.33 | NA | NA | NA | NA |
| AC018686.7 | 2q14.2 | NA | NA | NA | NA |
| AL356432.17 | 6q22.33 | NA | NA | NA | NA |
| NM_006408.2 | 7p21.3 | GO: 0005615: extracellular | NA | NA | Differentiation, associated with |
| | | space [evidence TAS] | | | ER positive tumors and interacts |
| | | | | | with metastasis genes |
| AY327584.1 | 1q21 (Amp) | NA | NA | NA | NA |
| NM_003579.2 | 1p32 (LOH) | GO: 0005634: nucleus [TAS] | GO: 0005524: ATP binding [IEA]; | GO: 0007126: meiosis [TAS]; | The protein encoded by this |
| | | | GO: 0003677: DNA binding [IEA]; | GO: 0006281: DNA repair [TAS]; | gene belongs to the DEAD-like |
| | | | GO: 0004386: helicase activity | GO: 0006310: DNA | helicase superfamily, and shares |
| | | | [IEA]; GO: 0016787: hydrolase | recombination [TAS];: | similarity with Saccharomyces |
| | | | activity [IEA] | GO: 0008151: cell growth and/or | cerevisiae Rad54, a protein |
| | | | | maintenance [IEA] | known to be involved in the |
| | | | | | homologous recombination and |
| | | | | | repair of DNA. This protein has |
| | | | | | been shown to play a role in |
| | | | | | homologous recombination |
| | | | | | related repair of DNA double- |
| | | | | | strand breaks. The binding of |
| | | | | | this protein to double-strand |
| | | | | | DNA induces a DNA topological |
| | | | | | change, which is thought to |
| | | | | | facilitate homologous DNA |
| | | | | | paring, and stimulate DNA |
| | | | | | recombination. |
| AY623117.1 | 1p33 (LOH) | GO: 0005634: nucleus [TAS] | GO: 0005524: ATP binding [IEA]; | GO: 0007126: meiosis [TAS]; | The protein encoded by this |
| | | | GO: 0003677: DNA binding [IEA]; | GO: 0006281: DNA repair [TAS]; | gene belongs to the DEAD-like |
| | | | GO: 0004386: helicase activity | GO: 0006310: DNA | helicase superfamily, and shares |
| | | | [IEA]; GO: 0016787: hydrolase | recombination [TAS];: | similarity with Saccharomyces |
| | | | activity [IEA] | GO: 0008151: cell growth and/or | cerevisiae Rad54, a protein |
| | | | | maintenance [IEA] | known to be involved in the |
| | | | | | homologous recombination and |
| | | | | | repair of DNA. This protein has |
| | | | | | been shown to play a role in |
| | | | | | homologous recombination |
| | | | | | related repair of DNA double- |
| | | | | | strand breaks. The binding of |
| | | | | | this protein to double-strand |
| | | | | | DNA induces a DNA topological |
| | | | | | change, which is thought to |
| | | | | | facilitate homologous DNA |
| | | | | | paring, and stimulate DNA |
| | | | | | recombination. |
| NM_005201.2 | 3p22 (Amp) | GO: 0005887: integral to plasma | GO: 0015026: coreceptor activity | GO: 0006935: chemotaxis | This gene encodes a member of |
| | | membrane | [evidence TAS] [pmid 9417093]; | [evidence TAS] [pmid | the beta chemokine receptor |
| | | | GO: 0016493: C-C chemokine | 10910894]; GO: 0007155: cell | family, which is predicted to be a |
| | | | receptor activity [evidence IEA]; | adhesion [evidence TAS] [pmid | seven transmembrane protein |
| | | | GO: 0001584: rhodopsin-like | 10910894]; GO: 0006955: | similar to G protein-coupled |
| | | | receptor activity [evidence IEA]; | immune response [evidence | receptors. Chemokines and their |
| | | | | TAS] [pmid 9670926]; | receptors are important for the |
| | | | | GO: 0007204: cytosolic calcium | migration of various cell types |
| | | | | ion concentration elevation | into the inflammatory sites. This |
| | | | | [evidence TAS] [pmid 9417093]; | receptor protein preferentially |
| | | | | GO: 0007186: G-protein coupled | expresses in the thymus. I-309, |
| | | | | receptor protein signaling | thymus activation-regulated |
| | | | | pathway [evidence TAS] [pmid | cytokine (TARC) and |
| | | | | 8816377] | macrophage inflammatory |
| | | | | | protein-1 beta (MIP-1 beta) have |
| | | | | | been identified as ligands of this |
| | | | | | receptor. Studies of this receptor |
| | | | | | and its ligands suggested its role |
| | | | | | in regulation of monocyte |
| | | | | | chemotaxis and thymic cell |
| | | | | | apoptosis. More specifically, this |
| | | | | | receptor may contribute to the |
| | | | | | proper positioning of activated T |
| | | | | | cells within the antigenic |
| | | | | | challenge sites and specialized |
| | | | | | areas of lymphoid tissues. This |
| | | | | | gene is located at the chemokine |
| | | | | | receptor gene cluster region. |
| BC014098.2 | 10q23.2 | GO: 0005737: cytoplasm | NA | NA | SNCG is required for efficient |
| | | [evidence IEA] | | | ER-alpha signaling in breast |
| | | | | | cancer. |
| AL831952.1 | 17q12 (Amp) | NA | NA | NA | NA |
| BC021275.2 | 5q23-5q31 | NA | NA | NA | NA |
| NM_002407.1 | 11q13 (Amp) | NA | GO: 0005497: androgen binding | NA | MGB2 markers may be useful for |
| | | | [evidence NAS] | | identifying micrometastases in |
| | | | | | sentinel lymph nodes of breast |
| | | | | | cancer patients. |
| NM_017697.2 | 8q22.1 | NA | GO: 0003723: RNA binding | NA | NA |
| | | | [evidence IEA] | | |
| NM_001008540.1 | 2q21 | GO: 0016021: integral to | GO: 0016493: C-C chemokine | GO: 0007186: G-protein coupled | CXCR4 is induced by NF-kappa |
| | | membrane [evidence IEA] | receptor activity [evidence IEA]; | receptor protein signaling | B and has a role in breast cancer |
| | | | GO: 0001584: rhodopsin-like | pathway [evidence IEA] | cell migration and metastasis. |
| | | | receptor activity [evidence IEA]; | | |
| | | | GO: 0016494: C—X—C chemokine | | |
| | | | receptor activity [evidence NAS] | | |
| | | | [pmid 9468539] | | |
| NM_000579.1 | 3p21 | GO: 0016021: integral to | GO: 0004872: receptor activity | GO: 0007186: G-protein coupled | CCR5 activity influences human |
| | | membrane [evidence IEA] | [evidence IEA]; GO: 0016493: C- | receptor protein signaling | breast cancer progression in a |
| | | | C chemokine receptor activity | pathway [evidence IEA] | p53-dependent manner |
| | | | [evidence IEA]; GO: 0001584: | | |
| | | | rhodopsin-like receptor activity | | |
| | | | [evidence IEA] | | |
| NM_004367.3 | 6q27 | GO: 0005887: integral to plasma | GO: 0016493: C-C chemokine | GO: 0007186: G-protein coupled | CCR6 on polarized intestinal |
| | | membrane [evidence TAS] | receptor activity [evidence IEA]; | receptor protein signaling | epithelial cells, alter specialized |
| | | [PMID: 9186513] | GO: 0004872: receptor activity | pathway [evidence IEA]; | intestinal epithelial cell functions, |
| | | | [evidence TAS] [PMID: | GO: 0019735: antimicrobial | including electrogenic ion |
| | | | 9186513]; GO: 0001584: | humoral response (sensu | secretion and possibly epithelial |
| | | | rhodopsin-like receptor activity | Vertebrata) [evidence TAS] | cell adhesion and migration |
| | | | [evidence IEA] | [PMID: 9186513]; GO: 0006928: | |
| | | | | cell motility [evidence TA] | |
| | | | | [PMID: 9186513]; GO: 0006968: | |
| | | | | cellular defense response | |
| | | | | [evidence TAS] [PMID: | |
| | | | | 10521347]; GO: 0006935: | |
| | | | | chemotaxis [evidence TAS] | |
| | | | | [PMID: 11001880]; GO: 0006959: | |
| | | | | humoral immune response | |
| | | | | [evidence TAS] [PMID: | |
| | | | | 11001880]; GO: 0007204: | |
| | | | | positive regulation of cytosolic | |
| | | | | calcium ion concentration | |
| | | | | [evidence TAS] [PMID: | |
| | | | | 9223454]; GO: 0007165: signal | |
| | | | | transduction [evidence TAS] | |
| | | | | [PMID: 9186513] | |
| NM_004591.1 | 2q33-q37 | GO: 0005615: extracellular space | GO: 0008009: chemokine activity | GO: 0019735: antimicrobial | Results describe the relationship |
| | | [evidence TAS] [pmid 9038201]; | [evidence TAS ] [pmid | humoral response (sensu | between cancer-related factors |
| | | | 10438902]; | Vertebrata) [evidence TAS] | and serum levels of macrophage |
| | | | | [pmid 9038201]; GO: 0007267: | inflammatory protein-3alpha in |
| | | | | cell-cell signaling [evidence TAS] | hepatocellular carcinoma. |
| | | | | [pmid 9038201]; GO: 0006935: | |
| | | | | chemotaxis [evidence TAS] | |
| | | | | [pmid 10438902]; GO: 0006954: | |
| | | | | inflammatory response [evidence | |
| | | | | TAS] [pmid 9129037]; | |
| | | | | GO: 0007165: signal transduction | |
| | | | | [evidence TAS] [pmid 9038201] | |
| NM_006564.1 | 3p21 | GO: 0005887: integral to plasma | GO: 0016493: C-C chemokine | GO: 0007186: G-protein coupled | NA |
| | | membrane [evidence TAS] [pmid | receptor activity [evidence IEA]; | receptor protein signaling | |
| | | 9166430] | GO: 0016494: C—X—C chemokine | pathway [evidence TAS] [pmid | |
| | | | receptor activity [evidence IEA]; | 9166430]; GO: 0019079: viral | |
| | | | GO: 0015026: coreceptor activity | genome replication [evidence | |
| | | | [evidence TAS] [pmid 9166430]; | TAS] [pmid 9230441] | |
| | | | GO: 0001584: rhodopsin-like | | |
| | | | receptor activity [evidence IEA]; | | |
| NM_178445.1 | 3q22 | GO: 0005887: integral to plasma | GO: 0016493: C-C chemokine | GO: 0007186: G-protein coupled | NA |
| | | membrane [evidence TAS] | receptor activity [evidence IEA]; | receptor protein signaling | |
| | | [PMID: 10767544] | GO: 0001584: rhodopsin-like | pathway [evidence TAS] [PMID: | |
| | | | receptor activity [evidence IEA] | 10734104]; GO: 0006935: | |
| | | | | chemotaxis [evidence TAS] | |
| | | | | [PMID: 10706668]; GO: 0006955: | |
| | | | | immune response [evidence | |
| | | | | TAS] [PMID: 10706668] | |
| NM_003965.3 | 3p21 | GO: 0016021: integral to | GO: 0016493: C-C chemokine | GO: 0007186: G-protein coupled | NA |
| | | membrane [evidence IEA]; | receptor activity [evidence IEA]; | receptor protein signaling | |
| | | GO: 0005887: integral to plasma | GO: 0004872: receptor activity | pathway [evidence IEA] | |
| | | membrane [evidence TAS] | [evidence IEA]; GO: 0001584: | [evidence TAS] [PMID: | |
| | | [PMID: 9473515] | rhodopsin-like receptor activity | 9473515]; GO: 0019735: | |
| | | | [evidence IEA] | antimicrobial humoral response | |
| | | | | (sensu Vertebrata) [evidence | |
| | | | | TAS] [PMID: 9473515]; | |
| | | | | GO: 0006935: chemotaxis | |
| | | | | [evidence TAS] [PMID: 9473515] | |
| NM_153758.1 | 1q32.2 | GO: 0005576: extracellular region | GO: 0003779: actin binding | GO: 0006915; apoptosis | NA |
| | | [evidence IEA] [PMID: | [evidence IEA]; GO: 0005125: | [evidence IEA]; GO: 0006955: | |
| | | 11196675] [evidence NAS] | cytokine activity [evidence IEA] | immune response [evidence IEA] | |
| | | [PMID: 11196675] | [PMID: 11196675] [evidence | [PMID: 11196675] [evidence | |
| | | | TAS] [PMID: 11196675]; | NAS] [PMID: 11196675]; | |
| | | | | GO: 0007165: signal transduction | |
| | | | | [evidence NAS] [PMID: | |
| | | | | 11196675] | |
| NM_004625.3 | 3p25 | GO: 0005576: extracellular | GO: 0005102: receptor binding | GO: 0007275: | Expression inversely associated |
| | | [evidence IEA]; GO: 0005615: | [evidence NAS] [pmid 8893824]; | development[evidence IEA]; | to ER in uterine leyoma |
| | | extracellular space [evidence | GO: 0004871: signal transducer | GO: 0009653: morphogenesis | |
| | | NR] | activity [evidence IEA] | [evidence TAS] [pmid 9161407]; | |
| | | | | GO: 0007267: cell-cell signaling | |
| | | | | [evidence NR]; GO: 0007548: sex | |
| | | | | differentiation [evidence TAS] | |
| | | | | [pmid 9790192]; GO: 0007165: | |
| | | | | signal transduction [evidence | |
| | | | | NAS] [pmid 8893824]; | |
| | | | | GO: 0007223: frizzled-2 signaling | |
| | | | | pathway [evidence IEA] | |
| NM_006988.3 | 21q21.2 | GO: 0005578: extracellular matrix | GO: 0008201: heparin binding | GO: 0007229: integrin-mediated | This gene encodes a disintegrin |
| | | (sensu Metazoa) [evidence IEA] | [evidence IEA]; GO: 0016787; | signaling pathway [evidence | and metalloproteinase with |
| | | | hydrolase activity [evidence IEA]; | TAS] [pmid 8995297]; | thrombospondin motifs-1 |
| | | | GO: 0005178: integrin binding | GO: 0006508: proteolysis and | (ADAMTS1), which is a member |
| | | | [evidence NR]; GO: 0004222: | peptidolysis [evidence IEA]; | of the ADAMTS protein family. |
| | | | metalloendopeptidase activity | GO: 0008285: negative regulation | Members of the family share |
| | | | [evidence IEA]; GO: 0008270: | of cell proliferation [evidence | several distinct protein modules, |
| | | | zinc ion binding [evidence IEA] | TAS] [pmid 10438512] | including a propeptide region, a |
| | | | | | metalloproteinase domain, a |
| | | | | | disintegrin-like domain, and a |
| | | | | | thrombospondin type 1 (TS) |
| | | | | | motif. Individual members of this |
| | | | | | family differ in the number of C- |
| | | | | | terminal TS motifs, and some |
| | | | | | have unique C-terminal domains. |
| | | | | | The protein encoded by this |
| | | | | | gene contains 2 disintegrin loops |
| | | | | | and 3 C-terminal TS motifs and |
| | | | | | has anti-angiogenic activity. The |
| | | | | | expression of this gene may be |
| | | | | | associated with various |
| | | | | | inflammatory processes as well |
| | | | | | as development of cancer |
| | | | | | cachexia. This gene is likely to |
| | | | | | be necessary for normal growth, |
| | | | | | fertility, and organ morphology |
| | | | | | and function. |
| NM_001571.2 | 19q13.3-q13.4 | GO: 0005634: nucleus [evidence | GO: 0003702: RNA polymerase II | GO: 0006355: regulation of | hIRF3 inhibited cell growth, |
| | | IEA] | transcription factor activity | transcription, DNA-dependent | blocked DNA synthesis, and |
| | | | [evidence TAS] [PMID: | [evidence IEA]; GO: 0006350: | induced apoptosis, while a |
| | | | 8524823]; GO: 0003712: | transcription [evidence IEA]; | dominant negative mutant |
| | | | transcription cofactor activity | GO: 0006366: transcription from | transformed 3T3 cells, implying |
| | | | [evidence TAS] [PMID: | Pol II promoter [evidence TAS] | that IRF3 may function as a |
| | | | 8524823]; GO: 0003700: | [PMID: 8524823] | tumor suppressor and its |
| | | | transcription factor activity | | dominant negative mutant may |
| | | | [evidence IEA] | | have a role in tumorigenesis. |
| NM_006804.2 | 17q11-q12 (Amp) | NA | NA | NA | NA |
| NM_022161.2 | 20q13.3 | NA | NA | NA | NA |
| NM_014452.3 | 6p21.1-12.2 | NA | NA | NA | NA |
| NM_001838.2 | 17q12-q21.2 (Amp) | NA | NA | NA | Overexpression of CCR7 mRNA |
| | | | | | in nonsmall cell lung cancer is |
| | | | | | associated with development of |
| | | | | | lymph node metastasis |
| NM_002989.2 | 9p13 | NA | NA | NA | Cathepsin D specifically cleaves |
| | | | | | this protein that is expressed in |
| | | | | | human breast cancer. |
| NM_004994.1 | 20q11.2-q13.1 | GO: 0005615: extracellular space | GO: 0016787: hydrolase activity | GO: 0030574: collagen | NA |
| | | [evidence TAS] [pmid 2551898]; | [evidence IEA]; GO: 0008270: | catabolism [evidence IEA] | |
| | | GO: 0005578: extracellular matrix | zinc ion binding [evidence TAS] | | |
| | | (sensu Metazoa) [evidence IEA] | [pmid 2551898]; GO: 0004229: | | |
| | | | gelatinase B activity [evidence | | |
| | | | IEA]; GO: 0008133: collagenase | | |
| | | | activity [evidence TAS] [pmid | | |
| | | | 2551898] | | |
| NM_001554.3 | 1p31-p22 | GO: 0005576: extracellular | GO: 0008201: heparin binding; | GO: 0006935: chemotaxis; | promotes tumor growth; |
| | | | GO: 0005520: insulin-like growth | GO: 0007155: cell adhesion; | increased Cyr61 expression is |
| | | | factor binding | GO: 0009653: morphogenesis | associated with an aggressive |
| | | | | [pmid 9135077]; GO: 0008283: | phenotype of breast cancer cells |
| | | | | cell proliferation [pmid 9135077]; | |
| | | | | GO: 0001558: regulation of cell | |
| | | | | growth | |
| NM_003219.1 | 5p15.33 (Amp) | GO: 0005634: nucleus [evidence | GO: 0003677: DNA binding | GO: 0006278: RNA-dependent | hTERT is transcriptionally |
| | | IEA]; GO: 0000781: | [evidence IEA]; GO: 0003723: | DNA replication [evidence IEA]; | regulated by raloxifene via an |
| | | chromosome, telomeric region | RNA binding [evidence IEA]; | GO: 0007004: telomerase- | estrogen-responsive element- |
| | | [evidence IC] [pmid 12135483]; | GO: 0016740: transferase activity | dependent telomere | dependent mechanism, which |
| | | GO: 0005697: telomerase | [evidence IEA]; GO: 0042162: | maintenance [evidence IEA] | inhibits E2-induced up-regulation |
| | | holoenzyme complex [evidence | telomeric DNA binding [evidence | | of telomerase activity. |
| | | IDA] [pmid 12135483] | TAS] [pmid 9288757]; | | Telomerase activity in |
| | | | GO: 0003964: RNA-directed DNA | | microdissected human breast |
| | | | polymerase activity [evidence | | cancer tissues: association with |
| | | | IEA]; GO: 0003721: telomeric | | p53, p21 and outcome. |
| | | | template RNA reverse | | |
| | | | transcriptase activity [evidence | | |
| | | | IEA] [evidence TAS] [pmid | | |
| | | | 14991929] | | |
| NM_001530.2 | 14q21-q24 (Amp) | GO: 0005634: nucleus [evidence | GO: 0004871: signal transducer | GO: 0042592: homeostasis | Hif-1alpha is expressed in the |
| | | IEA] [evidence IDA] [pmid | activity [evidence IEA]; | [evidence TAS] [pmid | majority of patients with node- |
| | | 15261140] | GO: 0003700: transcription factor | 15261140]; GO: 0007165: signal | positive breast cancer. There |
| | | | activity [evidence IEA]; | transduction [evidence IEA] | was significantly shorter distant |
| | | | GO: 0035035: histone acetyltransferase binding | [evidence NAS] [pmid | metastasis-free survival & |
| | | | [evidence IPI] [pmid 15261140]; | 15261140]; GO: 0001666: | disease-free survival in patients |
| | | | GO: 0046982: protein heterodimerization activity | response to hypoxia [evidence | with increased hif-1alpha |
| | | | [evidence TAS] [pmid 15261140]; | IDA] [pmid 15261140]; | expression. |
| | | | GO: 0003705: RNA polymerase II transcription | GO: 0006355: regulation of | |
| | | | factor activity, enhancer binding | transcription, DNA-dependent | |
| | | | [evidence TAS] [pmid 9079689] | [evidence IEA] | |
| NM_004360.2 | 16q22.1 | GO: 0016021: integral to | GO: 0005515: protein binding | GO: 0007155: cell adhesion | Recovery of cellular E-cadherin |
| | | membrane [evidence IEA] | [evidence IEA]; GO: 0005509: | [evidence IEA]; GO: 0007156: | precedes replenishment of |
| | | [evidence NAS] [pmid 8033105]; | calcium ion binding [evidence | homophilic cell adhesion | estrogen receptor and estrogen- |
| | | | IEA] | [evidence IEA] [evidence NAS] | dependent proliferation of breast |
| | | | | [pmid 8033105] | cancer cells rescued from a |
| | | | | | death stimulus. |
| NM_000125.1 | 6q25.1 | GO: 0005634: nucleus [evidence | GO: 0003677: DNA binding | GO: 0006350: transcription | patients that progressed to |
| | | NR] [evidence IEA] [evidence | [evidence IEA]; | [evidence IEA]; GO: 0016049: | breast cancer showed |
| | | NAS]; GO: 0016020: membrane | GO: 0005496: steroid binding | cell growth [evidence ISS] [pmid | significantly higher ER-alpha |
| | | [evidence NAS] [pmid | [evidence IEA]; GO: 0004872: | 9171231]; GO: 0007165: signal | expression in their HUT |
| | | 11960618]; GO: 0016585: | receptor activity [evidence IEA]; | transduction [evidence TAS] | (hyperplasia of usual type) foci |
| | | chromatin remodeling complex | GO: 0003700: transcription factor | [pmid 10749889] [evidence TAS] | compared with controls |
| | | [evidence NAS] [pmid | activity [evidence IEA] [evidence | [pmid 10970861]; GO: 0045839: | |
| | | 12351687]; | NAS] [pmid 8600444]; | negative regulation of mitosis | |
| | | | GO: 0030284: estrogen receptor | [evidence ISS] [pmid 12119130]; | |
| | | | activity [evidence NAS] [pmid | GO: 0030520: estrogen receptor | |
| | | | 3753802] [evidence TAS] [pmid | signaling pathway [evidence | |
| | | | 11004670]; GO: 0003707: steroid | NAS] [pmid 3753802]; | |
| | | | hormone receptor activity | GO: 0006355: regulation of | |
| | | | [evidence IEA] [evidence NAS] | transcription, DNA-dependent | |
| | | | [pmid 11004670]; GO: 0030235: | [evidence IEA]; [evidence NAS] | |
| | | | nitric-oxide synthase regulator | [pmid 3753802] | |
| | | | activity [evidence NAS] [pmid | | |
| | | | 12389206] | | |
| NM_000926.2 | 11q22-q23 | GO: 0005634: nucleus [evidence | GO: 0005496: steroid binding | GO: 0007267: cell-cell signaling | small, hormone-receptor-positive |
| | | IEA] | [evidence IEA]; GO: 0003700: | [evidence TAS] [pmid 1557371]; | breast cancers (with a theoretical |
| | | | transcription factor activity | GO: 0007165: signal transduction | good prognosis) may carry an |
| | | | [evidence IEA]; GO: 0003707: | [evidence TAS] [pmid 1557371]; | elevated risk of nodal |
| | | | steroid hormone receptor activity | GO: 0006366: transcription from | involvement if accumulation of |
| | | | [evidence IEA]; [evidence TAS] | Pol II promoter [evidence NR]; | uPA-PAI-1 complexes is shown |
| | | | [pmid 10840043]; | GO: 0006355: regulation of | inside their tumor cells by means |
| | | | | transcription, DNA-dependent | of immunohistochemistry. |
| | | | | [evidence IEA] | |
| NM_004448.2 | 17q11.2-q12; | GO: 0016020: membrane | GO: 0005524: ATP binding | GO: 0008283: cell proliferation | Circulating tumor cells in breast |
| | 17q21.1 (Amp) | [evidence IEA]; GO: 0016021: | [evidence IEA]; GO: 0004872: | [evidence TAS] [pmid | cancer were used to measure |
| | | integral to membrane [evidence | receptor activity [evidence IEA]; | 10851066]; GO: 0006468: protein | HER-2 gene amplification as an |
| | | IEA]; GO: 0005576: extracellular | GO: 0016740: transferase activity | amino acid phosphorylation | indicator of cancer progression. |
| | | region [evidence IDA] [pmid | [evidence IEA]; GO: 0043125: | [evidence IEA] [evidence TAS] | |
| | | 10485918] | ErbB-3 class receptor binding | [pmid 10851066]; GO: 0007169: | |
| | | | [evidence TAS] [pmid 9590694]; | transmembrane receptor protein | |
| | | | GO: 0005006: epidermal growth | tyrosine kinase signaling | |
| | | | factor receptor activity [evidence | pathway [evidence IEA] | |
| | | | IEA]; GO: 0004716: receptor | [evidence IDA] [pmid 10485918] | |
| | | | signaling protein tyrosine kinase | | |
| | | | activity [evidence TAS] [pmid | | |
| | | | 10851066]; GO: 0004715: non-membrane | | |
| | | | spanning protein tyrosine kinase activity | | |
| | | | [evidence IDA] [pmid 10485918] | | |
| NM_002417.2 | 10q25-qter | GO: 0005730: nucleolus | GO: 0005524: ATP binding | GO: 0000074: regulation of cell | Prognostic factors in surgical |
| | | [evidence NR] | [evidence IEA] | cycle [evidence TAS] [pmid | stage I endometrial carcinoma |
| | | | | 8227122] | |
| NM_001909.3 | 11p15.5 | GO: 0005764: lysosome | GO: 0004194: pepsin A activity | GO: 0006508: proteolysis and | Cathepsin D seems to facilitate |
| | | [evidence NAS]; GO: 0005576: | [evidence IEA]; GO: 0016787: | peptidolysis [evidence IEA] | early phases of tumor |
| | | extracellular region [evidence | hydrolase activity [evidence IEA]; | | progression such as cell |
| | | NAS] [pmid 14718574] | GO: 0004192: cathepsin D | | proliferation and local |
| | | | activity [evidence TAS] [pmid 3927292]; | | dissemination. |
| NM_199168.1 | 10q11.1 | GO: 0005576: extracellular region | GO: 0008009: chemokine activity | GO: 0007186: G-protein coupled | SDF-1alpha and its receptor |
| | | [evidence IEA] | [evidence TAS] [pmid | receptor protein signaling | chemokine receptor CXCR4 |
| | | | 10772939]; GO: 0008083: growth | pathway [evidence TAS] [pmid | induced transendothelial breast |
| | | | factor activity [evidence IEA] | 8752280]; GO: 0006874: calcium | cancer cell migration through |
| | | | | ion homeostasis [evidence TAS] | activation of the PI-3K/AKT |
| | | | | [pmid 10772939]; GO: 0007155: | pathway and Ca(2+)-mediated |
| | | | | cell adhesion [evidence TAS] | signaling. |
| | | | | [pmid 10198043]; GO: 0007267: | |
| | | | | cell-cell signaling [evidence NR]; | |
| | | | | GO: 0006935: chemotaxis | |
| | | | | [evidence TAS] [pmid | |
| | | | | 10620615]; GO: 0008015: | |
| | | | | circulation [evidence TAS] [pmid | |
| | | | | 10772939]; GO: 0006954: | |
| | | | | inflammatory response [evidence | |
| | | | | NR]; GO: 0008064: regulation of | |
| | | | | actin polymerization and/or | |
| | | | | depolymerization [evidence TAS] | |
| | | | | [pmid 10570282]; GO: 0009615: | |
| | | | | response to virus [evidence TAS] | |
| | | | | [pmid 10772939]; GO: 0007165: | |
| | | | | signal transduction [evidence | |
| | | | | TAS] [pmid 10491003] | |
| NM_012445.1 | 4p16.3 | GO: 0005615: extracellular | GO: 0005515: protein binding | GO: 0007275: development; | NA |
| | | space; GO: 0005578: | | GO: 0006955: immune response; | |
| | | extracellular matrix | | GO: 0007411: axon guidance | |
| | | | | [evidence TAS] [pmid | |
| | | | | 10512675]; GO: 0006935: | |
| | | | | chemotaxis; GO: 0030335: | |
| | | | | positive regulation of cell | |
| | | | | migration; GO: 0001569: | |
| | | | | patterning of blood vessels; | |
| | | | | GO: 0045766: positive regulation | |
| | | | | of angiogenesis; GO: 0007155: | |
| | | | | cell adhesion | |
| NM_022059.1 | 17p13 | GO: 0005576: extracellular region | GO: 0005125: cytokine activity | GO: 0006935: chemotaxis | NA |
| | | [evidence NAS] [PMID: | [evidence IEA]; | [evidence NAS] [PMID: | |
| | | 11017100]; GO: 0016021: | GO: 0005044: scavenger receptor | 11290797]; GO: 0048247: | |
| | | integral to membrane [evidence | activity [evidence TAS] [PMID: | lymphocyte chemotaxis | |
| | | NAS] [PMID: 11017100] [PMID: | 11060282] | [evidence NAS] [PMID: | |
| | | 11290797] | | 11017100]; GO: 0006898: | |
| | | | | receptor mediated endocytosis | |
| | | | | [evidence NAS] [PMID: | |
| | | | | 11060282] | |
| NM_002411.1 | 11q13 | GO: 0008372: | GO: 0005496: steroid binding | GO: 0000004: biological_process | Mammaglobin has a role in |
| | | cellular_component unknown | [evidence IEA] | unknown [evidence ND] | progression of breast cancer, as |
| | | [evidence ND] | | | shown by its expression in |
| | | | | | leukapheresis products |
| NM_000610.3 | 11p13 | GO: 0016021: integral to | GO: 0005518: collagen binding | GO: 0007155: cell adhesion | Data demonstrate that blockade |
| | | membrane [evidence IEA]; | [evidence NAS] [PMID: | [evidence IEA]; GO: 0016337: | of the ERK pathway suppressed |
| | | GO: 0016020: membrane | 2471973]; GO: 0005540: | cell-cell adhesion [evidence | the expression of matrix |
| | | [evidence IEA]; GO: 0005887: | hyaluronic acid binding [evidence | NAS] [PMID 1922057]; | metalloproteinases 3, 9, and 14, |
| | | integral to plasma membrane | IEA] [PMID: 1991450]; | GO: 0007160: cell-matrix | and CD44, and markedly |
| | | [evidence NAS] [PMID 1991450] | GO: 0005540: hyaluronic acid | adhesion [evidence NAS] [PMID | inhibited the invasiveness of |
| | | | binding [evidence NAS] [PMID: | 1922057] | tumor cells. |
| | | | 1991450]; GO: 0004872: receptor | | |
| | | | activity [evidenceIEA]; GO: 000: | | |
| | | | protein binding [evidenceIEA] | | |
| NM_013230.1 | 6q21 ?? | GO: 0005886: plasma membrane | GO: 0006959 humoral immune | The correlation between CD24 | |
| | | [evidence NAS] [pmid 7959762] | response [evidence TAS] [pmid | expression and invasiveness | |
| | | | 7959762] | was calculated to be highly | |
| | | | | significant | |
| NM_004475.1 | 17q11-q12 | GO: 0016600: flotillin complex | GO: 0005515 protein binding | GO: 0007155 cell adhesion | Flot-2 overexpression is |
| | | [evidence IEA]; GO: 0005886: | [evidence IEA] | [evidence NAS] [pmid 8051082]; | associated with melanoma |
| | | plasma membrane [evidence | | GO: 0008544 epidermis | progression, with increased |
| | | NAS] [pmid 1769667]; | | development [evidence TAS] | PAR-1 expression, and with |
| | | GO: 0016021: integral to | | [pmid 8051082] | transformation of SB2 melanoma |
| | | membrane [evidence IEA] | | | cells to a highly metastatic line. |
| NM_021102.2 | 19q13.1 | GO: 0005625: soluble fraction | GO: 0004867: serine-type | GO: 0006928: cell motility | Low level of HAI-2 is associated |
| | | [evidence TAS] [pmid 9115294]; | endopeptidase inhibitor activity | [evidence TAS] [pmid 9434156] | with breast cancer |
| | | GO: 0005576: extracellular region | [evidence TAS] [pmid 9434156] | | |
| | | [evidence TAS] [pmid 9346890]; | | | |
| | | GO: 0016021: integral to membrane | | | |
| | | [evidence TAS] [pmid 9434156] | | | |
| NM_003376.3 | 6p21.1 | GO: 0016020: membrane | GO: 0008201: heparin binding | GO: 0001525: angiogenesis | During tumor progression there |
| | | [evidence IEA]; GO: 0005578: | [evidence IEA]; [evidence IDA] | [evidence IEA], [evidence IDA] | is a change in the relative |
| | | extracellular matrix (sensu | [pmid 15001987]; GO: 0008083: | [pmid 11427521], [evidence | amounts of soluble VEGF-A |
| | | Metazoa) [evidence NAS] [pmid | growth factor activity [evidence | NAS] [pmid 15351965]; | receptor Flt-1 and VEGF-A in the |
| | | 14570917] | IEA]; [evidence NAS] [pmid | GO: 0007399: neurogenesis | circulation. Association between |
| | | | 11016853]; GO: 0050840: | [evidence ISS], [evidence TAS] | HER-2/neu and VEGF |
| | | | extracellular matrix binding | [pmid 15351965]; GO: 0016477: | expression supports the use of |
| | | | [evidence NAS] [pmid | cell migration [evidence NAS] | combination therapies directed |
| | | | 14570917]; GO: 0042803: protein | [pmid 15122338]; GO: 0008283: | against both HER-2/neu and |
| | | | homodimerization activity | cell proliferation [evidence IEA]; | VEGF for treatment of breast cancers. |
| | | | [evidence NAS] [pmid | GO: 0001570: vasculogenesis | |
| | | | 12127077]; GO: 0005172: | [evidence TAS] [pmid | |
| | | | vascular endothelial growth | 15015550]; GO: 0006950: | |
| | | | factor receptor binding [evidence | response to stress [evidence | |
| | | | TAS] [pmid 1711045] | TAS] [pmid 9202027]; | |
| | | | | GO: 0007165: signal transduction | |
| | | | | [evidence TAS] [pmid 1711045]; | |
| | | | | GO: 0000074: regulation of cell | |
| | | | | cycle [evidence IEA]; | |
| | | | | GO: 0050930: induction of | |
| | | | | positive chemotaxis [evidence | |
| | | | | NAS] [pmid 12744932]; | |
| | | | | GO: 0043066: negative regulation | |
| | | | | of apoptosis [evidence IMP] | |
| | | | | [pmid 10066377], [evidence IMP] | |
| | | | | [pmid 11461089]; GO: 0008284: | |
| | | | | positive regulation of cell | |
| | | | | proliferation [evidence TAS] | |
| | | | | [pmid 9202027]; GO: 0030949: | |
| | | | | positive regulation of vascular | |
| | | | | endothelial growth factor | |
| | | | | receptor signaling pathway [evidence | |
| | | | | NAS] [pmid 10066377] | |
| AY216715.1 | 10p11-q11 | NA | RAS-binding domain | NA | NA |
| NM_001908.3 | 8p22 | lysosome [goid 0005764] | cathepsin B activity [goid | proteolysis and peptidolysis [goid | NA |
| | | [evidence NR]; intracellular [goid | 0004213] [evidence TAS] [pmid | 0006508] [evidence TAS] [pmid | |
| | | 0005622] [evidence TAS] [pmid | 1645961] | 3463996] | |
| | | 1645961] | | | |
| L08044.1 | 21q22.3 | NA | NA | NA | NA |
| NM_003226.2 | 21q22.3 | extracellular region [goid | NA | digestion [goid 0007586] | NA |
| | | 0005576] [evidence TAS] [pmid | | [evidence TAS] [pmid 8454642]; | |
| | | 8454642] | | go_process: digestion [goid | |
| | | | | 0007586] [evidence TAS] [pmid | |
| | | | | 9043862]; go_process: defense | |
| | | | | response [goid 0006952] | |
| | | | | [evidence TAS] [pmid 8454642] | |
| AL391686.10 | 10p11.21 | NA | NA | NA | NA |

Genes within a region know to be amplified in cancer are indicated by (Amp) next to the chromosomal location;
Genes within a region know to have loss of heterozygosity (LOH) in cancer are indicated by (LOH) next to the chromosomal location;
NA = not available

In addition, 13 genes were selected for use as endogenous controls. Endogenous control candidates were selected from among those well-known in the literature as commonly constitutively expressed gene products across a wide range of tissues and biological conditions. See Kok, J B et al., Lab Invest. 2005 January; 85(1):154-9; and Janssens, N., et al., Mol. Diagn. 2004; 8(2):107-13 which are hereby incorporated by reference in their entirety.

TABLE 4
Endogenous controls.

| GenBank Accession | Abbreviated Name |
|---|---|
| NM_001101.2 | ACTB |
| NM_003194.2 | TBP |
| NM_003234.1 | TFRC |
| NM_000194.1 | HPRT1 |
| NM_004048.2 | B2M |
| NM_000190.2 | HMBS |
| NM_004168.1 | SDHA |
| NM_021009.2 | UBC |
| NM_002046.2 | GAPDH |
| NM_000181.1 | GUSB |
| NM_001002.3 | RPLPO |
| NM_012423.2 | RPL13A |
| NM_003406.2 | YWHAZ |
| D38112.1* | ATP6 |

*The ATP6 CDS is located at nucleotides [7941 . . . 8621] of D38112.1 “Homo sapiens mitochondrial DNA, complete sequence”

### Individuals and Sample Sets

Expression of gene products may be evaluated in primary tissues and/or lymph nodes; and alternatively in primary tissue and/or bone marrow samples. Additionally, expressions of gene products are evaluated in blood samples. In addition, primary tissues, lymph nodes, bone marrow and blood may be used in combination.

Samples were collect retrospectively for individuals with primary or metastatic breast cancer. Gene product expression profiles were evaluated on archival paraffin-preserved primary tissue from individuals who had metastatic breast cancer. As a control, primary tissues from individuals with no metastasis were evaluated.

In the studies above, both positive and negative groups of individuals have a minimum of 5-10 years follow-up information to evaluate the relation of gene product expression to disease outcome. Both groups have a representation of individuals with good outcome (no disease progression) 5-8 years after surgery, and poor outcome with disease progression (either metastatic disease or local recurrence) within 5 years of surgery.

Clinical information for all individuals is reported in an extensive Case Report Form (CRF) containing at least the following clinical information summarized in Tables 5 and 6 below: Individual ID; Demographics (Age and Menopausal Status); Lymph Node status; Estrogen Receptor status; Progesterone Receptor status; HER2 status; DNA ploidy; Clinical TNM Staging based on the modified AJCC/UICC TNM classification per CAP protocol (revision January 2004); Histopathological Type; Pathological and/or Nuclear Grade (Modified Bloom Richardson score); Pathological staging, pT size (Pathologic tumor size, size of the invasive component) based on the modified AJCC/UICC TNM classification per CAP protocol (revision January 2004); Treatment summary (date and type of surgery, chemotherapy received, radiotherapy received) and Clinical Outcome (date of evaluation, vitality at date of evaluation, disease progression status, months of disease free survival at date of evaluation and disease progression information). Additionally, the percentage of cells that are cancerous (Tum %) in the sample used for diagnosis and subsequent analysis is included.

Furthermore, when applicable, clinical information includes the percentage of cancerous cells per sample used for analysis.

TABLE 5
Diagnostic Case Information for Individuals

| | Demographics | | | | | | | | | | | | | | | | | |
|---|---|---|---|---|---|---|---|---|---|---|---|---|---|---|---|---|---|---|
| | | Men- | Diagnostic Summary | | | | | | | | | | | | | | | |
| | | opausal | Nodal | ER | PR | Her2 | DNA | Clinical Staging | | | Histopath | | Path | Path Staging | | | Grp | Tum |
| ID | Age | Status | Stat | Stat | Stat | Stat | Ploidy | T | N | M | Type | | Grade | T | N | M | Stag | % |
| 9 | 50 | Post- | LN− | ER− | PR− | Her2+ | A | NA | NA | NA | Inv | Duc | G3 | pT3 | pN0 | pMx | | |
| 34 | 36 | Pre- | LN− | ER+ | PR+ | Her2− | NA | T3 | N1 | M0 | IN | Duc | G2 | pT3 | PN0 | pMx | II B | 70 |
| 50 | 47 | Peri- | LN− | ER+ | PR− | Her2− | NA | T2 | N0 | M0 | Inv | Duc | G1 | pT2 | pN0 | pMx | II A | 60 |
| 55 | 40 | Pre- | LN− | ER+ | PR+ | Her2+ | NA | T2 | N0 | M0 | Inv | Duc | G3 | pT2 | pN0 | pMx | | 65 |
| 81 | 32 | Pre- | LN− | ER+ | PR+ | Her2− | NA | T3 | N0 | M0 | Inv | Duc | G1 | pT3 | pN0 | pMx | II B | 70 |
| 82 | 46 | NA | LN− | ER− | PR− | Her2− | NA | T3 | N1 | M0 | Inv | Duc | G2 | pT3 | pN0 | pMx | II B | |
| 97 | 32 | Pre- | LN− | ER− | PR− | Her2− | NA | T3 | N0 | M0 | Inv | Duc | G2 | pT3 | pN0 | pMx | II B | |
| 152 | 53 | Peri- | LN− | ER+ | PR+ | Her2+ | NA | T3 | N0 | M0 | Inv | Duc | G2 | pT2 | pN0 | pMx | | 70 |
| 173 | 46 | Pre- | LN− | ER+ | PR+ | NA | NA | T1 | N0 | M0 | Inv | Pap | G1 | pT1 | pN0 | pMx | I | 70 |
| 238 | 50 | Post- | LN− | ER+ | PR− | Her2+ | NA | T3 | N1 | M0 | Inv | Duc | G2 | pT2 | pN0 | pMx | II A | 60 |
| 277 | 70 | Post- | LN− | ER+ | PR+ | Her2− | NA | T2 | N0 | M0 | Inv | Duc | G2 | pT2 | pN0 | pMx | II A | 70 |
| 556 | 58 | Post- | LN− | ER+ | PR+ | Her2− | NA | T2 | N0 | M0 | Inv | Duc | G2 | pT1 | pN0 | pMx | I | 70 |
| 558 | 35 | Pre- | LN− | ER− | PR− | Her2− | NA | T2 | N1 | M0 | IS | Duc | G2 | pT3 | pN0 | pMx | II B | |
| 952 | 65 | Post- | LN− | ER+ | PR+ | Her2− | NA | T2 | N1 | M0 | Inv | Duc | G2 | pT2 | pN0 | pMx | II A | 70 |
| 983 | 75 | Pre- | LN− | ER+ | PR− | Her2− | NA | T3 | N0 | M0 | Inv | Duc | G1 | pT3 | pN0 | pMx | II B | 70 |
| 1009 | 40 | Pre- | LN− | ER+ | PR− | Her2− | NA | T2 | N0 | M0 | Inv | Duc | G1 | pT2 | pN0 | pMx | II A | 65 |
| 1103 | 44 | Pre- | LN− | ER+ | PR− | Her2− | D | NA | NA | NA | Inv | Duc | NA | pT1 | pN0 | pMx | | |
| 1105 | 70 | Post- | LN− | ER+ | PR+ | Her2− | NA | T2 | N0 | M0 | IS | Pap | G3 | pT2 | pN0 | pMx | II A | 60 |
| 1109 | 78 | Post- | LN− | ER+ | PR+ | Her2− | NA | T3 | N0 | M0 | Inv | Duc | G3 | pT2 | pN0 | pMx | II A | 90 |
| 1221 | 65 | Post- | LN− | ER+ | PR− | Her2− | T | T2 | N0 | M0 | Inv | Duc | G2 | pT2 | pN0 | pMx | II A | 60 |
| 1222 | 71 | Post- | LN− | ER+ | PR+ | Her2− | NA | NA | NA | NA | Inv | Duc | G2 | pT2 | pN0 | pMx | I | 80 |
| 1265 | 46 | Peri- | LN− | ER+ | PR+ | Her2− | NA | T3 | N0 | M0 | Inv | Duc | G1 | pT2 | pN0 | pMx | II A | 70 |
| 1275 | 60 | Post- | LN− | ER+ | PR+ | Her2+ | A | T2 | N0 | M0 | Inv | Duc | G2 | pT1 | pN0 | pMx | I | 60 |
| 1277 | 26 | Pre- | LN− | ER+ | PR+ | Her2− | A | T2 | N1 | M0 | Inv | Duc | G3 | pT2 | pN0 | pMx | II A | 70 |
| 1279 | 38 | Pre- | LN− | ER+ | PR+ | Her2− | NA | T2 | N1 | M0 | Inv | Duc | G2 | pT2 | pN0 | pMx | II A | 60 |
| 1280 | 52 | Post- | LN− | ER+ | PR+ | Her2+ | A | T2 | N0 | M0 | Inv | Duc | G3 | pT2 | pN0 | pMx | II A | 50 |
| 1281 | 59 | Post- | LN− | ER+ | PR+ | Her2− | NA | T2 | N1 | M0 | Inv | Duc | G2 | pT1 | pN0 | pMx | I | 50 |
| 1282 | 38 | Pre- | LN− | ER+ | PR+ | Her2− | NA | T2 | N0 | M0 | Inv | Duc | G3 | pT1 | pN0 | pMx | I | 60 |
| 1283 | 43 | Pre- | LN− | ER+ | PR+ | Her2+ | D | T1 | N1 | M0 | Inv | Duc | G2 | pT1 | pN0 | pMx | I | 60 |
| 1286 | 50 | Post- | LN− | ER+ | PR+ | Her2− | A | T2 | N1 | M0 | Inv | Duc | G2 | pT2 | pN0 | pMx | II A | 50 |
| 1298 | 52 | Post- | LN− | ER+ | PR+ | Her2− | NA | NA | NA | NA | Inv | Duc | G2 | pT2 | pN0 | pMx | II A | 60 |
| 1319 | 59 | Post- | LN− | ER+ | PR+ | Her2− | NA | T2 | N0 | M0 | Inv | Duc | G2 | pT3 | pN0 | pMx | II B | 70 |
| 1320 | 39 | Pre- | LN− | ER+ | PR+ | NA | NA | T2 | N0 | M0 | Inv | Duc | G2 | pT3 | pN0 | pMx | | |
| 1321 | 42 | Pre- | LN− | ER+ | PR+ | Her2− | NA | T2 | N0 | M0 | Inv | Duc | G2 | pT2 | pN0 | pMx | II A | 80 |
| 1322 | 44 | Pre- | LN− | ER+ | PR+ | Her2− | NA | T2 | N0 | M0 | Inv | Lob | G2 | pT2 | pN0 | pMx | II A | 70 |
| 1325 | 66 | NA | LN− | ER+ | PR+ | NA | NA | NA | NA | NA | Inv | Duc | G2 | pT3 | pN0 | pMx | II B | 50 |
| 1376 | 33 | NA | LN− | ER+ | PR+ | Her2+ | NA | NA | NA | NA | Inv | Duc | G2 | pT2 | pN0 | pMx | II A | 60 |
| 1377 | 38 | NA | LN− | ER+ | PR+ | Her2− | NA | NA | NA | NA | Inv | Duc | G3 | pT2 | pN0 | pMx | II A | 60 |
| 1379 | 42 | NA | LN− | ER+ | PR+ | Her2− | NA | NA | NA | NA | Inv | Duc | G2 | pT2 | pN0 | pMx | II A | 60 |
| 1386 | 48 | Post- | LN− | ER+ | PR+ | Her2+ | NA | T2 | N0 | M0 | Inv | Lob | G3 | pT1 | pN0 | pMx | I | |
| 1390 | 50 | Post- | LN− | ER+ | PR+ | Her2− | NA | T2 | N0 | M0 | Inv | Duc | G2 | pT2 | pN0 | pMx | | 60 |
| 1399 | 50 | Post- | LN− | ER+ | PR− | Her2+ | NA | T2 | N0 | M0 | Inv | Duc | G3 | pT3 | pN0 | pMx | II B | |
| 1464 | 53 | Post- | LN− | ER+ | PR+ | Her2− | NA | NA | NA | NA | Inv | Pap | G3 | pT2 | pN0 | pMx | II A | |
| 1469 | 70 | Post- | LN− | ER+ | PR+ | Her2− | NA | T2 | N0 | M0 | Inv | Lob | G2 | pT2 | pN0 | pMx | II A | |
| 1475 | 64 | Post- | LN− | ER+ | PR− | Her2− | NA | T2 | N0 | M0 | Inv | Duc | G1 | pT2 | pN0 | pMx | II A | |
| 1499 | 72 | Post- | LN− | ER+ | PR+ | Her2− | NA | NA | NA | NA | Inv | Duc | G3 | pTx | pN0 | pMx | II A/B | |
| 1502 | 64 | Post- | LN− | ER+ | PR+ | Her2− | NA | NA | NA | NA | Inv | Lob | G2 | pT1 | pN0 | pMx | I | |
| 1504 | 69 | Post- | LN− | ER+ | PR+ | Her2− | NA | T2 | N0 | M0 | Inv | Duc | G3 | pT2 | pN0 | pMx | II A | |
| 1561 | 50 | Pre- | LN− | ER+ | PR+ | Her2+ | NA | T2 | N0 | M0 | Inv | Pap | G3 | pT2 | pN0 | pMx | II A | |
| 1562 | 48 | Post- | LN− | ER− | PR− | Her2− | NA | T2 | N1 | M0 | Inv | Pap | G3 | pT2 | pN0 | pMx | II A | |
| 1642 | 65 | Post- | LN− | ER+ | PR− | Her2− | NA | T2 | N0 | M0 | Inv | Duc | G3 | pT1 | pN0 | pMx | I | |
| 1683 | 55 | Post- | LN− | ER+ | PR+ | NA | NA | T2 | N1 | M0 | Inv | Duc | G3 | pT2 | pN0 | pMx | II A | |
| 1846 | 37 | Pre- | LN− | ER+ | PR− | Her2− | NA | NA | NA | NA | Inv | Duc | G2 | pT2 | N0 | pMx | II A | |
| 1904 | 60 | Post- | LN− | ER+ | | | NA | T2 | N0 | M0 | Inv | Duc | G1 | pT2 | pN0 | pMx | II A | |
| 1905 | 55 | Post- | LN− | ER+ | | | NA | NA | NA | NA | Inv | Duc | G2 | pT2 | pN0 | pMx | II A | |

NA = information not available
Age is at time of diagnosis
For Ploidy: D = Diploid, T = Tetraploid, A = Aneuploid
For Histopathological Type: Inv = Invasive, IS = In Situ
For Pathological Grade: GX = Cannot be graded, G1 = Score 3-5, G2 Score 6-7, G3 = Score 8-9

TABLE 6
Treatment and Outcome Case Information for Individuals

| | Treatment Summary | | | | | | | Clinical Outcome | | | | |
|---|---|---|---|---|---|---|---|---|---|---|---|---|
| | Surgery | ChemoRx | | | | RadioRx | | Vital | Out- | | Progression | |
| ID | Type | NA | Adj | Met | HT | NA | Adj | Status | come | DFS | Desc | TTP |
| 9 | S | N | CAF | DEH | HT− | RT− | RT− | Alive | PD | NM | DM | 1 |
| 34 | L/S | N | N | NM | HT+ | RT− | RT− | Alive | DF | 108 | NM | NM |
| 50 | S | N | N | NM | HT+ | RT− | RT− | Alive | DF | 111 | NM | NM |
| 55 | M | CMF | CAF | NM | HT− | RT− | RT+ | Alive | DF | 60 | NM | NM |
| 81 | L/S | N | CAF | D | HT− | RT− | RT+ | Alive | PD | NM | O | 115 |
| 82 | M | N | CMF | CMF, CA | HT− | RT− | RT− | Alive | PD | NM | DM | 28 |
| 97 | S | N | N | N | HT− | RT− | RT− | Dead | PD | NM | DM | 48 |
| 152 | M | CA | CA | NM | HT− | RT− | RT+ | Alive | PD | NM | CLD | 29 |
| 173 | L | N | CA | NM | HT− | RT− | RT+ | Alive | DF | 58 | NM | NM |
| 238 | O | N | N | NM | HT+ | RT− | RT− | Alive | DF | 60 | NM | NM |
| 277 | L | N | N | NM | HT+ | RT− | RT+ | Alive | DF | 61 | NM | NM |
| 556 | S | N | N | NM | HT+ | RT− | RT− | Alive | DF | 84 | NM | NM |
| 558 | O | N | N | CMF | HT− | RT− | RT− | Dead | PD | NM | LR | 28 |
| 952 | S | N | CMF | NM | NA | RT− | NA | Alive | DF | 79 | NM | NM |
| 983 | S | N | N | NM | HT+ | RT− | RT− | Alive | DF | 77 | NM | NM |
| 1009 | L | N | FAC | NM | HT− | RT− | RT+ | Alive | DF | 76 | NM | NM |
| 1103 | S | N | FAC | NM | HT− | RT− | RT+ | Alive | PD | NM | CLD | 12 |
| 1105 | M | N | N | NM | HT+ | RT− | RT− | Alive | DF | 75 | NM | NM |
| 1109 | S | N | N | NM | HT+ | RT− | RT− | Alive | DF | 53 | NM | NM |
| 1221 | S | N | CMF | NM | HT+ | RT− | RT− | Alive | DF | 64 | NM | NM |
| 1222 | O | N | N | NM | HT+ | RT− | RT+ | Alive | DF | 71 | NM | NM |
| 1265 | M | N | CMF | NM | HT− | RT− | RT+ | Alive | DF | 85 | NM | NM |
| 1275 | M | N | CMF | NM | HT+ | RT− | RT− | Alive | DF | 60 | NM | NM |
| 1277 | M | N | FEC | DGCi | HT+ | RT− | RT+ | Alive | PD | NM | DM | 24 |
| 1279 | M | N | FEC | N | HT− | RT− | RT+ | Dead | PD | NM | DM | 68 |
| 1280 | M | N | FEC | N | HT+ | RT− | RT+ | Alive | PD | NM | CLD | 71 |
| 1281 | M | N | CMF | NM | HT+ | RT− | RT+ | Alive | DF | 70 | NM | NM |
| 1282 | O | N | FEC | NM | HT+ | RT− | RT+ | Alive | DF | 72 | NM | NM |
| 1283 | L | N | FEC | NM | HT+ | RT− | RT+ | Alive | DF | 79 | NM | NM |
| 1286 | M | N | CEF | N | HT+ | RT− | RT+ | Alive | PD | NM | LR | 10 |
| 1298 | M | N | CA | NM | HT+ | RT− | RT− | Alive | DF | 57 | NM | NM |
| 1319 | M | N | CMF | NM | HT+ | RT− | RT+ | Alive | DF | 69 | NM | NM |
| 1320 | M | N | FEC | NM | HT+ | RT− | RT+ | Alive | DF | 65 | NM | NM |
| 1321 | L | N | FEC | NM | HT+ | RT− | RT+ | Alive | DF | 76 | NM | NM |
| 1322 | O | N | N | NM | HT+ | RT− | RT+ | Alive | DF | 86 | NM | NM |
| 1325 | NA | N | NA | NM | NA | RT− | NA | Alive | DF | 76 | NM | NM |
| 1376 | NA | N | NA | NM | NA | RT− | NA | Alive | DF | 65 | NM | NM |
| 1377 | NA | N | NA | NM | NA | RT− | NA | Alive | DF | 83 | NM | NM |
| 1379 | NA | N | NA | NM | NA | RT− | NA | Alive | DF | 68 | NM | NM |
| 1386 | M | N | CAF | D, P, CA | HT+ | RT− | RT+ | Dead | PD | NA | DM | 18 |
| 1390 | O | N | CMF | N | HT+ | RT− | RT+ | Alive | PD | NM | DM | 39 |
| 1399 | M | N | N | CAF | HT− | RT− | RT− | Dead | PD | NA | DM | 18 |
| 1464 | S | N | N | NM | HT+ | RT− | RT− | Alive | DF | 60 | NM | NM |
| 1469 | S | N | N | NM | HT+ | RT− | RT− | Alive | DF | 93 | NM | NM |
| 1475 | M | N | N | NM | HT+ | RT− | RT+ | Alive | DF | 89 | NM | NM |
| 1499 | L/S | N | N | NM | HT+ | RT− | RT+ | Alive | DF | 65 | NM | NM |
| 1502 | S | N | NA | X | HT− | RT− | RT+ | Dead | PD | NA | DM | 31 |
| 1504 | S | N | CMF | NM | HT+ | RT− | RT− | Alive | DF | 60 | NM | NM |
| 1561 | S | N | CAF | D, P | HT+ | RT− | RT+ | Alive | PD | NA | DM | 45 |
| 1562 | S | N | N | Y | HT+ | RT− | RT− | Alive | PD | NM | DM | 47 |
| 1642 | S | N | N | CEF | HT+ | RT− | RT− | Alive | PD | NA | DM | 40 |
| 1683 | S | N | CAF | D, CA | HT+ | RT− | RT− | Dead | PD | NA | DM | 24 |
| 1846 | M | N | CAF | D | HT+ | RT− | RT+ | Dead | PD | NA | DM | 45 |
| 1904 | S | N | N | N | HT+ | RT− | RT+ | Dead | PD | NA | DM | 61 |
| 1905 | S | N | N | N | HT+ | RT− | RT+ | Dead | PD | NA | DM | 14 |

NA = information not available.
For Type of Surgery: S = Simple Mastectomy with axillary clearance, L = Lumpectomy with axillary clearance, M = Modified radical mastectomy, O = Other (specify in comment).
For Neo-adjuvant Chemotherapy (ChemoRx NA): C = cyclophosphamide, A = Adriamycin (doxorubicin), F = 5FU, E = epirubicin, M = methotrexate, D = docetaxel, P = paclitaxel, H = Herceptin (trastuzumab), G = gemcitabine, X = Xeloda (capecitabline), O = Others (specify in comment), Y = received treatment but regimen unknown, N = did not receive treatment.
For Adjuvant Chemotherapy (ChemoRx Adj): C = cyclophosphamide, A = Adriamycin (doxorubicin), F = 5FU, E = epirubicin, M = methotrexate, D = docetaxel, P = paclitaxel, H = Herceptin (trastuzumab), G = gemcitabine, X = Xeloda (capecitabline), O = Others (specify in comment), Y = received treatment but regimen unknown, N = did not receive treatment.
For Metastatic Chemotherapy (ChemoRx Met): C = cyclophosphamide, A = Adriamycin (doxorubicin), F = 5FU, E = epirubicin, M = methotrexate, D = docetaxel, P = paclitaxel, H = Herceptin (trastuzumab), G = gemcitabine, X = Xeloda (capecitabine), Ci = Cisplatin, O = Others (specify in comment), Y = received treatment but regimen unknown, NM = Not meaningful, N = Did not receive treatment.
For Hormone Therapy (ChemoRx HT): HT+ = Received treatment, HT− = Did not receive treatment.
For Neo-adjuvant Radiotherapy (RadioRx NA): RT+ = received treatment, RT− = did not receive treatment.
For Adjuvant Radiotherapy (RadioRx Adj): RT+ = received treatment, RT− = did not receive treatment.
For Clinical Outcome (as of date of assessment): DF = Disease-free, PD = Progressive disease Disease Free Survival (DFS) in months (at date of assessment): NM = Not meaningful (in event of disease progression), NK = Information not available.
For Progression Details (Progression, Desc): LR = Local recurrence, CLD Contra-lateral disease, DM = Distant metastasis, O = Others, NM = Not meaningful (no Disease Progression).
For Time to Progression in months (Progression, TTP) Relevant only in case of disease progression: NM = Not meaningful.

Differential expression of gene products from Tables 2a and 2b above identifies individuals with good outcome (disease free survival, DF, as no disease progression) and poor outcome with disease progression (progressive disease, PD, as either metastatic disease or local recurrence).

### Example 1b

### Prognosis Based on Gene Product Expression in Primary Tissue

### Primary Tissue Samples

As described above, the prognosis of individuals with breast cancer was determined based on gene product expression. Primary tissues from 45 individuals were evaluated for determining good or poor prognosis based on differential gene expression. The 20 individuals evaluated are ID numbers: 34, 50, 81, 173, 238, 277, 556, 952, 983, 1009, 1105, 1109, 1221, 1222, 1265, 1275, 1277, 1279, 1280, 1281, 1282, 1283, 1286, 1298, 1319, 1321, 1322, 1325, 1376, 1377, 1379, 1386, 1399, 1464, 1469, 1475, 1499, 1502, 1504, 1561, 1642, 1683, 1846, 1904 and 1905 which are characterized in tables 5 and 6 above. The results of the differential gene product expression analysis from the samples from these individuals are described below.

### Example 2

### Relative Quantitation of Gene Expression

Blood or Formalin Fixed Paraffin Embedded (FFPE) histological samples from the individuals described above were analyzed for gene expression by QPCR methodologies known to those of skill in the art, as exemplified below.

### FFPE Samples

Specifically, one FFPE block from a primary tumor resection from each individual was selected based on maximal tumor content. A narrow tumor content range was used to minimize the effects of the presence of non-cancer cells on the expression profile. Tumor content range is expected to be between 60 to 80% of cancer cells based on the characteristics of the samples in the sample bank.

Total RNA was extracted from two whole 20 micron sections from each FFPE block or from macro-dissected material. A total of 3-4 RNA samples from breast tissue from normal individuals and 3-4 total RNA samples from normal adjacent tissues (NAT) from pathologically normal breast tissues adjacent to a tumor from an individual with breast cancer were tested to obtain a baseline level of expression for each of the gene products tested. Prior to RNA extraction, paraffin was removed from samples by a deparaffinization step consisting of a xylene extraction followed by an ethanol wash. Kits for the extraction of RNA from FFPE samples such as the Optimun™ FFPE RNA Isolation Kit (Catalog #47000) from Ambion® Diagnostics (Austin, Tex.) are commercially available. Additionally, methodologies for processing FFPE samples are known to those of skill in the art, see Cronin et al. American Journal of Pathology, January 2004, Vol. 164, No. 1, pages 35-42. All measurements of gene products were normalized against endogenous controls.

### TaqMan™ Gene Expression Profiling

Removal of contaminating genomic DNA, quantitation of total RNA, measurements of residual genomic DNA contamination and preparation of cDNA by reverse transcription was performed prior to TaqMan™ gene expression profiling. TaqMan™ gene expression was performed on targets selected from Table 2 above.

Real-Time quantitative PCR with fluorescent Taqman® probes is a quantitation detection system utilizing the 5′-3′ nuclease activity of Taq DNA polymerase. The method uses an internal fluorescent oligonucleotide probe (Taqman®) labeled with a 5′ reporter dye and a downstream, 3′ quencher dye. During PCR, the 5′-3′ nuclease activity of Taq DNA polymerase releases the reporter, whose fluorescence can then be detected by the laser detector of a Realtime Quantitative PCR machine such as the Model 7000, 7700 or 7900 Sequence Detection System from PE Applied Biosystems (Foster City, Calif., USA). Amplification of an endogenous control(s) is used to standardize the amount of sample RNA added to the reaction and normalize for Reverse Transcriptase (RT) efficiency. Gene products from Table 4 above were used as endogenous control(s).

To calculate relative quantitation between all the samples studied, the target RNA levels for one sample can be used as the basis for comparative results (calibrator). Quantitation relative to the “calibrator” can be obtained using the comparative method (User Bulletin #2: ABI PRISM 7700 Sequence Detection System).

The tissue distribution and the level of the target gene are evaluated for every sample in normal and cancer tissues. Total RNA is extracted from normal tissues, cancer tissues, and from cancers and the corresponding matched adjacent tissues. Subsequently, first strand cDNA is prepared with reverse transcriptase and the polymerase chain reaction is done using primers and Taqman® probes specific to each target gene. The results are analyzed using the ABI PRISM 7700 Sequence Detector. The absolute numbers are relative levels of expression of the target gene in a particular tissue compared to the calibrator tissue.

One of ordinary skill can design appropriate primers using commercially available software such as Primer Express® 2.0 from Applied Biosystems (Foster City, Calif.) or Oligo® version 5 or 6 from Molecular Biology Insights, Inc (Cascade, Colo.). Criteria for designing primers are known to those of skill in the art, see Cronin et al. American Journal of Pathology, January 2004, Vol. 164, No. 1, pages 35-42.

The relative levels of expression of the gene in normal tissues versus other cancer tissues can then be determined. All the values are compared to the calibrator. Normal RNA samples are commercially available pools, originated by pooling samples of a particular tissue from different individuals. The expression of each gene was normalized against one or more endogenous controls as described above.

Alternatively, to compare expression profiles between specimens, normalization based on endogenous controls is used to correct for differences arising from variability in RNA quality and total quantity of RNA in each assay. A reference CT (threshold cycle) for each tested specimen is defined as the average measured CT of the endogenous controls. In an approach similar to what has been described by others, endogenous controls are selected for use from among several candidate reference genes tested in this assay. See Vandesompele J, et al., Genome Biol 2002, 3: RESEARCH0034. The endogenous controls selected for the final analysis show the lowest levels of expression variability among the individual specimens tested. An average of multiple gene products is used to minimize the risk of normalization bias that can result from variation in expression of any single reference gene. See Suzuki T, et al., Biotechniques 29:332-337 (2000). Relative mRNA level of a test gene within a tissue specimen is defined as 2^{ΔCT}+10.0, where ΔCT=CT (test gene)—CT (mean of endogenous controls). Unless indicated otherwise, normalized expression is represented on a scale in which the average expression of the endogenous controls is 10, corresponding to a mean CT of 30.7.

Table 7 below lists the components of each QPCR experiment performed on the genes described above. In some cases, multiple experiments have been designed for a single gene. The table includes the GenBank Accession for each gene, the SEQ ID NO and DDXS Accession for the amplified and detected portion of the gene, the DDXS nomenclature for the amplicon, the SEQ ID NO and DDXS Accession for the QPCR forward primer, the SEQ ID NO and DDXS Accession for the QPCR reverse primer and SEQ ID NO and DDXS Accession for the QPCR probe. Experiments are grouped by accession. For example, in a QPCR experiment for GenBank accession NM_##### the amplified and detected sequence is annotated as accession DEX0595_XXX.nt.1, the forward primer is DEX0595_XXX.nt.2, the reverse primer is DEX0595_XXX.nt.3 and the probe is DEX0595_XXX.nt.4.

TABLE 7

| | SEQ | DDXS Amplicon | | SEQ | DDXS Forward Primer |
|---|---|---|---|---|---|
| GenBank Accession | ID NO | Accession | DDXS Amplicon | ID NO | Accession |
| NM_001101.2 | 1 | DEX0595_001.nt.1 | ACTB.amp.1 | 2 | DEX0595_001.nt.2 |
| D38112.1 | 5 | DEX0595_002.nt.1 | ATP6.amp.1 | 6 | DEX0595_002.nt.2 |
| NM_004048.2 | 9 | DEX0595_003.nt.1 | B2M.amp.1 | 10 | DEX0595_003.nt.2 |
| NM_013230.1 | 13 | DEX0595_004.nt.1 | CD24.amp.1 | 14 | DEX0595_004.nt.2 |
| NM_000610.3 | 17 | DEX0595_005.nt.1 | CD44.amp.1 | 18 | DEX0595_005.nt.2 |
| NM_004360.2 | 21 | DEX0595_006.nt.1 | CDH1.amp.1 | 22 | DEX0595_006.nt.2 |
| NM_018098.4 | 25 | DEX0595_007.nt.1 | CLN176a.amp.1 | 26 | DEX0595_007.nt.2 |
| NM_018098.4 | 29 | DEX0595_008.nt.1 | CLN176b.amp.1 | 30 | DEX0595_008.nt.2 |
| NM_001909.3 | 33 | DEX0595_009.nt.1 | CTSD1.amp.1 | 34 | DEX0595_009.nt.2 |
| NM_199168.1 | 37 | DEX0595_010.nt.1 | CXCL12.amp.1 | 38 | DEX0595_010.nt.2 |
| NM_022059.1 | 41 | DEX0595_011.nt.1 | CXCL16.amp.1 | 42 | DEX0595_011.nt.2 |
| NM_006564.1 | 45 | DEX0595_012.nt.1 | DSH105.amp.1 | 46 | DEX0595_012.nt.2 |
| NM_004367.3 | 49 | DEX0595_013.nt.1 | DSH106.amp.1 | 50 | DEX0595_013.nt.2 |
| NM_022161.2 | 53 | DEX0595_014.nt.1 | DSH127.amp.1 | 54 | DEX0595_014.nt.2 |
| BC014098.2 | 57 | DEX0595_015.nt.1 | DSH140.amp.1 | 58 | DEX0595_015.nt.2 |
| NM_153758.1 | 61 | DEX0595_016.nt.1 | DSH141.amp.1 | 62 | DEX0595_016.nt.2 |
| NM_014452.3 | 65 | DEX0595_017.nt.1 | DSH176.amp.1 | 66 | DEX0595_017.nt.2 |
| NM_003965.3 | 69 | DEX0595_018.nt.1 | DSH209.amp.1 | 70 | DEX0595_018.nt.2 |
| NM_001908.3 | 73 | DEX0595_019.nt.1 | DSH223.amp.1 | 74 | DEX0595_019.nt.2 |
| NM_001571.2 | 77 | DEX0595_020.nt.1 | DSH371.amp.1 | 78 | DEX0595_020.nt.2 |
| NM_005201.2 | 81 | DEX0595_021.nt.1 | DSH375.amp.1 | 82 | DEX0595_021.nt.2 |
| NM_000579.1 | 85 | DEX0595_022.nt.1 | DSH51.amp.1 | 86 | DEX0595_022.nt.2 |
| NM_006988.3 | 89 | DEX0595_023.nt.1 | DSH607.amp.1 | 90 | DEX0595_023.nt.2 |
| NM_004591.1 | 93 | DEX0595_024.nt.1 | DSH73.amp.1 | 94 | DEX0595_024.nt.2 |
| NM_003579.2 | 97 | DEX0595_025.nt.1 | DSH811.amp.1 | 98 | DEX0595_025.nt.2 |
| AY623117.1 | 101 | DEX0595_026.nt.1 | DSH811a.amp.1 | 102 | DEX0595_026.nt.2 |
| NM_001838.2 | 105 | DEX0595_027.nt.1 | DSH859.amp.1 | 106 | DEX0595_027.nt.2 |
| NM_001008540.1 | 109 | DEX0595_028.nt.1 | DSH862.amp.1 | 110 | DEX0595_028.nt.2 |
| NM_002989.2 | 113 | DEX0595_029.nt.1 | DSH89.amp.1 | 114 | DEX0595_029.nt.2 |
| NM_178445.1 | 117 | DEX0595_030.nt.1 | DSH97.amp.1 | 118 | DEX0595_030.nt.2 |
| NM_006551.2 | 121 | DEX0595_031.nt.1 | ECM2.amp.1 | 122 | DEX0595_031.nt.2 |
| NM_002407.1 | 125 | DEX0595_032.nt.1 | ECM3.amp.1 | 126 | DEX0595_032.nt.2 |
| NM_000125.1 | 129 | DEX0595_033.nt.1 | ER.amp.1 | 130 | DEX0595_033.nt.2 |
| NM_004475.1 | 133 | DEX0595_034.nt.1 | ESA.amp.1 | 134 | DEX0595_034.nt.2 |
| NM_004475.1 | 137 | DEX0595_035.nt.1 | FLOT2.amp.1 | 138 | DEX0595_035.nt.2 |
| NM_002046.2 | 141 | DEX0595_036.nt.1 | GAPDH.amp.1 | 142 | DEX0595_036.nt.2 |
| NM_000181.1 | 145 | DEX0595_037.nt.1 | GUSB.amp.1 | 146 | DEX0595_037.nt.2 |
| NM_021102.2 | 149 | DEX0595_038.nt.1 | HAI-2.amp.1 | 150 | DEX0595_038.nt.2 |
| NM_004448.2 | 153 | DEX0595_039.nt.1 | HER2.amp.1 | 154 | DEX0595_039.nt.2 |
| NM_001530.2 | 157 | DEX0595_040.nt.1 | HIF1_alpha.amp.1 | 158 | DEX0595_040.nt.2 |
| NM_001530.2 | 161 | DEX0595_041.nt.1 | HIF1_alpha1.amp.1 | 162 | DEX0595_041.nt.2 |
| NM_000190.2 | 165 | DEX0595_042.nt.1 | HMBS.amp.1 | 166 | DEX0595_042.nt.2 |
| NM_000190.2 | 169 | DEX0595_043.nt.1 | HMBS2.amp.1 | 170 | DEX0595_043.nt.2 |
| NM_000194.1 | 173 | DEX0595_044.nt.1 | HPRT1.amp.1 | 174 | DEX0595_044.nt.2 |
| NM_002417.2 | 177 | DEX0595_045.nt.1 | Ki67.amp.1 | 178 | DEX0595_045.nt.2 |
| AL136163.15 | 181 | DEX0595_046.nt.1 | LNG186.amp.1 | 182 | DEX0595_046.nt.2 |
| NM_145016.2 | 185 | DEX0595_047.nt.1 | MAM004.amp.1 | 186 | DEX0595_047.nt.2 |
| AL157387.9 | 189 | DEX0595_048.nt.1 | MAM005.amp.1 | 190 | DEX0595_048.nt.2 |
| NM_001075.2, | 193 | DEX0595_049.nt.1 | MAM013.amp.1 | 194 | DEX0595_049.nt.2 |
| NM_001073.1 | | | | | |
| NM_001073.1 | 197 | DEX0595_050.nt.1 | MAM013b.amp.1 | 198 | DEX0595_050.nt.2 |
| AC018686.7 | 201 | DEX0595_051.nt.1 | MAM018.amp.1 | 202 | DEX0595_051.nt.2 |
| AC079414.6 | 205 | DEX0595_052.nt.1 | MAM021.amp.1 | 206 | DEX0595_052.nt.2 |
| AC079414.6 | 209 | DEX0595_053.nt.1 | MAM021a.amp.1 | 210 | DEX0595_053.nt.2 |
| AL356432.17 | 213 | DEX0595_054.nt.1 | MAM028.amp.1 | 214 | DEX0595_054.nt.2 |
| AL391686.10 | 217 | DEX0595_055.nt.1 | Mam029.amp.1 | 218 | DEX0595_055.nt.2 |
| AC093297.3 | 221 | DEX0595_056.nt.1 | MAM053.amp.1 | 222 | DEX0595_056.nt.2 |
| AY327584.1 | 225 | DEX0595_057.nt.1 | MAM096.amp.1 | 226 | DEX0595_057.nt.2 |
| XM_378356.1 | 229 | DEX0595_058.nt.1 | MAM109.amp.1 | 230 | DEX0595_058.nt.2 |
| NM_006408.2 | 233 | DEX0595_059.nt.1 | MAM111.amp.1 | 234 | DEX0595_059.nt.2 |
| NM_017697.2 | 237 | DEX0595_060.nt.1 | MAM113.amp.1 | 238 | DEX0595_060.nt.2 |
| NM_005449.3 | 241 | DEX0595_061.nt.1 | Mam121.amp.1 | 242 | DEX0595_061.nt.2 |
| NM_005449.3 | 245 | DEX0595_062.nt.1 | Mam121a.amp.1 | 246 | DEX0595_062.nt.2 |
| NM_005449.3 | 249 | DEX0595_063.nt.1 | MAM121v1.amp.1 | 250 | DEX0595_063.nt.2 |
| NM_016125.2 | 253 | DEX0595_064.nt.1 | MAM122b.amp.1 | 254 | DEX0595_064.nt.2 |
| NM_000305.1 | 257 | DEX0595_065.nt.1 | MAM123.amp.1 | 258 | DEX0595_065.nt.2 |
| AL831952.1 | 261 | DEX0595_066.nt.1 | MAM125.amp.1 | 262 | DEX0595_066.nt.2 |
| NM_006804.2 | 265 | DEX0595_067.nt.1 | Mam125a.amp.1 | 266 | DEX0595_067.nt.2 |
| NM_006573.3 | 269 | DEX0595_068.nt.1 | MAM127.amp.1 | 270 | DEX0595_068.nt.2 |
| AY358768.1 | 273 | DEX0595_069.nt.1 | MAM128V2.amp.1 | 274 | DEX0595_069.nt.2 |
| NM_016056.1 | 277 | DEX0595_070.nt.1 | MAM131.amp.1 | 278 | DEX0595_070.nt.2 |
| NM_002411.1 | 281 | DEX0595_071.nt.1 | MGB1.amp.1 | 282 | DEX0595_071.nt.2 |
| NM_004994.1 | 285 | DEX0595_072.nt.1 | MMP9.amp.1 | 286 | DEX0595_072.nt.2 |
| NM_024626.1 | 289 | DEX0595_073.nt.1 | OVR110a.amp.1 | 290 | DEX0595_073.nt.2 |
| NM_024626.1 | 293 | DEX0595_074.nt.1 | OVR110b.amp.1 | 294 | DEX0595_074.nt.2 |
| BC065717.1 | 297 | DEX0595_075.nt.1 | OVR110v1.amp.1 | 298 | DEX0595_075.nt.2 |
| NM_004625.3 | 301 | DEX0595_076.nt.1 | OVR212.amp.1 | 302 | DEX0595_076.nt.2 |
| NM_001554.3 | 305 | DEX0595_077.nt.1 | OVR235.amp.1 | 306 | DEX0595_077.nt.2 |
| NM_004625.3 | 309 | DEX0595_078.nt.1 | Ovr212a.amp.1 | 310 | DEX0595_078.nt.2 |
| NM_004625.3 | 313 | DEX0595_079.nt.1 | Ovr212c.amp.1 | 314 | DEX0595_079.nt.2 |
| AY216715.1 | 317 | DEX0595_080.nt.1 | OVR216.amp.1 | 318 | DEX0595_080.nt.2 |
| L08044.1 | 321 | DEX0595_081.nt.1 | Ovr226.amp.1 | 322 | DEX0595_081.nt.2 |
| NM_003226.2 | 325 | DEX0595_082.nt.1 | Ovr226v1.amp.1 | 326 | DEX0595_082.nt.2 |
| BC021275.2 | 329 | DEX0595_083.nt.1 | Pcan039b.amp.1 | 330 | DEX0595_083.nt.2 |
| NM_206925.1 | 333 | DEX0595_084.nt.1 | PCAN040.amp.1 | 334 | DEX0595_084.nt.2 |
| NM_022664.1, | 337 | DEX0595_085.nt.1 | PCAN043.amp.1 | 338 | DEX0595_085.nt.2 |
| U68187.1 | | | | | |
| NM_004385.2 | 341 | DEX0595_086.nt.1 | PCAN045.amp.1 | 342 | DEX0595_086.nt.2 |
| NM_033438.1 | 345 | DEX0595_087.nt.1 | PCAN047v1.amp.1 | 346 | DEX0595_087.nt.2 |
| NM_024042.2 | 349 | DEX0595_088.nt.1 | PCAN052.amp.1 | 350 | DEX0595_088.nt.2 |
| AE006464.1 | 353 | DEX0595_089.nt.1 | PCAN052v1.amp.1 | 354 | DEX0595_089.nt.2 |
| NM_000560.2 | 357 | DEX0595_090.nt.1 | PCAN056.amp.1 | 358 | DEX0595_090.nt.2 |
| AL391064.23 | 361 | DEX0595_091.nt.1 | Pcan056a.amp.1 | 362 | DEX0595_091.nt.2 |
| AY216715.1 | 365 | DEX0595_092.nt.1 | OVR216.amp.1 | 366 | DEX0595_092.nt.2 |
| NM_000560.2 | 369 | DEX0595_093.nt.1 | Pcan056b.amp.1 | 370 | DEX0595_093.nt.2 |
| NM_004864.1 | 373 | DEX0595_094.nt.1 | PCAN065.amp.1 | 374 | DEX0595_094.nt.2 |
| NM_032192.2 | 377 | DEX0595_095.nt.1 | PCAN070.amp.1 | 378 | DEX0595_095.nt.2 |
| NM_000926.2 | 381 | DEX0595_096.nt.1 | PR.amp.1 | 382 | DEX0595_096.nt.2 |
| NM_012445.1 | 385 | DEX0595_097.nt.1 | PRO108.amp.1 | 386 | DEX0595_097.nt.2 |
| NM_012445.1 | 389 | DEX0595_098.nt.1 | Pro108a.amp.1 | 390 | DEX0595_098.nt.2 |
| NM_012423.2 | 393 | DEX0595_099.nt.1 | RPL13A.amp.1 | 394 | DEX0595_099.nt.2 |
| NM_001002.3 | 397 | DEX0595_100.nt.1 | RPLPO_1.amp.1 | 398 | DEX0595_100.nt.2 |
| NM_004168.1 | 401 | DEX0595_101.nt.1 | SDHA.amp.1 | 402 | DEX0595_101.nt.2 |
| NM_004168.1 | 405 | DEX0595_102.nt.1 | SDHA2.amp.1 | 406 | DEX0595_102.nt.2 |
| NM_021102.2 | 409 | DEX0595_103.nt.1 | SPINT2.amp.1 | 410 | DEX0595_103.nt.2 |
| NM_003194.2 | 413 | DEX0595_104.nt.1 | TBP.amp.1 | 414 | DEX0595_104.nt.2 |
| NM_003219.1 | 417 | DEX0595_105.nt.1 | TERT.amp.1 | 418 | DEX0595_105.nt.2 |
| NM_003234.1 | 421 | DEX0595_106.nt.1 | TFRC.amp.1 | 422 | DEX0595_106.nt.2 |
| NM_021009.2 | 425 | DEX0595_107.nt.1 | UBC.amp.1 | 426 | DEX0595_107.nt.2 |
| NM_003376.3 | 429 | DEX0595_108.nt.1 | VEGF.amp.1 | 430 | DEX0595_108.nt.2 |
| NM_003406.2 | 433 | DEX0595_109.nt.1 | YWHAZ.amp.1 | 434 | DEX0595_109.nt.2 |

| | SEQ | DDXS Reverse Primer | SEQ | DDXS Probe |
|---|---|---|---|---|
| GenBank Accession | ID NO | Accession | ID NO | Accession |
| NM_001101.2 | 3 | DEX0595_001.nt.3 | 4 | DEX0595_001.nt.4 |
| D38112.1 | 7 | DEX0595_002.nt.3 | 8 | DEX0595_002.nt.4 |
| NM_004048.2 | 11 | DEX0595_003.nt.3 | 12 | DEX0595_003.nt.4 |
| NM_013230.1 | 15 | DEX0595_004.nt.3 | 16 | DEX0595_004.nt.4 |
| NM_000610.3 | 19 | DEX0595_005.nt.3 | 20 | DEX0595_005.nt.4 |
| NM_004360.2 | 23 | DEX0595_006.nt.3 | 24 | DEX0595_006.nt.4 |
| NM_018098.4 | 27 | DEX0595_007.nt.3 | 28 | DEX0595_007.nt.4 |
| NM_018098.4 | 31 | DEX0595_008.nt.3 | 32 | DEX0595_008.nt.4 |
| NM_001909.3 | 35 | DEX0595_009.nt.3 | 36 | DEX0595_009.nt.4 |
| NM_199168.1 | 39 | DEX0595_010.nt.3 | 40 | DEX0595_010.nt.4 |
| NM_022059.1 | 43 | DEX0595_011.nt.3 | 44 | DEX0595_011.nt.4 |
| NM_006564.1 | 47 | DEX0595_012.nt.3 | 48 | DEX0595_012.nt.4 |
| NM_004367.3 | 51 | DEX0595_013.nt.3 | 52 | DEX0595_013.nt.4 |
| NM_022161.2 | 55 | DEX0595_014.nt.3 | 56 | DEX0595_014.nt.4 |
| BC014098.2 | 59 | DEX0595_015.nt.3 | 60 | DEX0595_015.nt.4 |
| NM_153758.1 | 63 | DEX0595_016.nt.3 | 64 | DEX0595_016.nt.4 |
| NM_014452.3 | 67 | DEX0595_017.nt.3 | 68 | DEX0595_017.nt.4 |
| NM_003965.3 | 71 | DEX0595_018.nt.3 | 72 | DEX0595_018.nt.4 |
| NM_001908.3 | 75 | DEX0595_019.nt.3 | 76 | DEX0595_019.nt.4 |
| NM_001571.2 | 79 | DEX0595_020.nt.3 | 80 | DEX0595_020.nt.4 |
| NM_005201.2 | 83 | DEX0595_021.nt.3 | 84 | DEX0595_021.nt.4 |
| NM_000579.1 | 87 | DEX0595_022.nt.3 | 88 | DEX0595_022.nt.4 |
| NM_006988.3 | 91 | DEX0595_023.nt.3 | 92 | DEX0595_023.nt.4 |
| NM_004591.1 | 95 | DEX0595_024.nt.3 | 96 | DEX0595_024.nt.4 |
| NM_003579.2 | 99 | DEX0595_025.nt.3 | 100 | DEX0595_025.nt.4 |
| AY623117.1 | 103 | DEX0595_026.nt.3 | 104 | DEX0595_026.nt.4 |
| NM_001838.2 | 107 | DEX0595_027.nt.3 | 108 | DEX0595_027.nt.4 |
| NM_001008540.1 | 111 | DEX0595_028.nt.3 | 112 | DEX0595_028.nt.4 |
| NM_002989.2 | 115 | DEX0595_029.nt.3 | 116 | DEX0595_029.nt.4 |
| NM_178445.1 | 119 | DEX0595_030.nt.3 | 120 | DEX0595_030.nt.4 |
| NM_006551.2 | 123 | DEX0595_031.nt.3 | 124 | DEX0595_031.nt.4 |
| NM_002407.1 | 127 | DEX0595_032.nt.3 | 128 | DEX0595_032.nt.4 |
| NM_000125.1 | 131 | DEX0595_033.nt.3 | 132 | DEX0595_033.nt.4 |
| NM_004475.1 | 135 | DEX0595_034.nt.3 | 136 | DEX0595_034.nt.4 |
| NM_004475.1 | 139 | DEX0595_035.nt.3 | 140 | DEX0595_035.nt.4 |
| NM_002046.2 | 143 | DEX0595_036.nt.3 | 144 | DEX0595_036.nt.4 |
| NM_000181.1 | 147 | DEX0595_037.nt.3 | 148 | DEX0595_037.nt.4 |
| NM_021102.2 | 151 | DEX0595_038.nt.3 | 152 | DEX0595_038.nt.4 |
| NM_004448.2 | 155 | DEX0595_039.nt.3 | 156 | DEX0595_039.nt.4 |
| NM_001530.2 | 159 | DEX0595_040.nt.3 | 160 | DEX0595_040.nt.4 |
| NM_001530.2 | 163 | DEX0595_041.nt.3 | 164 | DEX0595_041.nt.4 |
| NM_000190.2 | 167 | DEX0595_042.nt.3 | 168 | DEX0595_042.nt.4 |
| NM_000190.2 | 171 | DEX0595_043.nt.3 | 172 | DEX0595_043.nt.4 |
| NM_000194.1 | 175 | DEX0595_044.nt.3 | 176 | DEX0595_044.nt.4 |
| NM_002417.2 | 179 | DEX0595_045.nt.3 | 180 | DEX0595_045.nt.4 |
| AL136163.15 | 183 | DEX0595_046.nt.3 | 184 | DEX0595_046.nt.4 |
| NM_145016.2 | 187 | DEX0595_047.nt.3 | 188 | DEX0595_047.nt.4 |
| AL157387.9 | 191 | DEX0595_048.nt.3 | 192 | DEX0595_048.nt.4 |
| NM_001075.2, | 195 | DEX0595_049.nt.3 | 196 | DEX0595_049.nt.4 |
| NM_001073.1 | | | | |
| NM_001073.1 | 199 | DEX0595_050.nt.3 | 200 | DEX0595_050.nt.4 |
| AC018686.7 | 203 | DEX0595_051.nt.3 | 204 | DEX0595_051.nt.4 |
| AC079414.6 | 207 | DEX0595_052.nt.3 | 208 | DEX0595_052.nt.4 |
| AC079414.6 | 211 | DEX0595_053.nt.3 | 212 | DEX0595_053.nt.4 |
| AL356432.17 | 215 | DEX0595_054.nt.3 | 216 | DEX0595_054.nt.4 |
| AL391686.10 | 219 | DEX0595_055.nt.3 | 220 | DEX0595_055.nt.4 |
| AC093297.3 | 223 | DEX0595_056.nt.3 | 224 | DEX0595_056.nt.4 |
| AY327584.1 | 227 | DEX0595_057.nt.3 | 228 | DEX0595_057.nt.4 |
| XM_378356.1 | 231 | DEX0595_058.nt.3 | 232 | DEX0595_058.nt.4 |
| NM_006408.2 | 235 | DEX0595_059.nt.3 | 236 | DEX0595_059.nt.4 |
| NM_017697.2 | 239 | DEX0595_060.nt.3 | 240 | DEX0595_060.nt.4 |
| NM_005449.3 | 243 | DEX0595_061.nt.3 | 244 | DEX0595_061.nt.4 |
| NM_005449.3 | 247 | DEX0595_062.nt.3 | 248 | DEX0595_062.nt.4 |
| NM_005449.3 | 251 | DEX0595_063.nt.3 | 252 | DEX0595_063.nt.4 |
| NM_016125.2 | 255 | DEX0595_064.nt.3 | 256 | DEX0595_064.nt.4 |
| NM_000305.1 | 259 | DEX0595_065.nt.3 | 260 | DEX0595_065.nt.4 |
| AL831952.1 | 263 | DEX0595_066.nt.3 | 264 | DEX0595_066.nt.4 |
| NM_006804.2 | 267 | DEX0595_067.nt.3 | 268 | DEX0595_067.nt.4 |
| NM_006573.3 | 271 | DEX0595_068.nt.3 | 272 | DEX0595_068.nt.4 |
| AY358768.1 | 275 | DEX0595_069.nt.3 | 276 | DEX0595_069.nt.4 |
| NM_016056.1 | 279 | DEX0595_070.nt.3 | 280 | DEX0595_070.nt.4 |
| NM_002411.1 | 283 | DEX0595_071.nt.3 | 284 | DEX0595_071.nt.4 |
| NM_004994.1 | 287 | DEX0595_072.nt.3 | 288 | DEX0595_072.nt.4 |
| NM_024626.1 | 291 | DEX0595_073.nt.3 | 292 | DEX0595_073.nt.4 |
| NM_024626.1 | 295 | DEX0595_074.nt.3 | 296 | DEX0595_074.nt.4 |
| BC065717.1 | 299 | DEX0595_075.nt.3 | 300 | DEX0595_075.nt.4 |
| NM_004625.3 | 303 | DEX0595_076.nt.3 | 304 | DEX0595_076.nt.4 |
| NM_001554.3 | 307 | DEX0595_077.nt.3 | 308 | DEX0595_077.nt.4 |
| NM_004625.3 | 311 | DEX0595_078.nt.3 | 312 | DEX0595_078.nt.4 |
| NM_004625.3 | 315 | DEX0595_079.nt.3 | 316 | DEX0595_079.nt.4 |
| AY216715.1 | 319 | DEX0595_080.nt.3 | 320 | DEX0595_080.nt.4 |
| L08044.1 | 323 | DEX0595_081.nt.3 | 324 | DEX0595_081.nt.4 |
| NM_003226.2 | 327 | DEX0595_082.nt.3 | 328 | DEX0595_082.nt.4 |
| BC021275.2 | 331 | DEX0595_083.nt.3 | 332 | DEX0595_083.nt.4 |
| NM_206925.1 | 335 | DEX0595_084.nt.3 | 336 | DEX0595_084.nt.4 |
| NM_022664.1, | 339 | DEX0595_085.nt.3 | 340 | DEX0595_085.nt.4 |
| U68187.1 | | | | |
| NM_004385.2 | 343 | DEX0595_086.nt.3 | 344 | DEX0595_086.nt.4 |
| NM_033438.1 | 347 | DEX0595_087.nt.3 | 348 | DEX0595_087.nt.4 |
| NM_024042.2 | 351 | DEX0595_088.nt.3 | 352 | DEX0595_088.nt.4 |
| AE006464.1 | 355 | DEX0595_089.nt.3 | 356 | DEX0595_089.nt.4 |
| NM_000560.2 | 359 | DEX0595_090.nt.3 | 360 | DEX0595_090.nt.4 |
| AL391064.23 | 363 | DEX0595_091.nt.3 | 364 | DEX0595_091.nt.4 |
| AY216715.1 | 367 | DEX0595_092.nt.3 | 368 | DEX0595_092.nt.4 |
| NM_000560.2 | 371 | DEX0595_093.nt.3 | 372 | DEX0595_093.nt.4 |
| NM_004864.1 | 375 | DEX0595_094.nt.3 | 376 | DEX0595_094.nt.4 |
| NM_032192.2 | 379 | DEX0595_095.nt.3 | 380 | DEX0595_095.nt.4 |
| NM_000926.2 | 383 | DEX0595_096.nt.3 | 384 | DEX0595_096.nt.4 |
| NM_012445.1 | 387 | DEX0595_097.nt.3 | 388 | DEX0595_097.nt.4 |
| NM_012445.1 | 391 | DEX0595_098.nt.3 | 392 | DEX0595_098.nt.4 |
| NM_012423.2 | 395 | DEX0595_099.nt.3 | 396 | DEX0595_099.nt.4 |
| NM_001002.3 | 399 | DEX0595_100.nt.3 | 400 | DEX0595_100.nt.4 |
| NM_004168.1 | 403 | DEX0595_101.nt.3 | 404 | DEX0595_101.nt.4 |
| NM_004168.1 | 407 | DEX0595_102.nt.3 | 408 | DEX0595_102.nt.4 |
| NM_021102.2 | 411 | DEX0595_103.nt.3 | 412 | DEX0595_103.nt.4 |
| NM_003194.2 | 415 | DEX0595_104.nt.3 | 416 | DEX0595_104.nt.4 |
| NM_003219.1 | 419 | DEX0595_105.nt.3 | 420 | DEX0595_105.nt.4 |
| NM_003234.1 | 423 | DEX0595_106.nt.3 | 424 | DEX0595_106.nt.4 |
| NM_021009.2 | 427 | DEX0595_107.nt.3 | 428 | DEX0595_107.nt.4 |
| NM_003376.3 | 431 | DEX0595_108.nt.3 | 432 | DEX0595_108.nt.4 |
| NM_003406.2 | 435 | DEX0595_109.nt.3 | 436 | DEX0595_109.nt.4 |

### Expression Results

Expression results for several gene products measured by QPCR in samples from 20 individuals are listed in Table 8 below. Data is presented as relative expression using a Human Reference sample as a calibrator, which is assigned a value of one (1) for all other samples to be calibrated against. All expression data is normalized using the geometric mean of 2 endogenous controls (HPRT1 and RPL13A).

TABLE 8
Gene product Expression Results

| | Gene Product ID | | | | | | | | | | |
|---|---|---|---|---|---|---|---|---|---|---|---|
| Indiv ID | AGR2 | CYR61 | SLAMF9 | ECT2 | ECM1 | RAD54-like | CCR8 | ESR1 | PGR | TERT | CDH1 |
| 34 | 2.42 | 1.28 | 0.05 | 0.10 | 0.03 | 0.15 | 3.49 | 10.59 | 20.34 | 0.07 | 2.04 |
| 50 | 0.61 | 0.03 | 0.02 | 0.14 | 0.04 | 0.06 | 1.31 | 4.82 | 0.26 | 0.03 | 2.17 |
| 81 | 1.03 | 0.25 | 0.10 | 0.10 | 0.03 | 0.51 | 10.82 | 3.58 | 13.29 | 0.49 | 6.97 |
| 173 | 2.26 | 0.20 | 0.01 | 0.12 | 0.37 | 0.08 | 6.53 | 4.84 | 7.12 | 0.00 | 5.88 |
| 238 | 2.37 | 1.54 | 0.01 | 0.21 | 0.01 | 0.07 | 0.16 | 23.86 | 0.00 | 0.00 | 3.77 |
| 277 | 0.11 | 2.57 | 0.41 | 0.80 | 0.03 | 0.00 | 1.54 | 21.81 | 7.43 | 0.01 | 13.12 |
| 556 | 5.68 | 1.25 | 0.32 | 0.12 | 0.19 | 0.01 | 0.20 | 14.63 | 23.81 | 0.04 | 5.82 |
| 952 | 3.53 | 0.16 | 0.07 | 0.09 | 2.15 | 0.19 | 1.07 | 15.17 | 7.57 | 0.05 | 3.52 |
| 983 | 1.47 | 8.14 | 0.21 | 0.48 | 0.49 | 0.03 | 2.16 | 9.21 | 4.82 | 0.00 | 5.87 |
| 1009 | 0.88 | 0.17 | 0.03 | 0.08 | 0.03 | 0.44 | 2.82 | 6.94 | 0.24 | 0.19 | 3.98 |
| 1105 | 4.97 | 2.82 | 0.38 | 0.22 | 0.01 | 0.11 | 1.16 | 16.00 | 11.53 | 0.04 | 1.75 |
| 1109 | 2.48 | 0.62 | 0.01 | 0.14 | 0.00 | 0.06 | 2.17 | 13.05 | 26.16 | 0.07 | 4.12 |
| 1221 | 2.39 | 1.04 | 0.05 | 0.11 | 0.10 | 0.21 | 4.16 | 2.92 | 3.57 | 0.01 | 1.43 |
| 1222 | 0.50 | 1.77 | 0.02 | 0.37 | 1.16 | 0.30 | 0.94 | 0.48 | 0.34 | 1.32 | 3.20 |
| 1265 | 0.76 | 0.20 | 0.03 | 0.35 | 0.01 | 0.13 | 0.43 | 29.14 | 67.42 | 0.00 | 2.54 |
| 1275 | 15.34 | 0.20 | 0.58 | 0.09 | 0.17 | 0.24 | 34.93 | 7.76 | 9.72 | 0.08 | 2.33 |
| 1277 | 0.01 | 0.74 | 0.05 | 0.37 | 0.10 | 0.01 | 25.41 | 0.06 | 0.24 | 0.19 | 0.99 |
| 1279 | 9.11 | 0.92 | 0.32 | 0.25 | 0.25 | 0.19 | 2.80 | 9.11 | 16.84 | 0.01 | 3.66 |
| 1280 | 1.24 | 95.52 | 4.77 | 0.56 | 0.70 | 0.20 | 23.53 | 1.94 | 2.86 | 0.17 | 31.65 |
| 1281 | 0.20 | 7.66 | 0.15 | 0.14 | 0.37 | 0.06 | 1.17 | 3.11 | 0.73 | 0.00 | 5.33 |
| 1282 | 0.00 | 0.34 | 0.01 | 1.49 | 0.68 | 3.36 | 9.61 | 0.04 | 0.02 | 0.18 | 0.71 |
| 1283 | 9.74 | 0.95 | 0.23 | 0.76 | 0.58 | 0.17 | 0.82 | 5.06 | 1.79 | 0.19 | 11.40 |
| 1286 | 1.24 | 4.11 | 0.01 | 0.04 | 0.02 | 0.11 | 0.84 | 4.53 | 0.78 | 0.02 | 6.02 |
| 1298 | 2.54 | 0.80 | 0.08 | 0.07 | 0.03 | 0.14 | 2.80 | 35.05 | 0.23 | 0.02 | 2.77 |
| 1319 | 3.08 | 0.11 | 0.02 | 0.21 | 0.86 | 0.13 | 0.10 | 9.34 | 28.08 | 0.03 | 1.41 |
| 1321 | 13.12 | 0.66 | 0.01 | 0.81 | 8.48 | 0.12 | 1.76 | 31.61 | 80.16 | 0.06 | 2.36 |
| 1322 | 4.89 | 0.96 | 0.00 | 0.05 | 0.25 | 0.06 | 0.55 | 3.61 | 11.14 | 0.00 | 5.75 |
| 1325 | 1.69 | 0.93 | 0.09 | 0.21 | 0.17 | 0.11 | 3.70 | 21.04 | 4.76 | 0.05 | 0.25 |
| 1376 | 4.18 | 0.54 | 0.28 | 0.29 | 0.04 | 0.37 | 3.41 | 2.19 | 24.09 | 0.07 | 1.23 |
| 1377 | 1.67 | 1.70 | 0.11 | 0.12 | 0.10 | 0.23 | 5.23 | 1.13 | 9.33 | 0.09 | 0.82 |
| 1379 | 6.81 | 3.60 | 0.59 | 0.09 | 0.78 | 0.10 | 4.90 | 3.52 | 10.91 | 0.07 | 0.50 |
| 1386 | 6.46 | 1.70 | 0.63 | 0.75 | 0.15 | 1.74 | 6.03 | 30.73 | 7.91 | 1.17 | 7.30 |
| 1399 | 1.42 | 0.46 | 0.02 | 0.19 | 1.06 | 8.18 | 0.35 | 3.46 | 0.31 | 0.17 | 5.38 |
| 1464 | 11.15 | 3.20 | 0.02 | 0.09 | 0.30 | 0.18 | 17.05 | 5.66 | 12.48 | 0.13 | 1.31 |
| 1469 | 4.06 | 0.47 | 0.10 | 0.11 | 0.09 | 0.07 | 1.33 | 33.74 | 21.08 | 0.01 | 4.70 |
| 1475 | 0.79 | 0.50 | 0.49 | 0.24 | 0.46 | 0.20 | 42.16 | 1.75 | 0.86 | 0.08 | 2.33 |
| 1499 | 12.41 | 5.20 | 1.47 | 1.87 | 1.23 | 0.95 | 11.74 | 92.83 | 171.80 | 0.12 | 18.56 |
| 1502 | 2.68 | 0.82 | 0.05 | 0.48 | 0.02 | 0.18 | 11.59 | 18.62 | 10.11 | 0.01 | 0.99 |
| 1504 | 3.42 | 0.07 | 0.01 | 0.19 | 0.01 | 0.14 | 0.78 | 12.87 | 11.33 | 0.00 | 1.11 |
| 1561 | 6.08 | 0.72 | 0.65 | 0.08 | 1.35 | 0.37 | 3.36 | 1.55 | 0.22 | 0.03 | 96.95 |
| 1642 | 0.06 | 0.47 | 0.00 | 0.22 | 0.33 | 0.48 | 2.90 | 23.68 | 0.10 | 0.02 | 14.59 |
| 1683 | 0.37 | 0.58 | 0.13 | 0.22 | 0.11 | 0.15 | 7.09 | 5.15 | 8.13 | 0.02 | 1.86 |
| 1846 | 2.36 | 1.03 | 0.21 | 0.23 | 6.01 | 0.25 | 5.81 | 2.86 | 13.88 | 1.43 | 1.54 |
| 1904 | 17.26 | 3.47 | 0.10 | 0.08 | 0.03 | 0.08 | 4.48 | 22.86 | 10.51 | 0.01 | 2.42 |
| 1905 | 16.01 | 1.31 | 0.37 | 0.45 | 0.08 | 0.38 | 10.49 | 20.79 | 6.31 | 0.03 | 3.01 |

| | Gene Product ID | | | | | | | | |
|---|---|---|---|---|---|---|---|---|---|
| Indiv ID | CTSD | B7-H4 | TNFSF13B | BXMAS2-10 | Mam005 | IRF3 | MMP9 | SCGB1D2 | CXCR6 |
| 34 | 1.01 | 204.20 | 1.37 | 0.91 | 1750.03 | 1.07 | 10.21 | 5462.45 | 13.95 |
| 50 | 0.80 | 0.48 | 0.21 | 0.01 | 1.03 | 0.07 | 9.12 | 6.84 | 1.40 |
| 81 | 2.00 | 78.36 | 6.34 | 0.01 | 14.87 | 1.02 | 27.51 | 28.07 | 12.33 |
| 173 | 1.76 | 323.25 | 2.12 | 12.58 | 2046.90 | 1.38 | 8.27 | 349.35 | 5.91 |
| 238 | 4.34 | 0.01 | 0.83 | 0.02 | 0.14 | 1.68 | 3.22 | 0.08 | 0.21 |
| 277 | 2.39 | 0.02 | 0.90 | 0.02 | 430.34 | 0.40 | 10.02 | 0.13 | 0.29 |
| 556 | 1.96 | 1.69 | 0.09 | 0.15 | 241.14 | 1.58 | 15.96 | 4.23 | 1.26 |
| 952 | 2.17 | 50.37 | 3.58 | 0.10 | 4.33 | 0.39 | 13.18 | 19.18 | 2.50 |
| 983 | 1.07 | 55.46 | 1.59 | 3.45 | 441.39 | 0.25 | 35.13 | 203.15 | 1.19 |
| 1009 | 1.63 | 0.49 | 3.07 | 0.01 | 358.86 | 0.93 | 6.51 | 2.95 | 2.34 |
| 1105 | 6.16 | 51.80 | 7.23 | 1.47 | 217.97 | 0.36 | 84.02 | 707.62 | 3.92 |
| 1109 | 0.71 | 3.62 | 2.38 | 1.02 | 53.12 | 0.36 | 7.82 | 6.04 | 3.42 |
| 1221 | 3.68 | 2.40 | 3.66 | 1.07 | 0.03 | 0.64 | 8.15 | 1246.66 | 5.85 |
| 1222 | 1.79 | 144.59 | 15.59 | 0.20 | 0.45 | 0.35 | 21.38 | 0.22 | 8.00 |
| 1265 | 1.25 | 59.59 | 2.35 | 0.02 | 0.02 | 0.57 | 9.26 | 0.82 | 3.85 |
| 1275 | 3.40 | 2.07 | 4.94 | 0.21 | 36.52 | 0.69 | 26.55 | 3.63 | 0.61 |
| 1277 | 1.11 | 1798.96 | 17.71 | 1.59 | 1.58 | 0.88 | 15.22 | 2.30 | 28.21 |
| 1279 | 1.63 | 86.66 | 4.52 | 1.28 | 470.90 | 1.01 | 7.89 | 122.53 | 5.60 |
| 1280 | 1.04 | 34.87 | 1.86 | 0.55 | 9.01 | 0.39 | 87.21 | 169.60 | 8.25 |
| 1281 | 4.06 | 78.65 | 2.69 | 109.23 | 76.93 | 0.29 | 44.52 | 6.31 | 1.40 |
| 1282 | 0.21 | 5.73 | 1.37 | 0.01 | 0.33 | 0.07 | 22.21 | 0.07 | 8.33 |
| 1283 | 2.03 | 2.36 | 2.82 | 0.41 | 509.84 | 0.55 | 0.78 | 180.11 | 1.19 |
| 1286 | 0.77 | 13.80 | 0.41 | 0.05 | 7.45 | 0.15 | 16.26 | 1.57 | 1.05 |
| 1298 | 1.44 | 7.57 | 1.37 | 0.03 | 497.38 | 0.40 | 97.12 | 0.19 | 3.29 |
| 1319 | 0.42 | 0.01 | 0.23 | 0.00 | 1.79 | 0.18 | 0.62 | 4.13 | 1.55 |
| 1321 | 15.00 | 0.03 | 3.08 | 0.00 | 1057.18 | 0.35 | 542.67 | 4952.85 | 6.05 |
| 1322 | 1.36 | 165.63 | 2.03 | 0.00 | 0.69 | 0.24 | 1808.46 | 460.60 | 3.92 |
| 1325 | 2.04 | 45.27 | 11.00 | 0.08 | 1511.15 | 0.60 | 4.58 | 62151.43 | 7.17 |
| 1376 | 0.66 | 84.18 | 15.15 | 0.03 | 2444.99 | 2.89 | 3.92 | 638.80 | 9.07 |
| 1377 | 1.00 | 163.67 | 33.43 | 1.13 | 421.47 | 1.00 | 6.61 | 65.53 | 3.23 |
| 1379 | 1.46 | 3.71 | 3.94 | 0.42 | 112.24 | 0.52 | 117.18 | 3.66 | 1.05 |
| 1386 | 1.71 | 146.24 | 3.05 | 0.03 | 2162.79 | 0.58 | 0.01 | 2.24 | 9.83 |
| 1399 | 2.14 | 3.39 | 5.45 | 2.21 | 183.83 | 0.68 | 34.76 | 88.42 | 4.85 |
| 1464 | 0.44 | 25.89 | 3.89 | 0.01 | 236.82 | 0.22 | 13.31 | 32.25 | 6.10 |
| 1469 | 0.75 | 27.93 | 13.44 | 1.26 | 738.59 | 0.61 | 14.39 | 75.33 | 5.87 |
| 1475 | 2.05 | 5.15 | 9.16 | 48.71 | 4.69 | 0.83 | 75.80 | 77.62 | 13.58 |
| 1499 | 6.37 | 21.39 | 9.34 | 1.24 | 8.57 | 2.18 | 21.41 | 66.19 | 17.66 |
| 1502 | 1.07 | 0.44 | 7.45 | 2.05 | 0.08 | 4.60 | 10.35 | 70.97 | 7.09 |
| 1504 | 0.56 | 0.16 | 2.74 | 0.00 | 190.77 | 0.36 | 4.38 | 5615.98 | 4.02 |
| 1561 | 3.49 | 41.33 | 2.41 | 4.88 | 113.69 | 0.32 | 70.37 | 29.99 | 1.90 |
| 1642 | 1.22 | 0.22 | 2.72 | 0.11 | 0.01 | 0.44 | 33.77 | 1.67 | 0.90 |
| 1683 | 2.28 | 56.31 | 4.68 | 1.28 | 2.89 | 0.63 | 50.10 | 21.54 | 10.30 |
| 1846 | 1.14 | 3.14 | 17.08 | 0.06 | 15.96 | 0.22 | 118.86 | 2410.08 | 8.13 |
| 1904 | 1.42 | 9.90 | 2.85 | 1.49 | 472.76 | 0.93 | 87.51 | 164.86 | 8.43 |
| 1905 | 1.10 | 36.90 | 1.80 | 4.15 | 3.39 | 0.41 | 14.48 | 916.08 | 4.43 |

The results in Table 8 show that the over-expression levels of the gene products RAD54L, CYR61, ECT2, CCR8, BXMAS2-10, ESR1, CXCR6, B7-H4, TERT, CDH1 and CTSD above of a particular threshold are indicative of poor outcome and recurrence of disease within 5 years of surgery. More particularly gene products RAD54L, CCR8, BXMAS2-10, CXCR6, CYR61, CDH1 and B7-H4 (borderline significance) under a particular threshold were indicative of poor outcome and recurrence of disease within 5 years of surgery. Statistical analysis was based on a student t-test. Additionally, the results in Table 8 indicate that combinations of two or more of the gene products listed in Table 2 or Table 7 can be used to determine likelihood of long-term survival and therapy response for an individual.

### Example 3

### Statistical Analyses

Normalized gene product expression values from the experiments described above are used to study the existence of correlation of each individual gene product with overall outcome. Gene products identified as relevant for the prediction of outcome are evaluated in a multivariate model as predictors of prognosis. Analyses include: Principal Component Analysis, supervised principal component analysis, support vector machines, classification algorithms; calculation of survival rates at 5 years by prognosis signature (independently by gene and by combination of genes); Kaplan-Meier analysis for survival or events at 5 years by prognosis signature (independently by gene and by combination of genes) including p-values; univariate Cox or logistic regressions for survival or events at 5 years by prognosis signature (independently by gene and by combination of genes) including p-values; and multivariate Cox or logistic regressions for survival or events at 5 years by prognosis signature using individual genes (selected from Survival Analysis 3) or gene combination and incorporating significant clinical variables. References and additional statistical methodologies can be found in Van De Vijver, et al., NEJM, Vol. 347, No. 25 Dec. 19, 2002.

### Univariate Testing

For comparison of individual gene product expression between disease free (DF) and progressive disease (PD) individuals two-sample Wilcoxon test, t-test and Wlech tests analyses are used. The Wilcoxon test is a non-parametric alternative to the two sample t-test which does not make any underlying distributional assumptions. Using these tests sets of genes from Table 2a, Table 2b or Table 7 showing the strongest gene product up-regulation in the PD population are identified. These gene sets preferably comprise 2, 3, 4, 5, 6, 7, 8, 9 or 10 genes. Most preferably these gene sets comprise 2, 3, 4, or 5 genes. Up-regulation or down-regulation of gene products from a gene set may be indicative of an individual falling in the progressive disease population.

Based on a Wilcoxon test of the values in Table 8, the gene products of RAD54L, CCR8, BXMAS2-10 and CXCR6 showed the strongest evidence of over-expression associated with progressive disease with p-values of 0.029, 0.035, 0.057 and 0.070, respectively.

### Univariate Cox Proportional Hazards Results

Evaluation of the association of individual gene product expression with survival time is effected using Cox proportional hazards regression. A set of preferably 2, 3, 4, 5 or 6 genes from Table 2a, Table 2b or Table 7 are identified. Over expression of these gene products is associated with survival time of individuals with progressive disease and can be used for staging and therapy selection. Ideally, over expression of the gene products is associated with survival time regardless of an individual's age, tumor stage, tumor grade or tumor size.

Based on a Cox proportional hazards regression analysis of values in Table 8, the gene products of RAD54L, B7-H4 and CDH1 showed the strongest association with survival with p-values of 0.024, 0.073 and 0.084, respectively. Specifically, RAD54L expression over the median value was associated with a decreased survival time.

### Survival Analyses

To conduct survival analyses gene product expression, select demographic and clinical variables are forced into a Cox proportional hazards model. To allow for selection of variables for inclusion, a penalized stepwise approach based on the Lasso and Least Angle Regression analyses may be employed. Use of conventional (unpenalized) stepwise approaches is undesirable in small sample settings. Using these methods gene sets from Table 2a, Table 2b or Table 7 may be identified as variables which are associated with survival time of individuals. Preferably the gene sets include 2, 3, 4 or 5 genes. Up-regulation or down-regulation of gene products from the gene set is associated with survival time for an individual.

Null and/or unstable results were obtained when all gene product expression, age, tumor grade and tumor size were forced into a Cox proportional hazards model. The first two genes selected via use of a penalized forward-stepwise approach (LARS-Lasso) were RAD54L and CDH1, which withstood cross validation.

### Classification

In order to identify gene product signatures from genes in Table 2a, Table 2b or Table 7 useful for discriminating between disease free and progressive disease individuals, two disparate classifications techniques are suitable: Nearest Shrunken Centroids method (generalizes prototype methods and diagonal linear discriminant analysis), and Random Forests (combines tree-based classifiers obtained using bootstrap resampling). These techniques are suitable because they are effective in small sample settings featuring potentially correlated predictors (e.g. gene products), are designed to avoid overfitting and provide interpretable output with respect to which gene are driving classification. Additionally, the shrunken centroids analysis has been previously employed to identify sets of gene that characterize selected groups based on gene product expression. See Tibshirani et al., PNAS 99(10):3 6567-6572 (2002).

### Nearest Shrunken Centroids

Using the nearest shrunken centroids method comparable classification rates are obtained for a range of shrinkage parameters and gene product expression values corresponding to models with 3, 4, 5, 6, 7, 8, 9 or 10 genes from Table 2a, Table 2b or Table 7. Misclassification rates are ideally below 30 percent. Perfectly classified DF individuals and misclassified PD individuals indicates a preponderance of DF individuals. By varying prior probabilities (equivalently misclassifications cots) balanced results with comparable misclassification rates are attainable.

Comparable misclassification rates (˜0.25) were obtained for a range of shrinking parameters and values corresponding to modules with 3 to 9 genes from Table 2a and Table 2b. In a 9 gene model, the gene product expression of B7-H4, CDH1, PR, CYR61, Mam005, SCGB1D2, CXCR6, MMP9 and BXMAS2-10 had an overall misclassification rate of 0.24. Disease free individuals were perfectly classified, and only individuals were misclassified, reflecting the preponderance of disease free individuals.

### Random Forests

Using the random forests method sets of gene from Table 2a, Table 2b or Table 7 are identified which are most important to the method for classification of individuals as DF or PD. Summaries of individual an individual gene's contributions to classification are provided via variable importance measures. As with the nearest shrunken centroids method misclassification rates are ideally below 30 percent. Additionally, similar misclassification rates between methods is indicative of the accuracy of each method.

The top nine gene products contributing to classification by random forests were CCR8, RAD54L, TERT, CDH1, BXMAS2-10, SLAMF9, CXCR6, MMP9 and PR.

Reasonable concordance between sets of gene from Table 2a, Table 2b or Table 7 identified by the nearest shrunken centroids method and random forests method such as CXCR6, CDH1, PR, MMP9 and BXMAS2-10 demonstrates that the differential expression of the gene products classify an individual as falling into the disease free or progressive disease groups.

### Results

The statistical analyses conducted demonstrate that the expression levels of the gene products of the genes of Table 2a and Table 2b are useful for discriminating between progressive disease and disease free survival groups for the prognosis of individuals with breast cancer. Specifically, differential of RAD54L, CCR8, CXCR6, BXMAS2-10, B7-H4, CDH1, PR and MMP9 gene products are useful for disease free prognosis, survival determination, and group classification.

### Example 4

### ROC Analysis of Marker Panels for Breast Cancer Prognosis

The ability of a test or assay to discriminate diseased cases from normal cases or to discriminate two different populations of patients with different characteristics is evaluated using Receiver Operating Characteristic (ROC) curve analysis (Metz, 1978; Zweig & Campbell, 1993). ROC curves can also be used to compare the diagnostic performance of two or more laboratory or diagnostic tests (Griner et al., 1981).

An ROC curve is generated by plotting the sensitivity against the specificity for each value. From the plot, the area under the curve (AUC) can be determined. The value for the area under the ROC curve (AUC) can be interpreted as follows: an area of 0.84, for example, means that a randomly selected positive result has a test value larger than that for a randomly chosen negative result 84% of the time (Zweig & Campbell, 1993). When the variable under study can not distinguish between two result groups, i.e. where there is no difference between the two distributions, the area will be equal to 0.5 (the ROC curve will coincide with the diagonal). When there is a perfect separation of the values of the two groups, i.e. there no overlapping of the distributions, the area under the ROC curve equals 1 (the ROC curve will reach the upper left corner of the plot).

The 95% confidence interval for the area can be used to test the hypothesis that the theoretical area is 0.5. If the confidence interval does not include the 0.5 value, then there is evidence that the laboratory test has the ability to distinguish between the two groups (Hanley & McNeil, 1982; Zweig & Campbell, 1993). The P-value is the probability that the observed sample Area under the ROC curve is found when in fact, the true (population) Area under the ROC curve is 0.5 (null hypothesis: Area=0.5). If P is low (P<0.05) then it can be concluded that the Area under the ROC curve is significantly different from 0.5 and that therefore there is evidence that the laboratory test does have an ability to distinguish between the two groups.

### ROC Analysis of Individual Gene Products

The sensitivity and specificity of each gene product to individually differentiate the disease free (DF; good prognosis) and progressive disease (PD; poor prognosis) populations was calculated through receiver operating characteristic (ROC) analysis. The gene products of four genes tested (RAD54L, CCR8, BXMAS2-10, CXCR6) had an AUC larger than 0.5 with a 95% confident interval. The P-values for the gene products of these genes showed that the expression results have the ability to distinguish between good and poor prognosis groups. Table 9 below shows the results of the Area Under the Curve (AUC) from the ROC analysis for the gene products in individuals with progressive disease (PD, n=14) versus individuals free of disease within 5 years (DF, n=31). The AUC values for RAD54L, CCR8, BXMAS2-10, and CXCR6 were 0.706, 0.699, 0.680 and 0.672, respectively. AUC values for gene products were calculated using the gene expression levels obtained by quantitative real time PCR as described previously in the TaqMan™ Gene Expression Profiling section and reported in Table 8.

TABLE 9
AUC Values for genes RAD54L, CCR8,
BXMAS2_10, and CXCR6.

| | | 95^{th} Confidence | |
|---|---|---|---|
| | AUC | Interval | P-value |
| RAD54L | 0.706 | 0.552 to 0.832 | 0.0197 |
| CCR8 | 0.699 | 0.544 to 0.827 | 0.0251 |
| BXMAS2_10 | 0.680 | 0.524 to 0.811 | 0.0468 |
| CXCR6 | 0.672 | 0.516 to 0.804 | 0.0590 |

These results demonstrate that gene products of the genes of Table 2a and Table 2b can distinguish between disease free and progressive disease groups and are useful for the prognosis of individuals with breast cancer.

### ROC Analysis of Combination of Gene Products for

The ability of a combination of gene products to differentiate disease free (DF; good prognosis) and progressive disease (PD; poor prognosis) populations was determined by receiver operating characteristic (ROC) analyses of various mathematical relationships. The area under the curve (AUC) was calculated using the mathematical relationship of the gene product expression results in Table 8 above for the gene products of the genes in Table 2a and Table 2b.

### Additive Combos

The sensitivity and specificity for two or more gene products of the genes in Table 2a and Table 2b in additive combination was calculated through (ROC analysis using the expression value of each gene listed in Table 8. Table 10 below shows the results of the Area Under the Curve (AUC) from the ROC analysis of gene product expression levels of RAD54L and CCR8 individually and in combination, and RAD54L and CXCR6 individually and in combination in individuals with progressive disease (PD, n=14) versus individuals free of disease within 5 years (DF, n=31)

TABLE 10
Additive Combination AUC Values.

| | AUC | | | | |
|---|---|---|---|---|---|
| | | | | RAD54L + | RAD54L + |
| | RAD54L | CCR8 | CXCR6 | CCR8 | CXCR6 |
| PD versus | 0.706 | 0.699 | 0.672 | 0.786 | 0.716 |
| DF | | | | | |

These results demonstrate that gene products of the genes of Table 2a and Table 2b in additive combination can distinguish individuals with progressive disease (PD, n=14) from individuals free of disease within 5 years and are useful the prognosis of individuals with breast cancer.

### Ratio and Multiplicative Combos

Another method for calculating the predictive value of the association of the gene product expression of two or more genes is to use the ratio of the expression values or the product of the multiplication of the expression values. Previous reports proposed the use of a two-gene expression ratio (Ma et al., Cancer Cell. 2004 June; 5(6):607-16) as predictor of recurrence in ER-positive, invasive breast cancer patients treated with tamoxifen and the use of an interactive gene expression index (IGEI) (DeMuth et al., Am J Respir Cell Mol. Biol. 1998 July; 19(1):18-24) to predict a malignant phenotype in human bronchial epithelial cells. The disclosure of Ma et al., and DeMuth et al., are hereby incorporated by reference. Table X below lists the calculated ratios and multiplicative results for gene product expression results in Table 8 above.

Table 11 below lists the gene product expression ratio and multiplicative values used in the ROC analyses below.

TABLE 11
Ratio and Multiplication Values.

| | | | | | | RAD54L × CCR8 × |
|---|---|---|---|---|---|---|
| Indiv ID | RAD54L/CCR8 | RAD54L/CXCR6 | CCR8/CXCR6 | RAD54L × CCR8 | CCR8 × CXCR6 | CXCR6 |
| 34 | 0.043425412 | 0.010856729 | 0.250008664 | 0.528331988 | 48.6640111 | 7.371117384 |
| 50 | 0.072783516 | 0.077495457 | 1.832045781 | 0.072783516 | 1.832045781 | 0.101653608 |
| 81 | 5.468160373 | 6.233268631 | 133.4746747 | 5.468160373 | 133.4746747 | 67.44938265 |
| 173 | 0.524014187 | 0.474597951 | 38.58436464 | 0.524014187 | 38.58436464 | 3.097705515 |
| 238 | 0.010987616 | 0.014167433 | 0.035065324 | 0.010987616 | 0.035065324 | 0.002336341 |
| 277 | 0.006943843 | 0.001291264 | 0.44335984 | 0.006943843 | 0.44335984 | 0.001993819 |
| 556 | 0.001706874 | 0.010565993 | 0.254656107 | 0.001706874 | 0.254656107 | 0.002143053 |
| 952 | 0.203699044 | 0.478451451 | 2.666309849 | 0.203699044 | 2.666309849 | 0.509763506 |
| 983 | 0.061806398 | 0.03409333 | 2.5634143 | 0.061806398 | 2.5634143 | 0.073495514 |
| 1009 | 1.239478574 | 1.027900059 | 6.60381635 | 1.239478574 | 6.60381635 | 2.900630779 |
| 1105 | 0.126783987 | 0.429742523 | 4.536808542 | 0.126783987 | 4.536808542 | 0.497177645 |
| 1109 | 0.129161181 | 0.203694337 | 7.421330092 | 0.129161181 | 7.421330092 | 0.441871871 |
| 1221 | 0.880774401 | 1.238686509 | 24.30774984 | 0.880774401 | 24.30774984 | 5.149741444 |
| 1222 | 0.283234059 | 2.400608262 | 7.555881102 | 0.283234059 | 7.555881102 | 2.266605531 |
| 1265 | 0.05740508 | 0.512096956 | 1.664153934 | 0.05740508 | 1.664153934 | 0.221181097 |
| 1275 | 8.501593821 | 0.148455248 | 21.30765615 | 8.501593821 | 21.30765615 | 5.185800358 |
| 1277 | 0.306364751 | 0.340152962 | 716.8639613 | 0.306364751 | 716.8639613 | 8.64320672 |
| 1279 | 0.529107706 | 1.058264312 | 15.70624788 | 0.529107706 | 15.70624788 | 2.965550624 |
| 1280 | 4.647072724 | 1.629943801 | 194.1447506 | 4.647072724 | 194.1447506 | 38.34766068 |
| 1281 | 0.068655119 | 0.082408927 | 1.633701542 | 0.068655119 | 1.633701542 | 0.096141292 |
| 1282 | 32.29487505 | 27.99828718 | 80.09831565 | 32.29487505 | 80.09831565 | 269.118918 |
| 1283 | 0.141499285 | 0.205641023 | 0.971531941 | 0.141499285 | 0.971531941 | 0.168135935 |
| 1286 | 0.090285896 | 0.112641559 | 0.890493989 | 0.090285896 | 0.890493989 | 0.095164458 |
| 1298 | 0.383050247 | 0.450080684 | 9.191958103 | 0.383050247 | 9.191958103 | 1.25885897 |
| 1319 | 0.012307563 | 0.198781521 | 0.148993595 | 0.012307563 | 0.148993595 | 0.019092282 |
| 1321 | 0.214119618 | 0.737367408 | 10.62940465 | 0.214119618 | 10.62940465 | 1.295461972 |
| 1322 | 0.035115592 | 0.250061621 | 2.160635269 | 0.035115592 | 2.160635269 | 0.137741322 |
| 1325 | 0.395714911 | 0.766876766 | 26.56030852 | 0.395714911 | 26.56030852 | 2.839033751 |
| 1376 | 1.261717468 | 3.357891362 | 30.90972447 | 1.261717468 | 30.90972447 | 11.44358093 |
| 1377 | 1.185275328 | 0.731360977 | 16.88950985 | 1.185275328 | 16.88950985 | 3.826344224 |
| 1379 | 0.490512263 | 0.104749199 | 5.126808859 | 0.490512263 | 5.126808859 | 0.513243966 |
| 1386 | 10.47556066 | 17.08457225 | 59.28884919 | 10.47556066 | 59.28884919 | 103.009482 |
| 1399 | 2.861710591 | 39.66178705 | 1.69456046 | 2.861710591 | 1.69456046 | 13.86843735 |
| 1464 | 3.13794969 | 1.121986685 | 103.9867251 | 3.13794969 | 103.9867251 | 19.13400091 |
| 1469 | 0.093987396 | 0.414854626 | 7.817159337 | 0.093987396 | 7.817159337 | 0.552086668 |
| 1475 | 8.460932451 | 2.724704925 | 572.3352548 | 8.460932451 | 572.3352548 | 114.8666886 |
| 1499 | 11.09821349 | 16.70014691 | 207.3353385 | 11.09821349 | 207.3353385 | 196.0303649 |
| 1502 | 2.136986476 | 1.307137683 | 82.18165302 | 2.136986476 | 82.18165302 | 15.15126837 |
| 1504 | 0.10520519 | 0.5443266 | 3.122376359 | 0.10520519 | 3.122376359 | 0.422854528 |
| 1561 | 1.226270945 | 0.695189298 | 6.394557126 | 1.226270945 | 6.394557126 | 2.334801662 |
| 1642 | 1.404866783 | 0.433238492 | 2.598717841 | 1.404866783 | 2.598717841 | 1.257652487 |
| 1683 | 1.091944855 | 1.585898017 | 73.0628774 | 1.091944855 | 73.0628774 | 11.24828644 |
| 1846 | 1.440796585 | 2.014702286 | 47.27809499 | 1.440796585 | 47.27809499 | 11.71485073 |
| 1904 | 0.34717728 | 0.653010357 | 37.82350385 | 0.34717728 | 37.82350385 | 2.928306705 |
| 1905 | 3.995750956 | 1.685488363 | 46.43154343 | 3.995750956 | 46.43154343 | 17.68351704 |

Table 12 below shows the results of the Area Under the Curve (AUC) from the ROC analyses for the ratios of gene product expression values for gene RAD54L/CCR8, RAD54L/CXCR6 and CCR8/CXCR6 in individuals with progressive disease (PD, n=14) versus individuals free of disease within 5 years (DF, n=31).

TABLE 12
AUC Values for ratios of gene products.

| | | 95^{th} Confidence | |
|---|---|---|---|
| | AUC | Interval | P-value |
| RAD54L/CCR8 | 0.816 | 0.672 to 0.915 | <0.0001 |
| RAD54L/CXCR6 | 0.753 | 0.602 to 0.869 | 0.0005 |
| CCR8/CXCR6 | 0.774 | 0.625 to 0.885 | 0.0001 |

Table 13 below shows the results of the Area Under the Curve (AUC) from the ROC analyses for the ratios of gene product expression values for gene RAD54L x CCR8, CCR8 x CXCR6 and RAD54L x CCR8 x CXCR6 in individuals with progressive disease (PD, n=14) versus individuals free of disease within 5 years (DF, n=31).

TABLE 13
AUC Values for multiplication of gene products.

| | | 95^{th} Confidence | |
|---|---|---|---|
| | AUC | Interval | P-value |
| RAD54L × CCR8 | 0.790 | 0.643 to 0.897 | <0.0001 |
| CCR8 × CXCR6 | 0.728 | 0.575 to 0.850 | 0.0029 |
| RAD54L × CCR8 × | 0.772 | 0.623 to 0.883 | 0.0001 |
| CXCR6 | | | |

### Results of ROC Analyses

The results from the ROC analyses of the gene product expression from different genes demonstrate that the expression of gene products of the genes from Table 2a and Table 2b used individually are useful for differentiating disease free (DF; good prognosis) and progressive disease (PD; poor prognosis) individuals. Furthermore, when the individual gene product expression values for the genes of Table 2a and Table 2b are used in additive combination for the ROC analysis the AUC is higher than the individual gene product AUCs as demonstrated in Table 10. Similarly, when the individual gene product expression values for the genes of Table 2a and Table 2b are used in mathematical relationships such as ratios or multiplication in different combination of two or more of the genes to obtain a new value that is used in the ROC analysis, we obtain higher AUC values compared to the AUC of each individual gene as demonstrated in Table 12 and Table 13.

These results demonstrate that the gene products of the genes of Table 2a and Table 2b, including RAD54L, CCR8, BXMAS2-10 and CXCR6, alone or in combination, are useful for differentiating individuals with progressive disease (PD) versus individuals free of disease within 5 years (DF) and prognosing them into poor outcome (progressive disease) and good outcome (disease free survival) groups.

### Example 5

### Bone Marrow Samples

The prognosis of an individual with breast cancer can be determined based on the gene product expression of a bone marrow sample. Bone marrow samples are usually obtained through aspiration. The bone marrow samples are often aspirated from the upper iliac crest, the posterior iliac crest, or the anterosuperior iliac spine. In the aspiration process, the individual's skin is incised and 2-5 mL of bone marrow is aspirated.

Using the gene products of Table 2a and 2b and the methods of Example 2 and 3, bone marrow samples from each individual are processed for analysis of gene products according to methods known by those of skill in the art. For example, see Barbaric D, et al., J. Clin. Pathol. 55:865-867 (2002).

Bone marrow samples from healthy individuals are used to determine a baseline level of expression for each of the gene products tested. All measurements of gene products are normalized against endogenous controls.

Specific markers that can be used individually or in combination to detect and/or predict breast cancer for an individual include BXMAS2-10, Mam005, WNT7A, SLAMF9, B7-H4, SCGB2A1, HIF1A, SCGB1D2, UGT2B11, Mam021, Mam018, Mam029 and Mmgb.

Specific markers that are used to determine cancerous cells in the bone marrow of an individual regularly include CK-19 (see Slade M J, et al., Int. J. Cancer 114(1):94-100 (2005)) and mammaglobin (see Ooka M, et al., Breast Cancer Res. Treat. 67(2): 169-75 (2001).

### Example 6

### Blood Samples

The prognosis of an individual with breast cancer can be determined based on the gene product expression of a peripheral blood sample. Peripheral blood samples are collected after consent from the individuals is obtained. For individuals with cancer, blood samples are often collected after surgery, and for individuals without cancer the blood can be collected at anytime.

Using the gene products of Table 2a and 2b and the methods of Example 2 and 3, blood samples from each individual are processed for analysis of gene products according to methods known by those of skill in the art. From each individual and control donor 10 ml of blood (in PaxGene tubes) is collected. RNA is extracted from blood samples by methods known by those of skill in the art, or by use of commercially available kits such as Qiagene RNA collection kits which utilize the Qiagene RNA collection procedure.

For analysis of RNA, an amplification step may be used to improve sensitivity using commercially available kits such as the Ovation™ System from Nugen™ (San Carlos, Calif.). Additionally, emerging amplification methodologies such as Whole Transcriptome Amplification (WTA) which does not demonstrate a 3′ bias as seen in other RNA detection methodologies may be utilized. Available WTA services and forthcoming commercially available WTA kits include Ribo-SPIA™ WTA from Nugen™ and the TransPlex™ Whole Transcriptome Amplification Kits from Rubicon Genomics (Ann Arbor, Mich.). See Nugen™ website nugenteclinologies with the extension .com/technology-wt-spia.htm of the world wide web and Rubicon Genetics website rubicongenomics with the extension .com/web/OmniPlexWTAKits.html of the world wide web.

Blood samples from healthy individuals are used to determine a baseline level of expression for each of the gene products tested. All measurements of gene products are normalized against endogenous controls.

Specific markers that can be used individually or in combination to detect and/or predict breast cancer for an individual include BXMAS2-10, Mam005, WNT7A, SLAMF9, B7-H4, SCGB2 μl, HIF1A, SCGB1D2, UGT2B11, Mam021, Mam018, Mam029 and Mmgb.

Specific markers that are used to determine cancerous cells in the peripheral blood of an individual regularly include CK-1 g (see Stathopoulou A, et al., J. Clin. Oncol., 20(16):3404-3412 (2002)) and mammaglobin (see Cerveira N, et al., Int. J. Cancer 108(4):592-595 (2004)). In addition to these individual markers, several multi-marker sets are also used to detect cancerous cells in an individual's peripheral blood. These multi-marker sets consist of mammaglobin and beta-hCG (see Fabisiewicz A, et al., Acta Biochim. Pol. 51(3):747-755 (2004)); CK-19 and beta-hCG (see Hu X C, et al., Anticancer Res. 21(1A):421-424 (2001)); beta-hCG, c-Met, MAGE-A3 and GalNAc-T (see Taback B, et al., Cancer Res. 61(24):8845-8850 (2001)); and p1B, PS2, CK-19 and EGP2 (see Bosma A J, et al., Clin. Cancer Res. 8(6):1871-1877 (2002)).

### Example 7

### Lymph Nodes

The prognosis of an individual with breast cancer can be determined based on the gene product expression of a lymph node sample. Lymph node samples are collected through several methods. Individuals found to have breast cancer undergo an axillary lymph node dissection (lymph node is surgically removed) or they have a sentinel lymphandenectomy performed. In order to obtain non-cancerous lymph nodes, oftentimes individuals having surgeries such as a cholecystectomy or a tonsillectomy are asked to provide samples of their lymph nodes.

Using the gene products of Table 2a and 2b and the methods of Example 2 and 3, lymph node samples from each individual are processed for analysis of gene products according to methods known by those of skill in the art.

Lymph node samples from healthy individuals are used as controls and to determine a baseline level of expression for each of the gene products tested. All measurements of gene products are normalized against endogenous controls.

Specific markers that can be used individually or in combination to detect and/or predict breast cancer for an individual include BXMAS2-10, Mam005, WNT7A, SLAMF9, B7-H4, SCGB2A1, HIF1A, SCGB1D2, UGT2B11, Mam021, Mam018, Mam029 and Mmgb.

Specific markers that are used to determine cancerous cells in the lymph nodes of an individual include mammaglobin (see Min C J, et al., Cancer Res. 58(20):4581-4584 (1998) and Ooka M, et al., Oncol. Rep. 7(3):561-566 (2000)) and CEA (see Min C J, et al., Cancer Res. 58(20):4581-4584 (1998) and Mori M, et al., Cancer Res. 55(15):3417-3420 (1995)). In addition to these individual markers, several multi-marker sets are also used to detect cancerous cells in an individual's lymph nodes. These multi-marker sets consist of mam, PIP, CK-19, mamB, MUC-1, and CEA (see Mitas M, et al., Int. J. Cancer 93(2):162-171 (2001)); mam, B305D, GABApi, and B726P (see Zehentner B K, et al., Clin. Chem. 48(8):1225-1231 (2002)); and mapsin, CK-19 and mam (see Manzotti M, et al., Int. J. Cancer 95(5):307-312 (2001)).

We claim:

1. A method for determining the prognosis for an individual having breast cancer comprising: determining the expression level of a plurality of gene products of the genes in Table 2a in a sample from an individual relative to a control, wherein the differential expression of a plurality of gene products relative to a control is indicative of the individual's prognosis.

2. The method of claim 1 wherein the expression level of a plurality of gene products of the genes in Table 2b is also determined and the differential expression of a plurality of gene products relative to a control is indicative of the individual's prognosis.

3. The method of claim 1 wherein the plurality of gene products comprises at least two gene products.

4. The method of claim 1 wherein the plurality of gene products comprises at least four gene products.

5. The method of claim 1 wherein the plurality of gene products comprises at least six gene products.

6. The method of claim 1 wherein the plurality of gene products comprises at least eight gene products.

7. The method of claim 2 wherein the gene products are selected from the group comprising RAD54L, CYR61, ECT2, CCR8, BXMAS2-10, ESR1, CXCR6, B7-H4, TERT, CDH1 and CTSD.

8. The method of claim 7 wherein the gene products are selected from the group comprising RAD54L, CCR8, BXMAS2-10, CXCR6, CYR61, CDH1 and B7-H4.

9. The method of claim 7 wherein over-expression of CYR61, ECT2, CCR8, ESR1, B7-H4, TERT, and CDH1 gene products are indicative of a poor prognosis.

10. The method of claim 8 wherein over-expression of RAD54L, CCR8, BXMAS2-10, CXCR6, CYR61, CDH1 and B7-H4 gene products are indicative of a good prognosis.

11. The method of claim 7 wherein under-expression of ER and CTSD gene products are indicative of a poor prognosis.

12. The method of claim 1 wherein over-expression of some gene products from Table 2a are indicative of a good prognosis.

13. The method of claim 1 wherein under-expression of some gene products from Table 2a are indicative of a good prognosis.

14. The method of claim 2 wherein over-expression of some gene products from Table 2b are indicative of a poor prognosis.

15. The method of claim 2 wherein under-expression of some gene products from Table 2b are indicative of a poor prognosis.

16. The method of claim 1 where in the gene product is a RNA.

17. The method of claim 16 wherein the gene product expression level is determined by quantitative PCR.

18. The method of claim 1 wherein the gene product is a polypeptide.

19. The method of claim 18 wherein the gene product expression is determined by an assay comprising one or more antibodies.

20. The method of claim 1 wherein the sample is selected from the group consisting of tissues, cells and bodily fluids.

21. The method of claim 20 wherein the tissues or cells are from a fixed, waxed embedded specimen from said individual.

22. A method for improving the prognosis for an individual having breast cancer comprising modulating levels of a plurality of gene products in Table 2a or Table 2b.

23. The method of claim 22 wherein the plurality of gene products comprises at least two gene products.

24. The method of claim 22 wherein the plurality of gene products comprises at least four gene products.

25. The method of claim 22 wherein the plurality of gene products comprises at least six gene products.

26. The method of claim 22 wherein the plurality of gene products comprises at least eight gene products.

27. The method of claim 22 wherein modulating levels of gene products comprises increasing levels of gene products whose over-expression is associated with a good prognosis.

28. The method of claim 27 wherein the gene products are selected from the group comprising the gene products of Table 2a.

29. The method of claim 22 wherein modulating levels of gene products comprises decreasing levels of gene products whose under-expression is associated with a good prognosis.

30. The method of claim 29 wherein the gene products are selected from the group comprising the gene products of Table 2a.

31. The method of claim 22 wherein modulating levels of gene products comprises decreasing levels of gene products whose over-expression is associated with a poor prognosis.

32. The method of claim 22 wherein modulating levels of gene products comprises increasing levels of gene products whose under-expression is associated with a poor prognosis.

33. The method of claim 22 comprising administering to said individual an appropriate agonist or antagonist for a gene product of Table 2a which will improve the prognosis of said individual.

34. An isolated nucleic acid molecule comprising:
(a) a nucleic acid molecule consisting essentially of a nucleic acid sequence of Table 7;
(b) a nucleic acid molecule that selectively hybridizes to the nucleic acid molecule of (a); or
(c) a nucleic acid molecule having at least 95% sequence identity to the nucleic acid molecule of (a).

35. The nucleic acid molecule according to claim 34, wherein the nucleic acid molecule is cDNA.

36. The nucleic acid molecule according to claim 34, wherein the nucleic acid molecule is genomic DNA.

37. The nucleic acid molecule according to claim 34, wherein the nucleic acid molecule is RNA.

38. The nucleic acid molecule according to claim 34, wherein the nucleic acid molecule is a mammalian nucleic acid molecule.

39. The nucleic acid molecule according to claim 38, wherein the nucleic acid molecule is a human nucleic acid molecule.

40. A set of three isolated nucleic acid molecules wherein:
(a) each nucleic acid molecule consists essentially of a nucleic acid sequence encoding a portion of gene product described in Table 2a or Table 2b and (i) the first nucleic acid molecule is a forward primer 15 to 30 base pairs in length; (ii) the second nucleic acid molecule is a reverse primer 15 to 30 base pairs in length; and (iii) the third nucleic acid molecule is a probe 15-30 base pairs in length; such that the forward primer and reverse primer produce an amplicon detectable by the probe wherein the amplicon bridges two exons and is 60 to 100 base pairs in length;
(b) each nucleic acid molecule selectively hybridizes to one of the three nucleic acid molecules of (a); or
(c) each nucleic acid molecule has at least 95% sequence identity to the one of the three nucleic acid molecules of (a).

41. The set of nucleic acid molecules of claim 40 wherein the amplicon bridges at least two exons.

42. A method for determining the presence of a gene product of Table 2a or Table 2b in a sample, comprising the steps of:
(a) contacting the sample with a nucleic acid molecule of Table 7 under conditions in which the nucleic acid molecule will selectively hybridize to a gene product of Table 2a or Table 2b; and
(b) detecting hybridization of the nucleic acid molecule to a gene product of Table 2a or Table 2b in the sample, wherein the detection of the hybridization indicates the presence of a gene product of Table 2a or Table 2b in the sample.

43. The method of claim 2 wherein the presence of a plurality of gene products of Table 2a or Table 2b are detected in a sample.

44. A method for determining the presence of a cancer specific protein in a sample, comprising the steps of:
(a) contacting the sample with a suitable reagent under conditions in which the reagent will selectively interact with the cancer specific protein comprising an amino acid sequence with at least 95% sequence identity to the polypeptide encoded by a gene product in Table 2a or Table 2b; and
(b) detecting the interaction of the reagent with a cancer specific protein in the sample, wherein the detection of binding indicates the presence of a cancer specific protein in the sample.

45. The method of claim 44 wherein the presence of a plurality of gene products of Table 2a or Table 2b are detected in a sample.

46. A method for diagnosing or monitoring the presence and metastases of breast cancer in an individual, comprising the steps of:
(a) determining an amount of:
(i) a nucleic acid molecule consisting essentially of a nucleic acid sequence that encodes an amino acid sequence of a gene product in Table 2a or Table 2b;
(ii) a nucleic acid molecule consisting essentially of a nucleic acid sequence of a gene product in Table 2a or Table 2b;
(iii) a nucleic acid molecule consisting essentially of a nucleic acid sequence of Table 7;
(iv) a nucleic acid molecule that selectively hybridizes to the nucleic acid molecule of (i), (ii) or (iii);
(v) a nucleic acid molecule having at least 95% sequence identity to the nucleic acid molecule of (i), (ii) or (iii);
(vi) a polypeptide comprising an amino acid sequence with at least 95% sequence identity to the polypeptide encoded by a gene product in Table 2a or Table 2b; or
(vii) a polypeptide comprising an amino acid sequence encoded by a nucleic acid molecule having at least 95% sequence identity to a nucleic acid molecule consisting essentially of a nucleic acid sequence of a gene product of Table 2a or Table 2b;
in a sample and;
(b) comparing the amount of the determined nucleic acid molecule or the polypeptide in the sample of the individual to the amount of the cancer specific marker in a normal control; wherein a difference in the amount of the nucleic acid molecule or the polypeptide in the sample compared to the amount of the nucleic acid molecule or the polypeptide in the normal control is associated with the presence of breast cancer.

47. The method of claim 46 wherein a plurality of:
(a) a nucleic acid molecule consisting essentially of a nucleic acid sequence that encodes an amino acid sequence of a gene product in Table 2a or Table 2b;
(b) a nucleic acid molecule consisting essentially of a nucleic acid sequence of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (i), (ii) or (iii);
(e) a nucleic acid molecule having at least 95% sequence identity to the nucleic acid molecule of (i), (ii) or (iii);
(f) a polypeptide comprising an amino acid sequence with at least 95% sequence identity to the polypeptide encoded by a gene product in Table 2a or Table 2b; or
(g) a polypeptide comprising an amino acid sequence encoded by a nucleic acid molecule having at least 95% sequence identity to a nucleic acid molecule consisting essentially of a nucleic acid sequence of a gene product of Table 2a or Table 2b;
is determined in a sample.

48. A kit for detecting a risk of cancer or presence of cancer in an individual, said kit comprising a means for determining the presence of:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequences of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c); or
(e) a nucleic acid molecule having at least 95% sequence identity to the nucleic acid molecule of (a), (b) or (c); or
(f) a polypeptide comprising an amino acid sequence with at least 95% sequence identity to the polypeptide encoded by a gene product in Table 2a or Table 2b; or
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a or Table 2b;
in a sample.

49. The kit of claim 48 wherein said kit comprises the means for detecting the presence of a plurality of:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequences of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c); or
(e) a nucleic acid molecule having at least 95% sequence identity to the nucleic acid molecule of (a), (b) or (c); or
(f) a polypeptide comprising an amino acid sequence with at least 95% sequence identity to the polypeptide encoded by a gene product in Table 2a or Table 2b; or
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a or Table 2b;
in a sample.

50. A method of treating an individual with breast cancer, comprising the step of administering a composition comprising:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequence of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c); or
(e) a nucleic acid molecule having at least 95% sequence identity to the nucleic acid molecule of (a), (b) or (c); or
(f) a polypeptide comprising an amino acid sequence with at least 95% sequence identity to the polypeptide encoded by a gene product in Table 2a or Table 2b;
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a or Table 2b; or
(h) an appropriate agonist or antagonist for a gene product of Table 2a or Table 2b;
to an individual in need thereof, wherein said administration induces a therapeutic response against the breast cancer cell expressing the nucleic acid molecule or polypeptide.

51. The method of claim 50 wherein the composition comprises a plurality of:
(a) a nucleic acid molecule consisting essentially of a nucleic acid sequence that encodes an amino acid sequence of the polypeptide encoded by a gene product in Table 2a or Table 2b;
(b) a nucleic acid molecule consisting essentially of a nucleic acid sequence of a gene product in Table 2a or Table 2b;
(c) a nucleic acid molecule consisting essentially of a nucleic acid sequence of Table 7;
(d) a nucleic acid molecule that selectively hybridizes to the nucleic acid molecule of (a), (b) or (c); or
(e) a nucleic acid molecule having at least 95% sequence identity to the nucleic acid molecule of (a), (b) or (c); or
(f) a polypeptide comprising an amino acid sequence with at least 95% sequence identity to the polypeptide encoded by a gene product in Table 2a or Table 2b;
(g) a polypeptide comprising an amino acid sequence encoded by a nucleic acid molecule having at least 95% sequence identity to a nucleic acid molecule comprising a nucleic acid sequence of a nucleic acid molecule consisting essentially of a nucleic acid sequence of a gene product of Table 2a or Table 2b; or
(h) an appropriate agonist or antagonist for a gene product of Table 2a or Table 2b;
